(19) United States
(12) Patent Application Publication
(10) Pub. No.: US 2022/0402901 A1
(43) Pub. Date: Dec. 22, 2022

(54) SUBSTITUTED N-HETEROCYCLIC CARBOXAMIDES AS ACID CERAMIDASE INHIBITORS AND THEIR USE AS MEDICAMENTS

(71) Applicants: Bial - R&D Investments, S.A., Coronado (S.Romão e. S.Mamede) (PT); Fondazione Istituto Italiano di Tecnologia, Genova (IT)

(72) Inventors: Renato T. Skerlj, West Newton, MA (US); Rita Scarpelli, Genova (IT); Vincenzo Cilibrasi, Genova (IT); Samantha Caputo, Genova (IT)

(21) Appl. No.: 17/760,873

(22) PCT Filed: Sep. 17, 2020
(86) PCT No.: PCT/US2020/051303

§ 371 Date: Mar. 16, 2022

Related U.S. Application Data
(60) Provisional application No. 62/901,384, filed on Sep. 17, 2019.

(51) Int. Cl. C07D 413/04; C07D 451/02; C07D 491/107; C07D 487/04; C07D 471/10

(52) U.S. Cl. CPC C07D 413/04; C07D 451/02; C07D 491/107; C07D 487/04; C07D 471/10

(57) ABSTRACT

The invention provides substituted N-heterocyclic carboxamides and related compounds, compositions containing such compounds, medical kits, and methods for using such compounds and compositions to treat a medical disorder, e.g., cancer, lysosomal storage disorder, neurodegenerative disorder, inflammatory disorder, in a patient.

### CROSS-REFERENCE TO RELATED APPLICATIONS

This application claims the benefit of U.S. Provisional Patent Application No. 62/901,384 filed on Sep. 17, 2019, the entire contents of which are incorporated by reference herein.

### FIELD OF THE INVENTION

The invention provides substituted N-heterocyclic carboxamides and related compounds, compositions containing such compounds, medical kits, and methods for using such compounds and compositions to treat medical disorders in a patient.

### BACKGROUND

Sphingolipids, in addition to serving roles in cell membrane structure and dynamics, also serve important signaling functions, for example, in the control of cell growth, cell differentiation, and cell death, and so are important for cell homeostasis and development. Zeidan el al. (2010) CURR. MOL. MED. 10, 454, Proksch et al. (2011) J. LIPIDS Article ID 971618. Ceramide, a key member of this lipid class, has attracted attention in view of its impact on the replication and differentiation of neoplastic cells. Furuya et al. (2011) CANCER METASTASIS REV. 30, 567. For example, lower levels of ceramide have been discovered in several types of human tumors relative to normal tissue, where the level of ceramide appears to correlate inversely with the degree of malignant progression. Realini et al. (2013) J. MOL. BIOL. 56, 3518.

Acid ceramidase (AC, also known as N-acylsphingosine amidohydrolase-1, or ASAH-1) is a cysteine amidase that catalyzes the hydrolysis of ceramide into sphingosine and fatty acid. Acid ceramidase is believed to be involved in the regulation of ceramide levels in cells and modulates the ability of this lipid messenger to influence the survival, growth and death of certain tumor cells. Doan et al. (2017) ONCOTARGET 8(68), 112662-74. Furthermore, acid ceramidase enzymes are abnormally expressed in various types of human cancer (e.g., prostate, head and neck, and colon) and serum AC levels are elevated in patients with melanoma relative to control subjects. Realini et al. (2015) J. BIOL. CHEM. 291 (5), 2422-34.

In addition, acid ceramidase enzymes have been implicated in a number of other disorders, including, inflammation (for example, rheumatoid arthritis and psoriasis), pain, inflammatory pain, and various pulmonary disorders. See, International Application Publication No. WO2015/173169. Furthermore, acid ceramidase enzymes have been identified as a target for the treatment of certain lysosomal storage disorders (for example, Gaucher's, Fabry's, Krabbe, Tay Sachs), and neurodegenerative disorders (for example, Alzheimers, Parkinson's, Huntington's, and amytrophic lateral sclerosis). See, International Application Publication Nos. WO2016/210116 and WO2016/210120.

Despite the efforts to develop acid ceramidase inhibitors for use in the treatment of various disorders there is still a need for new acid ceramidase inhibitors.

### SUMMARY

The invention provides substituted N-heterocyclic carboxamides and related compounds, compositions containing such compounds, medical kits, and methods for using such compounds and compositions to treat medical disorders, for example, cancer (such as melanoma), a lysosomal storage disorder (such as Krabbe disease, Fabry disease, Tay-Sachs disease, Pompe disease, Hunter's syndrome, Niemnann Pick disease Types A and B, Gaucher disease), a neurodegenerative disease (such as Alzheimer's disease, Parkinson's disease, Huntington's disease, amyotrophic lateral sclerosis, and Lewy body disease), an inflammatory disorder, and pain. Various aspects and embodiments of the invention are described in further detail below.

In one aspect, provided herein is a compound of formula (I):

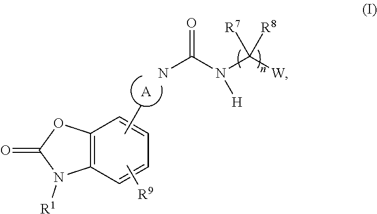

or a pharmaceutically acceptable salt thereof, wherein:

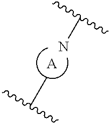

is a monocyclic or bicyclic (e.g., fused, spiro, bridged) heterocyclylene containing at least one N (including the depicted nitrogen) that is optionally substituted (e.g., with one or more substituents each independently selected from C₁₋₆alkyl and oxo); R is selected from the group consisting of hydrogen, C₁₋₆alkyl, C₁₋₆alkylene-NR^{a}₂, C₁₋₆alkylene-OR^{c}, 3-7 membered heterocyclyl, phenyl, C₃₋₇cycloalkyl, and 5-6 membered heteroaryl; R⁷ and R⁸ are independently, for each occurrence, selected from the group consisting of hydrogen, C₁₋₆alkyl, C₁₋₆haloalkyl, and halogen; or R⁷ and R⁸ can be taken together to form C₃₋₇cycloalkylene; R⁹ is selected from the group consisting of hydrogen, C₁₋₆alkyl, and halogen; R^{a} is hydrogen or C₁₋₆alkyl; R^{c} is selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂; and n is an integer selected from 0 to 6, wherein when n is an integer selected from 1 to 6, W is selected from the group consisting of hydrogen, halogen, phenyl, 5-6 membered heteroaryl, C₃₋₇cycloalkyl, 3-7 membered heterocyclyl, O—(C₁₋₆alkyl), O—(C₁₋₆haloalkyl), —O-phenyl, and O(C₁₋₆alkylene)-phenyl; and when n is 0, W is selected from the group consisting of hydrogen, C₃₋₇cycloalkyl, 3-7 membered saturated heterocyclyl, O—(C₁₋₆alkyl), O—(C₁₋₆haloalkyl), —O-phenyl, and O—(C₁₋₆alkylene)-phenyl;
wherein any aforementioned 3-7 membered heterocyclyl and phenyl are optionally substituted, and wherein the compound is not a compound selected from the group consisting of:

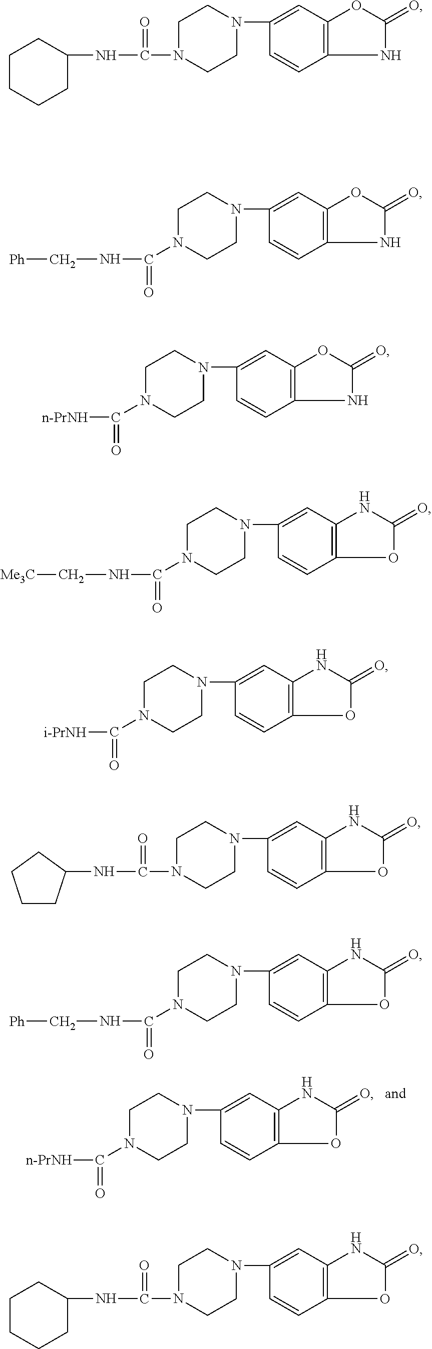

or a pharmaceutically acceptable salt thereof.

In some embodiments, the compound is a compound of formula (I-a):

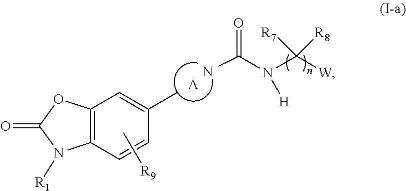

or a pharmaceutically acceptable salt thereof, wherein the variables are as defined herein.

In some embodiments, the compound is a compound of formula (I-b):

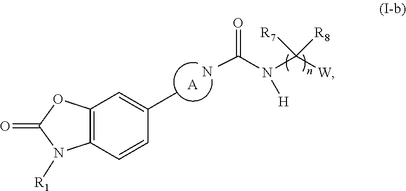

or a pharmaceutically acceptable salt thereof, wherein the variables are as defined herein.

In some embodiments, the compound is a compound of formula (I-c):

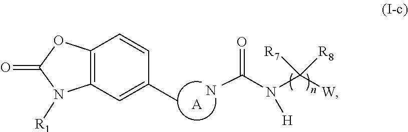

or a pharmaceutically acceptable salt thereof, wherein the variables are as defined herein.

In some embodiments, the compound is a compound of (I-d):

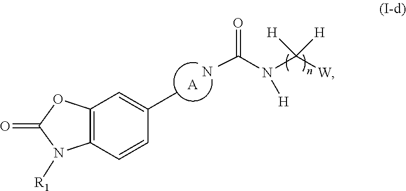

or a pharmaceutically acceptable salt thereof, wherein the variables are as defined herein.

In some embodiments, the compound is a compound of formula (II):

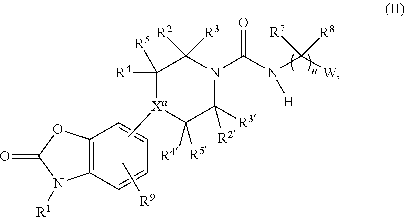

or a pharmaceutically acceptable salt thereof, wherein the variables are as defined herein.

In some embodiments, the compound is a compound of formula (II-a):

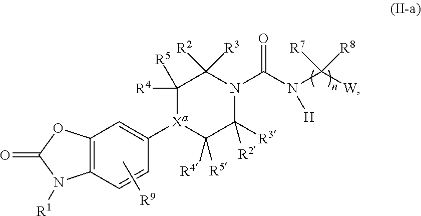

or a pharmaceutically acceptable salt thereof, wherein the variables are as defined herein.

In some embodiments, the compound is a compound of formula (III):

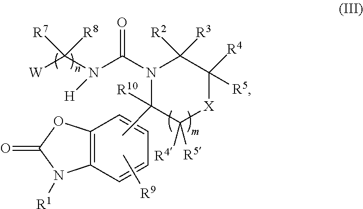

or a pharmaceutically acceptable salt thereof, wherein the variables are as defined herein.

In some embodiments, the compound is a compound of formula (III-a):

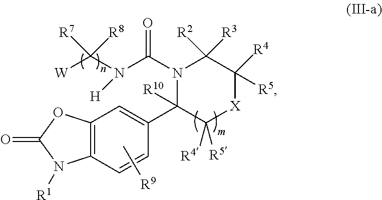

or a pharmaceutically acceptable salt thereof, wherein the variables are as defined herein.

In some embodiments, the compound is a compound of formula (IV):

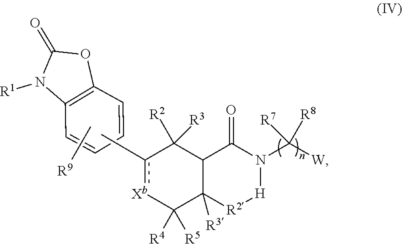

or a pharmaceutically acceptable salt thereof, wherein the variables are as defined herein.

In some embodiments, the compound is a compound of formula (IV-a):

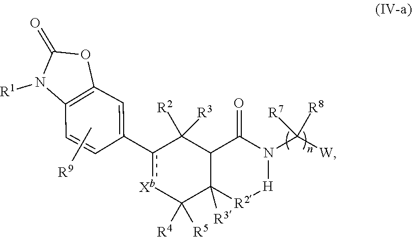

or a pharmaceutically acceptable salt thereof wherein the variables are as defined herein.

In some embodiments, the compound is a compound of formula (IV-b):

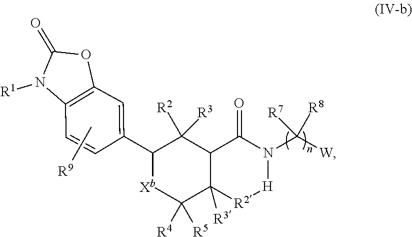

or a pharmaceutically acceptable salt thereof, wherein the variables are as defined herein.

In another aspect, provided herein is a pharmaceutical composition comprising a compound disclosed herein (e.g., a compound of Formula (I), e.g., a compound of Formula (I-a), (I-b), (I-c), (I-d), (II), (II-a), (III), (III-a), (IV), (IV-a), or (IV-b) and a pharmaceutically acceptable carrier.

In another aspect, the invention provides a method of treating a subject with cancer and in need thereof, the method comprising administering to the subject a therapeutically effective amount of a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition disclosed herein.

In another aspect, the invention provides a method of treating a subject with a lysosomal storage disorder and in need thereof, the method comprising administering to the subject a therapeutically effective amount of a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition disclosed herein.

In another aspect, the invention provides a method of treating a subject with a neurodegenerative disorder and in need thereof, the method comprising administering to the subject a therapeutically effective amount of a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition disclosed herein.

In another aspect, the invention provides a method of treating a subject with an inflammatory disorder and in need thereof, the method comprising administering to the subject a therapeutically effective amount of a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition disclosed herein.

In another aspect, the invention provides a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition as disclosed herein for use in a method of treating a subject with cancer and in need thereof, the method comprising administering to the subject a therapeutically effective amount of the compound or the pharmaceutical composition.

In another aspect, the invention provides a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition as disclosed herein for use in a method of treating a subject with a lysosomal storage disorder and in need thereof, the method comprising administering to the subject a therapeutically effective amount of the compound or the pharmaceutical composition.

In another aspect, the invention provides a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition as disclosed herein for use in a method of treating a subject with a neurodegenerative disorder and in need thereof, the method comprising administering to the subject a therapeutically effective amount of the compound or the pharmaceutical composition.

In another aspect, the invention provides a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition as disclosed herein for use in a method of treating a subject with an inflammatory disorder and in need thereof, the method comprising administering to the subject a therapeutically effective amount of the compound or the pharmaceutical composition.

In another aspect, the invention provides use of a compound (e.g., a compound of Formula (f), e.g., a compound of Formula (I-a), (I-b), (I-c), (I-d), (II), (I-a), (III), (III-a), (IV), (IV-a), or (I V-b) or a pharmaceutical composition as disclosed herein for the manufacture of a medicament for treating a subject with cancer and in need thereof, the method comprising administering to the subject a therapeutically effective amount of the compound or the pharmaceutical composition.

In another aspect, the invention provides use of a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition as disclosed herein for the manufacture of a medicament for treating a subject with a lysosomal storage disorder and in need thereof, the method comprising administering to the subject a therapeutically effective amount of the compound or the pharmaceutical composition.

In another aspect, the invention provides use of a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition as disclosed herein for the manufacture of a medicament for treating a subject with a neurodegenerative disorder and in need thereof, the method comprising administering to the subject a therapeutically effective amount of the compound or the pharmaceutical composition.

In another aspect, the invention provides use of a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition as disclosed herein for the manufacture of a medicament for treating a subject with an inflammatory disorder and in need thereof, the method comprising administering to the subject a therapeutically effective amount of the compound or the pharmaceutical composition.

### DETAILED DESCRIPTION

The invention provides substituted N-heterocyclic carboxamides and related compounds, compositions containing such compounds, medical kits, and methods for using such compounds and compositions to treat medical disorders in a patient. The practice of the present invention employs, unless otherwise indicated, conventional techniques of organic chemistry, pharmacology, cell biology, and biochemistry. Such techniques are explained in the literature, such as in “Comprehensive Organic Synthesis” (BM. Trost & I. Fleming, eds., 1991-1992); “Current protocols in molecular biology” (F. M. Ausubel et al., eds., 1987, and periodic updates); and “Current protocols in immunology” (J. E. Coligan et al, eds., 1991), each of which is herein incorporated by reference in its entirety. Various aspects of the invention are set forth below in sections; however, aspects of the invention described in one particular section are not to be limited to any particular section.

### I. Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein should be construed according to the standard rules of chemical valency known in the chemical arts.

The terms “a” and “an” as used herein mean “one or more” and include the plural unless the context is inappropriate.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like.

The term “hydroxyalkyl” refers to an alkyl group that is substituted with at least one hydroxyl group. For example, exemplary hydroxyalkyl groups include —CH₂OH, —C(H)(OH)CH₃, and the like. In certain embodiments, the hydroxyalkyl is an alkyl group that is substituted with just one hydroxyl group.

The term “cyanoalkyl” refers to an alkyl group that is substituted with one cyano group.

The term “heteroalkyl” as used herein refers to an “alkyl” group in which at least one carbon atom has been replaced with a heteroatom (e.g., an O, N, or S atom). The heteroalkyl may be, for example, an —O—C₁-C₁₀alkyl group, an —C₁-C₆alkylene-O—C₁-C₆alkyl group, or a C₁-C₆ alkylene-OH group. In certain embodiments, the “heteroalkyl” may be 2-8 membered heteroalkyl, indicating that the heteroalkyl contains from 2 to 8 atoms selected from the group consisting of carbon, oxygen, nitrogen, and sulfur. In yet other embodiments, the heteroalkyl may be a 2-6 membered, 4-8 membered, or a 5-8 membered heteroalkyl group (which may contain, for example, 1 or 2 heteroatoms selected from the group oxygen and nitrogen). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂alkenyl, C₂-C₁₀alkenyl, and C₂-C₆alkenyl, respectively. Exemplary alkenyl groups include vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, and the like.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂alkynyl, C₂-C₁₀alkynyl, and C₂-C₆alkynyl, respectively. Exemplary alkynyl groups include ethynyl, prop-1-yn-1-yl, and but-1-yn-1-yl.

The term “cycloalkyl” refers to a monovalent saturated cyclic, bicyclic, bridged cyclic (e.g., adamantyl), or spirocyclic hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of an cycloalkyl group. An exemplary cycloalkylene group is

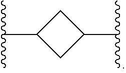

The term “cycloalkenyl” as used herein refers to a monovalent unsaturated cyclic, bicyclic, or bridged cyclic (e.g., adamantyl) hydrocarbon group of 3-12, 3-8, 4-8, or 4-6 carbons containing one carbon-carbon double bond, referred to herein, e.g., as “C₄₋₈cycloalkenyl,” derived from a cycloalkane. Exemplary cycloalkenyl groups include, but are not limited to, cyclohexenes, cyclopentenes, and cyclobutenes. Unless specified otherwise, cycloalkenyl groups are optionally substituted at one or more ring positions with, for example, alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkenyl group is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The term “aralkyl” refers to an alkyl group substituted with an aryl group.

The term “bicyclic carbocyclyl that is partially unsaturated” refers to a bicyclic carbocyclic group containing at least one double bond between ring atoms and at least one ring in the bicyclic carbocyclic group is not aromatic. Representative examples of a bicyclic carbocyclyl that is partially unsaturated include, for example:

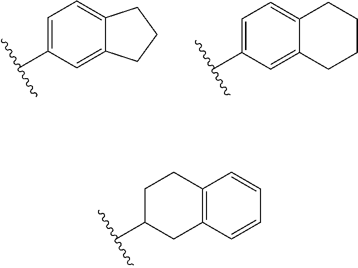 [SMILES: *c1ccc2c(c1)CCC2 ; *c1ccc2c(c1)CCCC2 ; *C1CCc2ccccc2C1]

The terms ortho, meta and para are art-recognized and refer to 1,2-, 1,3- and 1,4-disubstituted benzenes, respectively. For example, the names 1,2-dimethylbenzene and ortho-trimethylbenzene are synonymous.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may be, for example, mono-, bi-, or other multi-cyclic ring systems. A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isooxazolidinyl, rnorpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, oxo, phosphate, phosphonato, phosphinato, sulfate, sulfide, sulfonamido, sulfonyl and thiocarbonyl. In certain embodiments, the heterocyclyl group is not substituted, i.e., it is unsubstituted.

The term “bicyclic heterocyclyl” refers to a fused, bridged, or spirocyclic heterocyclyl group that contains two rings. Representative examples of a bicyclic heterocyclyl include, for example:

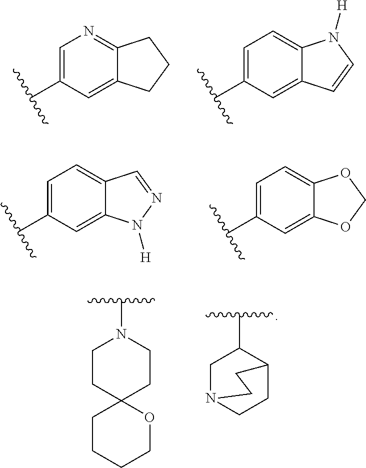 [SMILES: *c1cnc2c(c1)CCC2 ; *c1ccc2[nH]ccc2c1 ; *c1ccc2cn[nH]c2c1 ; *c1ccc2c(c1)OCO2 ; *N1CCC2(CCCCO2)CC1 ; *C1CN2CCC1CC2]

In certain embodiments, the bicyclic heterocyclyl is an carbocyclic ring fused to partially unsaturated heterocyclic ring, that together form a bicyclic ring structure having 8-10 ring atoms (e.g., where there are 1, 2, 3, or 4 heteroatoms selected from the group consisting of nitrogen, oxygen, and sulfur).

The term “hetero heterocyclylene” refers to a diradical of a heterocycloalkyl group. An exemplary hetero heterocyclylene group is

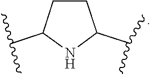

The hetero heterocyclylene may contain, for example, 3-6 ring atom (i.e., a 3-6 membered hetero heterocyclylene). In certain embodiments, the hetero heterocyclylene is a 3-6 membered heterocycloalkylene containing 1, 2, or 3 three heteroatoms selected from the group consisting of oxygen, nitrogen, and sulfur.

The term “bicyclic heterocyclylene” refers to a diradical of a bicyclic heterocyclyl group.

The term “heteroaryl” is art-recognized and refers to aromatic groups that include at least one ring heteroatom. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted. In certain embodiments, the heteroaryl group is a 5- to 10-membered ring structure, alternatively a 5- to 6-membered ring structure, whose ring structure includes 1, 2, 3, or 4 heteroatoms, such as nitrogen, oxygen, and sulfur.

The terms “amine” and “amino” are art-recognized and refer to both unsubstituted and substituted amines, e.g., a moiety represented by the general formula —N(R⁵⁰)(R⁵¹), wherein R⁵⁰ and R⁵¹ each independently represent hydrogen, alkyl, cycloalkyl, heterocyclyl, alkenyl, aryl, aralkyl, or —(CH₂)ₘ—R⁶¹; or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, R⁵⁰ and R⁵¹ each independently represent hydrogen, alkyl, alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R₆₁, where in and R₆₁ are described above. The term “haloalkoxyl” refers to an alkoxyl group that is substituted with at least one halogen. For example, —O—CH₂F, —O—CHF₂, —O—CF₃, and the like. In certain embodiments, the haloalkoxyl is an alkoxyl group that is substituted with at least one fluoro group. In certain embodiments, the haloalkoxyl is an alkoxyl group that is substituted with from 1-6, 1-5, 1-4, 2-4, or 3 fluoro groups.

Any aryl (e.g., phenyl), cycloalkyl (e.g., C₃₋₇cycloalkyl), heterocyclyl (e.g., 3-7 membered heterocyclyl), heteroaryl (e.g., 5-6 membered heteroaryl) may be optionally substituted unless otherwise states. In some embodiments, Any aryl (e.g., phenyl), cycloalkyl (e.g., C₃₋₇cycloalkyl), heterocyclyl (e.g., 3-7 membered heterocyclyl), heteroaryl (e.g., 5-6 membered heteroaryl) may be optionally substituted with 1-4 substituents independently for each occurrence selected from the group consisting of halogen, C₁₋₆alkyl, C₁₋₆haloalkyl, C₁₋₆alkoxy, cyano, N(R^{aa})₂, —CH₂N(R^{aa})₂, and hydroxyl, wherein R^{aa} is independently for each occurrence hydrogen or C₁₋₆alkyl.

The term “carbamate” as used herein refers to a radical of the form —R_{g}OC(O)N(Rₕ)—, —R_{g}OC(O)N(Rₕ)Rᵢ—, or —OC(O)NRₕRᵢ, wherein R_{g}, Rₕ and Rᵢ are each independently alkoxy, aryloxy, alkyl, alkenyl, alkynyl, amide, amino, aryl, arylalkyl, carboxy, cyano, cycloalkyl, ester, ether, formyl, halogen, haloalkyl, heteroaryl, heterocyclyl, hydroxyl, ketone, nitro, sulfide, sulfonyl, or sulfonamide. Exemplary carbamates include arylcarbamates and heteroaryl carbamates, e.g., wherein at least one of R_{g}, Rₕ and Rᵢ are independently aryl or heteroaryl, such as phenyl and pyridinyl.

The term “carbonyl” as used herein refers to the radical —C(O)—.

The term “carboxamido” as used herein refers to the radical —C(O)NRR′, where R and R′ may be the same or different. R and R′ may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” as used herein refers to a radical of the form —RₐC(O)N(R_{b})—, —RₐC(O)N(R_{b})R_{c}—, —C(O)NR_{b}R_{c}, or —C(O)NH₂, wherein Rₐ, R_{b} and R_{c} are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro. The amide can be attached to another group through the carbon, the nitrogen, R_{b}, R_{c}, or Rₐ. The amide also may be cyclic, for example R_{b} and R_{c}, Rₐ and R_{b}, or Rₐ and R_{c} may be joined to form a 3- to 12-membered ring, such as a 3- to 10-membered ring or a 5- to 6-membered ring.

The term “amidino” as used herein refers to a radical of the form —C(═NR)NR′R″ where R, R′, and R″ are each independently alkyl, alkenyl, alkynyl, amide, aryl, arylalkyl, cyano, cycloalkyl, haloalkyl, heteroaryl, heterocyclyl, hydroxyl, ketone, or nitro.

The term “alkanoyl” as used herein refers to a radical —O—CO-alkyl.

The term “oxo” is art-recognized and refers to a “═O” substituent. For example, a cyclo pentane substituted with an oxo group is cyclo pentanone.

The term “sulfonamide” or “sulfonamido” as used herein refers to a radical having the structure —N(Rᵣ)—S(O)₂—Rₛ— or —S(O)₂—N(Rᵣ)Rₛ, where Rᵣ, and Rₛ can be, for example, hydrogen, alkyl, aryl, cycloalkyl, and heterocyclyl. Exemplary sulfonamides include alkylsulfonamides (e.g., where Rₛ is alkyl), arylsulfonamides (e.g., where Rₛ is aryl), cycloalkyl sulfonamides (e.g., where Rₛ is cycloalkyl), and heterocyclyl sulfonamides (e.g., where Rₛ is heterocyclyl), etc.

The term “sulfonyl” as used herein refers to a radical having the structure RᵤSO₂—, where Rᵤ can be alkyl, aryl, cycloalkyl, and heterocyclyl, e.g., alkylsulfonyl. The term “alkylsulfonyl” as used herein refers to an alkyl group attached to a sulfonyl group.

In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. In some embodiments, an optional substituent may be selected from the group consisting of: C₁₋₆alkyl, cyano, halogen, —O—C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(Rₐ)₂, wherein R^{a} is hydrogen or C₁₋₆alkyl. In some embodiments, an optional substituent may be selected independently for each occurrence from the group consisting of CH₂N(R^{a})₂, cyano, C₁₋₆alkyl, halogen, and —O—C₁₋₆alkyl, wherein R″ is hydrogen or C₁₋₆alkyl. In some embodiments, an optional substituent may be selected independently for each occurrence from the group consisting of C₁₋₆alkyl, halogen, —O—C₁₋₆alkyl, and —C₁₂N(R². In some embodiments, an optional substituent may be selected independently for each occurrence from the group consisting of C₁₋₆alkyl, halogen, and O—(C₁₋₆alkyl).

The symbol “ [symbol] ” indicates a point of attachment.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “s,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol [symbol] denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵B, ¹⁸O, ¹⁷O, ³¹P, ³²P, ³⁵S, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in, e.g., the Examples herein by substituting an isotopically labeled reagent for a non-isotopically labeled reagent.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Abbreviations as used herein include O-(7-azabenzotriazol-1-yl)-N,N,N′,N′-tetramethyluronium hexafluorophosphate (HATU); diisopropylethylamine (DIPEA); dimethylformamide (DMF); methylene chloride (DCM); tert-butoxycarbonyl (Boc); tetrahydrofuran (THF); trifluoroacetic acid (TFA); triethylamine (TEA), Boc anhydride ((Boc)₂O); dimethylsulfoxide (DMSO); diisopropylethyl amine (DIEA); methyl tert-butyl ether (MTBE); 1,2-dichloroethene (DEC); 4-dimethylaminopyridine (DMAP); bis(trimethylsilyl)amine (HMDS); 1,2-dimethylethylenediamine (DMEDA); carbonyldiimidazole (CDI); pentane (PE); flash column chromatography (FCC); supercritical fluid chromatography (SFC); acetonitrile (ACN); acetic acid (AcOH); ammonium acetate (NH₄OAc); ethylene bridged hybrid (BEH); broadband inverse (BBI); cyclohexane (Cy); dichloroethane (DCE); dimethylamine (NHMe₂); dimethylcyclohexanedicarboxylate (DMCD); ethanol (EtOH); ethylene acetate (EA); in situ chemical oxidation (ISCO); methanol (MeOH); methylmagnesium bromide (MeMgBr); mass spectrometry, electrospray (MS (ES)); methyl tert-butyl ether (MTBE); methyl iodide (MeI); nuclear magnetic resonance spectroscopy (NMR); [1,1′-bis(diphenylphosphino)ferrocene]dichloropalladium(II), complex with di chloromethane (PdCl₂(dppf)-DCM); photodiode array (PDA); potassium acetate (KOAc); p-toluenesulfonic acid (p-TsOH); room temperature (RT); sodium acetate (NaOAc); sodium triacetoxyborohydride (NaBH(AcO)₃); solid phase extraction (SPE); thin layer chromatography (TLC); triethylamine (Et₃N); and ultra performance liquid chromatography/mass spectrometry (UPLC/MS).

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also include the specific quantitative value itself, unless specifically stated otherwise. As used herein, the term “about” refers to a +10% variation from the nominal value unless otherwise indicated or inferred.

It should be understood that the order of steps or order for performing certain actions is immaterial so long as the present invention remain operable. Moreover, two or more steps or actions may be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20. Additional examples include that the phrase “optionally substituted with 1-5 substituents” is specifically intended to individually disclose a chemical group that can include 0, 1, 2, 3, 4, 5, 0-5, 0-4, 0-3, 0-2, 0-1, 1-5, 1-4, 1-3, 1-2, 2-5, 2-4, 2-3, 3-5, 3-4, and 4-5 substituents.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Substituted N-Heterocyclic Carboxamides and Related Compounds

It has been discovered that the active site (binding site) of human acid ceramidase (ASAH-1), as determined by x-ray crystallography, contains a plurality of hydration sites, each of which is occupied by a single molecule of water and whose position and energetics (which incorporates the enthalpy, entropy, and free energy values associated with each water molecule) have been calculated. Each of these water molecules has a stability rating (namely, a numerical calculation which incorporates the enthalpy, entropy, and free energy values associated with each water molecule), which provides a measurable value associated with the relative stability of water molecules that occupy hydration sites in the binding pocket of the acid ceramidase enzyme. Water molecules occupying hydration sites in the binding pocket of acid ceramidase having a stability rating of >2.5 kcal/mol are referred to as unstable waters. It is contemplated that the displacement or disruption of an unstable water molecule (i.e., a water molecule having a stability rating of greater than 2.5 kcal/mol), or replacement of a stable water molecule (i.e., a water molecule having a stability rating of less than 1 kcal/mol), by an inhibitor results in tighter binding of that inhibitor. Accordingly, it is contemplated that an inhibitor designed to displace one or more unstable water molecules (namely, a water molecule having a stability rating of greater than 2.5 kcal/mol) may bind more tightly to the binding pocket and, therefore, will be a more potent inhibitor as compared to an inhibitor that does not displace unstable water molecules. Certain of the compounds described herein were designed to displace one or more unstable water molecules in the binding pocket.

### Compounds

One aspect of the invention provides substituted N-heterocyclic carboxamides and related compounds. The substituted N-heterocyclic carboxamides and related compounds are contemplated to be useful in the methods, compositions, and kits described herein. In certain embodiments, the substituted N-heterocyclic carboxamides or related compound is a compound embraced by Formula I:

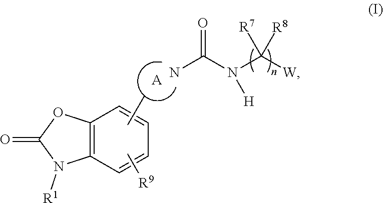

or a pharmaceutically acceptable salt thereof, wherein:

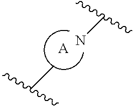

is a monocyclic or bicyclic (e.g., fused, spiro, bridged) heterocyclylene containing at least one N (including the depicted nitrogen) that is optionally substituted (e.g., with one or more substituents each independently selected from C₁₋₆alkyl and oxo);

R¹ is selected from the group consisting of hydrogen, C₁₋₆alkyl, C₁₋₆alkylene-NR^{a}₂, C₁₋₆alkylene-OR^{c}, 3-7 membered heterocyclyl, phenyl, C₃₋₇cycloalkyl, and 5-6 membered heteroaryl;

R⁷ and R⁸ are independently, for each occurrence, selected from the group consisting of hydrogen, C₁₋₆alkyl, C₁₋₆haloalkyl, and halogen; or R⁷ and R⁸ can be taken together to form C₃₋₇cycloalkylene;

R⁹ is selected from the group consisting of hydrogen, C₁₋₆alkyl, and halogen;

R^{a} is hydrogen or C₁₋₆alkyl;

R^{c} is selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂;

n is an integer selected from 0 to 6, wherein

when n is an integer selected from 1 to 6, W is selected from the group consisting of hydrogen, halogen, phenyl, 5-6 membered heteroaryl, C₃₋₇cycloalkyl, 3-7 membered heterocyclyl, O—(C₁₋₆alkyl), O—(C₁₋₆haloalkyl), —O-phenyl, and O—(C₁₋₆alkylene)-phenyl; and

when n is 0, W is selected from the group consisting of hydrogen, C₃₋₇cycloalkyl, 3-7 membered saturated heterocyclyl, O—(C₁₋₆alkyl), O—(C₁₋₆haloalkyl), —O-phenyl, and O—(C₁₋₆alkylene)-phenyl;

wherein any aforementioned 3-7 membered heterocyclyl and phenyl are optionally substituted,
and wherein the compound is not a compound selected from the group consisting of:

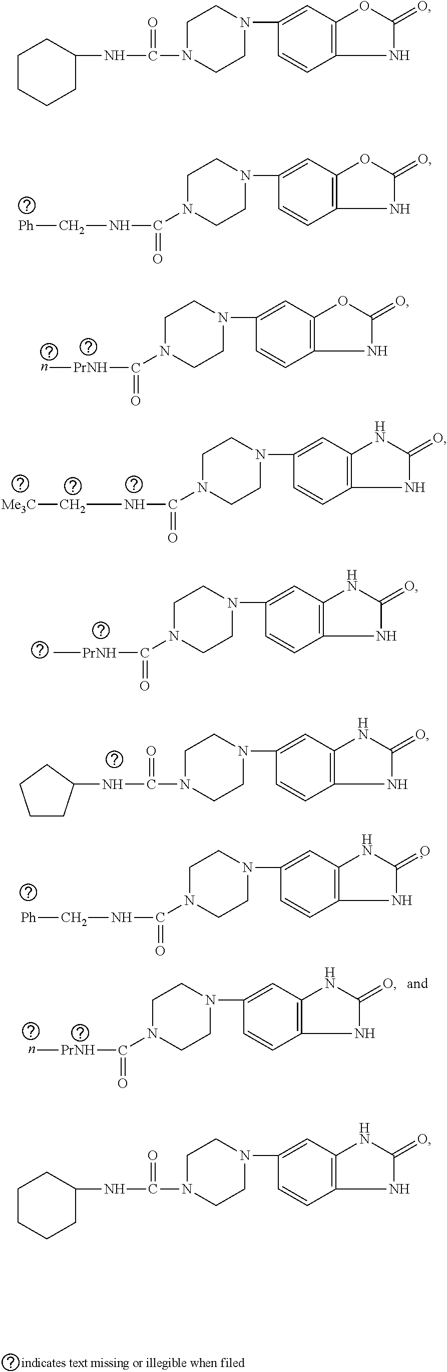

or a pharmaceutically acceptable salt thereof.

In some embodiments, the compound is a compound of formula (I-a):

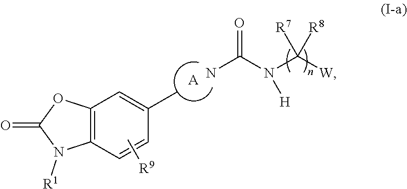

or a pharmaceutically acceptable salt thereof, wherein the variables are as defined, for example, in the compound of formula (I).

In each of the foregoing compounds of formula (I), (I-a), (I-b), (I-c), or (I-d),

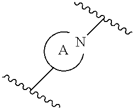

is a monocyclic heterocyclylene.

In each of the foregoing compounds of formula (I), (I-a), (I-b), (I-c), or (I-d),

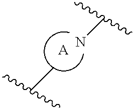

is selected from the group consisting of:

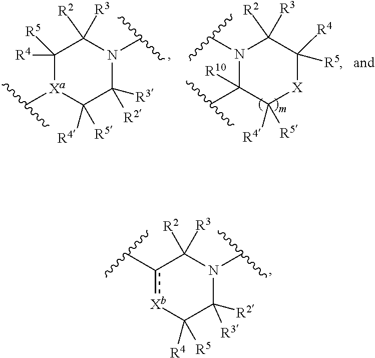

wherein

R², R³, R^{2′} and R^{3′} are independently selected from hydrogen or C₁₋₆alkyl, or

R² and R³ or R^{2′} and R^{3′} can be taken together to form C₃₋₇cycloalkylene, 3-7 membered heterocyclylene, or oxo; or

R^{4′} and R^{5′} are independently selected from hydrogen and C₁₋₆alkyl, or R^{4′} and R^{5′} can be taken together to form oxo;

R^{4′} and R^{5′} are independently selected from hydrogen and C₁₋₆alkyl, or R^{4′} and R^{5′} can be taken together to form oxo;

X is selected from the group consisting of CH₂, NR^{a}, and O;

X^{a} is selected from CH or N;

R¹⁰ is hydrogen or methyl;

R^{a} is hydrogen or C₁₋₆alkyl;

[symbol] indicates a single bond or double bond (e.g., [symbol] indicates a single bond in formula (I-b) or (I-c), and when a single bond, X^{b} is selected from the group consisting of CH₂, NR^{a}, and O, and when a double bond, X^{b} is CH; and

m is or 1.

In some embodiments, the compound is a compound of formula (I-b):

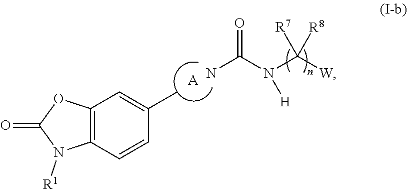

or a pharmaceutically acceptable salt thereof, wherein the variables are as defined herein.

In some embodiments, the compound is a compound of formula (I-c):

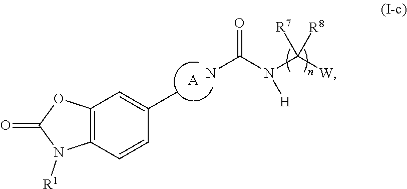

or a pharmaceutically acceptable salt thereof, wherein the variables are as defined herein.

In some embodiments, the compound is a compound of (I-d):

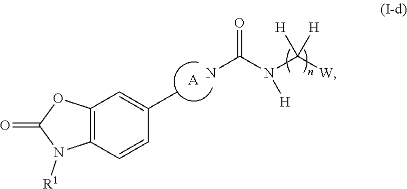

or a pharmaceutically acceptable salt thereof, wherein the variables are as defined herein.

In each of the foregoing compounds of formula (I), (I-a), (I-b), (I-c), or (I-d), X^{a} is CH.

In each of the foregoing compounds of formula (I), (I-a), (I-b), (I-c), or (I-d), X^{b} is CH₂ or CH.

In each of the foregoing compounds of formula (I), (I-a), (I-b), (I-c), or (I-d), X is CH₂.

In each of the foregoing compounds of formula (I), (I-a), (I-b), (I-c), or (I-d), X is O.

In each of the foregoing compounds of formula (I), (I-a), (I-b), (I-c), or (I-d), at least one of R² and R³ is methyl.

In each of the foregoing compounds of formula (I), (I-a), (I-b), (I-c), or (I-d), at least one of R², R³, R^{2′} and R^{3′} is methyl.

In each of the foregoing compounds of formula (I), (I-a), (I-b), (I-c), or (I-d), R² and R³ are methyl.

In each of the foregoing compounds of formula (I), (I-a), (I-b), (I-c), or (I-d), R² and R³ are methyl, and RT and R³ are hydrogen.

In each of the foregoing compounds of formula (I), (I-a), (I-b), (I-c), or (I-d), R² and R³ are independently hydrogen or methyl.

In each of the foregoing compounds of formula (I), (I-a), (I-b), (I-c), or (I-d), R², R³, and R^{2′} and R^{3′} are independently hydrogen or methyl,

In each of the foregoing compounds of formula (I), (I-a), (I-b), (I-c), or (I-d), R² and R³ are taken together to form C₃₋₇cycloalkylene, 3-7 membered heterocyclylene, or oxo.

In each of the foregoing compounds of formula (I), (I-a), (I-b), (I-c), or (I-d), R² and R³ are taken together to form cyclopropylene, 4-membered heterocyclylene, or oxo.

In each of the foregoing compounds of formula (I), (I-a), (I-b), (I-c), or (I-d), R² and R^{2′} are taken together to form a 5-7-membered heterocycle, and R³ and R^{3′} are hydrogen.

In each of the foregoing compounds of formula (I), (I-a), (I-b), (I-c), or (I-d), R⁴ and R⁵ are hydrogen or taken together to form oxo.

In each of the foregoing compounds of formula (I), (I-a), (I-b), (I-c), or (I-d), R⁴, R⁹, R^{4′} and R^{5′} are hydrogen.

In each of the foregoing compounds of formula (I), (I-a), (I-b), (I-c), or (I-d), R² and R³ are methyl, and R^{2′}, R^{3′}, R⁴, R⁵, R^{4′} and R^{5′} are hydrogen.

In each of the foregoing compounds of formula (I), (I-a), (I-b), (I-c), or (I-d), R² and R³ are methyl, and R⁴, R⁵, R^{4′} and R^{5′} are hydrogen.

In each of the foregoing compounds of formula (I), (I-a), (I-b), (I-c), or (I-d), R² and R³ are methyl, R^{2′}, R^{3′}, R⁴, and R⁵ are hydrogen, and R^{4′} and R^{5′} are taken together to form oxo.

In each of the foregoing compounds of formula (I), (I-a), (I-b), (I-c), or (I-d), m is 1.

In each of the foregoing compounds of formula (I), (I-a), (I-b), (I-c), or (I-d), R¹⁰ is hydrogen.

In some embodiments, the compound is a compound of formula (II):

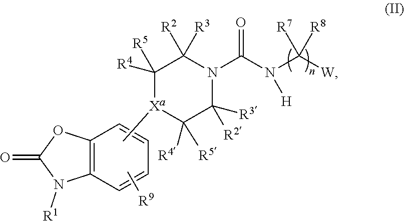

or a pharmaceutically acceptable salt thereof, wherein the variables are as defined, for example, in the compounds of formula (I) and (I-a).

In some embodiments, the compound is a compound of formula (II-a):

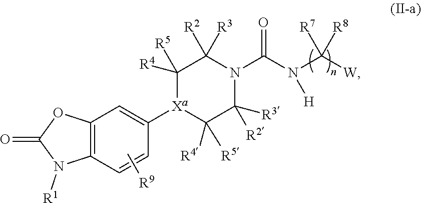

or a pharmaceutically acceptable salt thereof, wherein the variables are as defined, for example, in the compounds of formula (I) and (I-a).

In some embodiments, the compound is a compound of formula (III):

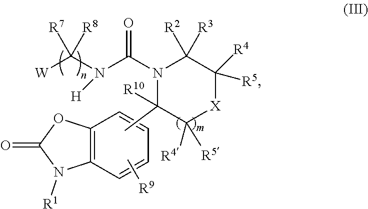

or a pharmaceutically acceptable salt thereof, wherein the variables are as defined, for example, in the compounds of formula (I) and (I-a).

In some embodiments, the compound is a compound of formula (III-a):

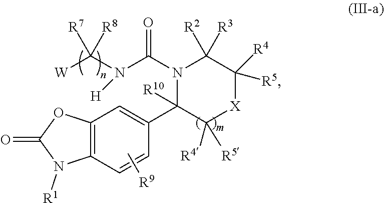

or a pharmaceutically acceptable salt thereof, wherein the variables are as defined, for example, in the compounds of formula (I) and (I-a).

In some embodiments, the compound is a compound of formula (IV):

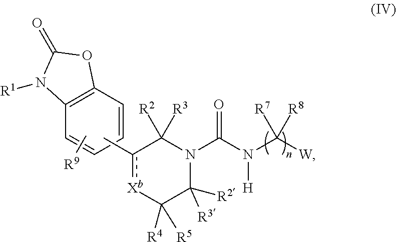

or a pharmaceutically acceptable salt thereof, wherein the variables are as defined, for example, in the compounds of formula (I) and (I-a).

In some embodiments, the compound is a compound of formula (IV-a):

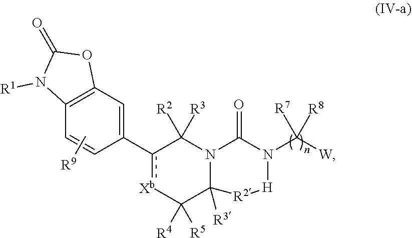

or a pharmaceutically acceptable salt thereof, wherein the variables are as defined, for example, in the compounds of formula (I) and (I-a).

In some embodiments, the compound is a compound of formula (IV-b):

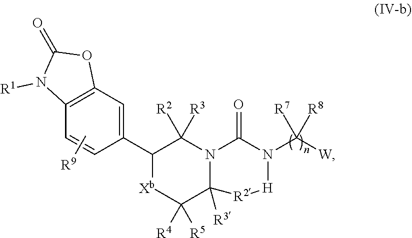

or a pharmaceutically acceptable salt thereof, wherein the variables are as defined, for example, in the compounds of formula (I) and (I-a).

In each of the foregoing compounds of formula (I), (I-a), (I-b), (I-c), and (I-d),

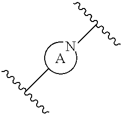

is a bicyclic heterocyclylene.

In each of the foregoing compounds of formula (I), (I-a), (I-b), (I-c), and (I-d),

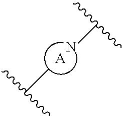

is a spiro-, fused-, or bridged-bicyclic heterocyclylene.

In each of the foregoing compounds of formula (I), (I-a), (I-b), (I-c), and (I-d),

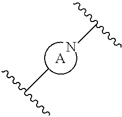

is selected from the group consisting of

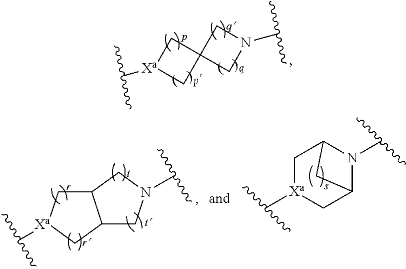

wherein X^{a} is selected from N or CH; p, p′, q, q′, r, r′, t, t′, and s are independently selected from 1 or 2.

In each of the foregoing compounds of formula (I), (I-a), (I-b), (I-c), and (I-d),

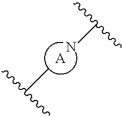

is selected from the group consisting of

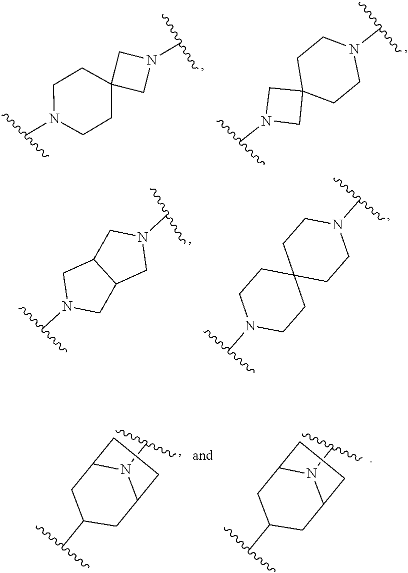 [SMILES: *N1CCC2(CC1)CN(*)C2 ; *N1CCC2(CC1)CN(*)C2 ; *N1CC2CN(*)CC2C1 ; *N1CCC2(CC1)CCN(*)CC2 ; *C1CC2CCC(C1)N2* ; *C1CC2CCC(C1)N2*]

In each of the foregoing compounds of formula (I), (I-a), (I-b), (I-c), and (Id),

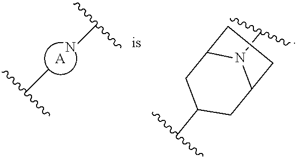

In each of the foregoing compounds of formula (I), (I-a), (I-b), (I-c), (II), (II-a), (III), (III-a), (IV), (IV-a), and (IV-b), R¹ is selected from the group consisting of hydrogen, C₁₋₆alkyl, C₁₋₆alkylene-NR^{a}₂, and 3-7 membered heterocyclyl optionally substituted with methyl.

In each of the foregoing compounds of formula (I), (I-a), (I-b), (I-c), (II), (II-a), (III), (III-a), (IV), (IV-a), and (IV-b), R¹ is hydrogen or C₁₋₆alkyl.

In each of the foregoing compounds of formula (I), (I-a), (I-b), (I-c), (II), (II-a), (III), (III-a), (IV), (IV-a), and (IV-b), R₁ is selected from methyl and hydrogen.

In each of the foregoing compounds of formula (I), (I-a), (I-b), (I-c), (II), (II-a), (III), (III-a), (IV), (IV-a), and (IV-b), R¹ is C₁₋₆alkyl.

In each of the foregoing compounds of formula (I), (I-a), (I-b), (I-c), (I-d), (II), (II-a), (III), (III-a), (IV), (IV-a), and (IV-b), R¹ is methyl.

In each of the foregoing compounds of formula (I), (I-a), (I-b), (I-c), (II), (II-a), (III), (III-a), (IV), (IV-a), and (IV-b), R¹ is selected from the group consisting of methyl, hydrogen, —CH₂CH₂N(CH₃)₂, and

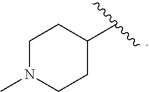 [SMILES: *C1CCN(C)CC1]

In each of the foregoing compounds of formula (I), (I-a), (I-b), (I-c), (II), (II-a), (III), (III-a), (IV), (V-a), and (IV-b), R¹ is selected from the group consisting of methyl, hydrogen, and —CH₂CH₂N(CH₃)₂.

In each of the foregoing compounds of formula (I), (I-a), (I-b), (I-c), (II), (II-a), (III), (II-a), (IV), (IV-a), and (IV-b), R⁷ and R⁸ are independently hydrogen or methyl.

In each of the foregoing compounds of formula (I), (I-a), (I-b), (I-c), (II), (II-a), (III), (III-a), (IV), (IV-a), and (IV-b), R⁷ and R⁸ are both hydrogen.

In each of the foregoing compounds of formula (I), (I-a), (II), (I-a), (III), (III-a), (IV), (IV-a), and (IV-b), R⁹ is hydrogen.

In each of the foregoing compounds of formula (I), (I-a), (I-b), (I-c), (II), (II-a), (III), (III-a), (IV), (IV-a), and (IV-b), W is selected from the group consisting of methyl, phenyl, 5-6 membered heteroaryl, C₃₋₇cycloalkyl, 3-7 membered heterocyclyl, O—(C₁₋₆alkyl), and O—(C₁₋₆alkylene)-phenyl, wherein each aforementioned phenyl is optionally substituted with 1-3 substituents independently selected from the group consisting of C₁₋₆alkyl, halogen, and O—(C₁₋₆alkyl).

In each of the foregoing compounds of formula (I), (I-a), (I-b), (I-c), (II), (II-a), (III), (III-a), (IV), (IV-a), and (IV-b), W is selected from the group consisting of methyl, phenyl, pyridazinyl, imidazolyl, cyclohexyl, ethoxy, methoxy, cyclopropyl, and —O—CH₂-phenyl, wherein each aforementioned phenyl is optionally substituted with 1-3 substituents independently selected from the group consisting of hydrogen, and methyl.

In each of the foregoing compounds of formula (I), (I-a), (I-b), (I-c), (I), (II-a), (III), (III-a), (IV), (IV-a), and (IV-b), W is selected from the group consisting of methyl, phenyl, pyridazinyl, cyclohexyl, ethoxy, methoxy, cyclopropyl, and —O—CH₂-phenyl, wherein each aforementioned phenyl is optionally substituted with 1-3 substituents independently selected from the group consisting of C₁₋₆alkyl, halogen, and O—(C₁₋₆alkyl).

In each of the foregoing compounds of formula (I), (I-a), (I-b), (I-c), (II), (II-a), (III), (III-a), (IV), (IV-a), and (IV-b), W is selected from the group consisting of methyl, phenyl, cyclopropyl, and —O—CH₂-phenyl.

In each of the foregoing compounds of formula (I), (I-a), (I-b), (I-c), (II), (II-a), (III), (I-a), (IV), (IV-a), and (I-b), W is methyl or phenyl.

In each of the foregoing compounds of formula (I), (I-a), (I-b), (I-c), (I), (II-a), (III), (III-a), (IV), (IV-a), and (IV-b), W is phenyl.

In each of the foregoing compounds of formula (I), (I-a), (I-b), (I-c), (II), (II-a), (III), (III-a), (IV), (IV-a), and (IV-b), W is methyl.

In each of the foregoing compounds of formula (I), (I-a), (I-b), (I-c), (II), (II-a), (III), (III-a), (IV), (IV-a), and (IV-b), n is 2, 3, or 4.

In each of the foregoing compounds of formula (I), (I-a), (I-b), (I-c), (II), (II-a), (III), (III-a), (IV), (IV-a), and (IV-b), n is 0, 1, 2, 3, or 4. In some embodiments, n is 2, 3, or 4.

In some embodiments, n is 0. In some embodiments, n is 1. In some embodiments, n is 2. In some embodiments, n is 3. In some embodiments, n is 4. In some embodiments n is 5. In some embodiments, n is 6.

In each of the foregoing compounds of formula (I), (I-a), (I-b), (I-c), (II), (II-a), (III), (III-a), (IV), (IV-a), and (IV-b), any aforementioned phenyl or 3-7 membered heterocyclyl is optionally substituted with 1-4 substituents independently, for each occurrence, selected from the group consisting of C₁₋₆alkyl, halogen, —O—C₁₋₆alkyl, and —CH₂N(Rₐ)₂, wherein R^{a} is as defined herein.

In each of the foregoing compounds of formula (I), (I-a), (I-b), (I-c), (II), (II-a), (III), (III-a), (IV), (IV-a), and (IV-b), any aforementioned phenyl or 3-7 membered heterocyclyl at W is optionally substituted with 1-3 substituents independently, for each occurrence, selected from the group consisting of C₁₋₆alkyl, halogen, —O—C₁₋₆alkyl, and —CH₂N(Rₐ)₂, wherein R^{a} is as defined herein.

In each of the foregoing compounds of formula (I), (I-a), (I-b), (I-c), (II), (II-a), (III), (III-a), (IV), (IV-a), and (IV-b), any aforementioned phenyl at W is optionally substituted with 1-2 of methyl.

In certain embodiments, the compound is a compound described in the Examples, or a pharmaceutically acceptable salt thereof.

In certain other embodiments, the compound is one of the compounds listed in Table 1 or a pharmaceutically acceptable salt thereof.

### Methods of Preparing Compounds

Methods for preparing compounds described herein are illustrated in the following synthetic schemes. These schemes are given for the purpose of illustrating the invention, and should not be regarded in any manner as limiting the scope or the spirit of the invention. Starting materials shown in the schemes can be obtained from commercial sources or can be prepared based on procedures described in the literature.

The synthetic route illustrated in Scheme 1 depicts an exemplary procedure for preparing substituted N-heterocyclic carboxamides. A compound of formula A (wherein the variables are as described herein) is treated with isocyanate of formula B (wherein the variables are as described herein) in the presence of a catalytic amount of DMAP in a polar solvent such as acetonitrile to afford a compound of formula I. An isocyanate of formula B (wherein the variables are as described herein) is commercially available or can be prepared from commercially available compounds according to the procedures known to the skilled in the art.

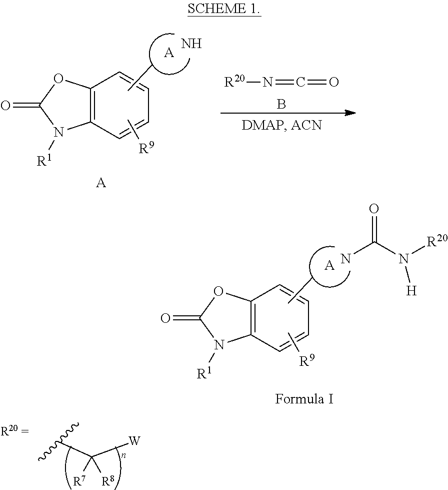

The synthetic route illustrated in Scheme 2 depicts another exemplary procedure for preparing substituted N-heterocyclic carboxamides. In the first step, a compound of formula A (wherein the variables are as described herein) is treated with an activating agent (e.g., triphosgene, phenylchloroformate, p-nitrophenylchloroformate, 1,1′-carbonyldiimidazole) with a base (e.g., TEA, DIPEA, pyridine) in an organic solvent (DCM, THF, ACN) and the resulting intermediate is treated with an amine of formula C (wherein the variables are as described herein) to afford a compound of formula I.

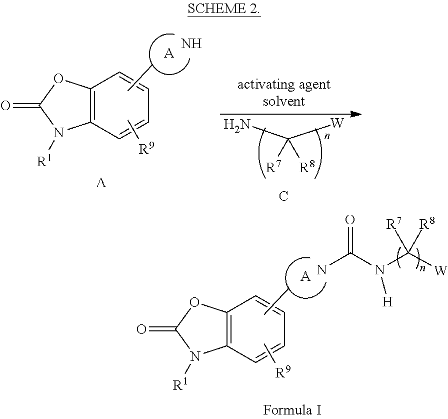

The reaction procedures in Schemes 1 and 2 are contemplated to be amenable to preparing a wide variety of substituted N-heterocyclic carboxamide compounds having different substituents. Furthermore, if a functional group that is part of the substituents would not be amenable to a reaction condition described in Schemes 1 and 2, it is contemplated that the functional group can first be protected using standard protecting group chemistry and strategies, and then the protecting group is removed after completing the desired synthetic transformation. See, for example, Greene, T. W.; Wuts, P. G. M. Protective Groups in Organic Synthesis, 2^{nd} ed.; Wiley: New York, 1991, for further description of protecting chemistry and strategies.

### III. Pharmaceutical Compositions

The invention provides pharmaceutical compositions comprising a compound described herein e.g. a compound of Formula (I), e.g., a compound of Formula (I-a), (I-b), (I-c), (I-d), (II), (II-a), (III), (III-a), (IV), (IV-a), or (IV-b)) or related organic compound described herein. In certain embodiments, the pharmaceutical compositions preferably comprise a therapeutically-effective amount of one or more of a compound described herein, e.g., a compound of Formula (I), e.g., a compound of Formula (I-a), (I-b), (I-c), (I-d), (II, (II-a), (III), (III-a), (IV), (IV-a), or (IV-b), formulated together with one or more pharmaceutically acceptable carriers. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration.

The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention, e.g., a compound of Formula (I), e.g., a compound of Formula (I-a), (I-b), (I-c), (I-d), (II), (II-a), (III), (III-a), (IV), (IV-a), or (IV-b).

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcelluloses, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

### IV. Methods of Use

Sphingolipids are a family of membrane lipids derived from the aliphatic amino alcohol sphingosine and its related sphingoid bases. They are present in eukaryote membranes, where they exert important structural roles in the regulation of fluidity and subdomain structure of the lipid bilayer. In addition to serving roles in cell membrane structure and dynamics, sphingolipids also serve important signaling functions, for example, in the control of cell growth, cell differentiation, and cell death, and can be important for cell homeostasis and development. Zeidan et al. (2010) supra, Proksch et al. (2011) supra. Ceramide, a key member of this lipid class, has attracted attention in view of its impact on the replication and differentiation of neoplastic cells. Furuya et al. (2011) supra. For example, lower levels of ceramide have been discovered in several types of human tumors relative to normal tissue, where the level of ceramide appears to correlate inversely with the degree of malignant progression. Realini et al. (2013) supra.

Acid ceramidase is a cysteine amidase that catalyzes the hydrolysis of ceramide into sphingosine and fatty acid, and is believed to be involved in the regulation of ceramide levels in cells and modulates the ability of this lipid messenger to influence the survival, growth and death of certain tumor cells. Id. Furthermore, acid ceramidase enzymes are abnormally expressed in various types of human cancer (e.g., prostate, head and neck, and colon) and serum AC levels are elevated in patients with melanoma relative to control subjects. Id.

In addition, acid ceramidase enzymes have been implicated in a number of other disorders, including, inflammation (for example, rheumatoid arthritis and psoriasis), pain, inflammatory pain, and various pulmonary disorders. See, International Application Publication No. WO2015/173169. Furthermore, acid ceramidase enzymes have been identified as a target for the treatment of certain lysosomal storage disorders (for example, Gaucher's, Fabry's, Krabbe, Tay Sachs), and neurodegenerative disorders (for example, Alzheimers, Parkinson's, Huntington's, and amytrophic lateral sclerosis). See, International Application Publication Nos. WO2016/210116 and WO2016/210120.

It is contemplated that the compounds, compositions, and methods disclosed herein can be used to treat various disorders associated or correlated with elevated levels of acid ceramidase activity. The invention provides administering to a subject in need thereof an effective amount of a compound or composition disclosed herein, either alone or in a combination with another therapeutic agent to treat the disorder.

In certain embodiments, the compound or composition used in one or more of the methods described herein is one of the generic or specific compounds described in Section II, such as a compound of Formula (I), a compound embraced by one of the further embodiments describing definitions for certain variables of Formula (I), a compound of Formula (I-a), (I-b), (I-c), (I-d), (II), (II-a), (III), (III-a), (IV), (IV-a), or (IV-b), or a compound embraced by one of the further embodiments describing definitions for certain variables of Formula (I), (I-a), (II), (II-a), (III), (III-a), (IV), (IV-a), or (IV-b).

In certain embodiments, a method or composition described herein, is administered in combination with one or more additional therapies, e.g., surgery, radiation therapy, or administration of another therapeutic preparation. In certain embodiments, the additional therapy may include an additional therapeutic agent. The invention embraces combination therapy, which includes the administration of a compound described herein, e.g., a compound of Formula (I) (e.g., a compound of Formula (I-a), (I-b), (I-c), (I-d), (II), (II-a), (III), (III-a), (IV), (IV-a), or (IV-b)) or composition described herein and a second treatment and/or agent as part of a specific treatment regimen intended to provide the beneficial effect from the co-action of the foregoing. The beneficial effect of the combination may include pharmacokinetic or pharmacodynamic co-action resulting from the foregoing combination of agents and/or treatments.

The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

1. Cancer, Inflammatory and Other Disorders

The compositions and methods disclosed herein can be used to treat various disorders associated or otherwise correlated with elevated levels of acid ceramidase activity. Exemplary disorders include cancer, inflammation, pain and inflammatory pain, or a pulmonary disease.

In certain embodiments, the compositions and methods disclosed herein can be used to treat cancer or inhibit cancer growth in a subject in need thereof. The invention provides a method of treating a cancer in a subject. The method comprises administering to the subject an effective amount of a compound (e.g., a compound of Formula (I) (e.g., a compound of Formula (I-a), (I-b), (I-c), (I-d), (II), (II-a), (III), (III-a), (IV), (IV-a), or (IV-b)) or a pharmaceutical composition disclosed herein, either alone or in a combination with another therapeutic agent to treat the cancer in the subject.

Exemplary cancers include, but are not limited to, pre-malignant conditions, for example hyperplasia, metaplasia or dysplasia, cancer metastasis, benign tumors, angiogenesis, hyperproliferative disorders and benign dysproliferative disorders. The treatment may be prophylactic or therapeutic. The subject to be treated may be human or a non-human animal (e.g., a non-human primate or a non-human mammal).

In certain embodiments, a compound disclosed herein, e.g., a compound of Formula (I), e.g., a compound of Formula (I-a), (I-b), (I-c), (I-d), (II), (II-a), (II), (III-a), (IV), (IV-a), or (IV-b), or a pharmaceutical composition containing such a compound, can be used to treat a disorder involving primary and/or metastatic neoplastic disease.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CML), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma)

In certain embodiments, the present invention provides a compound disclosed herein, e.g., a compound of Formula (I), e.g., a compound of Formula (I-a), (I-b), (I-c), (I-d), (II), (II-a), (III), (III-a), (IV), (IV-a), or (IV-b), or a pharmaceutical composition disclosed herein for the use in the treatment and/or prevention of brain cancer, breast cancer, colon cancer, head and neck cancer, liver cancer, lung cancer (e.g., alveolar cancer), pancreatic cancer, prostate cancer, skin cancer (e.g., melanoma).

It is contemplated that the compounds disclosed can be used in combination with other treatments and/or therapeutic agents. The invention embraces combination therapy, which includes the administration of a compound described herein, e.g., a compound of Formula (I), e.g., a compound of Formula (I-a), (I-b), (I-c), (I-d), (II), (II-a), (III), (III-a), (IV), (IV-a), or (IV-b), or related compound described herein and a second treatment and/or agent as part of a specific treatment regimen intended to provide the beneficial effect from the co-action of these therapeutic agents. The beneficial effect of the combination may include pharmacokinetic or pharmacodynamic co-action resulting from the combination of therapeutic agents.

In certain embodiments, a compound or pharmaceutical composition described herein, is administered in combination with one or more additional cancer therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g. a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodialator, a statin, an anti-inflammatory agent (e.g. methotrexate), or an NSAID. In certain embodiments, the additional therapy may include a combination of therapeutics of different classes.

In certain embodiments, a method or pharmaceutical composition described herein is administered in combination with a checkpoint inhibitor. The checkpoint inhibitor may, for example, be selected from a PD-1 antagonist, PD-L1 antagonist, CTLA-4 antagonist, adenosine A2A receptor antagonist, B7-H3 antagonist, 137-1-14 antagonist, BTLA antagonist, KIR antagonist, LAG3 antagonist, TIM-3 antagonist, VISTA antagonist or TIGIT antagonist.

In certain embodiments, the checkpoint inhibitor is a PD-1 or PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described in U.S. Pat. Nos. 8,728,474 and 9,073,994, and EP Patent No. 1537878131, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), duvalumab (AstraZeneca), MEDI4736, avelumab, and BMS 936559 (Bristol Myers Squibb Co.).

In certain embodiments, a compound or pharmaceutical composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

Exemplary cytotoxic agents that can be administered in combination with a compound or pharmaceutical composition described herein include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAT-A, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a compound or pharmaceutical composition described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids

In certain embodiments, a compound disclosed herein, e.g., a compound of Formula (I), e.g., a compound of Formula (I-a), (I-b), (I-c), (I-d), (II), (II-a), (III), (III-a), (IV), (IV-a), or (IV-b), or a pharmaceutical composition containing such a compound, can be used to treat an inflammatory condition, such as rheumatoid arthritis and ulcerative cholitis. The invention provides a method of treating an inflammatory condition. The method comprises administering to the subject an effective amount of a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition disclosed herein, either alone or in a combination with another therapeutic agent to treat the inflammatory condition in the subject.

As used herein, an inflammatory condition is a disease or condition characterized, in whole or in part, by inflammation or an inflammatory response in the patient. Typically, one or more of the symptoms of the inflammatory disease or condition is caused or exacerbated by an inappropriate, misregulated, or overactive inflammatory response. Inflammatory diseases or conditions may be chronic or acute. In certain embodiments, the inflammatory disease or condition is an autoimmune disorder.

Inflammatory conditions treatable using a compound or pharmaceutical composition disclosed herein may be characterized, for example, based on the primary tissue affected, the mechanism of action underlying the condition, or the portion of the immune system that is misregulated or overactive. Examples of inflammatory conditions, as well categories of diseases and conditions are provided herein. In certain embodiments, examples of inflammatory conditions that may be treated include inflammation of the lungs, joints, connective tissue, eyes, nose, bowel, kidney, liver, skin, central nervous system, vascular system, heart, or adipose tissue. In certain embodiments, inflammatory conditions which may be treated include inflammation due to the infiltration of leukocytes or other immune effector cells into affected tissue. In certain embodiments, inflammatory conditions which may be treated include inflammation mediated by IgE antibodies. Other relevant examples of inflammatory conditions which may be treated by the present disclosure include inflammation caused by infectious agents, including but not limited to viruses, bacteria, fungi, and parasites. In certain embodiments, the inflammatory condition that is treated is an allergic reaction. In certain embodiments, the inflammatory condition is an autoimmune disease.

Inflammatory lung conditions include asthma, adult respiratory distress syndrome, bronchitis, pulmonary inflammation, pulmonary fibrosis, and cystic fibrosis (which may additionally or alternatively involve the gastro-intestinal tract or other tissue(s)). Inflammatory joint conditions include rheumatoid arthritis, rheumatoid spondylitis, juvenile rheumatoid arthritis, osteoarthritis, gouty arthritis and other arthritic conditions. Inflammatory eye conditions include uveitis (including iritis), conjunctivitis, scleritis, and keratoconjunctivitis sicca. Inflammatory bowel conditions include Crohn's disease, ulcerative colitis, inflammatory bowel disease, and distal proctitis. Inflammatory skin conditions include conditions associated with cell proliferation, such as psoriasis, eczema, and dermatitis (e.g., eczematous dermatitides, topic and seborrheic dermatitis, allergic or irritant contact dermatitis, eczema craquelee, photoallergic dermatitis, phototoxicdermatitis, phytophotodermatitis, radiation dermatitis, and stasis dermatitis). Inflammatory conditions of the endocrine system include, but are not limited to, autoimmune thyroiditis (Hashimoto's disease), Type I diabetes, inflammation in liver and adipose tissue associated with Type II diabetes, and acute and chronic inflammation of the adrenal cortex. Inflammatory conditions of the cardiovascular system include, but are not limited to, coronary infarct damage, peripheral vascular disease, myocarditis, vasculitis, revascularization of stenosis, atherosclerosis, and vascular disease associated with Type II diabetes. Inflammatory conditions of the kidney include, but are not limited to, glomerulonephritis, interstitial nephritis, lupus nephritis, nephritis secondary to Wegener's disease, acute renal failure secondary to acute nephritis, Goodpasture's syndrome, post-obstructive syndrome and tubular ischemia. Inflammatory conditions of the liver include, but are not limited to, hepatitis (arising from viral infection, autoimmune responses, drug treatments, toxins, environmental agents, or as a secondary consequence of a primary disorder), obesity, biliary atresia, primary biliary cirrhosis and primary sclerosing cholangitis. In certain embodiments, the inflammatory condition is an autoimmune disease, for example, rheumatoid arthritis, lupus, alopecia, autoimmune pancreatitis, Celiac disease, Behcet's disease, Cushing syndrome, and Grave's disease. In certain embodiments, the inflammatory condition is a rheumatoid disorder, for example, rheumatoid arthritis, juvenile arthritis, bursitis, spondylitis, gout, scleroderma, Still's disease, and vasculitis.

In certain embodiments, the present invention provides a compound disclosed herein, e.g., a compound of Formula (I), e.g., a compound of Formula (I-a), (I-b), (I-c), (I-d), (II), (II-a), (III), (III-a), (IV), (IV-a), or (IV-b), or a pharmaceutical composition containing a compound disclosed herein for use in the treatment of a pain syndrome, disorder, disease or condition characterized by nociceptive pain, neuropathic pain, inflammatory pain, non-inflammatory pain, pain associated with acute conditions such as post-operative or post-traumatic stress disorders, pain associated with chronic conditions such as diabetes. The invention provides a method of treating pain. The method comprises administering to the subject an effective amount of a compound (e.g., a compound of Formula (I), e.g., a compound of Formula (I-a), (I-b), (I-c), (I-d), (IT), (II-a), (III), (III-a), (IV), (IV-a), or (IV-b) or a pharmaceutical composition disclosed herein, either alone or in a combination with another therapeutic agent to treat the pain in the subject.

A compound or composition described herein can be useful for the treatment (including prevention and/or alleviation) of chronic and/or acute pain, in particular non-inflammatory musculoskeletal pain such as back pain, fibromyalgia and myofascial pain, more particularly for reduction of the associated muscular hyperalgesia or muscular allodynia. Non-limiting examples of types of pain that can be treated by a compound or composition disclosed includes chronic conditions such as musculoskeletal pain, including fibromyalgia, myofascial pain, back pain, pain during menstruation, pain during osteoarthritis, pain during rheumatoid arthritis, pain during gastrointestinal inflammation, pain during inflammation of the heart muscle, pain during multiple sclerosis, pain during neuritis, pain during AIDS, pain during chemotherapy, tumor pain, headache, CPS (chronic pain syndrome), central pain, neuropathic pain such as trigeminal neuralgia, shingles, stamp pain, phantom limb pain, temporomandibular joint disorder, nerve injury, migraine, post-herpetic neuralgia, neuropathic pain encountered as a consequence of injuries, amputation infections, metabolic disorders or degenerative diseases of the nervous system, neuropathic pain associated with diabetes, pseudesthesia, hypothyroidism, uremia, vitamin deficiency or alcoholism; and acute pain such as pain after injuries, postoperative pain, pain during acute gout or pain during operations, such as jaw surgery.

In certain embodiments, the present invention provides a compound disclosed herein, e.g., a compound of Formula (I), e.g., a compound of Formula (I-a), (I-b), (I-c), (I-d), (II), (II-a), (III), (III-a), (IV), (IV-a), or (IV-b), or a pharmaceutical composition disclosed herein for use in the treatment of a pulmonary disease, such as asthma, chronic obstructive pulmonary disease (COPD), adult respiratory disease, acute respiratory distress syndrome, chronic bronchitis, and emphysema. The invention provides a method of treating a pulmonary disease. The method comprises administering to the subject an effective amount of a compound (e.g., a compound of Formula (I), e.g., a compound of Formula (I-a), (I-b), (I-c), (Id), (II), (II-a), (III), (III-a), (IV), (IV-a), or (IV-b) or a pharmaceutical composition disclosed herein, either alone or in a combination with another therapeutic agent to treat the pulmonary disease in the subject.

### II. Lysosomal Storage Disorders

Lysosomal storage disorders (LSDs) are a group of more than 50 clinically-recognized, rare inherited metabolic disorders that result from defects in lysosomal function (Walkley, J. (2009) INHERIT. METAB. DIS., 32(2): 181-9). LSDs are caused by dysfunction of the cell's lysosomes, which are heterogeneous subcellular organelles containing specific hydrolases that allow targeted processing or degradation of proteins, nucleic acids, carbohydrates, and lipids (HARRISON'S PRINCIPLES OF INTERNAL MEDICINE, 16^{th} Edition, vol. II, Chapter 20, pp. 2315-2319). The lysosome encloses an acidic environment and contains enzymes that catalyze the hydrolysis of biological macromolecules.

Individually, LSDs occur with incidences of less than 1:100,000, however, as a group the incidence is as high as 1 in 1,500 to 7,000 live births (Staretz-Chachain, et al. (2009) PEDIATRICS, 123(4): 1191-207). LSDs typically are caused by inborn genetic errors. Affected individuals generally appear normal at birth, however the diseases are progressive. The development of clinical disease may not occur until years or decades later, but is typically fatal.

It is believed that sphingosine-containing analogs (for example, glucosylsphingosine, galactosphingosine, lactosylsphingosine, GB3-sphingosine, and GM2-sphingosine) may accumulate in cells of subjects with certain lysosomal storage disorders or LSDs (for example, Gauchers disease, Krabbe disease, multiple sclerosis, Fabry's disease, and Tay Sachs disease, respectively) and that the accumulation of these sphingosine-containing analogs may contribute to the disease phenotype. See, e.g., International Application Publication No. WO2016/210116. Given that such sphingosine-containing analogs are often produced by acid ceramidase enzymes in the lysosomal compartments of cells in subjects with LSDs, the accumulation of the sphingosine-containing analogs to detrimental levels can be prevented or reduced by the use of an effective amount of one or more of the acid ceramidase inhibitors described herein.

In certain embodiments, a compound (e.g., a compound of Formula (I), e.g., a compound of Formula (I-a), (I-b), (I-c), (I-d), (II), (II-a), (III), (III-a), (IV), (IV-a), or (IV-b) or pharmaceutical composition containing a compound disclosed herein can be used to treat a LSD in a subject in need thereof. The invention provides a method of treating a LSD in a subject. The method comprises administering to the subject an effective amount of a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition disclosed herein, either alone or in a combination with another therapeutic agent to treat the LSD in the subject.

Exemplary LSDs include, for example, Krabbe disease, Fabry disease, Tay-Sachs disease, Sandhoff Variant A, or B, Pompe disease, Hunter's syndrome, Niemann Pick disease Types A and B, and Gaucher's disease.

It is contemplated that the compounds disclosed can be used in combination with other treatments and/or therapeutic agents. The invention embraces combination therapy, which includes the administration of a compound described herein, e.g., a compound of Formula (I), e.g., a compound of Formula (I-a), (T-b), (I-c), (I-d), (II), (II-a), (III), (III-a), (IV), (IV-a), or (IV-b), or related compound described herein and a second treatment and/or agent as part of a specific treatment regimen intended to provide the beneficial effect from the co-action of these therapeutic agents. Exemplary second agents for use in treating Gaucher disease include, for example, imiglucerase (CEREZYME®), taliglucerase alfa (ELELYSO®), velaglucerase alfa (VPR IV®), eliglustat (CERDELGA®), and miglustat (ZAVESCA®) or a glucocerebrosidase activator such as one or more of the compounds described in International Application Publication No. WO2012/078855. Exemplary second agents for use in treating Fabry disease include, for example, alpha-galactosidase A (FABRAZYME®). Additional acid ceramidase inhibitors for use in combination therapies include, for example, those described in International Patent Application Publications WO 2015/173168 and WO 2015/173169, each of which are hereby incorporated by reference.

### III. Neurodegenerative Disorders

Neurodegenerative disorders often are associated with reduction in the mass and/or volume of the brain, which may be due to the atrophy and/or death of brain cells, which are far more profound than those in a healthy subject that are attributable to aging. Neurodegenerative disorders can evolve gradually, after a long period of normal brain function, due to progressive degeneration (e.g., nerve cell dysfunction and death) of specific brain regions. Alternatively, neurodegenerative disorders can have a quick onset, such as those associated with trauma or toxins. The actual onset of brain degeneration may precede clinical expression by many years.

Examples of neurodegenerative disorders include, for example, Alzheimer's disease, Parkinson's disease, Huntington's disease, amyotrophic lateral sclerosis (ALS; also known as Lou Gehrig's disease or motor neuron disease), multiple sclerosis, and diffuse Lewy body disease. Once clinical expression occurs, the neurodegenerative disorder may be associated with impairment of motor function, for example, as observed in subjects with Parkinson's disease, Huntington's disease multiple sclerosis, or ALS. Alternatively or in addition, neurodegenerative disorders may be associated with cognitive impairment and/or the loss of cognitive function, for example, as observed in subjects with Alzheimer's disease.

Alzheimer's disease is a central nervous system (CNS) disorder that results in memory loss, unusual behavior, personality changes, and a decline in thinking abilities. These losses are related to the death of specific types of brain cells and the breakdown of connections and their supporting network (e.g., glial cells) between them. The earliest symptoms include loss of recent memory, faulty judgment, and changes in personality. Parkinson's disease is a CNS disorder that results in uncontrolled body movements, rigidity, tremor, and dyskinesia, and is associated with the death of brain cells in an area of the brain that produces dopamine. ALS (motor neuron disease) is a CNS disorder that attacks the motor neurons, components of the CNS that connect the brain to the skeletal muscles. Huntington's disease is another neurodegenerative disease that causes uncontrolled movements, loss of intellectual faculties, and emotional disturbance.

It has been observed that subjects with certain mutant alleles in genes encoding β-glucocerebrosidase activity (the GBA gene; Aharon-Peretz (2004) NEW. ENG. J. MED. 351: 1972-1977; Gan-Or et al. (2008) NEUROLOGY 70:2277-2283; Gan-Or et al. (2015) NEUROLOGY 3:880-87) and sphinomyelinase activity (the SMPD1 gene, Gan-Or et al. (2013) NEUROLOGY 80:1606-1610) have been associated with, and identified as a risk factor for, Parkinson's Disease. As a result defects with, or deficiencies in the activities of these enzymes, as in the case of Gaucher's disease and Niemann Pick types A and B, can cause an accumulation of glucosylceramide and sphingomyelin, which can then be converted to glucosylsphingosine or lyso-sphingomyelin, respectively, via acid ceramidase activity. The accumulation of glucosylsphingosine or lyso-sphingomyelin may thus be implicated in the development of Parkinson's disease. It is contemplated that the administration of an acid ceramidase inhibitor, which slows down, stops or reverses the accumulation of glucosylsphingosine and/or lyso-sphingomyelin can be used to treat Parkinson's Disease. For example, an acid ceramidase inhibitor can be used to improve motor and/or memory impairments symptomatic of Parkinson's disease.

Similarly, it has been observed that lactosylceramide (LacCer) is upregulated in the central nervous system of mice during chronic experimental autoimmune encephalomyelitis (EAE), a model of multiple sclerosis (Lior et al. (2014) NATURE MEDICINE 20:1147-1156.). It is contemplated that the increase in LacCer may also result in the an increase in lactosylsphingosine (LacSph) via conversion by an acid ceramidase (a lactosylceramide to lactosylsphingosine converting enzyme). Given the accumulation of lactosylsphingosine to a toxic or otherwise detrimental level or concentration in the lysosomal compartments of cells in subjects with multiple sclerosis, it is contemplated that the administration of an acid ceramidase inhibitor can reduce the accumulation of lactosylsphingosine thereby treating multiple sclerosis, which includes ameliorating a symptom associated with multiple sclerosis.

It has been observed that the level and activity of acid ceramidase can be elevated in subjects with Alzheimer's disease (Huang et al. (2004) EUROPEAN J. NEUROSCI. 20:3489-3497). Given that the accumulation of sphingosine or sphingosine analogs to a toxic or otherwise detrimental level or concentration in the lysosomal compartments of cells in subjects with Alzheimer's disease, it is contemplated that the administration of an acid ceramidase inhibitor can reduce the accumulation of the sphingosine or sphingosine analogs thereby treating Alzheimer's disease, which includes ameliorating a symptom associated with Alzheimer's disease.

Furthermore, given that a number of the foregoing neurodegenerative disorders, for example, Alzheimer's disease, are associated with a level of cognitive impairment and/or some decrease or loss of cognitive function, it is contemplated that the administration of an effective of an acid ceramidase inhibitor to a subject in need thereof may be reduce, stabilize, or reverse cognitive impairment and/or the loss of cognitive function. Cognitive function generally refers to the mental processes by which one becomes aware of, perceives, or comprehends ideas. Cognitive function involves all aspects of perception, thinking, learning, reasoning, memory, awareness, and capacity for judgment. Cognitive impairment generally refers to conditions or symptoms involving problems with thought processes. This may manifest itself in one or more symptoms indicating a decrease in cognitive function, such as impairment or decrease of higher reasoning skills, forgetfulness, impairments to memory, learning disabilities, concentration difficulties, decreased intelligence, and other reductions in mental functions.

Cognitive function and cognitive impairment may be readily evaluated using tests well known in the art. Performance in these tests can be compared over time to determine whether a treated subject is improving or whether further decline has stopped or slowed, relative to the previous rate of decline of that patient or compared to an average rate of decline. Tests of cognitive function, including memory and learning for evaluating human patients are well known in the art and regularly used to evaluate and monitor subjects having or suspected of having cognitive disorders such as Alzheimer's disease including the clock-drawing test (Agrell & Dehlin (1998) AGE & AGING 27.399-403). Even in healthy individuals, these and other standard tests of cognitive function can be readily used to evaluate beneficial affects over time.

In certain embodiments, a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition containing a compound disclosed herein can be used to treat a neurodegenerative disorder in a subject in need thereof. The invention provides a method of treating a neurodegenerative disorder in a subject. The method comprises administering to the subject an effective amount of a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition disclosed herein, either alone or in a combination with another therapeutic agent to treat the neurodegenerative disorder in the subject.

Exemplary neurodegenerative disorders include, for example, Alzheimer's disease, Parkinson's disease, Huntington's disease, amyotrophic lateral sclerosis, Lewy body disease, dementia (e.g., frontotemporal dementia), multisystem atrophy, multiple sclerosis, epilepsy, bipolar disorder, schizophrenia, anxiety disorders (e.g., a panic disorder, social anxiety disorder or generalized anxiety disorder) or progressive supranuclear palsy.

It is contemplated that the compounds disclosed can be used in combination with other treatments and/or therapeutic agents. The invention embraces combination therapy, which includes the administration of a compound described herein, e.g., a compound of Formula (I), e.g., a compound of Formula (I-a), (I-b), (I-c), (I-d), (II), (II-a), (III), (III-a), (IV), (IV-a), or (IV-b), or related compound described herein and a second treatment and/or agent as part of a specific treatment regimen intended to provide the beneficial effect from the co-action of these therapeutic agents.

During the treatment of Parkinson's disease, the acid ceramidase inhibitor can be administered in combination with carbidopa and/or levadopa, a dopamine agonist, a monoamine oxidase B inhibitor, a catchetol O-methyltransferase inhibitor, an anticholingeric, or amantadine. During the treatment of Alzheimer's disease, the acid ceramidase inhibitor can be administered in combination with a cholinesterase inhibitor and/or menantine. During the treatment of Huntington's disease, the acid ceramidase inhibitor can be administered in combination with tetrabenazine; an antipsychotic drug such as haloperidol, chlorpromazine, quetiapine, risperidone, and olanzapine; a chorea-suppressing medication such as amantadine, levetiracetam, and clonazempam; an antidepressant such as citalopram, fluoxetine, and sertraline; and a mood-stabilizing drug such as valproate, carbamazepine, and lamotrigine.

During the treatment of amyotrophic lateral sclerosis, the acid ceramidase inhibitor can be administered in combination with riluzole; an agent for ameliorating muscle cramps and spasms such as cyclobenzaprine HCl, metaxalone, and robaxin; an agent for ameliorating spasticity such as tizanidine HCl, baclofen, and dantrolene; an agent for ameliorating fatigue such as caffeine, caffeine citrate, or caffeine benzoate injection; an agent for ameliorating excessive salivation such as glycopyrrolate, propantheline, amitriptyline, nortriplyline HCl and scopolamine; an agent for ameliorating excessive phlegm such as guaifenesin, albuterol inhalation, and acetylcysteine; an agent for ameliorating pain such as an opioid; an anticonvulsant or antiepileptic; a serotonin reuptake inhibitor; an antidepressant; an agent for ameliorating sleep disorders such as a benzodiazepine, a non-benzodiazepine hypnotic, a melatonin receptor stimulator, an anti-narcoleptic, and an orexin receptor antagonist; and an agent pseudobulbar affect such as dextromethorphan/quinidine.

During the treatment of multiple sclerosis, the acid ceramidase inhibitor can be administered in combination with a corticosteroid, P interferon, glatiramer acetate, dimethyl fumarate, fingolimod, teriflunomide, natalizumab, mitoxantrone, baclofen, and tizanidine. During the treatment of diffuse Lewy body disease, the acid ceramidase inhibitor can be administered in combination with a cholinesterase inhibitor, a Parkinson's disease medication such as carbidopa and/or levodopa, and an anti-psychotic medication such as quetiapine and olanzapine.

During the treatment of multisystem atrophy, the acid ceramidase inhibitor can be administered in combination with a medication to raise blood pressure such as fludrocortisone, psyridostigmine, midodrine, and droxidopa; and a Parkinson's disease medication such as carbidopa and/or levodopa. During the treatment of fronitotemporal dementia, the acid ceramidase inhibitor can be administered in combination with an antidepressant, a selective serotonin reuptake inhibitor, and an antipsychotic. During the treatment of progressive upranuclear palsy, the acid ceramidase inhibitor can be administered in combination with a Parkinson's disease medication such as carbidopa and/or levodopa. It is understood that other combinations would be known be those skilled in the art.

### V. Kits for Use in Medical Applications

Another aspect of the invention provides a kit for treating a disorder. The kit comprises: i) instructions for treating a medical disorder, such as, cancer (such as melanoma), a lysosomal storage disorder (such as Krabbe disease, Fabry disease, Tay-Sachs disease, Pompe disease, Hunter's syndrome, Niemann Pick disease Types A and B. Gaucher disease), a neurodegenerative disease (such as Alzheimer's disease, Parkinson's disease, Huntington's disease, and amyotrophic lateral sclerosis), an inflammatory disorder, and pain; and ii) a compound described herein or related organic compound described herein, such as a compound of Formula (I), e.g., a compound of Formula (f-a), (I-b), (I-c), (I-d), (II), (II-a), (III), (III-a), (IV), (IV-a), or (IV-b) or a composition described herein. The kit may comprise one or more unit dosage forms containing an amount of a compound described herein or related organic compound described herein, such as a compound of Formula (I), e.g., a compound of Formula (I-a), (I-b), (I-c), (I-d), (II), (I-a), (III), (III-a), (IV), (IV-a), or (IV-b) that is effective for treating said medical disorder, for example, cancer (such as melanoma), lysosomal storage disorder (such as Krabbe disease, Fabry disease, Tay-Sachs disease, Pompe disease, Hunter's syndrome, Niemann Pick disease Types A and B, Gaucher disease), neurodegenerative disease (such as Alzheimer's disease, Parkinson's disease, Huntington's disease, and amyotrophic lateral sclerosis), an inflammatory disorder, and pain.

The description above describes multiple aspects and embodiments of the invention, including substituted benzimidazole carboxamides and related organic compounds, compositions comprising a substituted benzimidazole carboxamides or related organic compounds, methods of using the substituted benzimidazole carboxamides or related organic compounds, and kits. The patent application specifically contemplates all combinations and permutations of the aspects and embodiments. For example, the invention contemplates treating a medical disorder such as Gaucher disease, Parkinson's disease, Lewy body disease, dementia, or multiple system atrophy in a human patient by administering a therapeutically effective amount of a compound described herein, e.g., a compound of Formula (I), e.g., a compound of Formula (I-a), (I-b), (I-c), (I-d), (II), (II-a), (III), (III-a), (IV), (IV-a), or (IV-b), or a composition comprising such a compound. Further, for example, the invention contemplates a kit for treating a medical disorder such as cancer (such as melanoma), lysosomal storage disorder (such as Krabbe disease, Fabry disease, Tay-Sachs disease, Pompe disease, Hunter's syndrome, Niemann Pick disease Types A and B, Gaucher disease), and neurodegenerative disease (such as Alzheimer's disease, Parkinson's disease, Huntington's disease, amyotrophic lateral sclerosis, Levy body disease, dementia, and multiple system atrophy), inflammatory disorder, and pain and ii) a compound described herein or related organic compound described herein, such as a compound of Formula (I), e.g., a compound of Formula (I-a), (I-b), (I-c), (I-d), (II), (II-a), (III), (III-a), IV), (IV-a), or (IV-b), or a composition comprising such a compound.

In another aspect, the invention provides a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition as disclosed herein for use in a method of treating a subject with cancer and in need thereof, the method comprising administering to the subject a therapeutically effective amount of the compound or the pharmaceutical composition.

In another aspect, the invention provides a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition as disclosed herein for use in a method of treating a subject with a lysosomal storage disorder and in need thereof, the method comprising administering to the subject a therapeutically effective amount of the compound or the pharmaceutical composition.

In another aspect, the invention provides a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition as disclosed herein for use in a method of treating a subject with a neurodegenerative disorder and in need thereof the method comprising administering to the subject a therapeutically effective amount of the compound or the pharmaceutical composition.

In another aspect, the invention provides a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition as disclosed herein for use in a method of treating a subject with an inflammatory disorder and in need thereof, the method comprising administering to the subject a therapeutically effective amount of the compound or the pharmaceutical composition.

In another aspect, the invention provides use of a compound (e.g., a compound of Formula (I), e.g., a compound of Formula (I-a), (I-b), (I-c), (I-d), (II), (II-a), (III), (II-a), (IV), (IV-a), or (IV-b) or a pharmaceutical composition as disclosed herein for the manufacture of a medicament for treating a subject with cancer and in need thereof, the method comprising administering to the subject a therapeutically effective amount of the compound or the pharmaceutical composition.

In another aspect, the invention provides use of a compound (e.g., a compound of Formula (f), e.g., a compound of Formula (I-a), (I-b), (I-c), (I-d), (II), (II-a), (III), (III-a), (IV), (IV-a), or (IV-b) or a pharmaceutical composition as disclosed herein for the manufacture of a medicament for treating a subject with a lysosomal storage disorder and in need thereof, the method comprising administering to the subject a therapeutically effective amount of the compound or the pharmaceutical composition.

In another aspect, the invention provides use of a compound (e.g., a compound of Formula (I), e.g., a compound of Formula (I-a), (I-b), (I-c), (I-d), (II), (II-a), (III), (III-a), (IV), (IV-a), or (IV-b) or a pharmaceutical composition as disclosed herein for the manufacture of a medicament for treating a subject with a neurodegenerative disorder and in need thereof, the method comprising administering to the subject a therapeutically effective amount of the compound or the pharmaceutical composition.

In another aspect, the invention provides use of a compound (e.g., a compound of Formula (I), e.g., a compound of Formula (I-a), (I-b), (I-c), (I-d), (II), (II-a), (III), (III-a), (IV), (I V-a), or (IV-b) or a pharmaceutical composition as disclosed herein for the manufacture of a medicament for treating a subject with an inflammatory disorder and in need thereof, the method comprising administering to the subject a therapeutically effective amount of the compound or the pharmaceutical composition.

### EXAMPLES

The invention now being generally described, will be more readily understood by reference to the following examples, which are included merely for purposes of illustration of certain aspects and embodiments of the present invention, and are not intended to limit the invention. In certain instances, the amount of compound produced by the procedure is stated along with the yield, which may be presented in the format of the procedure produced the title compound (10 mg; 90%) which means that 10 mg of the title compound was obtained and that corresponds to a yield of 90%.

### Preparation of N-Heterocyclic Carboxamide Compounds

A-heterocyclic carboxamide compounds were prepared based on general procedures described in Part I below.

### Part I—General Procedures

### General Procedure A: Synthesis of Compounds of Formula VIIa-1.

Step 1: Synthesis of Compounds of Formula VIa-1.

To a cooled −78° C. solution of HMDS (1.5 eq., 1.0 M in THF) in anhydrous THE (0.1 M) was added n-BuLi (1.5 eq, 2.5 M in hexane) dropwise. The solution was stirred for 20 min, then added dropwise via cannula to a cooled −78° C. solution of the appropriate ketone Va-j (1.0 eq.) in anhydrous THF (0.1 M) under N₂ atmosphere. The reaction mixture was stirred at −78° C. for 2 h, then N-chloro-2-pyridyl)bis(trifluoromethanesulfonimide) (2.0 eq.) in anhydrous THE (0.1 M) was added dropwise. The reaction mixture was stirred at −78° C. for 2 h and allowed to warm to RT. After 1 h, the reaction mixture was diluted with EA, washed with 10% aq. NaOH solution, brine and dried over Na₂SO₄. After evaporation of the solvent, the residue was purified by flash chromatography (SiO₂) eluting with Cy/EA.

Step 2: Synthesis of Compounds of Formula VI′a-i.

To a solution of the compound of Step 1 of Formula VIa-i (1.0 eq.) in 1,4-dioxane (0.1 M, previously degassed under N₂ atmosphere) was added bis(pinacolato)diboron (1.2 eq.), KOAc (2.0 eq.), PdCl₂(dppf)-DCM complex (0.2 eq.) and the reaction mixture was stirred at 90° C. for 1 h under N₂ atmosphere. The corresponding boronic ester of Formula VI′a-i was used in situ in the next step.

Step 3: Synthesis of Compounds of Formula VIIa-1.

To a mixture of the compound of Step 2 of Formula VI′a-i (1.0 eq.) in 1,4-dioxane (0.2 M, previously degassed under N₂ atmosphere) was added 5-bromo-2-nitrophenol or 2-benzyloxy-4-bromo-1-nitro-benzene (1.1 eq.), Pd catalyst (0.01 eq.) and Na₂CO₃ (2.0 eq., 2M aqueous solution). The reaction mixture was stirred at 90° C. on under N₂ atmosphere. Then, the reaction mixture was cooled to RT, diluted with EA and washed with saturated aq. NH₄Cl solution, brine and dried over Na₂SO₄. After evaporation of the solvent, the residue was purified by flash chromatography (SiO₂) eluting with Cy/EA.

### General Procedure B: Synthesis of Compounds of Formula VIIIa-k, XXXIIa-k and XXXII′a-c.

Step 1:

Method A: To a suspension of the appropriate 2-nitrophenols VIIa-I, or XXXI a-k, or XXXI′a-c (1.0 eq.) in MeOH (0.4 M) was added 10% Pd/C (0.25 eq.) and cyclohexene (30 eq.) and the mixture was stirred at reflux for 5 h. The suspension was filtered through a pad of Celite and the filtrate was quickly evaporated under reduced pressure. The residue was used in the next step without further purification.

Method B: A suspension of the appropriate 2-nitrophenols VIIa-1, or XXXI a-k, or XXXI′a-c (1.0 eq.) in MeOH (0.4 M) was hydrogenated with the H-Cube apparatus using 10% Pd/C catalyst at 60° C. and full H₂ mode. After complete conversion (UPLC/MS analysis monitoring), the solvent was evaporated under reduced pressure. The residue was used in the next step without further purification.

Method C: To a solution of the appropriate 2-nitrophenols VIIa-1, or XXXI a-k, or XXXI′a-c (1.0 eq.) in THF (0.4 M) and saturated aq. NH₄Cl solution (8.0 eq.) was added Zn solid (8.0 eq) portionwise and the mixture was stirred at RT for 15 min. The suspension was filtered through a pad of Celite and the filtrate was dried over Na₂SO₄. After evaporation of the solvent, the residue was used in the next step without purification.

Method E: To a solution of the appropriate 2-nitrophenols VIIa-1, or XXXI a-k, or XXXI′a-c (1.0 eq.) in EtOH (0.1 M) was added 10% Pd/C (0.2 eq) followed by the addition of Et₃SiH (10.0 eq.). The reaction mixture was stirred at RT for 15 min, filtered through a pad of Celite and after concentration the residue was used in the next step without purification.

Step 2:

To a solution of the compound of Step 1 of Formula VIIa-1, or XXXI a-k, or XXXI′a-c (1.0 eq.) in EA, or ACN, or DMF (0.3 M) was added CDI (1.5 eq.) and the reaction mixture was stirred at RT for 2 h. Then, the solvent was reduced in vacuo and the residue was dissolved in EA, washed with H₂O, brine and dried over Na₂SO₄. After evaporation of the solvent, the residue was purified by column chromatography (SiO₂), eluting with Cy/EA.

### General Procedure C: Synthesis of Compounds of Formula XIa-m, XXXVa-m, XXXV′a-c and XIIa-m, XXXVIa-m, XXXVI′a-c.

Step 1: To a solution (0.2 M) of compound of Formula IXa-k, or XXXIIa-k, or XXXIII′a-c (1.0 eq.) in anhydrous DMF was added the appropriate alkyl halide (1.5 eq.) and K₂CO₃ (0.75 eq.) and the reaction mixture was stirred at RT for 3 h. The reaction mixture was diluted with DCM, washed with brine and dried over Na₂SO₄. After evaporation of the solvent, the residue was used in the next step without further purification.

Step 2: To a suspension of the compound of Step 1 of Formula XIa-m, or XXXVa-m, or XXXV′a-c (1.0 eq.) in 1,4-dioxane (0.1 M) was added HCl (30 eq, 4M in 1,4-dioxane) and the reaction mixture was stirred at RT for 2 h. After evaporation of the solvent, the residue was used in the next step without further purification.

### General Procedure D: Synthesis of Compounds of Formula I

Method A: To a stirred solution of the appropriate amine of Formula Xa-c, or XIIa-m, or XXXIVa-b, or XXXVIa-m, or XXXVI′a-c (1.0 eq.) and Et₃N (4.0 eq.) in anydrous ACN (0.2 M) was added the appropriate isocyanate of Formula A (1.1 eq.). The reaction mixture was diluted DCM, washed with brine and dried over Na₂SO₄. After evaporation of the solvent, the residue was purified by column chromatography (SiO₂), eluting with Cy/EA or DC M/MeOH.

Method B: To a stirred solution of triphosgene (0.33 eq.) in dry DCM (0.2 M) was added the appropriate amine of Formula C (1.0 eq.) and anydrous Et₃N (2.0 eq.) at 0° C. The resulting mixture was stirred at RT for 30 min under N₂ atmosphere and then added to a solution of the appropriate compound of Formula Xa-c, or XIIa-m, or XXXIVa-b, or XXXVIa-m, or XXXVIa-c (1.0 eq.) and anydrous Et₃N (1.0 eq.) in anydrous DCM (0.2 M). The reaction mixture was stirred under N₂ atmosphere at RT for 30 min and then diluted with DCM, washed with saturated aq. NH₄Cl solution, brine and dried over Na₂SO₄. After evaporation of the solvent, the residue was purified by column chromatography (SiO₂), eluting with Cy/EA or DCM/MeOH.

Method C: To a stirred solution of the appropriate amine of Formula Xa-c, or XIIa-n, or XXXIVa-b, or XXXVIa-m, or XXXVI′a-c (1.0 eq.) in THF and ACN (1:1, 0.1M) was added Et₃N (or DIPEA, or pyridine, 1.2 eq.) followed by the addition of phenylchloroformate (or p-nitrophenylchloroformate, or CDI, 1.1 eq.). The reaction was stirred at RT overnight, then, diluted with DCM, washed with 1-20, brine and dried over Na₂SO₄. After evaporation of the solvent, the residue was taken up in THF (0.1 M) and added dropwise to a solution of the appropriate amine of Formula C (1.0 eq.) and Et₃N (or DIPEA, or pyridine, 1.0 eq.). The reaction mixture was stirred at RT for 2 h and then diluted with DCM, washed with saturated aq. NH₄Cl solution, brine and dried over Na₂SO₄. After evaporation of the solvent, the residue was purified by column chromatography (SiO₂), eluting with Cy/EA or DCM/MeOH.

### General Procedure E: Synthesis of Compounds of Formula XXXIi-k.

To a solution of the appropriate amine of Formula XXXi-k (1.0 eq.), 2-benzyloxy-4-bromo-1-nitrobenzene (1.2 eq.), K₃PO₄ (2.0 eq.), DMEDA (or DMCD, 0.2 eq.) in 1,4-dioxane (0.2 M, previously degassed under N₂ atmosphere) was added CuI (0.1 eq.) under N₂ atmosphere. The reaction mixture was stirred at reflux for 48 h. Then, cooled to RT, diluted with EA and washed with saturated aq. NaHCO₃ solution, brine and dried over Na₂SO₄. After evaporation of the solvent, the residue was purified by flash chromatography (SiO₂) eluting with Cy/EA.

### General Procedure F: Synthesis of Compounds of Formula XXXIa-h, or XXXI′a-c.

To a solution of the appropriate amine of Formula XXXa-h, or XXX′a-c (1.0 eq.) in ACN (0.2 M) was added DIPEA (1.3 eq.) and 5-fluoro-1-nitrophenol (1.3 eq.). The reaction mixture was stirred at 70° C. overnight. Then, cooled to RT, and diluted with DCM, washed with saturated aq. NH₄Cl solution, brine and dried over Na₂SO₄. After evaporation of the solvent, the residue was purified by column chromatography (SiO₂), eluting with Cy/EA, Cy/MTBE or DCM/MeOH.

### General Procedure G: Synthesis of Compounds of Formula XXIIa-e.

To a cooled −78° C. solution of 6-bromo-3H-1,3-benzoxazol-2-one (or 7-bromo-3H-1,3-benzoxazol-2-one, 1.0 eq.) in anydrous THF (0.1 M) was added MeMgBr (1.5 eq., 3.0 M in Et₂O). After 30 min, n-BuLi (4.5 eq., 2.5 M in hexanes) was added and the reaction mixture was stirred at −78° C. for 30 min. A solution of appropriate ketone Va-b (2.4 eq.) or appropriate N-Boc lactam XXa-f (2.0-5.0 eq), or appropriate Weinreb amide XXIa-b (2.0-5.0 eq) in anhydrous THF (3.0 M) was then added dropwise at −78° C. and the reaction mixture allowed to warm to RT. After 1 h, the reaction mixture was quenched with saturated aq. NH₄Cl solution and extracted with EA. The organic phase was washed with brine and dried over Na₂SO₄. After evaporation of the solvent, the residue was purified by column chromatography (SiO₂), eluting with Cy/EA.

General purification and analytical methods. Automated column chromatography purifications were done using a Teledyne ISCO apparatus (CombiFlash® Rf) with pre-packed silica gel columns of different sizes (from 4 g until 120 g). Mixtures of increasing polarity of Cy and EA or DCM and MeOH were used as eluents. TLC analyses were performed using Supelco silica gel on TLC Al foils 0.2 mm with fluorescence indicator 254 nm. Purifications of basic compounds were done using IST ISOLUTE® SCX packed into SPE cartridges (SCX). ¹H experiments were run on a Bruker Avance III 400 system (400.13 MHz for 1H), equipped with a BBI probe and Z-gradient coil. Spectra were acquired at 300 K, using deuterated dimethylsulfoxide (DMSO-d&) or deuterated chloroform (CDCl₃) as solvents. Chemical shifts for ¹H were recorded in parts per million using the residual nondeuterated solvent as the internal standard (for DMSO-d₆: 2.50 ppm, 11H for CDCl₃: 7.26 ppm, ¹H). Data are reported as follows: chemical shift (ppm), multiplicity (indicated as: bs, broad singlet; s, singlet; d, doublet; t, triplet; q, quartet; p, quintet, sx, sextet; m, multiplet and combinations thereof), coupling constants (J) in Hertz (Hz) and integrated intensity. UPLC/MS analyses were run on a Waters ACQUITY UPLC/MS system consisting of a SQD (Single Quadropole Detector) Mass Spectrometer equipped with an Electrospray Ionization interface and a Photodiode Array Detector. PDA range was 210-400 nm. Analyses were performed on an ACQUITY UPLC BEH C18 column (50×2.1 mmID, particle size 1.7 μm) with a VanGuard BEH C₁₈ pre-column (5×2.1 mmID, particle size 1.7 μm). Mobile phase was either 10 mM NH₄OAc in H₂O at pH 5 adjusted with AcOH (A) and 10 mM NH₄OAc in ACN-H₂O (95:5) at pH 5 (B). Electrospray ionization in positive and negative mode was applied. Analyses were performed with method A or B. Method A: Gradient: 5 to 100% B over 3 min. Flow rate 0.5 mL/min. Temperature 40° C. Method B: Gradient: 50 to 100% B over 3 min. Flow rate 0.5 mL/min. Temperature 40° C. Method C: Gradient: 0 to 100% B over 3 min. Flow rate 0.5 mL/min. Temperature 40° C. Hydrogenation reactions were also performed using H-Cube® (H-Cube) continuous hydrogenation equipment (SS-reaction line version), employing disposable catalyst cartridges (CatCart®) preloaded with the required heterogeneous catalyst. Microwave heating was performed using Explorer®-48 positions instrument (CEM). All final compounds displayed ≥95% purity as determined by NMR and UPLC/MS analysis.

The structures of the compounds of formula Xa-c and XIIa-n are as shown below:

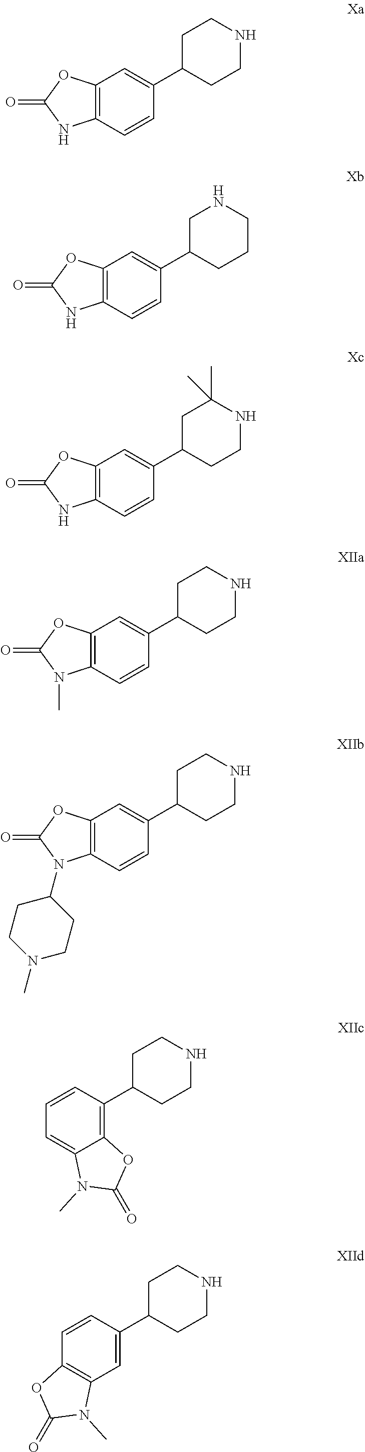

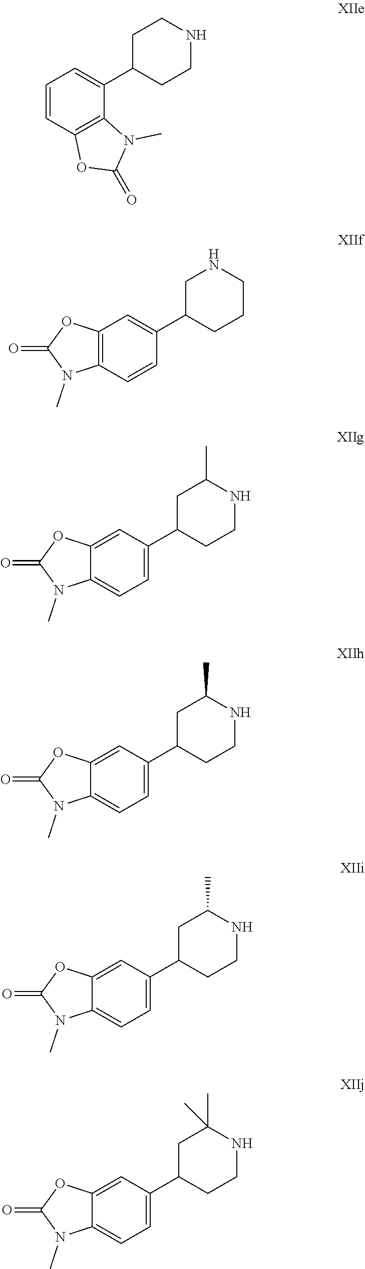 [SMILES: Cn1c(=O)oc2cccc(C3CCNCC3)c21 ; Cn1c(=O)oc2cc(C3CCCNC3)ccc21 ; CC1CC(c2ccc3c(c2)oc(=O)n3C)CCN1 ; C[C@@H]1CC(c2ccc3c(c2)oc(=O)n3C)CCN1 ; C[C@H]1CC(c2ccc3c(c2)oc(=O)n3C)CCN1 ; Cn1c(=O)oc2cc(C3CCNC(C)(C)C3)ccc21]

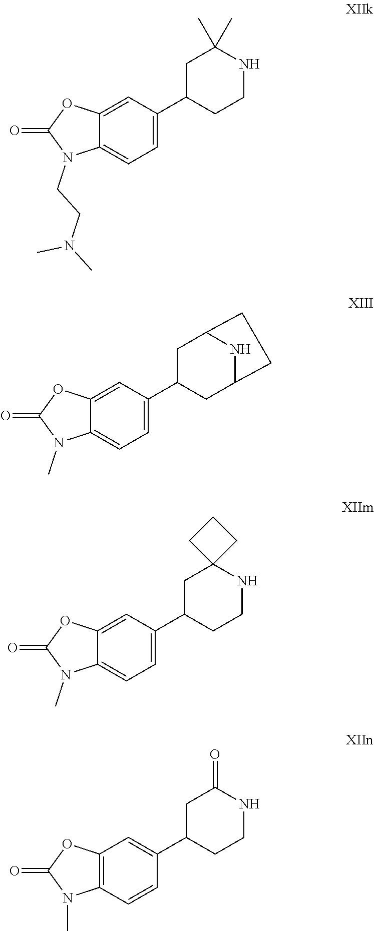 [SMILES: CN(C)CCn1c(=O)oc2cc(C3CCNC(C)(C)C3)ccc21 ; Cn1c(=O)oc2cc(C3CC4CCC(C3)N4)ccc21 ; Cn1c(=O)oc2cc(C3CCNC4(CCC4)C3)ccc21 ; Cn1c(=O)oc2cc(C3CCNC(=O)C3)ccc21]

The structures for the compounds of formula XXVa-f are as shown below.

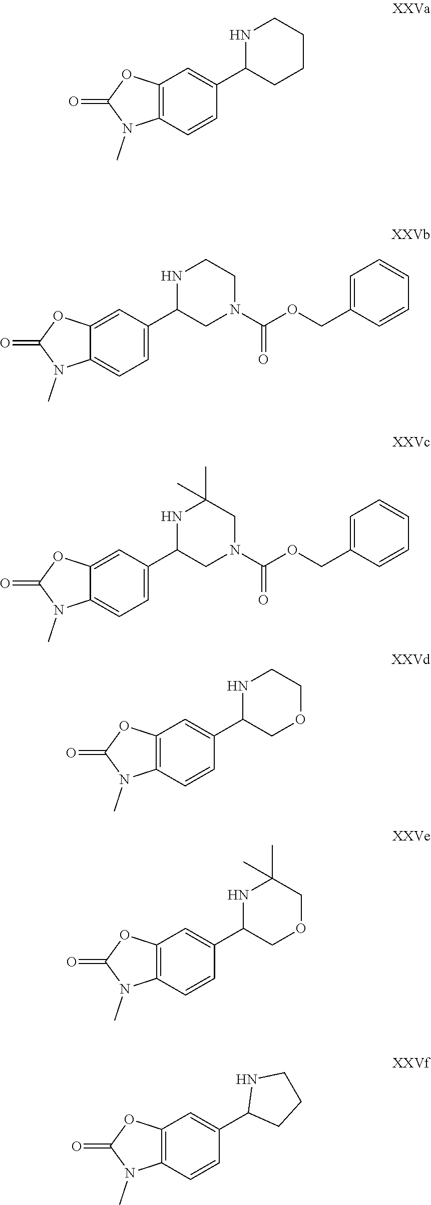 [SMILES: Cn1c(=O)oc2cc(C3CCCCN3)ccc21 ; Cn1c(=O)oc2cc(C3CN(C(=O)OCc4ccccc4)CCN3)ccc21 ; Cn1c(=O)oc2cc(C3CN(C(=O)OCc4ccccc4)CC(C)(C)N3)ccc21 ; Cn1c(=O)oc2cc(C3COCCN3)ccc21 ; Cn1c(=O)oc2cc(C3COCC(C)(C)N3)ccc21 ; Cn1c(=O)oc2cc(C3CCCN3)ccc21]

The structures for the compounds of formula XXXIVa-b and XXXVIa-m are as shown below:

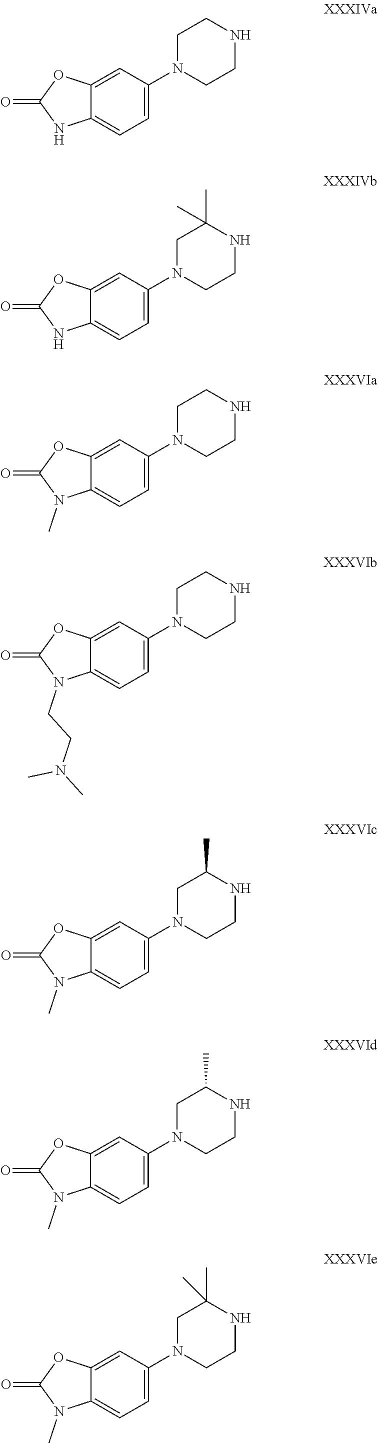

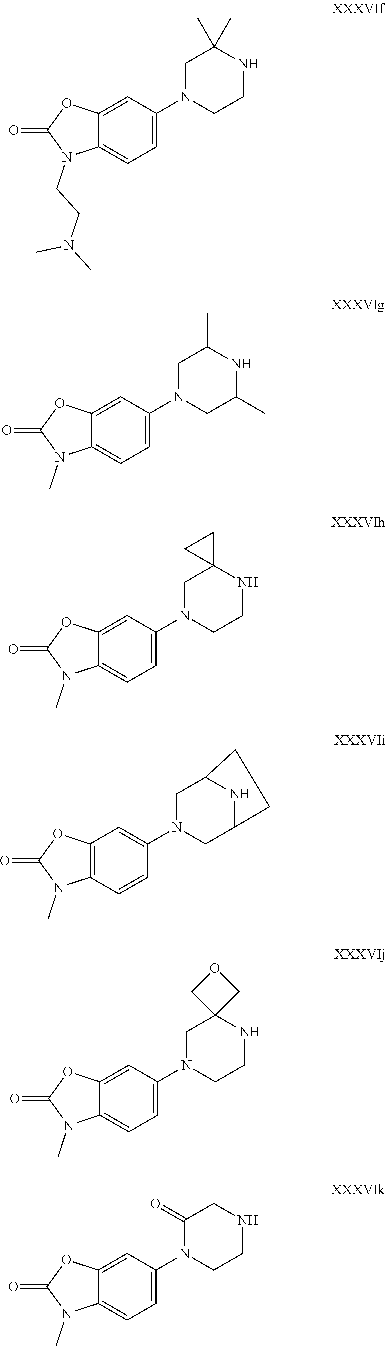 [SMILES: CN(C)CCn1c(=O)oc2cc(N3CCNC(C)(C)C3)ccc21 ; CC1CN(c2ccc3c(c2)oc(=O)n3C)CC(C)N1 ; Cn1c(=O)oc2cc(N3CCNC4(CC4)C3)ccc21 ; Cn1c(=O)oc2cc(N3CC4CCC(C3)N4)ccc21 ; Cn1c(=O)oc2cc(N3CCNC4(COC4)C3)ccc21 ; Cn1c(=O)oc2cc(N3CCNCC3=O)ccc21]

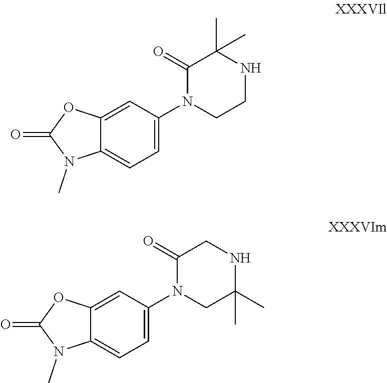 [SMILES: Cn1c(=O)oc2cc(N3CCNC(C)(C)C3=O)ccc21 ; Cn1c(=O)oc2cc(N3CC(C)(C)NCC3=O)ccc21]

The structures of the compounds of formula XXXVI′a-c are as shown below:

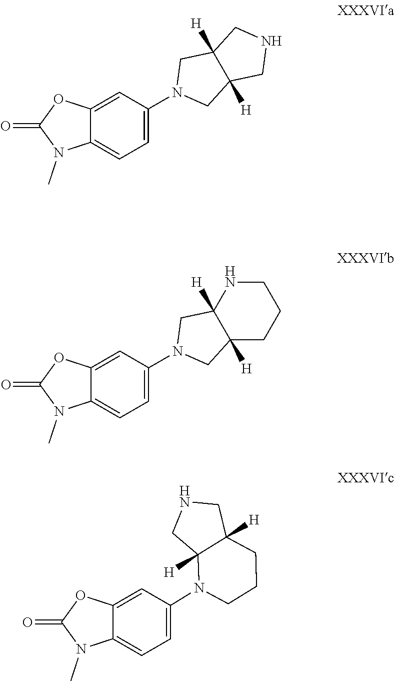 [SMILES: Cn1c(=O)oc2cc(N3C[C@H]4CNC[C@H]4C3)ccc21 ; Cn1c(=O)oc2cc(N3C[C@H]4CCCN[C@H]4C3)ccc21 ; Cn1c(=O)oc2cc(N3CCC[C@H]4CNC[C@H]43)ccc21]

The structures of the compounds of formula XXXIV″a-b and XXXVI″a-c are as shown below:

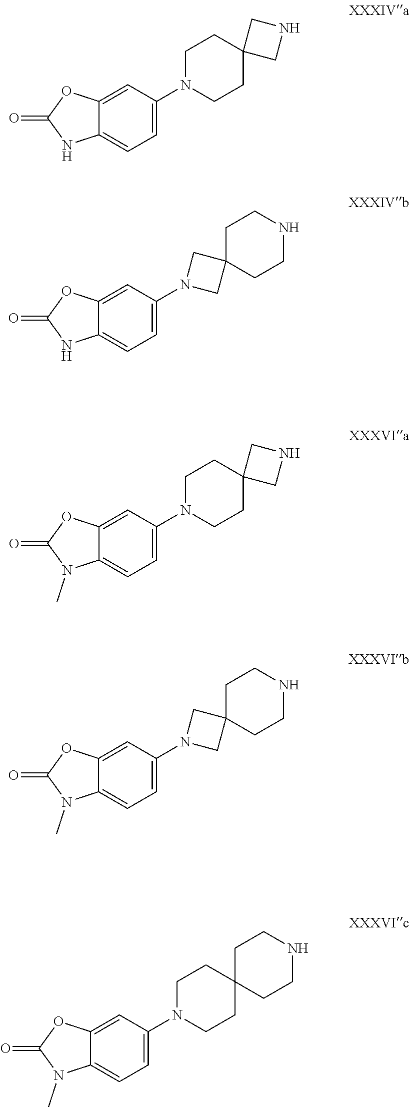 [SMILES: O=c1[nH]c2ccc(N3CCC4(CC3)CNC4)cc2o1 ; O=c1[nH]c2ccc(N3CC4(CCNCC4)C3)cc2o1 ; Cn1c(=O)oc2cc(N3CCC4(CC3)CNC4)ccc21 ; Cn1c(=O)oc2cc(N3CC4(CCNCC4)C3)ccc21 ; Cn1c(=O)oc2cc(N3CCC4(CCNCC4)CC3)ccc21]

The structures of the compounds of formula Va-j are as shown below:

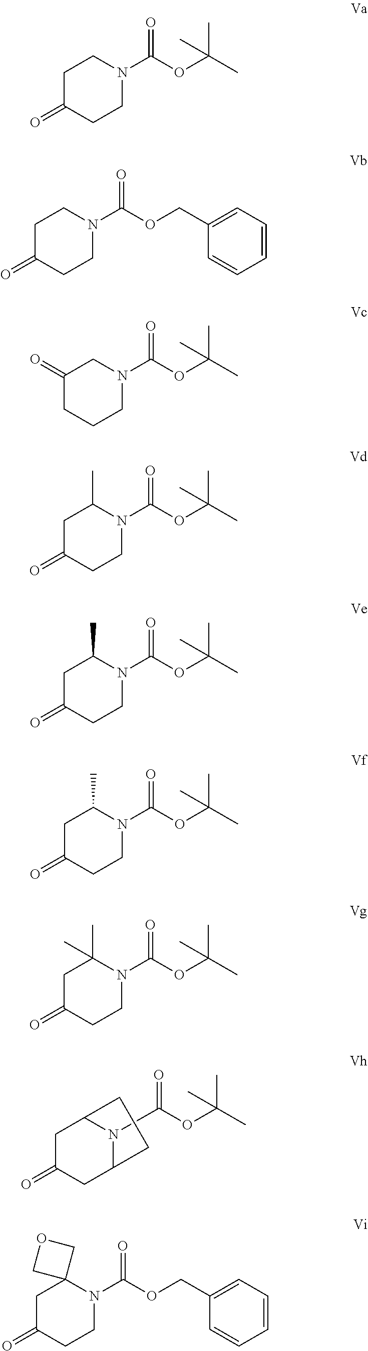

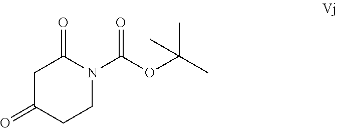

The structures of compounds of formula VIa-i are as shown below:

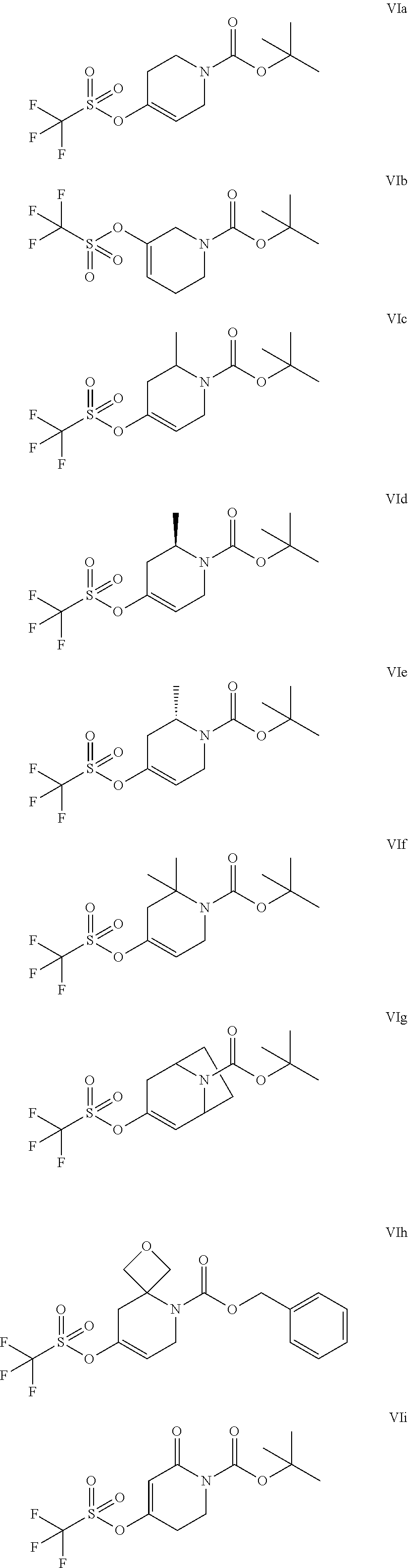

The structures of the compounds of formula VI′a-i are as shown below:

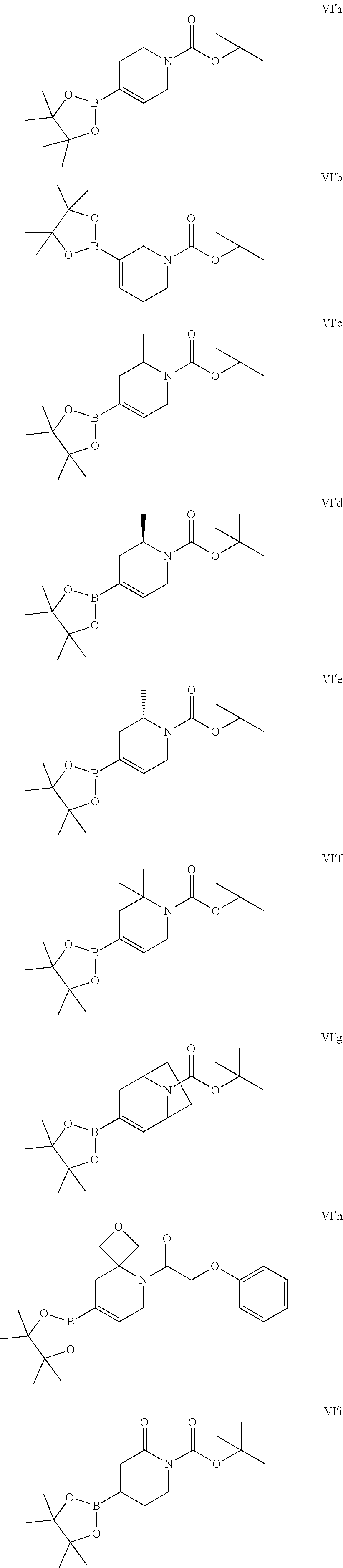

The structures of the compounds of formula VIIa-1 are as shown below:

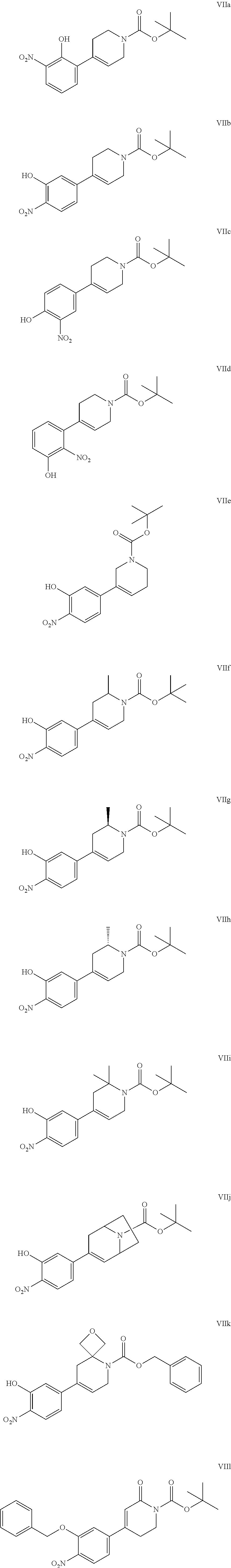

The structures of the compounds of formula VIIIa-k are as shown below:

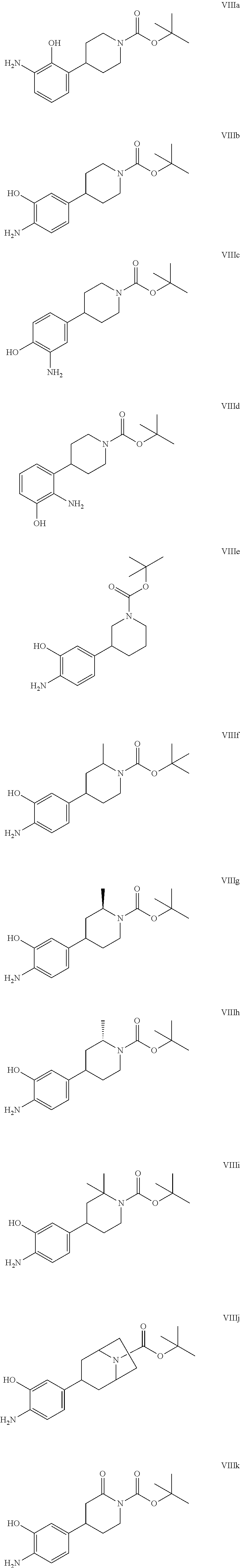

The structure of the compound of formula VII′a is as shown below:

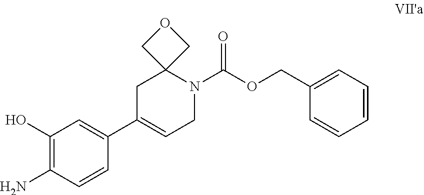 [SMILES: Nc1ccc(C2=CCN(C(=O)OCc3ccccc3)C3(COC3)C2)cc1O]

The structures of the compounds of formula IXa-k are as shown below:

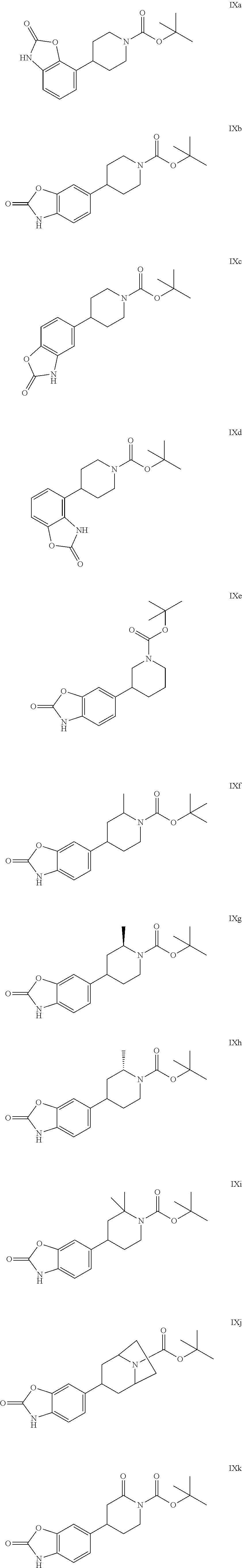

The structures of the compounds of formula Xa-c are as shown below:

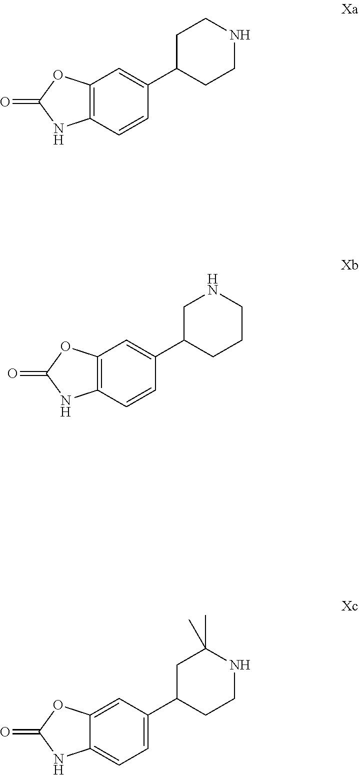 [SMILES: O=c1[nH]c2ccc(C3CCNCC3)cc2o1 ; O=c1[nH]c2ccc(C3CCCNC3)cc2o1 ; CC1(C)CC(c2ccc3[nH]c(=O)oc3c2)CCN1]

The structures of the compounds of formula XIa-m are as shown below:

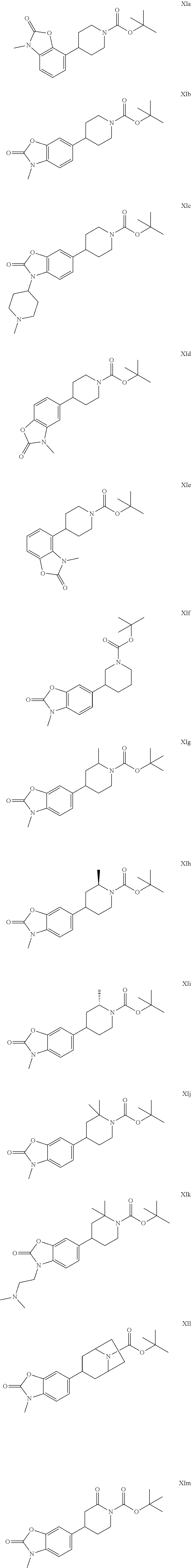

The structures of the compounds of formula XIIa-n are as shown below:

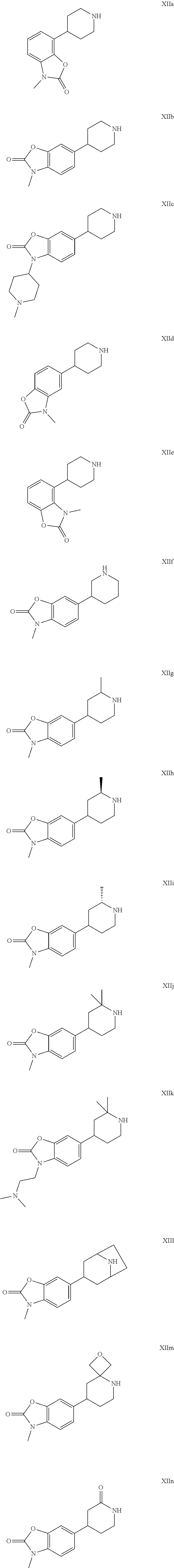

The structures of the compounds of formula XIIIa-c, XIVa-b, XVa-b, XVIa-b and XVIIa-b are as shown below:

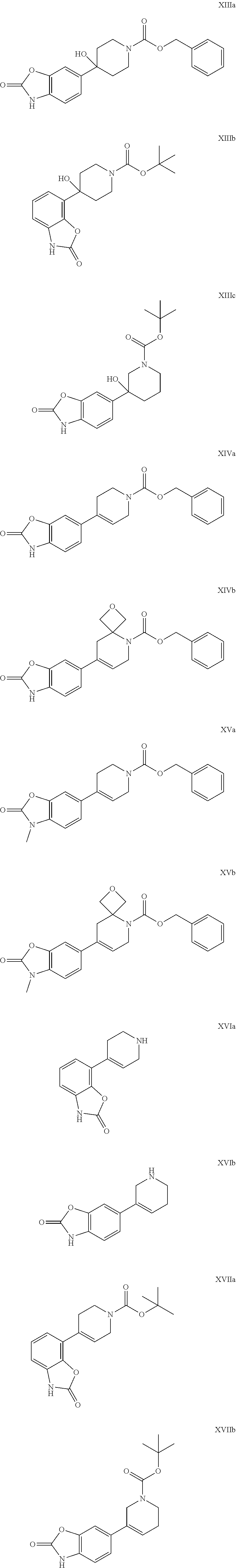

The structures of the compounds of formula XVII′a and XVII″a are as shown below:

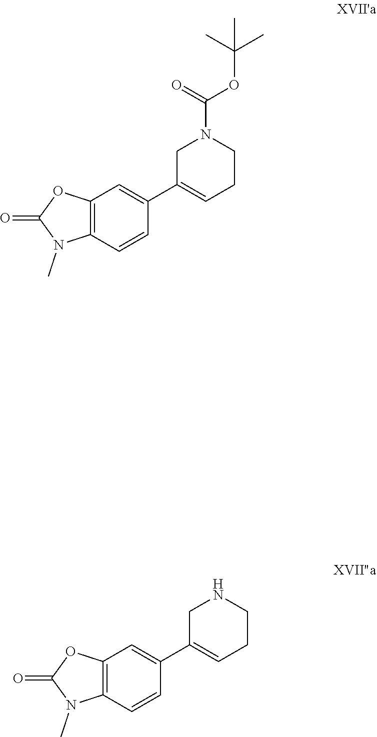 [SMILES: Cn1c(=O)oc2cc(C3=CCCN(C(=O)OC(C)(C)C)C3)ccc21 ; Cn1c(=O)oc2cc(C3=CCCNC3)ccc21]

The structures of the compounds of formula XXa-g are as shown below:

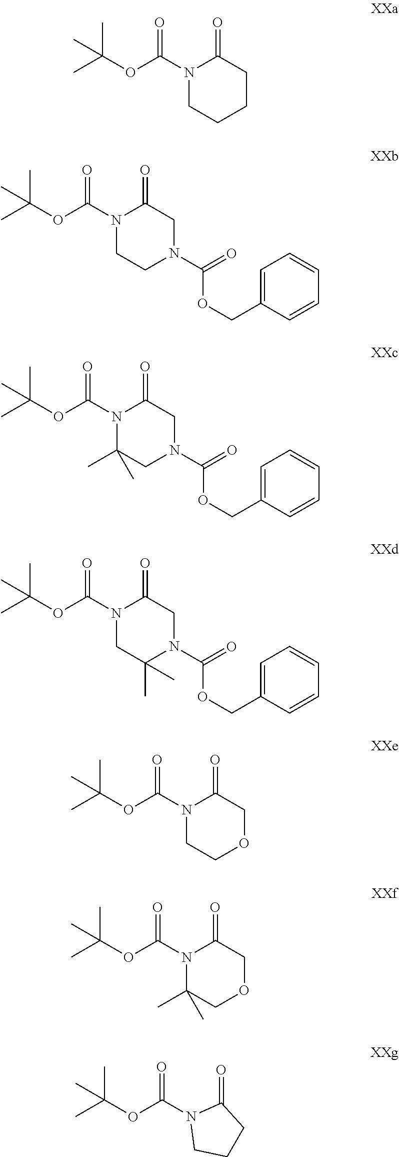

The structures of the compounds of formula XXIa-b are as shown below:

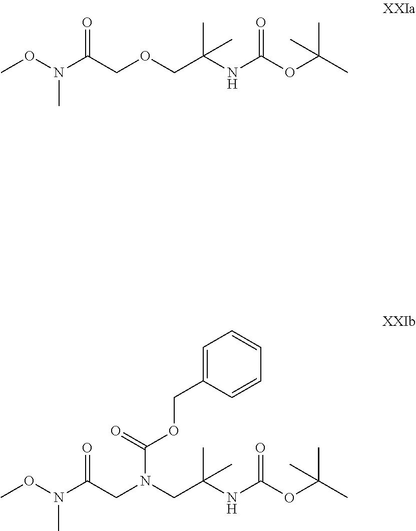

The structures of the compounds of formula XXIIa-f are as shown below:

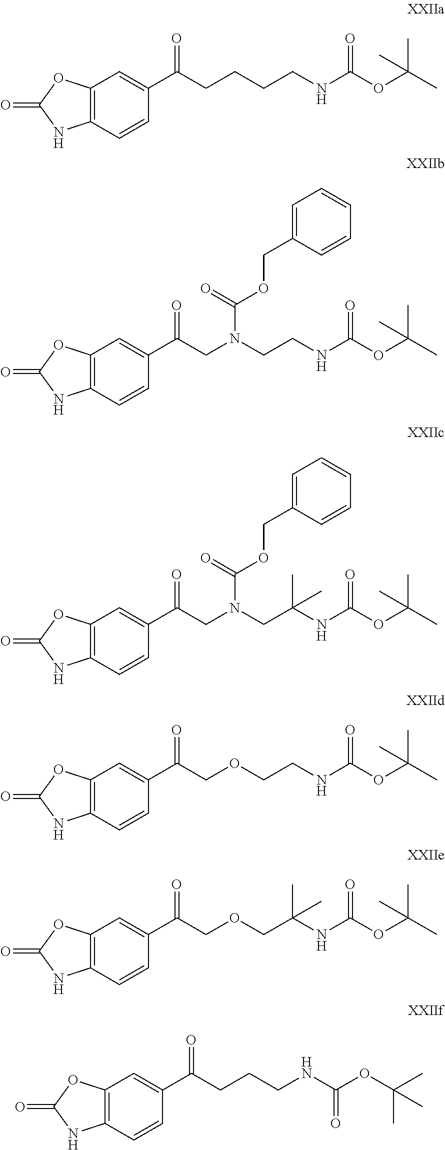 [SMILES: CC(C)(C)OC(=O)NCCCCC(=O)c1ccc2[nH]c(=O)oc2c1 ; CC(C)(C)OC(=O)NCCN(CC(=O)c1ccc2[nH]c(=O)oc2c1)C(=O)OCc1ccccc1 ; CC(C)(CN(CC(=O)c1ccc2[nH]c(=O)oc2c1)C(=O)OCc1ccccc1)NC(=O)OC(C)(C)C ; CC(C)(C)OC(=O)NCCOCC(=O)c1ccc2[nH]c(=O)oc2c1 ; CC(C)(COCC(=O)c1ccc2[nH]c(=O)oc2c1)NC(=O)OC(C)(C)C ; CC(C)(C)OC(=O)NCCCC(=O)c1ccc2[nH]c(=O)oc2c1]

The structures of the compounds of formula XXIIIa-e are as shown below:

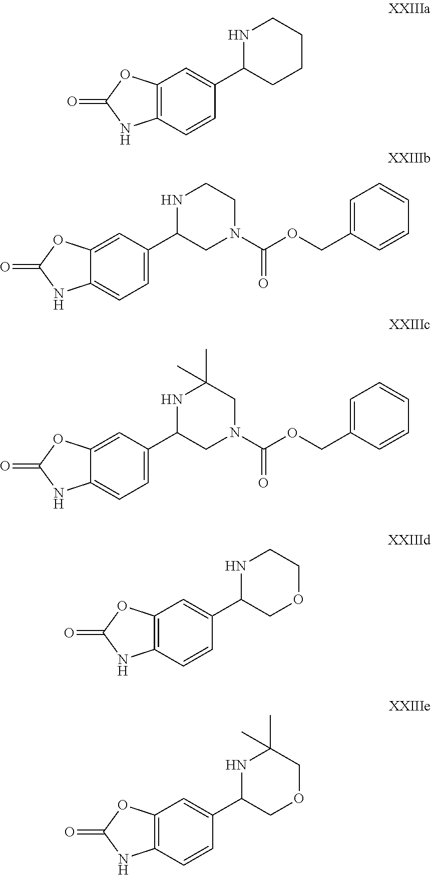 [SMILES: O=c1[nH]c2ccc(C3CCCCN3)cc2o1 ; O=C(OCc1ccccc1)N1CCNC(c2ccc3[nH]c(=O)oc3c2)C1 ; CC1(C)CN(C(=O)OCc2ccccc2)CC(c2ccc3[nH]c(=O)oc3c2)N1 ; O=c1[nH]c2ccc(C3COCCN3)cc2o1 ; CC1(C)COCC(c2ccc3[nH]c(=O)oc3c2)N1]

The structures of the compounds of formula XXIVa-f are as shown below:

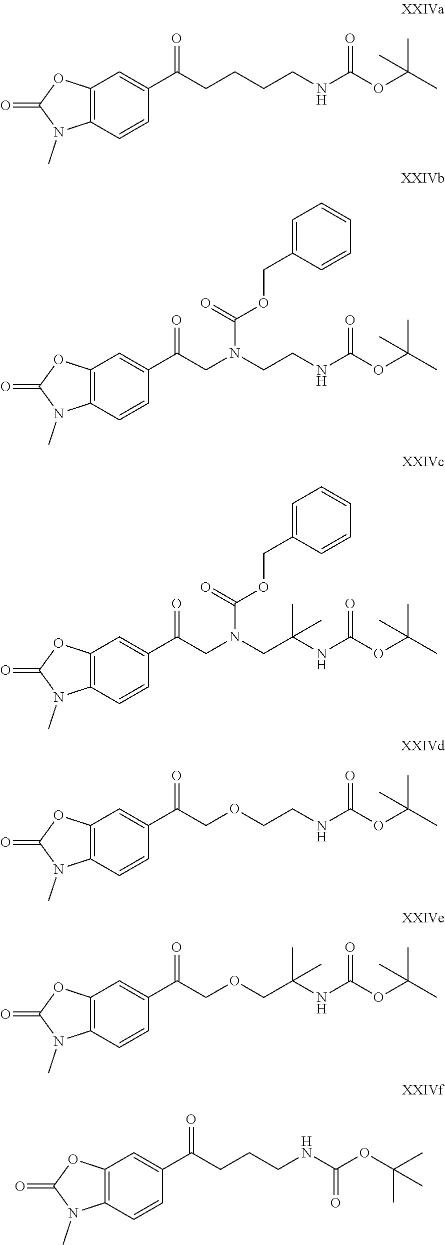 [SMILES: Cn1c(=O)oc2cc(C(=O)CCCCNC(=O)OC(C)(C)C)ccc21 ; Cn1c(=O)oc2cc(C(=O)CN(CCNC(=O)OC(C)(C)C)C(=O)OCc3ccccc3)ccc21 ; Cn1c(=O)oc2cc(C(=O)CN(CC(C)(C)NC(=O)OC(C)(C)C)C(=O)OCc3ccccc3)ccc21 ; Cn1c(=O)oc2cc(C(=O)COCCNC(=O)OC(C)(C)C)ccc21 ; Cn1c(=O)oc2cc(C(=O)COCC(C)(C)NC(=O)OC(C)(C)C)ccc21 ; Cn1c(=O)oc2cc(C(=O)CCCNC(=O)OC(C)(C)C)ccc21]

The structures of the compounds of formula XXVIa, XXVIIa, XXVIIa-b are as shown below:

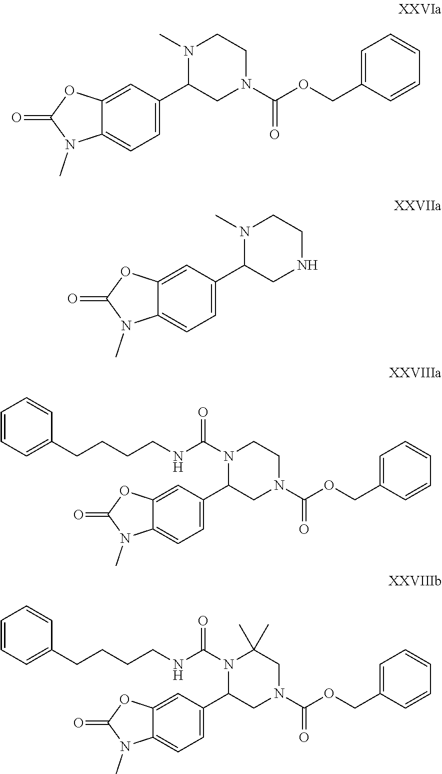 [SMILES: CN1CCN(C(=O)OCc2ccccc2)CC1c1ccc2c(c1)oc(=O)n2C ; CN1CCNCC1c1ccc2c(c1)oc(=O)n2C ; Cn1c(=O)oc2cc(C3CN(C(=O)OCc4ccccc4)CCN3C(=O)NCCCCc3ccccc3)ccc21 ; Cn1c(=O)oc2cc(C3CN(C(=O)OCc4ccccc4)CC(C)(C)N3C(=O)NCCCCc3ccccc3)ccc21]

The structures of the compounds of formula XXXa-k are as shown below:

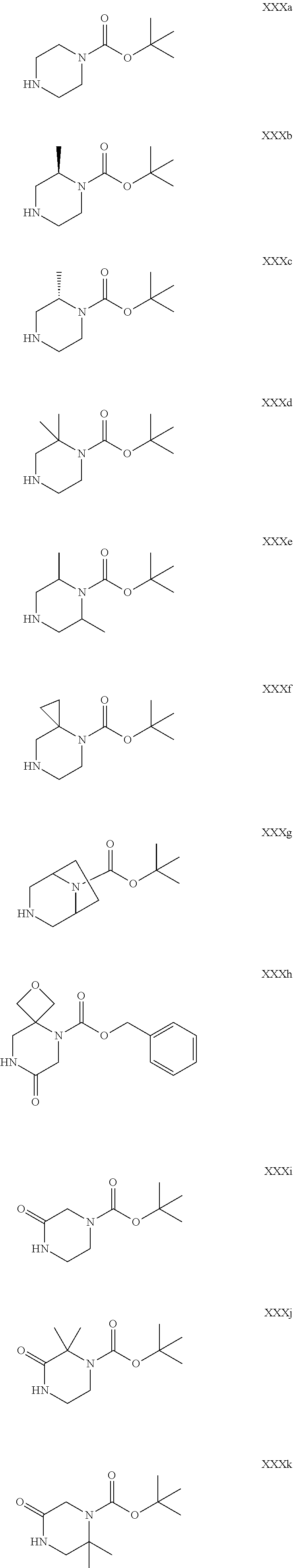

The structures of the compounds of formula XXX′a-c are as shown below:

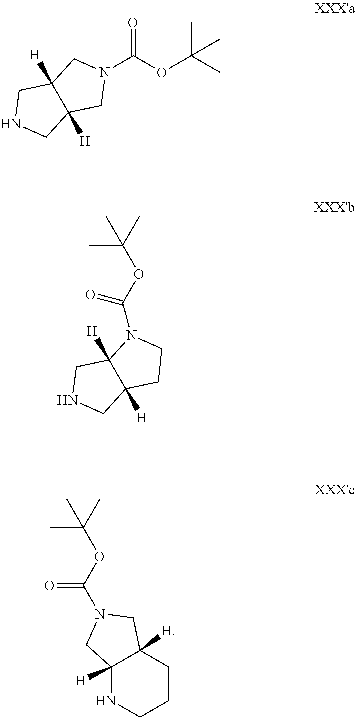

The structures of the compounds of formula XXX″a-c are as shown below:

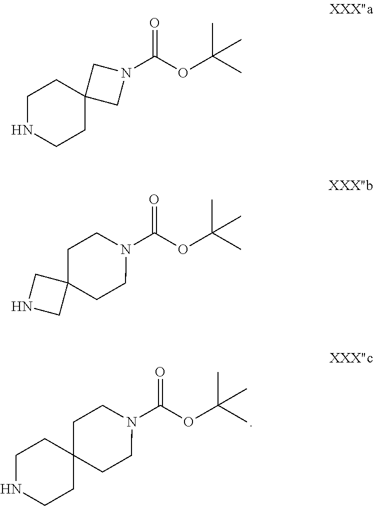 [SMILES: CC(C)(C)OC(=O)N1CC2(CCNCC2)C1 ; CC(C)(C)OC(=O)N1CCC2(CC1)CNC2 ; CC(C)(C)OC(=O)N1CCC2(CCNCC2)CC1]

The structures of the compounds of formula XXXIa-k are as shown below:

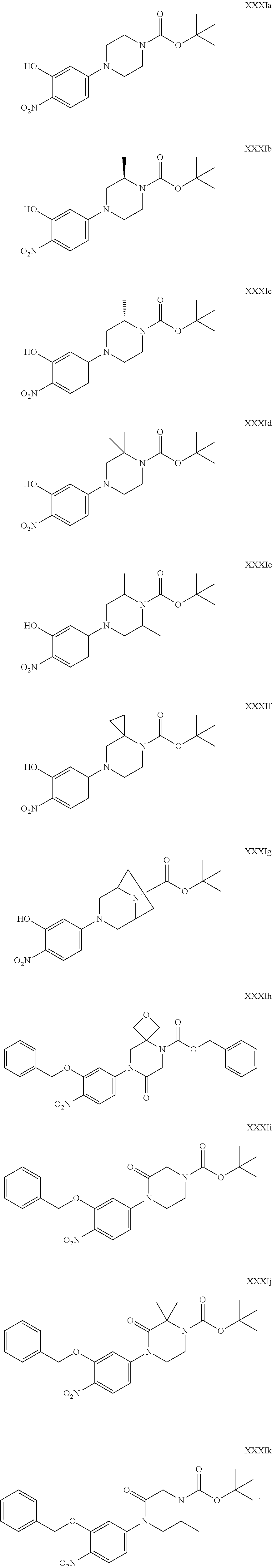

The structures of the compounds of formula XXXI′a-c are as shown below:

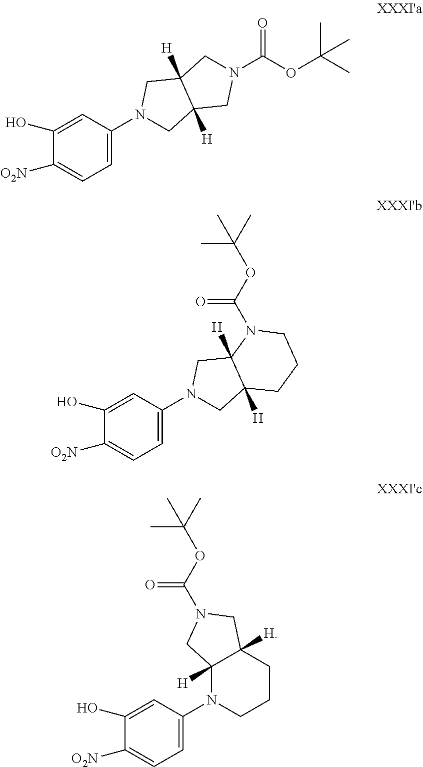

The structures of the compounds of formula XXXVII″a-c are as shown below:

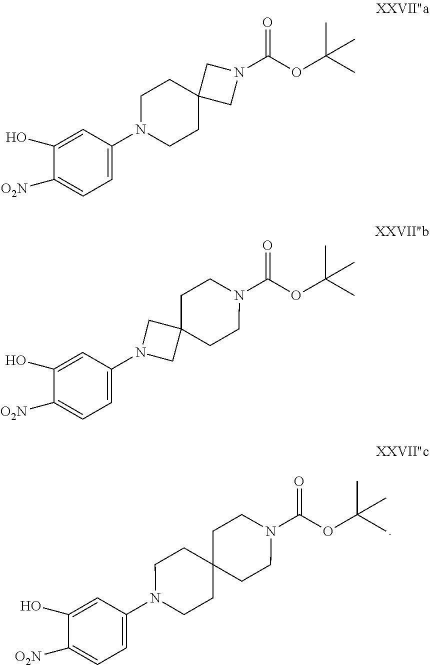 [SMILES: CC(C)(C)OC(=O)N1CC2(CCN(c3ccc([N+](=O)[O-])c(O)c3)CC2)C1 ; CC(C)(C)OC(=O)N1CCC2(CC1)CN(c1ccc([N+](=O)[O-])c(O)c1)C2 ; CC(C)(C)OC(=O)N1CCC2(CC1)CCN(c1ccc([N+](=O)[O-])c(O)c1)CC2]

The structures of the compounds of formula XXXIIa-k are as shown below:

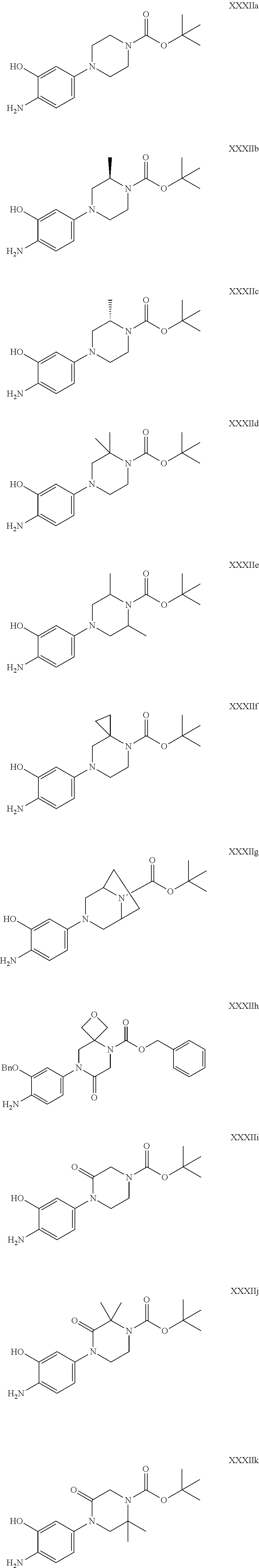

The structures of the compounds of formula XXXII′a-c are as shown below:

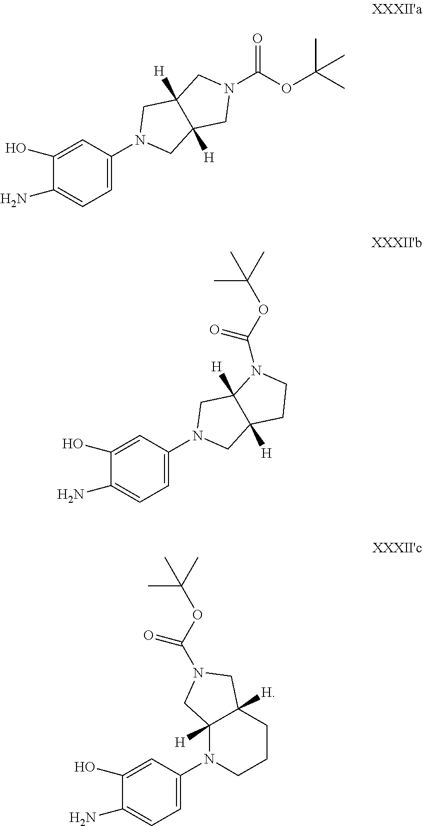

The structures of the compounds of formula XXXII″a-c are as shown below:

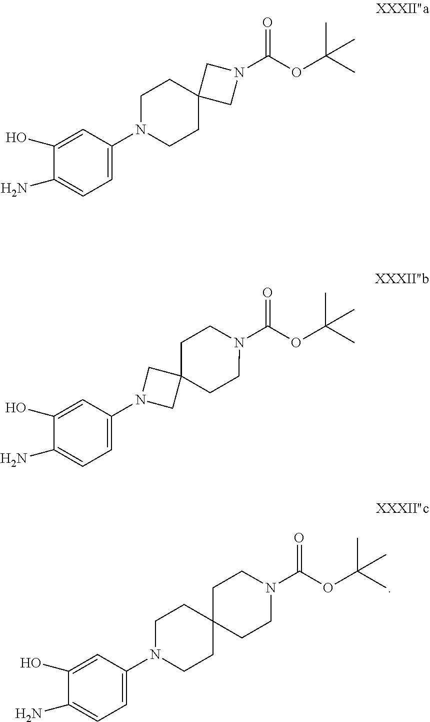 [SMILES: CC(C)(C)OC(=O)N1CC2(CCN(c3ccc(N)c(O)c3)CC2)C1 ; CC(C)(C)OC(=O)N1CCC2(CC1)CN(c1ccc(N)c(O)c1)C2 ; CC(C)(C)OC(=O)N1CCC2(CC1)CCN(c1ccc(N)c(O)c1)CC2]

The structures of the compounds of formula XXXIIIa-j are as shown below:

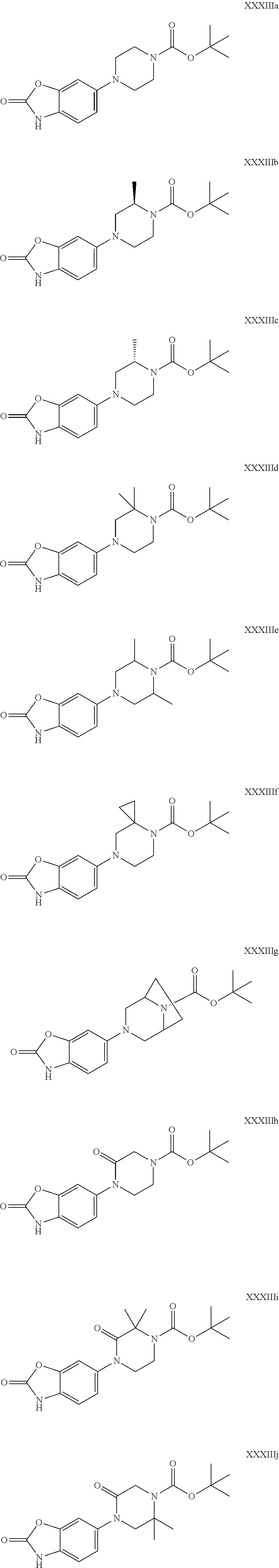

The structures of the compounds of formula XXXVa-1 are as shown below:

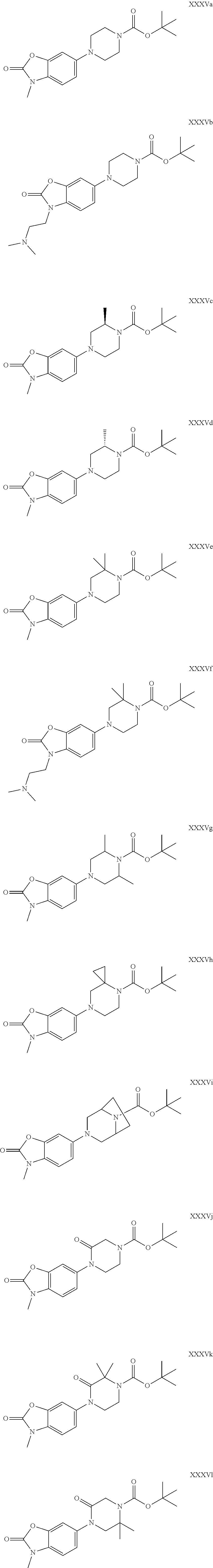

The structures of the compounds of formula XXXVIIa and XXXVIIIa are as shown below:

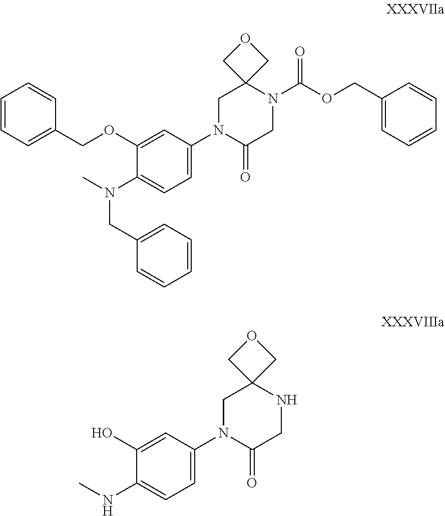 [SMILES: CN(Cc1ccccc1)c1ccc(N2CC3(COC3)N(C(=O)OCc3ccccc3)CC2=O)cc1OCc1ccccc1 ; CNc1ccc(N2CC3(COC3)NCC2=O)cc1O]

The structures of the compounds of formula XXXVIa-c, XXXIV′a-c, and XXXV′a-c are as shown below:

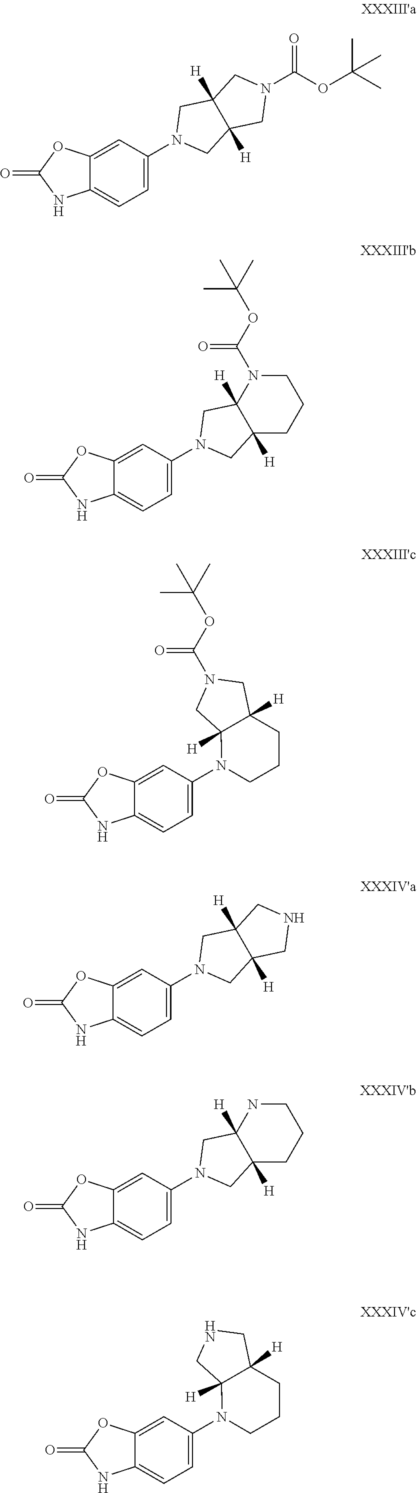 [SMILES: CC(C)(C)OC(=O)N1C[C@@H]2CN(c3ccc4[nH]c(=O)oc4c3)C[C@@H]2C1 ; CC(C)(C)OC(=O)N1CCC[C@@H]2CN(c3ccc4[nH]c(=O)oc4c3)C[C@@H]21 ; CC(C)(C)OC(=O)N1C[C@@H]2CCCN(c3ccc4[nH]c(=O)oc4c3)[C@@H]2C1 ; O=c1[nH]c2ccc(N3C[C@H]4CNC[C@H]4C3)cc2o1 ; O=c1[nH]c2ccc(N3C[C@H]4CCCN[C@H]4C3)cc2o1 ; O=c1[nH]c2ccc(N3CCC[C@H]4CNC[C@H]43)cc2o1]

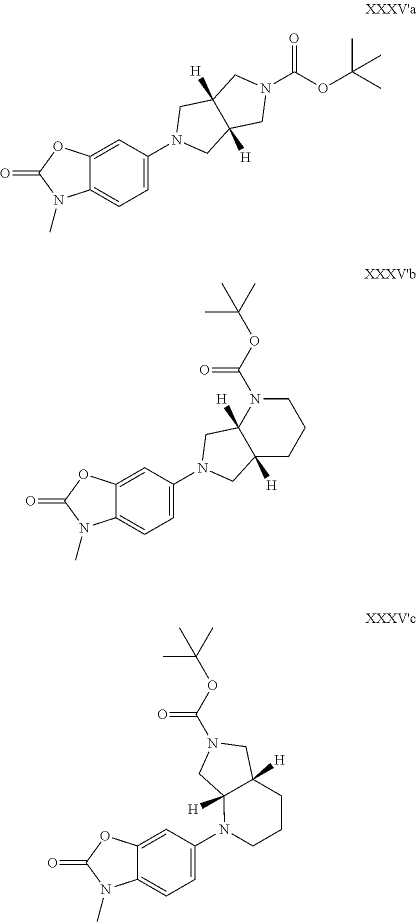 [SMILES: Cn1c(=O)oc2cc(N3C[C@H]4CN(C(=O)OC(C)(C)C)C[C@H]4C3)ccc21 ; Cn1c(=O)oc2cc(N3C[C@H]4CCCN(C(=O)OC(C)(C)C)[C@H]4C3)ccc21 ; Cn1c(=O)oc2cc(N3CCC[C@H]4CN(C(=O)OC(C)(C)C)C[C@H]43)ccc21]

The structures of the compounds of formula XXXIII″a-c and XXXV″a-c are as shown below:

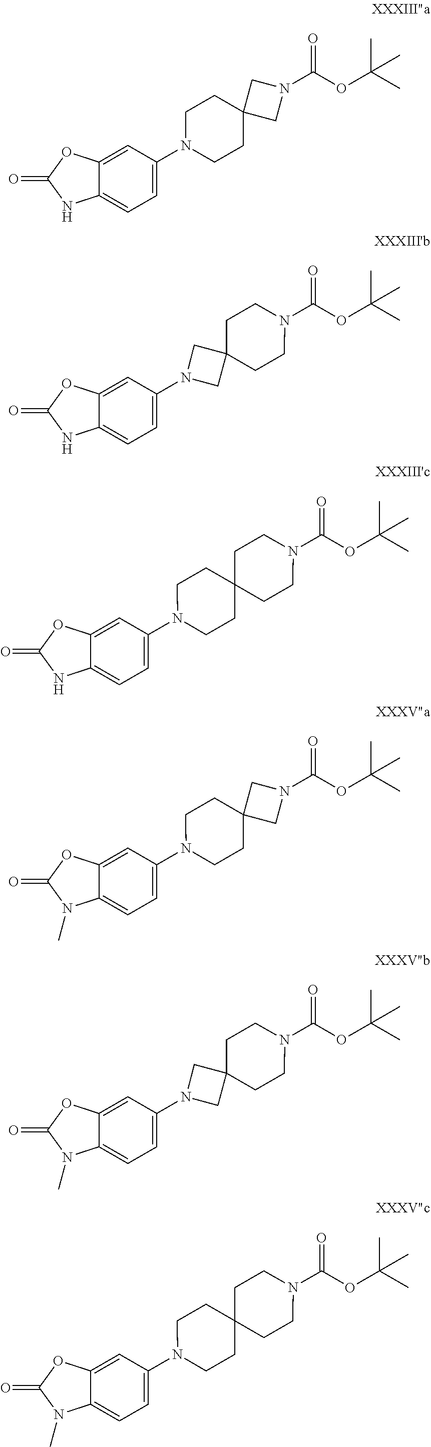 [SMILES: CC(C)(C)OC(=O)N1CC2(CCN(c3ccc4[nH]c(=O)oc4c3)CC2)C1 ; CC(C)(C)OC(=O)N1CCC2(CC1)CN(c1ccc3[nH]c(=O)oc3c1)C2 ; CC(C)(C)OC(=O)N1CCC2(CC1)CCN(c1ccc3[nH]c(=O)oc3c1)CC2 ; Cn1c(=O)oc2cc(N3CCC4(CC3)CN(C(=O)OC(C)(C)C)C4)ccc21 ; Cn1c(=O)oc2cc(N3CC4(CCN(C(=O)OC(C)(C)C)CC4)C3)ccc21 ; Cn1c(=O)oc2cc(N3CCC4(CCN(C(=O)OC(C)(C)C)CC4)CC3)ccc21]

The structures of the compounds of formula XXXVIa-m are as shown below:

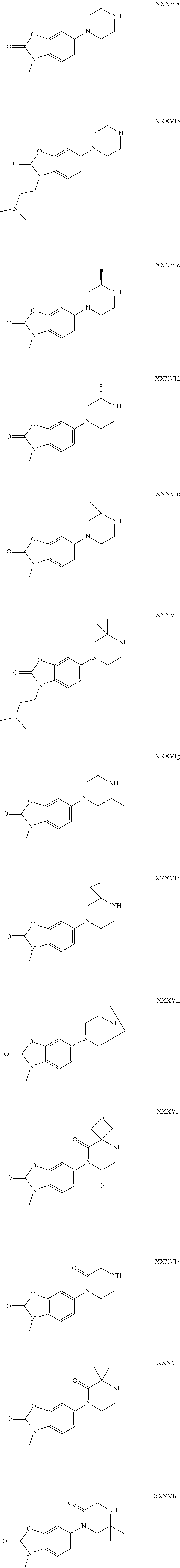

The structures of the compounds of formula XXXVI′a-c and XXXVI″a-c are as shown below:

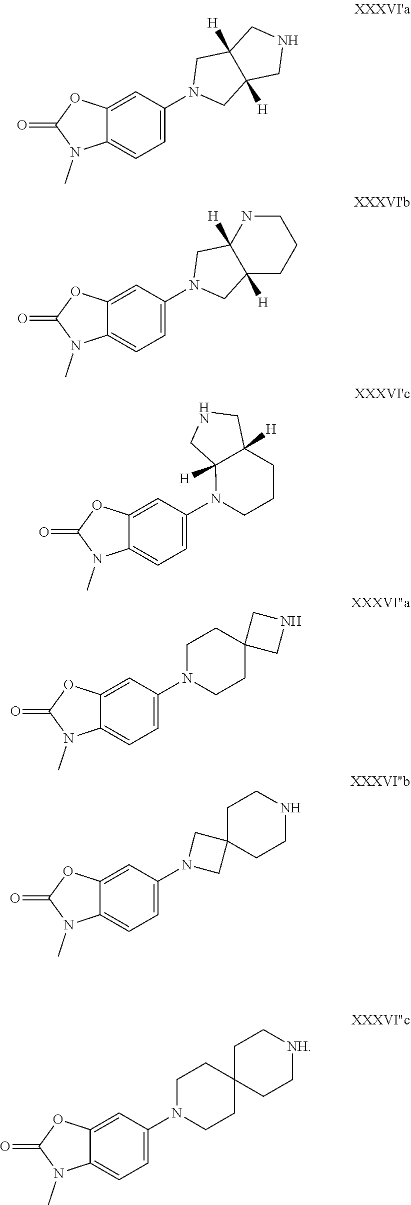 [SMILES: Cn1c(=O)oc2cc(N3C[C@H]4CNC[C@H]4C3)ccc21 ; Cn1c(=O)oc2cc(N3C[C@H]4CCCN[C@H]4C3)ccc21 ; Cn1c(=O)oc2cc(N3CCC[C@H]4CNC[C@H]43)ccc21 ; Cn1c(=O)oc2cc(N3CCC4(CC3)CNC4)ccc21 ; Cn1c(=O)oc2cc(N3CC4(CCNCC4)C3)ccc21 ; Cn1c(=O)oc2cc(N3CCC4(CCNCC4)CC3)ccc21]

### Part II—Preparation of Specific N-Heterocyclic Compounds

Exemplary procedures for preparing specific N-heterocyclic carboxamides are provided below. The following examples describe the multistep synthesis of imidazole carboxamides and intermediates. The various steps, including the synthesis of intermediates, are discussed in more detail below.

### Example 1: 4-(2-Oxo-3H-1,3-benzoxazol-6-yl)-N-(4-phenylbutyl)piperidine-1-carboxamide

### tert-Butyl 4-(3-hydroxy-4-nitrophenyl)-3,6-dihydro-2H-pyridine-1-carboxylate (VIIb)

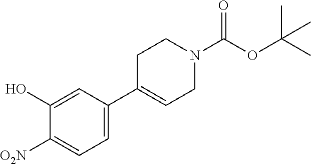 [SMILES: CC(C)(C)OC(=O)N1CC=C(c2ccc([N+](=O)[O-])c(O)c2)CC1]

Following general procedure A (step 3), VIa (2.5 g, 8.07 mmol) and 5-bromo-2-nitrophenol (1.6 g, 7.34 mmol) afforded V11b as a yellow solid (1.93 g, 82%). ¹H NMR (400 MHz, DMSO-d₆) δ 10.89 (s, 1H), 7.91 (d, J=8.6 Hz, 1H), 7.18-7.00 (m, 2H), 6.36 (bs, 1H), 4.04 (d, J=2.9 Hz, 2H), 3.54 (t, J=5.7 Hz, 2H), 2.49-2.39 (m, 2H), 1.43 (s, 9H) UPLC/MS (method B): Rt 1.48 min. MS (ES) C₁₆H₂₀N₂O₅ requires 320, found 321 [M+H]⁺.

### tert-Butyl 4-(4-amino-3-hydroxy-phenyl)piperidine-1-carboxylate (VIIIb)

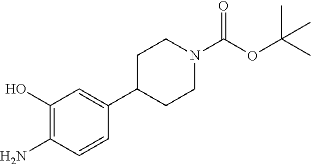 [SMILES: CC(C)(C)OC(=O)N1CCC(c2ccc(N)c(O)c2)CC1]

Following general procedure B (method A), VIIb (0.250 g, 0.780 mmol) afforded VIIIb which was used in the next step without further purification. UPLC/MS (method A): Rt 1.98 min. MS (ES) C₁₆H₂₄N₂O₃ requires 292, found 293 [M+H]⁺.

### tert-Butyl 4-(4-amino-3-hydroxyphenyl)piperidine-1-carboxylate (IXb)

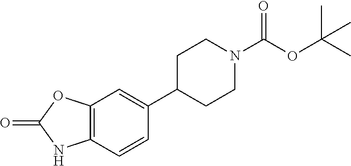 [SMILES: CC(C)(C)OC(=O)N1CCC(c2ccc3[nH]c(=O)oc3c2)CC1]

Following general procedure B, (method A, step 2), VIb (4.2 g, 13.11 mmol) afforded IXb as a white solid (3.9 g, 95%). UPLC/MS (method A): Rt 2.17 min. MS (ES) C₁₇H₂₂N₂O₄ requires 318, found 319 [M+H]⁺.

### 6-(4-Piperidyl)-3H-1,3-benzoxazol-2-one hydrochloride (Xa)

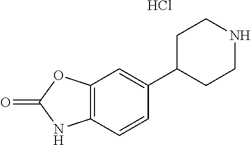 [SMILES: Cl ; O=c1[nH]c2ccc(C3CCNCC3)cc2o1]

Following general procedure C (step 2), IXb (3.9 g, 12.45 mmol) afforded Xa as a white solid (3.1 g, 99%). UPLC/MS (method A): Rt 0.91 min. MS (ES) C₁₂H₁₄N₂O₂ requires 218, found 219 [M+H]⁺.

### 4-(2-Oxo-3H-1,3-benzoxazol-6-yl)-N-(4-phenylbutyl)piperidine-1-carboxamide

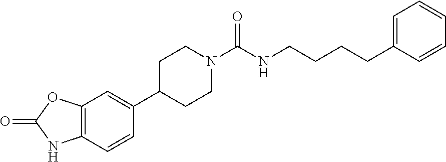 [SMILES: O=C(NCCCCc1ccccc1)N1CCC(c2ccc3[nH]c(=O)oc3c2)CC1]

Following general procedure D (Method A), Xa (0.280 g, 1.28 mmol). and 4-phenylbutyl isocyanate (0.247 g, 1.41 mmol) afforded the title compound as a white solid (0.050 g, 16%). ¹H NMR (400 MHz, CDCl₃) δ 9.48 (bs, 1H), 7.31-7.26 (m, 2H), 7.24-7.14 (m, 3H), 7.11-6.91 (m, 3H), 4.63 (br s, 1H), 4.08 (d, J=12.9 Hz, 2H), 3.40-3.21 (m, 2H), 2.90 (t, J=12.6 Hz, 2H), 2.78-2.58 (m, 3H), 1.94-1.83 (m, 2H), 1.74-1.54 (m, 6H) UPLC/MS (method A): Rt 2.30 min. MS (ES) C₂₃H₂₇N₃O₃ requires 393, found 394 [M+H]⁺.

### Example 2: 4-(3-Methyl-2-oxo-1,3-benzoxazol-6-yl)-N-(4-phenylbutyl)piperidine-1-carboxamide

### Benzyl 4-hydroxy-4-(2-oxo-3H-1,3-benzoxazol-6-yl)piperidine-1-carboxylate (XIIIa)

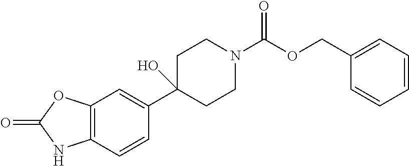 [SMILES: O=C(OCc1ccccc1)N1CCC(O)(c2ccc3[nH]c(=O)oc3c2)CC1]

Following general procedure G, Vb (1.0 g 4.49 mmol) and 6-bromo-3H-1,3-benzoxazol-2-one (0.4 g, 1.87 mmol) afforded XIIIa as a white solid (0.39 g, 57%). UPLC/MS (method A): Rt 1.83 min. MS (ES) C₂₀H₂₀N₂O₅ requires 368, found 369 [M+H]⁺.

### Benzyl 4-(2-oxo-3H-1,3-benzoxazol-6-yl)-3,6-dihydro-2H-pyridine-1-carboxylate (XIVa)

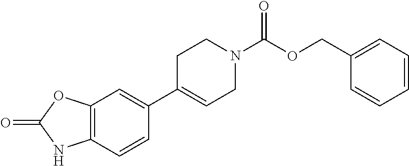 [SMILES: O=C(OCc1ccccc1)N1CC=C(c2ccc3[nH]c(=O)oc3c2)CC1]

To a solution of XIIIa (0.380 g, 1.03 mmol.) in THF (0.1 M) was added p-TsOH (0.191 g, 1.03 mmol) and the reaction mixture was stirred at reflux for 4b. Then, the reaction mixture was quenched with saturated aq. NaHCO₃ solution, extracted with EA, washed with brine, dried over Na₂SO₄ and concentrated to afford XIVa which was used in the next step without further purification (0.330 g, 91%). ¹H NMR (400 MHz, DMSO-d₆) δ 7.40-7.30 (m, 7H), 7.22 (dd, J=8.2, 1.7 Hz, 1H), 7.05 (d, J=8.2 Hz, 1H), 6.13 (bs, 1H), 5.12 (s, 2H), 4.15-4.05 (m, 2H), 3.65-3.55 (m, 2H). UPLC/MS (method A): Rt 2.15 min. MS (ES) C₂₀H₁₈N₂O₄ requires 350, found 351 [M+H]⁺.

### Benzyl 4-(3-methyl-2-oxo-1,3-benzoxazol-6-yl)-3,6-dihydro-2H-pyridine-1-carboxylate (XVa)

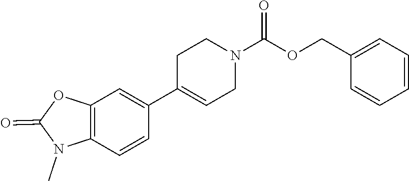 [SMILES: Cn1c(=O)oc2cc(C3=CCN(C(=O)OCc4ccccc4)CC3)ccc21]

Following general procedure C (step 1), XIVa (0355 g, 1.01 mmol) and CH₃I (0.215 g, 1.52 mmol) afforded XVa which was used in the next step without further purification. UPLC/MS (method A): Rt 2.31 min. MS (ES) C₂₁H₂₀N₂O₄ requires 364, found 365 [M+H]⁺.

### Benzyl 4-(2-oxo-3H-1,3-benzoxazol-6-yl)piperidine-1-carboxylate (XIIb)

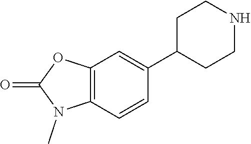 [SMILES: Cn1c(=O)oc2cc(C3CCNCC3)ccc21]

A suspension of XVa (0.360 g, 0.98 mmol, 1.0 eq.) in T1-F/MeOH (8:2, 0.1 M) was hydrogenated with the 1H-Cube apparatus using 10% Pd/C catalyst at RT. After complete conversion the solvent was evaporated under reduced pressure. The residue was used in the next step without further purification. ¹H NMR (400 MHz, DMSO-d₆) δ 7.21 (d, J=1.4 Hz, 1H), 7.15 (d, J=8.0 Hz, 1H), 7.09 (dd, J=8.1, 1.5 Hz, 1H), 3.32 (s, 3H), 3.30-3.10 (bs, 1H), 3.05-2.95 (m, 2H), 2.65-2.55 (m, 3H), 1.75-1.65 (m, 2H), 1.60-1.4) (m, 2H). UPLC/MS (method A): Rt 1.10 min. MS (ES) C₁₃H₁₆NO₂ requires 232, found 233 [M+H]⁺.

### 4-(3-Methyl-2-oxo-1,3-benzoxazol-6-yl)-N-(4-phenylbutyl)piperidine-1-carboxamide

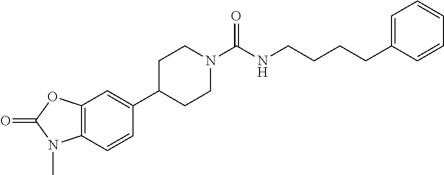 [SMILES: Cn1c(=O)oc2cc(C3CCN(C(=O)NCCCCc4ccccc4)CC3)ccc21]

Following general procedure D (Method A), XIIb (0.09 g, 0.39 mmol) and 4-phenylbutyl isocyanate (0.075 g, 0.43 mmol) afforded the title compound as a white solid (0.104 g, 65%). H NMR (400 MHz, CDCl₃) δ 7.35-7.25 (m, 2H), 7.25-7.15 (m, 3H), 7.07 (d, J=1.6 Hz, 1H), 7.05 (dd, J=8.0, 1.6 Hz, 1H), 6.91 (d, J=7.9 Hz, 1H), 4.80-4.40 (m, 1H), 4.15-4.05 (m, 1H), 3.40 (s, 3H), 3.30 (t, J=7.1 Hz, 2H), 2.89 (td, J=12.9, 2.6 Hz, 2H), 2.80-2.60 (m, 3H), 1.90-1.80 (m, 2H), 1.75-1.50 (m, 6H). UPLC/MS (method A): Rt 2.21 min. MS (ES) C₂₄H₂₉N₃O₃ requires 407, found 408 [M+H]⁺.

### Example 3: N-iso-Butyl 4-(3-methyl-2-oxo-1,3-benzoxazol-6-yl)piperidine-1-carboxamide

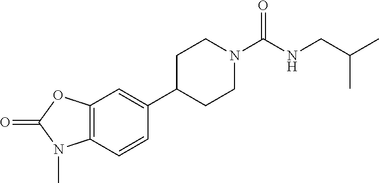 [SMILES: CC(C)CNC(=O)N1CCC(c2ccc3c(c2)oc(=O)n3C)CC1]

Following general procedure D (Method B), XIIb (0.063 g, 0.23 mmol) and isobutyl amine (0.050 g, 0.69 mmol) afforded the title compound as a white solid (0.058 g, 76%). ¹H NMR (400 MHz, CDCl₃) δ 7.05 (d, J=1.6 Hz, 1H), 7.03 (dd, J=8.1, 1.6 Hz, 1H), 6.90 (s, 1H), 4.70-4.50 (m, 1H), 4.15-4.05 (m, 1H), 3.38 (s, 3H), 3.30 (t, J=7.1 Hz, 2H), 2.88 (td, J=13.1, 2.6 Hz, 2H), 2.75-2.45 (m, 1H), 1.90-1.80 (m, 2H), 1.80-1.70 (m, 1H), 1.70-1.50 (m, 3H), 0.92 (d, J=6.7 Hz, 6H). UPLC/MS (method A): Rt 1.88 min. MS (ES) C₁₈H₂₅N₃O₃ requires 331, found 332 [M+H]⁺.

### Example 4: N-(2-Cyclopropylethyl)-4-(3-methyl-2-oxo-1,3-benzoxazol-6-yl)piperidine-1-carboxamide

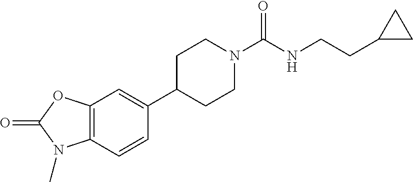 [SMILES: Cn1c(=O)oc2cc(C3CCN(C(=O)NCCC4CC4)CC3)ccc21]

Following general procedure D (Method C), XIIb (0.030 g, 0.064 mmol) and 2-cyclopropylethanamine hydrochloride (0.020 g, 0.168 mmol) afforded the title compound as a white solid (0.011 g, 50%). ¹H NMR (400 MHz, CDCl₃) δ 7.11-7.03 (m, 2H), 6.91 (d, J=8.0 Hz, 1H), 4.85 (br s, 1H), 4.13-4.01 (m, 2H), 3.41 (s, 3H), 3.38 (t, J=7.0 Hz, 2H), 2.93 (td, J=12.9, 2.6 Hz, 2H), 2.73 (tt, J=12.2, 3.6 Hz, 1H), 1.95-1.85 (m, 2H), 1.80-1.63 (m, 3H), 1.47 (q, J=7.0 Hz, 2H), 0.78-0.65 (m, 1H), 0.54-0.45 (m, 2H), 0.15-0.08 (m, 2H). UPLC/MS (method A): Rt 1.82 min. MS (ES) C₁₉H₂₅N₃O₃ requires 343, found 344 [M+H]⁺.

### Example 5: N-Cyclohexyl-4-(3-methyl-2-oxo-1,3-benzoxazol-6-yl)piperidine-1-carboxamide

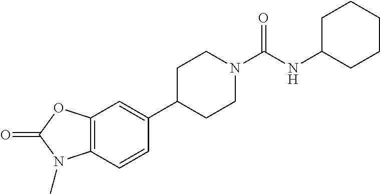 [SMILES: Cn1c(=O)oc2cc(C3CCN(C(=O)NC4CCCCC4)CC3)ccc21]

Following general procedure D (Method A), XIIb (0.050 g, 0.19 mmol) and cyclohexyl isocyanate (0.026 g, 0.21 mmol) afforded the title compound as a white solid (0.045 g, 68%). ¹H NMR (400 MHz, CDCl₃) δ 7.17-7.00 (m, 2H), 6.91 (d, J=7.9 Hz, 1H), 4.44 (bs, 1H), 4.08 (d, J=13.0 Hz, 2H), 3.79-3.59 (m, 1H), 3.41 (s, 3H), 2.89 (t, J=12.0 Hz, 2H), 2.71 (tt, J=12.2, 3.7 Hz, 1H), 2.07-1.94 (m, 2H), 1.93-1.81 (m, 2H), 1.78-1.59 (m, 5H), 1.49-1.32 (m, 2H), 1.26-1.05 (m, 3H). UPLC/MS (method A): Rt 1.99 min. MS (ES) C₂₀H₂₇N₃O₃ requires 357, found 358 [M+H]⁺.

### Example 6: 4-[3-(I-Methyl-4-piperidyl)-2-oxo-1,3-benzoxazol-6-y]-N-(4-phenylbutyl)piperidine-1-carboxamide

### tert-Butyl 4-[3-hydroxy-4-[(1-methyl-4-piperidyl)amino]phenyl]piperidine-1-carboxylate

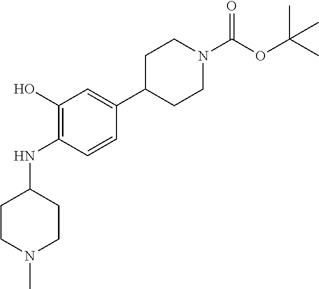 [SMILES: CN1CCC(Nc2ccc(C3CCN(C(=O)OC(C)(C)C)CC3)cc2O)CC1]

To a solution of VIIb (0.228 g, 0.78 mmol) in DCE (0.2 M), NaOAc (0.032 g, 0.39 mmol), glacial AcOH (0.02 mL, 039 mmol), N-methyl-4-piperidone (0.132 g, 1.17 mmol) and NaBH(AcO)₃ (0.25 g, 1.17 mmol) were added and the mixture was stirred at RT for 2 h and then diluted with EA, washed with saturated aq. NaHCO₃ solution, brine and dried over Na₂SO₄. After evaporation of the solvent, the residue was used in the next step without further purification. UPLC/MS (method A): Rt 1.83 min. MS (ES) C₂₂H₃₅N₃O₃ requires 389, found 390 [M+H]⁺.

### tert-Butyl 4-[3-(1-methyl-4-piperidyl)-2-oxo-1,3-benzoxazol-6-yl]piperidine-1-carboxylate (XIc)

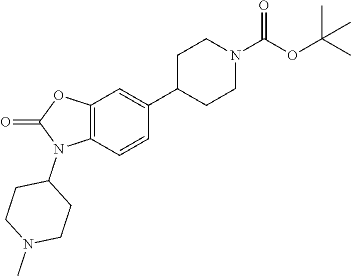 [SMILES: CN1CCC(n2c(=O)oc3cc(C4CCN(C(=O)OC(C)(C)C)CC4)ccc32)CC1]

Following general procedure B (step 2), tert-butyl 4-[3-hydroxy-4-[(1-methyl-4-piperidyl)amino]phenyl]piperidine-1-carboxylate (0.220 g, 0.780 mmol) afforded XIc as a white solid (0.170 g, 52%). ¹H NMR (400 MHz, CDCl₃) δ 7.33-7.27 (m, 1H), 7.08 (d, J=1.6 Hz, 1H), 7.02 (dd, J=8.2, 1.7 Hz, 1H), 4.40-4.16 (m, 3H), 3.13 (d, J=11.5 Hz, 2H), 2.91-2.75 (m, 2H), 2.73-2.64 (m, 1H), 2.57-2.48 (m, 2H), 2.44 (s, 3H), 2.37-2.20 (m, 2H), 1.98-1.77 (m, 4H), 1.68-1.53 (m, 2H), 1.50 (s, 9H). UPLC/MS (method A): Rt 1.98 min. MS (ES) C₂₃H₃₃N₃O₄ requires 415, found 416 [M+H]⁺.

### 3-(1-Methyl-4-piperidyl)-6-(4-piperidyl)-1,3-benzoxazol-2-one dihydrochloride (XIIc)

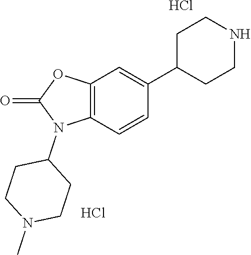 [SMILES: Cl ; CN1CCC(n2c(=O)oc3cc(C4CCNCC4)ccc32)CC1 ; Cl]

Following general procedure C (step 2), XIc (0.170 g, 0.409 mmol) afforded XIIc as a brownish solid (0.158 g, 990). ¹H NMR (400 MHz, DMSO-d₆) δ 11.41 (s, 1H), 9.35-8.85 (m, 2H), 7.75 (d, J=8.2 Hz, 1H), 7.24 (d, J=1.5 Hz, 1H), 7.08 (dd, J=8.3, 1.6 Hz, 1H), 4.47 (tt, J=12.4, 4.3 Hz, 1H), 3.56-3.47 (m, 2H), 3.35-3.29 (m, 2H), 3.20 (dt, J=13.8, 10.8 Hz, 2H), 3.04-2.84 (m, 3H), 2.86-2.69 (m, 5H), 2.06-1.95 (m, 2H), 1.96-1.85 (m, 4H). UPLC/MS (method A): Rt 1.14 min. MS (ES) C₁₃H₁₆N₂O₂ requires 315, found 316 [M+H]⁺.

### 4-[3-(1-Methyl-4-piperidyl)-2-oxo-1,3-benzoxazol-6-yl]-N-(4-phenylbutyl)piperidine-1-carboxamide

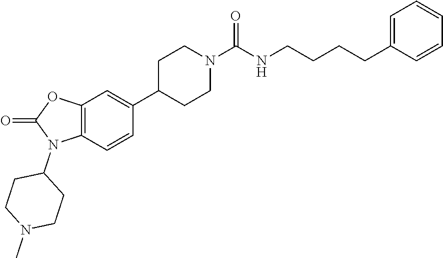 [SMILES: CN1CCC(n2c(=O)oc3cc(C4CCN(C(=O)NCCCCc5ccccc5)CC4)ccc32)CC1]

Following general procedure D (Method A), XIIc (0.060 g, 0.16 mmol) and 4-phenylbutyl isocyanate (0.029 g, 0.17 mmol) afforded the title compound as a white solid (0.035 g, 46%). ¹H NMR (400 MHz, CDCl₃) δ 7.33-7.26 (m, 2H), 7.26-7.13 (m, 4H), 7.07 (s, 1H), 7.00 (d, J=8.2 Hz, 1H), 4.51 (s, 1H), 4.34-4.16 (m, 1H), 4.07 (d, J=13.1 Hz, 2H), 3.29 (q, J=6.6 Hz, 2H), 3.06 (d, J=11.5 Hz, 2H), 2.87 (t, J=12.1 Hz, 2H), 2.77-2.60 (m, 3H), 2.54-2.33 (m, 5H), 2.25-2.10 (m, 2H), 1.94-1.78 (m, 4H), 1.74-1.52 (m, 6H). UPLC/MS (method A): Rt 2.25 min. MS (ES) C₂₉H₃₈N₄O₃ requires 490, found 491 [M+H]⁺.

### Example 7: 4-(3-Methyl-2-oxo-1,3-benzoxazol-7-yl)-N-(4-phenylbutyl)piperidine-1-carboxamide

### tert-Butyl 4-hydroxy-4-(2-oxo-3H-1,3-benzoxazol-7-yl)piperidine-1-carboxylate (XIIIb)

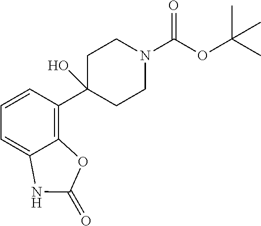 [SMILES: CC(C)(C)OC(=O)N1CCC(O)(c2cccc3[nH]c(=O)oc23)CC1]

Following general procedure G, Va (2.2 g, 11.21 mmol) and 7-bromo-3H-1,3-benzoxazol-2-one (1.0 g, 4.67 mmol) afforded XIIIb as a white solid (1.07 g, 68%). UPLC/MS (method A): Rt 1.75 min. MS (ES) C₁₇H₂₂N₂O₅ requires 334, found 335 [M+H]⁺.

### 7-(1,2,3,6-Tetrahydropyridin-4-yl)-3H-1,3-benzoxazol-2-one (XVIa)

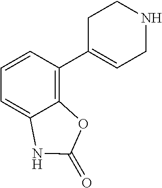 [SMILES: O=c1[nH]c2cccc(C3=CCNCC3)c2o1]

To a solution of XIIb (1.0 g, 3.21 mmol) in toluene (0.1 M) was added TFA (5.0 mL, 29.94 mmol) and the reaction mixture was stirred at reflux for 2 h. Then, the reaction mixture was quenched with saturated aq. NaHCO₃ solution, extracted with EA, washed with brine, dried over Na₂SO₄ and concentrated to afford XVIa which was used in the next step without further purification. UPLC/MS (method A): Rt 0.95 min. MS (ES) C₁₂H₁₂N₂O₂ requires 216, found 217 [M+H]⁺.

### tert-Butyl 4-(2-oxo-3H-1,3-benzoxazol-7-yl)-3,6-dihydro-2H-pyridine-1-carboxylate (XVIIa)

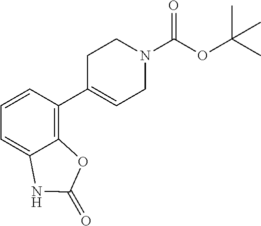 [SMILES: CC(C)(C)OC(=O)N1CC=C(c2cccc3[nH]c(=O)oc23)CC1]

To a solution of XVIa (0.172 g, 0.79 mmol) in THF (0.1 M) was added Boc₂O (0.190 g, 0.87 mmol in the presence of Et₃N (0.167 mL, 1.19 mmol) and the reaction mixture was stirred at RT for 1 h. Then, the reaction mixture was diluted with EA, washed with saturated aqueous NaHCO₃ solution, brine, dried over Na₂SO₄ and concentrated to afford XVIIa as a white solid (0.227 g, 90%). ¹H NMR (400 MHz, DMSO-d₆) δ 11.66 (s, 1H), 7.15-7.10 (m, 1H), 7.06 (dd, J=8.1, 1.4 Hz, 1H), 6.99 (dd, J=7.5, 1.3 Hz, 1H), 6.40-6.28 (m, 1H), 4.15-3.95 (m, 1H), 3.55 (t, J=5.7 Hz, 2H), 1.43 (s, 9H). UPLC/MS (method A): Rt 2.14 min. MS (ES) C₁₇H₂₀N₂O₁ requires 316, found 317 [M+H]⁺.

### tert-Butyl 4-(2-oxo-3H-1,3-benzooxazol-7-yl)piperidine-1-carboxylate (IXa)

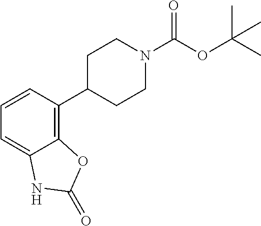 [SMILES: CC(C)(C)OC(=O)N1CCC(c2cccc3[nH]c(=O)oc23)CC1]

A suspension of XVIIa (0.225 g, 0.71 mmol, 1.0 eq.) in MeOH (0.1 M) was hydrogenated with the H-Cube apparatus using 10% Pd/C catalyst at RT and full H₂ mode. After complete conversion the solvent was evaporated under reduced pressure and the residue was used in the next step without further purification. 1H NMR (400 MHz DMSO-d₆) δ 11.57 (s, 1H), 7.08 (t, J=7.8 Hz, 1H), 6.96 (dd, J=8.3, 1.3 Hz, 1H), 6.93 (dd, J=7.6, 1.2 Hz, 1H), 4.20-3.95 (m, 2H), 3.10-2.70 (m, 3H), 1.80-1.70 (m, 2H), 1.68-1.52 (m, 2H), 142 (s, 9H). UPLC/MS (method A): Rt 2.17 min. MS (ES) C₁₇H₂₂N₂O₄ requires 318, found 319 [M+H]⁺.

### tert-Butyl 4-(3-methyl-2-oxo-1,3-benzoxazol-7-yl)piperidine-1-carboxylate (XIa)

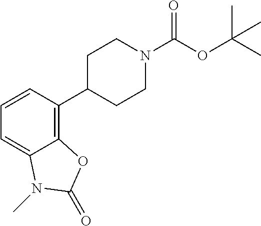 [SMILES: Cn1c(=O)oc2c(C3CCN(C(=O)OC(C)(C)C)CC3)cccc21]

Following general procedure C (step 1), IXa (0.219 g, 0.69 mmol) and MeI (0.18 g, 1.04 mmol) afforded XIa which was used in the next step without further purification. ¹H NMR (400 MHz, DMSO-d₆) δ 7.18 (t, J=7.8 Hz, 1H), 7.10 (dd, J=7.8, 1.3 Hz, 1H), 7.03 (dd, J=7.9, 1.3 Hz, 1H), 4.15-4.00 (m, 2H), 3.33 (s, 3H), 3.10-2.70 (m, 3H), 3.05-2.75 (m, 3H), 1.80-1.70 (m, 2H), 1.70-1.55 (m, 2H), 1.43 (s, 9H). UPLC/MS (method A): Rt 2.38 min. MS (ES) C₁₈H₂₄N₂O₄ requires 332, found 333 [M+H]⁺.

### 3-Methyl-7-(4-piperidyl)-1,3-benzoxazol-2-one hydrochloride (XIa)

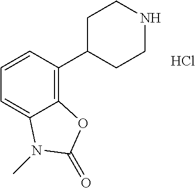 [SMILES: Cn1c(=O)oc2c(C3CCNCC3)cccc21 ; Cl]

Following general procedure C (step 2), XIa (0.212 g, 0.64 mmol) afforded XIIa which was used in the next step without further purification. ¹H NMR (400 MHz, DMSO-d₆) δ 9.09 (bs, 1H), 8.89 (bs, 1H), 7.22 (t, J=7.8 Hz, 1H), 7.15 (dd, J=7.8, 1.2 Hz, 1H), 6.99 (dd, J=7.9, 1.3 Hz, 1H), 3.40-3.35 (m, 1H), 3.34 (s, 3H), 3.20-3.10 (m, 1H), 3.10-3.95 (m, 2H), 2.10-1.95 (m, 4H). UPLC/MS (method A): Rt 112 min. MS (ES) C₁₃H₁₆N₂O₂ requires 232, found 233 [M+H]⁺.

### 4-(3-Methyl-2-oxo-1,3-benzoxazol-7-yl)-N-(4-phenylbutyl)piperidine-1-carboxamide

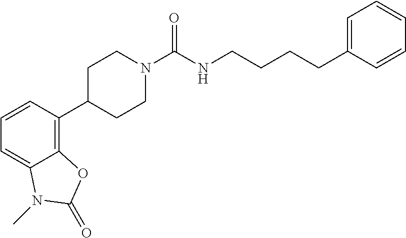 [SMILES: Cn1c(=O)oc2c(C3CCN(C(=O)NCCCCc4ccccc4)CC3)cccc21]

Following general procedure D (Method A), XIIa (0.080 g, 0.30 mmol) and 4-phenylbutyl isocyanate (0.033 g, 0.33 mmol) afforded the title compound as a white solid (0.045 g, 88%). ¹H NMR (400 MHz, CDCl₃) δ 7.35-7.25 (m, 2H), 7.24-7.11 (m, 4H), 6.97 (dd, J=8.0, 1.1 Hz, 1H), 6.86 (dd, J=7.8, 1.1 Hz, 1H), 4.65-4.45 (m, 1H), 4.15-4.00 (m, 2H), 3.42 (s, 3H), 3.32-3.25 (m, 2H), 3.15-3.05 (m, 1H), 3.00-2.85 (m, 2H), 2.75-2.65 (m, 2H), 200-1.85 (m, 2H), 1.85-1.65 (m, 6H), 1.65-1.50 (m, 2H). UPLC/MS (method A): Rt 2.27 min. MS (ES) C₂₄H₂₉N₃O₃ requires 407, found 408 [M+H]⁺.

### Example 8: N-iso-Butyl-4-(3-methyl-2-oxo-1,3-benzoxazol-7-yl)piperidine-1-carboxamide

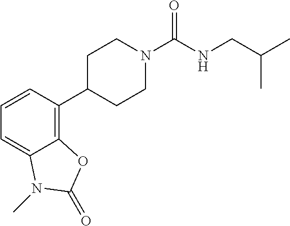 [SMILES: CC(C)CNC(=O)N1CCC(c2cccc3c2oc(=O)n3C)CC1]

Following general procedure D (Method B), using XIIa (0.080 g, 0.29 mmol) and isobutyl amine (0.064 g, 0.736 mmol) afforded the title compound as a white solid (0.065 g, 68%). ¹H NMR (400 MHz, CDCl₃) δ 7.14 (t, J=7.9 Hz, 1H), 6.99-6.91 (m, 1H), 6.83 (dd, 1=7.8, 1.1 Hz, 1H), 4.70-4.50 (m, 1H), 4.15-4.00 (m, 2H), 3.39 (s, 3H), 3.15-3.00 (m, 3H), 2.95-2.85 (m, 2H), 1.95-1.85 (m, 2H), 1.85-1.60 (m, 3H), 0.92 (d, J=6.7 Hz, 6H). UPLC/MS (method A): Rt 1.92 min. MS (ES) C₁₈H₂₅N₃O₃ requires 331, found 332 [M+H]⁺.

### Example 9: 4-(3-Methyl-2-oxo-1,3-benzoxazol-5-yl)-N-(4-phenylbutyl)piperidine-1-carboxamide

### tert-Butyl 4-(4-hydroxy-3-nitrophenyl)-3,6-dihydro-2H-pyridine-1-carboxylate (VIIc)

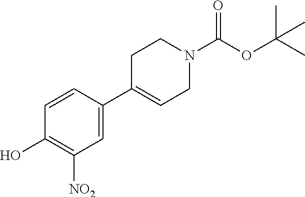 [SMILES: CC(C)(C)OC(=O)N1CC=C(c2ccc(O)c([N+](=O)[O-])c2)CC1]

Following general procedure A (step 3), VIa (0.92 g, 2.98 mmol) and 4-bromo-2-nitrophenol (0.50 g, 2.29 mmol) afforded VIIc as a yellow oil (0.68 g, 92%). ¹H NMR (400 MHz, CDCl₃) δ 10.56 (s, 1H), 8.09 (d, J=2.3 Hz, 1H), 7.67 (dd, J=8.8, 2.4 Hz, 1H), 7.16 (d, J=8.8 Hz, 1H), 6.10 (bs, 1H), 4.17-4.04 (m, 2H), 3.68 (t, J=5.7 Hz, 2H), 2.60-2.38 (m, 2H), 1.52 (s, 9H). UPLC/MIS (method B): Rt 1.05 min. MS (ES) C₁₆H₂₀N₂O₅ requires 320, found 321 [M+H]⁺.

### tert-Butyl 4-(3-amino-4-hydroxyphenyl)piperidine-1-carboxylate (VIIIc)

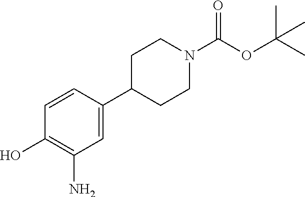 [SMILES: CC(C)(C)OC(=O)N1CCC(c2ccc(O)c(N)c2)CC1]

Following general procedure B (method A), VIIc (0300 g, 0.937 mmol) afforded VIIIc which was used in the next step without further purification. UPLC/MS (method A): Rt 1.98 min. MS (ES) C₁₆H₂₄N₂O₃ requires 292, found 293 [M+H]⁺.

### tert-Butyl 4-(2-oxo-3H-1,3-benzoxazol-5-y)piperidine-1-carboxylate (IXc)

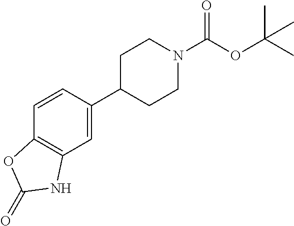 [SMILES: CC(C)(C)OC(=O)N1CCC(c2ccc3oc(=O)[nH]c3c2)CC1]

Following general procedure B (step 2), VIIIc (0.277 g, 0.95 mmol) afforded IXc as a colorless oil (0.220 g, 74%). ¹H NMR (400 MHz, CDCl₃) δ 8.11 (s, 1H), 7.15 (d, J=8.3 Hz, 1H), 6.97 (dd, J=8.4, 1.7 Hz, 1H), 6.92 (d, J=1.7 Hz, 1H), 4.28 (dd, J=10.6, 3.0 Hz, 2H), 2.82 (td, J=13.2, 2.6 Hz, 2H), 2.68 (tt, J=12.2, 3.6 Hz, 1H), 1.90-1.80 (m, 2H), 1.66-1.59 (m, 2H) 1.51 (s, 9H). UPLC/MS (method A): Rt 2.20 min. MS (ES) CH₁₇H₂₂N₂O₄ requires 318, found 319 [M+H]⁺.

### tert-Butyl 4-(3-methyl-2-oxo-1,3-benzoxazol-5-yl)piperidine-1-carboxylate (XId)

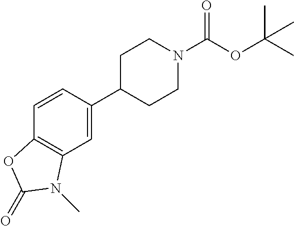 [SMILES: Cn1c(=O)oc2ccc(C3CCN(C(=O)OC(C)(C)C)CC3)cc21]

Following general procedure C (step 1), IXc (0.220 g, 0.691 mmol) and MeI (0.146 g, 1.037 mmol) afforded XId which was used in the next step without purification. ¹H NMR (400 MHz, CDCl₃) δ 7.14 (d, J=8.2 Hz, 1H), 6.97 (d, J=8.0 Hz, 1H), 6.82 (s, 1H), 4.29 (d, J=12.1 Hz, 2H), 3.41 (s, 3H), 2.83 (t, J=12.4 Hz, 2H), 2.77-2.63 (m, 1H), 1.95-1.78 (m, 2H), 1.75-1.57 (m, 2H), 1.51 (s, 9H). UPLC/MS (method B): Rt 1.20 min. NIS (ES) C₁₈H₂₄N₂O₄ requires 332, found 333 [M+H]⁺.

### 3-Methyl-5-(4-piperidyl)-1,3-benzoxazol-2-one hydrochloride (XIId)

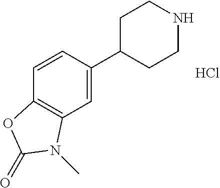 [SMILES: Cn1c(=O)oc2ccc(C3CCNCC3)cc21 ; Cl]

Following general procedure C (step 2), XId (0.210 g 0.632 mmol) afforded XIId as a brownish solid (0.160 g, 940%). ¹H NMR (400 MHz, DMSO-d₆) δ 9.41-8.89 (m, 2H), 7.29 (d, J=8.2 Hz, 1H), 7.11 (d, J=1.7 Hz, 1H), 6.99 (dd, J=8.3, 1.7 Hz, 1H), 3.40-3.35 (m, 2H), 3.34 (s, 3H), 3.08-2.81 (m, 3H), 2.01-1.81 (m, 4H). UPLC/MS (method A): Rt 1.14 min. MS (ES) C₁₃H₁₆N₂O₂ requires 232, found 233 [M+H]⁺.

### 4-(3-Methyl-2-oxo-1,3-benzoxazol-5-yl)-N-(4-phenylbutyl)piperidine-1-carboxamide

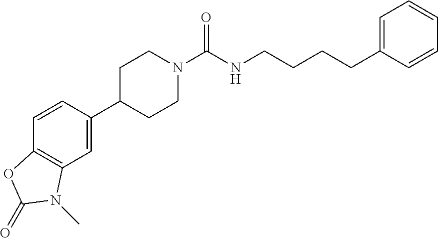 [SMILES: Cn1c(=O)oc2ccc(C3CCN(C(=O)NCCCCc4ccccc4)CC3)cc21]

Following general procedure D (method A), XIId (0.060 g, 0.19 mmol) and 4-phenylbutyl isocyanate (0.036 g, 0.21 mmol) afforded the title compound as a white solid (0.042 g, 54%). ¹H NMR (400 MHz, CDCl₃) δ 7.35-7.27 (m, 2H), 7.25-7.17 (m, 3H), 7.14 (d, J=8.2 Hz, 1H), 6.96 (dd, J=8.3, 1.7 Hz, 1H), 6.81 (d, J=1.7 Hz, 1H), 4.59 (br s, 1H), 4.08 (d, J=13.0 Hz, 2H), 3.41 (s, 3H), 3.31 (t, J=7.1 Hz, 2H), 2.91 (td, J=12.9, 2.6 Hz, 2H), 2.80-2.60 (m, 3H), 1.98-1.83 (m, 2H), 1.81-1.51 (m, 6H). UPLC/MS (method A): Rt 2.25 min. MS (ES) (24H29N₃O₃ requires 407, found 408 [M+H]⁺.

### Example 10: 4-(3-Methyl-2-oxo-1,3-benzooxazol-4-yl)-NV-(4-phenylbutyl)piperidine-1-carboxamide

### tert-Butyl 4-(3-hydroxy-2-nitrophenyl)-3,6-dihydro-2H-pyridine-1-carboxylate (VIId)

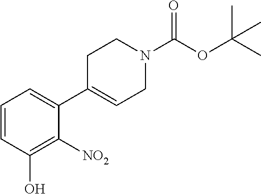 [SMILES: CC(C)(C)OC(=O)N1CC=C(c2cccc(O)c2[N+](=O)[O-])CC1]

Following general procedure A (step 3), VI′a (1.85 g, 5.96 mmol) and 3-bromo-2-nitrophenol (1.0 g, 4.59 mmol) afforded VIId as a pale yellow solid (1.42 g, 97%). ¹H NMR (400 MHz, CDCl₃) δ 10.20 (s, 1H), 7.46 (t, J=7.9 Hz, 1H), 7.11 (dd, J=8.5, 1.5 Hz, 1H), 6.78 (dd, J=7.5, 1.4 Hz, 1H), 5.56 (bs, 1H), 4.13-3.88 (m, 2H), 3.68 (t, J=5.5 Hz, 2H), 2.28 (s, 2H), 1.52 (s, 9H). UPLC/M S (method B): Rt 1.05 min. MS (ES) C₁₆H₂₀N₂O₅ requires 320, found 321 [M+H]⁺.

### tert-Butyl 4-(2-amino-3-hydroxy-phenyl)piperidine-1-carboxylate (VIIId)

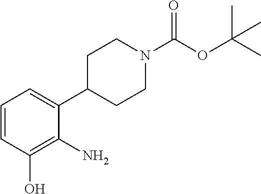 [SMILES: CC(C)(C)OC(=O)N1CCC(c2cccc(O)c2N)CC1]

Following general procedure 1B (method A), VIId (0.510 g, 1.56 mmol) afforded VIIId which was used in the next step without further purification. UPLC/MS (method A): Rt 2.01 min. MS (ES) C₁₆H₂₄N₂O₃ requires 292, found 293 [M+H]⁺.

### tert-Butyl 4-(2-oxo-3H-1,3-benzoxazol-4-yl)piperidine-1-carboxylate (IXd)

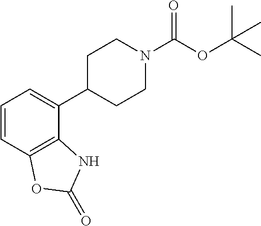 [SMILES: CC(C)(C)OC(=O)N1CCC(c2cccc3oc(=O)[nH]c23)CC1]

Following general procedure B (step 2), VIId (0.465 g, 1.59 mmol) afforded IXd as a white solid (0.190 g, 37%). ¹H NMR (400 MHz, CDCl₃) δ 10.53 (s, 1H), 7.14-7.09 (m, 2H), 7.07-7.00 (m, 1H), 4.31 (d, J=13.1 Hz, 2H), 3.05-2.74 (m, 3H), 1.90 (d, J=10.9 Hz, 2H), 1.79-1.62 (m, 2H), 1.52 (s, 9H). UPLC/MS (method A): Rt 2.24 min. MS (ES) C₁₇H₂₂N₂O₄ requires 318, found 336 [M+NH₄].

### tert-Butyl 4-(3-methyl-2-oxo-1,3-benzoxazol-4-yl)piperidine-1-carboxylate (XIe)

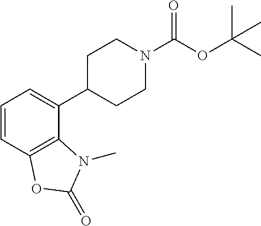 [SMILES: Cn1c(=O)oc2cccc(C3CCN(C(=O)OC(C)(C)C)CC3)c21]

Following general procedure C (step 1), IXd (0.16 g, 0.487 mmol) and MeI (0.10 g, 0.73 mmol) afforded XIe which used in the next step without further purification. ¹H NMR (400 MHz, CDCl₃) δ 7.15-6.99 (m, 3H), 4.33 (d, J=13.3 Hz, 2H), 3.67 (s, 3H), 3.29-3.16 (m, 1H), 2.90-2.78 (m, 2H), 1.92-1.70 (m, 4H), 1.51 (s, 9H). UPLC/MS (method B): Rt 1.20 min. MS (ES) C₁₈H₂₄N₂O₄ requires 332, found 333 [M+H]⁺.

### 3-Methyl-4-(4-piperidyl)-1,3-benzoxazol-2-one hydrochloride (XIIe)

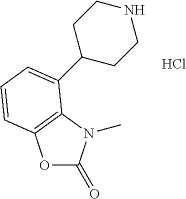 [SMILES: Cn1c(=O)oc2cccc(C3CCNCC3)c21 ; Cl]

Following general procedure C (step 2) XIe (0.154 g, 0.46 mmol) afforded XIIe as a white solid (0.120 g 97%). ¹H NMR (400 MHz, DMSO-d₆) δ 9.38-9.00 (m, 2H), 7.22 (dd, J=7.8, 1.2 Hz, 1H), 7.15 (t, J=7.9 Hz, 1H), 7.08 (dd, J=8.0, 1.3 Hz, 1H), 3.59 (s, 3H), 3.55-3.43 (m, 1H), 3.38-3.31 (m, 2H), 3.21-2.98 (m, 2H), 2.14-1.80 (m, 4H). UPLC/MS (method B): Rt 1.14 min. MS (ES) C₁₃H₃₆N₂O₂ requires 232, found 233 [M+H]⁺.

### 4-(3-Methyl-2-oxo-1,3-benzoxazol-4-yl)-N-(4-phenylbutyl)piperidine-1-carboxamide

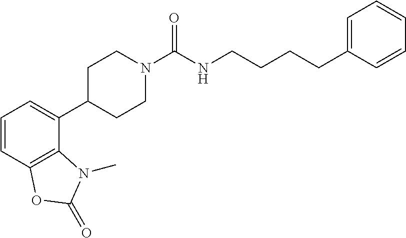 [SMILES: Cn1c(=O)oc2cccc(C3CCN(C(=O)NCCCCc4ccccc4)CC3)c21]

Following general procedure D (Method A), XIIe (0.050 g, 0.16 mmol) and 4-phenylbutyl isocyanate (0.030 g, 0.17 mmol) afforded the title compound as a white solid (0.057 g, 89%). ¹H NMR (400 MHz, CDCl₃) δ 7.55-6.79 (m, 9H), 4.54 (s, 1H), 4.13 (d, J=13.0 Hz, 2H), 3.66 (s, 3H), 3.423.12 (m, 3H), 2.91 (t, J=12.7 Hz, 2H), 2.68 (t, J=7.5 Hz, 2H), 2.00-1.46 (m, 9H). UPLC/MS (method A): Rt 2.27 min. MS (ES) C₂₄H₂₉N₃O₃ requires 407, found 408 [M+H]⁺.

### Example 11: 3-(2-Oxo-3H-1,3-benzoxazol-6-yl)-V-(4-phenylbutyl)piperidine-1-carboxamide

### 6-Piperidin-1-ium-3-yl-3H-1,3-benzoxazol-2-one hydrochloride (Xb)

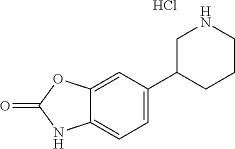 [SMILES: Cl ; O=c1[nH]c2ccc(C3CCCNC3)cc2o1]

Following general procedure C (step 2), IXe (0.192 g, 0.6 mmol) afforded Xb as a white solid (0.150 g, 98%) and used in the next step without further purification. UPLC/MS (method A): Rt 1.06 min. MS (ES) C₁₂H₁₅N₂O₂ requires 219, found 220 [M+H]⁺.

### 3-(2-Oxo-3H-1,3-benzoxazol-6-yl)-N-(4-phenylbutyl)piperidine-1-carboxamide

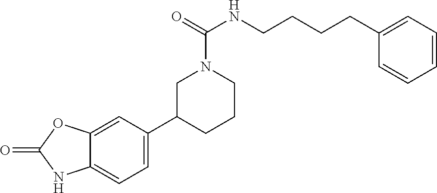 [SMILES: O=C(NCCCCc1ccccc1)N1CCCC(c2ccc3[nH]c(=O)oc3c2)C1]

Following general procedure D (Method A), Xb (0.06 g, 0.31 mmol) and 4-phenylbutyl isocyanate (0.05 g, 0.31 mmol) afforded the title compound as a white solid (0.060 g, 49%). ¹H NMR (400 MHz, DMSO-d₆) δ 11.51 (s, 1H), 7.30-7.11 (m, 6H), 7.06-6.95 (m, 2H), 6.46 (bs, 1H), 4.08-3.89 (m, 2H), 3.08-3.00 (m, 2H), 2.74-2.53 (m, 5H), 1.90-1.81 (m, 1H), 1.69-1.34 (m, 7H). UPLC/MS (method B): Rt 0.79 min. MS (ES) C₂₃H₂₇N₃O₃ requires 393, found 394 [M+H]⁺.

### Example 12: 3-(3-Methyl-2-oxo-1,3-benzoxazol-6-yl)-N-(4-phenylbutyl)piperidine-1-carboxamide

### tert-Butyl 5-(trifluoromethylsulfonyloxy)-3,6-dihydro-2H-pyridine-1-carboxylate (VIb)

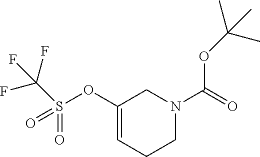 [SMILES: CC(C)(C)OC(=O)N1CCC=C(OS(=O)(=O)C(F)(F)F)C1]

Following general procedure A (step 1), Vc (1.0 g, 5.02 mmol) afforded VIb as a colorless oil (0.90 g, 54%). ¹H NMR (400 MHz, CDCl₃) δ 5.92 (t, =4.0 Hz, 1H), 4.13-3.83 (n, 2H), 3.49 (t, J=5.6 Hz, 2H), 2.38-2.09 (m, 2H), 1.47 s, 9H). UPLC/MS (method B): Rt 1.77 min. MS (ES) C₁₁H₁₆F₃NO₅S requires 331, found 332 [M+H]⁺.

### tert-Butyl 5-(3-hydroxy-4-nitrophenyl)-3,6-dihydro-2H-pyridine-1-carboxylate (VIIe)

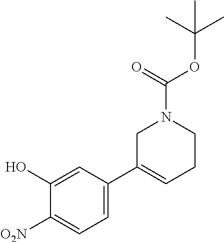 [SMILES: CC(C)(C)OC(=O)N1CCC=C(c2ccc([N+](=O)[O-])c(O)c2)C1]

Following general procedure A, VIb (0.90 g 2.7 mmol) and 5-bromo-2-nitrophenol (0.650 g, 3.27 mmol) afforded VIIe as a yellow solid (0.460 g, 55%). ¹H NMR (400 MHz, CDCl₃) δ 10.64 (s, 1H), 8.06 (d, J=8.9 Hz, 1H), 7.10 (s, 1H), 7.01 (dd, J=8.9, 2.0 Hz, 1H), 6.49-6.43 (m, 1H), 4.26 (s, 2H), 3.56 (t, J=5.7 Hz, 2H), 2.37 (s, 2H), 1.50 (s, 9H). UPLC/MS (method B): Rt 1.86 min. MS (ES) C₁₆H₂₀N₂O₅ requires 320, found 321 [M+H]⁺.

### tert-Butyl 3-(4-amino-3-hydroxyphenyl)piperidine-1-carboxylate (VIIIe)

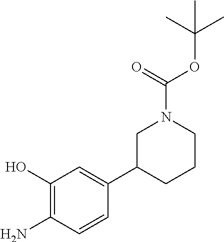 [SMILES: CC(C)(C)OC(=O)N1CCCC(c2ccc(N)c(O)c2)C1]

Following general procedure B (Method A), VIIe (0.45 g, 1.41 mmol) afforded VIIIe which was used in the next step without further purification. UPLC/MS (method B): Rt 0.73 min. MS (ES) C₁₆H₂₄N₂O₃ requires 292, found 293 [M+H]⁺.

### tert-Butyl 3-(2-oxo-3H-1,3-benzoxazol-6-yl)piperidine-1-carboxylate (IXe)

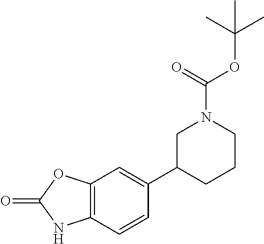 [SMILES: CC(C)(C)OC(=O)N1CCCC(c2ccc3[nH]c(=O)oc3c2)C1]

Following general procedure B (step 2), VIIe (0.270 g, 0.92 mmol) afforded IXe as a white solid (0.190 g, 64%). ¹H NMR (400 MHz, CDCl₃) δ 10.04 (s, 1H), 7.02 (s, 1H), 6.99-6.91 (m, 2H), 4.20-4.03 (m, 2H), 279-2.57 (m, 3H), 2.00-1.92 (m, 1H), 1.77-1.68 (m, 1H), 1.63-1.46 (m, 2H), 1.44 (s, 9H). UPLC/MS (method A): Rt 0.94 min. MS (ES) C₁₇H₂₂N₂O₄ requires 318, found 319 [M+H]⁺.

### tert-Butyl 3-(3-methyl-2-oxo-1,3-benzoxazol-6-yl)piperidine-1-carboxylate (XIf)

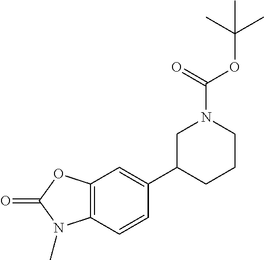 [SMILES: Cn1c(=O)oc2cc(C3CCCN(C(=O)OC(C)(C)C)C3)ccc21]

Following general procedure C (step 1), IXf (0.400 g, 1.25 mmol) and MeI (0.27 g, 1.88 mmol) afforded XIf which was used in the next step without purification, ¹H NMR (400 MHz, CDCl₃) δ 7.10-7.08 (m, 1H), 7.06 (dd, J=8.0, 1.5 Hz, 1H), 6.89 (d, J=8.0 Hz, 1H), 4.24-4.05 (m, 2H), 3.38 (s, 3H), 2.81-2.61 (m, 3H), 2.01 (d, J=9.2 Hz, 1H), 1.80-1.72 (m, 1H), 1.68-1.53 (m, 2H), 1.47 (s, 91H). UPLC/MS (method A): Rt 2.37 min. MS (ES) C₁₈H₂₄N₂O₄ requires 332, found 333 [M+H]⁺.

### 3-Methyl-6-piperidin-1-ium-3-yl-1,3-benzoxazol-2-one hydrochloride (XIIf)

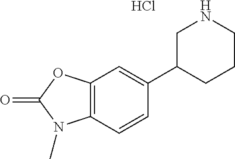 [SMILES: Cl ; Cn1c(=O)oc2cc(C3CCCNC3)ccc21]

Following general procedure C (step 2), XIf (0.340 g, 1.024 mmol) afforded XIIf as a white solid (0.260 g, 99%). ¹H NMR (400 MHz. DMSO-d₆) δ 9.16 (d, J=85.0 Hz, 2H), 7.33 (d, J=1.3 Hz, 1H), 7.22 (d, J=8.0 Hz, 1H), 7.15 (dd, J=8.1, 1.5 Hz, 1H), 3.31-3.20 (m, 2H), 3.09-2.96 (m, 2H), 2.94-2.77 (m, 1H), 1.93-1.61 (m, 41H). UPLC/MS (method A): Rt 1.16 min. MS (ES) C₁₃H₁₆N₂O₂ requires 233, found 234 [M+H]⁺.

### 3-(3-Methyl-2-oxo-1,3-benzoxazol-6-yl)-N-(4-phenylbutyl)piperidine-1-carboxamide

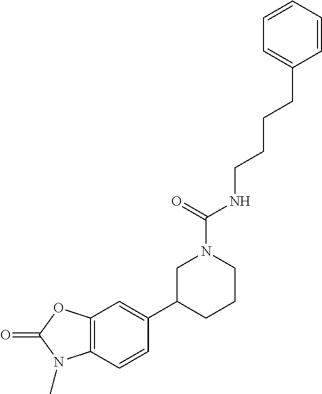 [SMILES: Cn1c(=O)oc2cc(C3CCCN(C(=O)NCCCCc4ccccc4)C3)ccc21]

Following general procedure D (Method A), XIIf (0.08 g, 0.32 mmol) and 4-phenylbutyl isocyanate (0.06 g, 0.37 mmol) afforded the title compound as a white solid (0.107 g, 79%). ¹H NMR (400 MHz, CDCl₃) δ 7.30-7.23 (m, 2H), 7.21-7.13 (m, 3H), 7.10-7.04 (m, 2H), 6.92-6.85 (i, 1H), 4.52 (bs, 1H), 4.11-3.98 (m, 1H), 3.88 (d, J=12.8 Hz, 1H), 3.38 (s, 3H), 3.26 (t, J=7.1 Hz, 2H), 2.88-2.78 (m, 1H), 2.78-2.69 (m, 2H), 2.64 (t, J=7.5 Hz, 2H), 2.13-1.98 (m, 1H), 1.79 (ddd, J=13.6, 8.5, 5.5 Hz, 1H), 1.72-1.49 (m, 6H). UPLC/MS (method A): Rt 2.29 min. MS (ES) C₂₄H₂₉N₃O₃ requires 407, found 408 [M+H]⁺.

### Example 13: 3-(3-Methy-2-oxo-1,3-benzoxazol-6-yl)-N-pentyl-piperidine-1-carboxamide

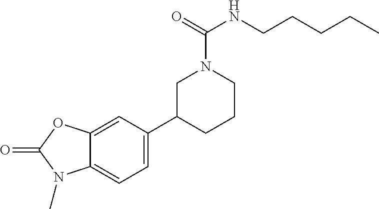 [SMILES: CCCCCNC(=O)N1CCCC(c2ccc3c(c2)oc(=O)n3C)C1]

Following general procedure D (Method A), XIIf (0.060 g, 0.22 mmol) and n-pentyl isocyanate (0.053 g, 0.45 mmol) afforded the title compound as white solid (0.040 g, 53%). ¹H NMR (400 MHz, CDCl₃) δ 7.11-7.08 (m, 2H), 6.89 (d, J=7.9 Hz, 1H), 4.60 (bs, 1H), 4.04 (t, J=9.3 Hz, 1H), 3.89 (d, J=13.1 Hz, 1H), 3.39 (s, 3H), 3.23 (t, J=7.2 Hz, 2H), 2.85 (td, J=12.8, 2.9 Hz, 1H), 2.80-2.71 (m, 2H), 2.11-1.98 (m, 1H), 1.89-1.57 (m, 3H), 1.51 (p, J=7.3 Hz, 2H), 1.32 (tp, J=7.2, 4.2, 3.5 Hz, 4H), 0.90 (t, J=6.9 Hz, 3H). UPLC/MS (method A): Rt 2.29 min. MS (ES) C₁₉H₂₇N₃O₃ requires 345, found 346 [M+H]⁺.

### Example 14: N-(2-Ethoxyethyl)-3-(3-methyl-2-oxo-1,3-benzoxazol-6-yl)piperidine-1-carboxamide

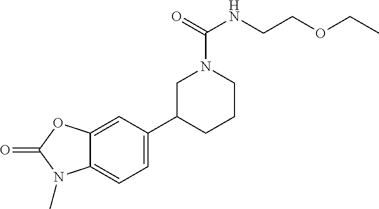 [SMILES: CCOCCNC(=O)N1CCCC(c2ccc3c(c2)oc(=O)n3C)C1]

Following general procedure D (Method A), XIIf (0.090 g, 0.33 mmol) and 1-ethoxy-2-isocyanate (0.090 g, 0.99 mmol) afforded the title compound as a white powder (0.015 g, 13%). ¹H NMR (400 MHz, CDCl₃) δ 7.14-7.04 (m, 2H), 6.89 (d, J=7.9 Hz, 1H), 3.75 (t, J=14.7 Hz, 2H), 3.39 (s, 3H), 3.22 (qd, J=7.0, 2.3 Hz, 4H), 2.88-2.78 (m, 2H), 2.78-2.67 (m, 1H), 2.09-1.97 (m, 1H), 1.89-1.74 (m, 1H), 1.72-1.54 (m, 2H), 1.13 (t, J=7.1 Hz, 6H). UPLC/MS (method A): Rt 1.66 min. MS (ES) C₁₈H₂₅N₃O₄ requires 347, found 348 [M+H]⁺.

### Example 15: 5-(3-methyl-2-oxo-1,3-benzoxazol-6-yl)-N-(4-phenylbutyl)-3,6-dihydro-2H-pyridine-1-carboxamide

### tert-Butyl 3-hydroxy-3-(2-oxo-3H-1,3-benzoxazol-6-yl)piperidine-1-carboxylate (XIIIc)

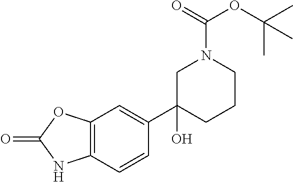 [SMILES: CC(C)(C)OC(=O)N1CCCC(O)(c2ccc3[nH]c(=O)oc3c2)C1]

Following general procedure G, Vc (2.52 g, 12.63 mmol) and 6-bromo-3H-1,3-benzoxazol-2-one (2.46 g, 12.34 mmol) afforded XIIIc as a white solid (0.950 g, 69%). ¹H NMR (400 MHz, DMSO-d₆) δ 11.54 (bs, 1H), 7.41 (d, J=1.5 Hz, 1H), 7.30 (dd, J=8.2, 1.6 Hz, 1H), 7.04 (d, J=8.2 Hz, 1H), 5.00 (s, 1H), 3.83-3.48 (m, 2H), 3.04-2.84 (m, 1H), 1.97-1.60 (m, 4H) 1.39 (s, 9H), 0.86 (t, J=6.8 Hz, 1H). UPLC/MS (method A): Rt 1.76 min. MS (ES) C₁₇H₂₂N₂O₅ requires 334, found 333 [M−H]⁻.

### 6-(1,2,3,6-Tetrahydropyridin-5-yl)-3H-1,3-benzoxazol-2-one (XVIb)

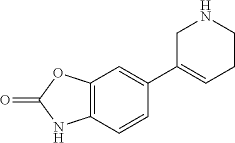 [SMILES: O=c1[nH]c2ccc(C3=CCCNC3)cc2o1]

To a solution of XIIIc (0.93 g, 2.69 mmol) in toluene (0.1 M) p-TsOH (0.850 g, 4.49 mmol) was added and the reaction mixture was stirred at reflux for 1 h. Then, the reaction mixture was quenched with saturated aqueous NaHCO₃ solution, extracted with EA, washed with brine, dried over Na₂SO₄ and concentrated to afford XVIb which was used in the next step without purification. UPLC/MS (method A): Rt 0.83 min. MS (ES) C₁₂H₁₂N₂O₂ requires 216, found 215 [M−H]⁻.

### tert-Butyl 5-(2-oxo-3H-1,3-benzoxazol-6-yl)-3,6-dihydro-2H-pyridine-1-carboxylate (XVIIb)

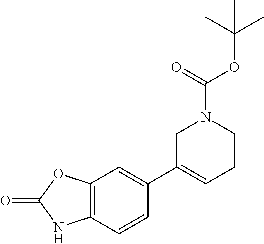 [SMILES: CC(C)(C)OC(=O)N1CCC=C(c2ccc3[nH]c(=O)oc3c2)C1]

To a solution of XVIb (0.580 g, 2.69 mmol) and Et₃N (1.50 mL, 10.76 mmol in DCM (0.1M) Boc₂O (0.590 g, 3.0 mmol) was added and the reaction mixture was stirred at RT for 10 min. Then, the reaction mixture was diluted with EA, washed with saturated aq. NaHCO₃ solution, brine, dried over Na₂SO₄ and concentrated to afford example XVIIb as a white solid (0.340 g, 40%). ¹H NMR (400 MHz, CDCl₃) δ 8.60 (bs, 1H), 7.22 (s, 1H), 7.18-7.11 (m, 1H), 7.00 (d, J=8.2 Hz, 1H), 6.31-5.82 (m, 1H), 4.31-4.20 (m, 2H), 3.55 (t, J=5.8 Hz, 2H), 2.32 (d, J=4.0 Hz, 2H), 1.50 (s, 9H). UPLC/MS (method A): Rt 2.13 min. MS (ES) C₁₇H₂₀N₂O₄ requires 316, found 317 [M+H]⁺.

### tert-Butyl 5-(3-methyl-2-oxo-1,3-benzoxazol-6-yl)-3,6-dihydro-2H-pyridine-1-carboxylate (XVII′a)

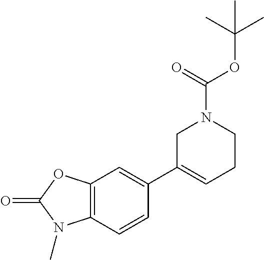 [SMILES: Cn1c(=O)oc2cc(C3=CCCN(C(=O)OC(C)(C)C)C3)ccc21]

Following general procedure C, XVII′ (0.06 g, 0.24 mmol) and MeI (0.05 g, 0.36 mmol) afforded XVII′a which was used in the next step without purification. UPLC/MS (method A): Rt 2.29 min. MS (ES) C₁₈H₂₂N₂O₄ requires 330, found 331 [M+H]⁺.

### 3-Methyl-6-(1,2,3,6-tetrahydropyridin-5-yl)-1,3-benzoxazol-2-one hydrochloride (XVI″a)

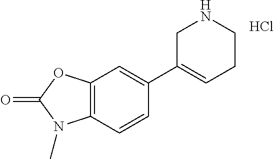 [SMILES: Cn1c(=O)oc2cc(C3=CCCNC3)ccc21 ; Cl]

Following general procedure C, XVII′ (0.058 g, 0.17 mmol) afforded XVI″a, which was used in the next step without further purification. UPLC/MS (method A): Rt 1.16 min. MS (ES) C₁₃H₁₄N₂O₂ requires 230, found 231 [M+H]⁺.

### 5-(3-Methyl-2-oxo-1,3-benzoxazol-6-yl)-N-(4-phenylbutyl)-3,6-dihydro-2H-pyridine-1-carboxamide

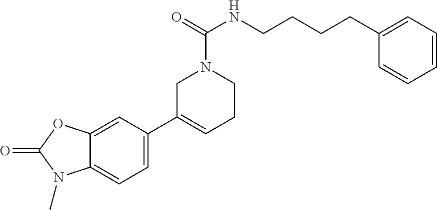 [SMILES: Cn1c(=O)oc2cc(C3=CCCN(C(=O)NCCCCc4ccccc4)C3)ccc21]

Following general procedure D (Method A), XVI″ (0.047 g, 0.17 mmol) and 4-phenylbutyl isocyanate (0.06 g, 0.34 mmol) afforded the title compound as a white solid (0.039 g, 57%). ¹H NMR (400 MHz, CDCl₃) δ 7.46-7.08 (m, 8H), 6.93 (d, J=8.0 Hz, 1H), 6.19-6.14 (m, 1H), 4.21 (s, 2H), 3.52 (t, J=5.6 Hz, 2H), 3.42 (s, 3H), 3.32 (t, J=6.9 Hz, 2H), 2.67 (t, J=7.4 Hz, 2H), 2.36 (s, 2H), 1.70 (dt, J=14.8, 7.0 Hz, 2H), 1.61 (q, J=7.3 Hz, 2H). UPLC/MS (method A): Rt 2.29 min. MS (ES) C₂₄H₂₇N₃O₃ requires 405, found 406 [M+H]⁺.

### Example 16: 2-methyl-4-(3-methyl-2-oxo-1,3-benzoxazol-6-yl)-N-(4-phenylbutyl)piperidine-1-carboxamide

### tert-Butyl-2-methyl-4-(trifluoromethylsulfonyloxy)-3,6-dihydro-2H-pyridine-1-carboxylate (VIc)

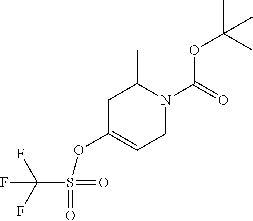 [SMILES: CC1CC(OS(=O)(=O)C(F)(F)F)=CCN1C(=O)OC(C)(C)C]

Following general procedure A (step 1), Vd (0.213 g, 1.0 mmol afforded Vic as a colorless oil (1:1 regioisomeric mixture, 0.290 g, 84%). ¹H NMR (400 MHz, CDCl₃) δ 5.78-5.67 (m, 1H), 4.83-4.56 (m, 1H), 4.42 (d, J=18.9 Hz, 0.5H), 4.35-4.17 (m, 0.5H), 3.69-3.57 (m, 0.5H), 2.99 (t, J1=12.7 Hz, 0.5H), 2.81 (ddq, J=16.9, 6.7, 3.4 Hz, 0.5H), 2.58 (dddt, J=17.1, 11.5, 5.8, 2.7 Hz, 0.5H), 2.25-2.15 (m, 0.5H), 2.11-2.05 (m, 0.5H), 1.47 (s, 4.5H), 1.47 (s, 4.5H), 1.24 (d, J=6.8 Hz, 1.5H), 1.18 (d, J=6.9 Hz, 1.5H). UPLC/MS (method B): Rt 1.59 min. MS (ES) C₁₂H₁₈F₃NO₅S requires 345, found 346 [M+H]⁺.

### tert-Butyl-4-(3-hydroxy-4-nitrophenyl)-2-methyl-3,6-dihydro-2H-pyridine-1-carboxylate (VIIf)

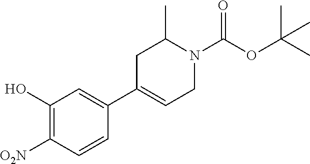 [SMILES: CC1CC(c2ccc([N+](=O)[O-])c(O)c2)=CCN1C(=O)OC(C)(C)C]

Following general procedure A, VIc (0.290 g, 0.84 mmol) and 5-bromo-2-nitrophenol (0.087 g, 0.76 mmol) afforded VIIf as a yellow solid (6:4 regioisomeric mixture, 0.206 g, 73%). ¹H NMR (400 MHz, CDCl₃) δ 10.66 (s, 0.4H), 10.65 (s, 0.6H), 8.06 (d, J=8 Hz, 0.4H), 8.05 (d, J=8 Hz, 0.6H), 7.12-7.08 (m, 1H), 7.05-6.98 (m, 1H), 6.29-6.23 (m, 0.4H), 6.23-6.16 (m, 0.6H), 4.79-4.57 (m, 1H), 4.51-4.37 (m, 0.4H), 4.34-4.20 (m, 0.6H), 3.80-3.71 (m, 0.4H), 3.03-2.89 (m, 0.6H), 2.85-2.75 (m, 0.4H), 2.63-2.50 (m, 0.6H), 2.37-2.29 (m, 0.6H), 2.24 (d, J=16.6 Hz, 0.4H), 1.49 (s, 5.4H), 1.49 (s, 3.6H), 1.28 (d, J=6.8 Hz, 1.8H), 1.15 (d, J=6.8 Hz, 1.2H). UPLC/MS (method B): Rt 1.59 min. MS (ES) C₁₇H₂₂N₂O₅ requires 334, found 333 [M−H]⁻.

### tert-Butyl 4-(4-amino-3-hydroxyphenyl)-2-methylpiperidine-1-carboxylate (VIIf)

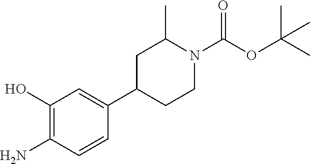 [SMILES: CC1CC(c2ccc(N)c(O)c2)CCN1C(=O)OC(C)(C)C]

Following general procedure B, (Method A, step 1), VIIf (0.2 g, 0.6 mmol) afforded VIIf which was used in the next step without further purification. UPLC/MS (method A): Rt 2.04 min. MS (ES) C₁₇H₂₆N₂O₃ requires 306, found 307 [M+H]⁺.

### tert-Butyl 2-methyl-4-(2-oxo-3H-1,3-benzoxazol-6-yl)piperidine-1-carboxylate (IXf)

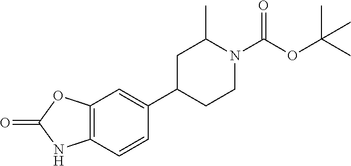 [SMILES: CC1CC(c2ccc3[nH]c(=O)oc3c2)CCN1C(=O)OC(C)(C)C]

Following general procedure B (step 2), VIIf (0.184 g, 0.6 mmol) afforded IXf as a colorless oil (0.157 g, 79%). UPLC/MS (method B): Rt 0.97 min. MS (ES) C₁₈H₂₄N₂O₄ requires 332, found 333 [M+H]⁺.

### tert-Butyl 2-methyl-4-(3-methyl-2-oxo-1,3-benzoxazol-6-yl)piperidine-1-carboxylate (XIg)

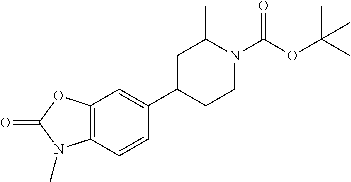 [SMILES: CC1CC(c2ccc3c(c2)oc(=O)n3C)CCN1C(=O)OC(C)(C)C]

Following general procedure C (step 1), IXf (0.157 g, 0.47 mmol) and MeI (0.1 g, 0.705 mmol) afforded XIg which was used in the next step without purification. UPLC/MS (method B): Rt 1.28 min. MS (ES) C₁₉H₂₆N₂O₄ requires 346, found 347 [M+H]⁺.

### 3-Methyl-6-(2-methyl-4-piperidyl]-1,3-benzoxazol-2-one hydrochloride (XIIg)

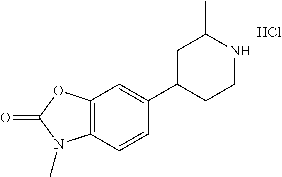 [SMILES: CC1CC(c2ccc3c(c2)oc(=O)n3C)CCN1 ; Cl]

Following general procedure C (step 2), XIg (0.163 g, 0.47 mmol) afforded XIIg which was used in the next step without further purification. UPLC/MS (method A): Rt 1.12 min. MS (ES) C₁₄H₁₈N₂O₂ requires 246, found 247 [M+H]⁺.

### 2-Methyl-4-(3-methyl-2-oxo-1,3-benzoxazol-6-yl)-N-(4-phenylbutyl)piperidine-1-carboxamide

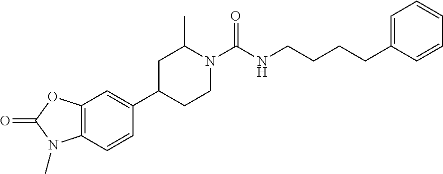 [SMILES: CC1CC(c2ccc3c(c2)oc(=O)n3C)CCN1C(=O)NCCCCc1ccccc1]

Following general procedure D (Method A), XIIg (0.140 g, 0.40 mmol) and 4-phenylbutyl isocyanate (0.09 g, 0.52 mmol) afforded the title compound as a white solid (70:30, cis/trans diastereomeric mixture, 0.140 g, 71%). ¹H NMR (400 MHz, DMSO-d₆) δ 7.31-7.06 (m, 8H), 6.38 (t, J=5.6 Hz, 0.3H), 6.28 (t, J=5.6 Hz, 0.7H), 4.44-4.31 (m, 0.3H), 3.93-3.79 (m, 1H), 3.62 (ddd, J=13.8, 7.1, 3.4 Hz, 0.7H), 3.33 (s, 3H), 3.20-3.10 (m, 0.7H), 3.10-2.96 (m, 1.3H), 2.96-2.81 (m, 0.3H), 2.70-2.62 (m, 0.7H), 2.58 (t, J=7.7 Hz, 2H), 1.98 (dq, J=15.7, 7.8 Hz, 0.7H), 1.83-1.33 (m, 7.3H), 1.13 (d, J=6.8 Hz, 0.9H), 1.08 (d, J=6.2 Hz, 2H). UPLC/MS (method B): Rt 1.06 min. MS (ES) C₂₅H₃₁N₃O₃ requires 421, found 422 [M+H]⁺.

### Example 17: (2R)-2-Methyl-4-(3-methyl-2-oxo-1,3-benzoxazol-6-yl)-N-(4-phenylbutyl)piperidine-1-carboxamide

### tert-Butyl-(2R)-2-methyl-4-(trifluoromethylsulfonyloxy)-3,6-dihydro-2H-pyridine-1-carboxylate (VId)

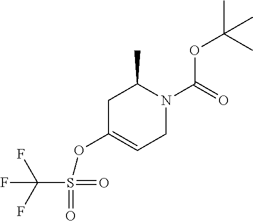 [SMILES: C[C@@H]1CC(OS(=O)(=O)C(F)(F)F)=CCN1C(=O)OC(C)(C)C]

Following general procedure A, Ve (0.427 g, 2.0 mmol) afforded VId as a colorless oil (1:1 regioisomeric mixture, 0.5 g, 73%). ¹H NMR (400 MHz, CDCl₃) δ 5.81-5.58 (m, 1H), 4.84-4.54 (m, 1H), 4.48-4.34 (m, 0.5H), 4.33-4.10 (m, 0.5H), 3.69-3.58 (m, 0.5H), 2.98 (t, J=11.3 Hz, 0.5H), 2.80 (ddq, J=16.7, 6.7, 3.4 Hz, 0.5H), 2.58 (dddd, J=14.5, 11.5, 6.2, 3.1 Hz, 0.5H), 2.20 (dd, J=16.8, 3.4 Hz, 0.5H), 2.13-1.92 (m, 0.5H), 1.51-1.43 (m, 9H), 1.25-1.19 (m, 1.5H), 1.17 (d, J=6.9 Hz, 1.5H). UPLC/MS (method B): Rt 1.59 min. MS (ES) C₁₂H₁₈F₃NO₅S requires 345, found 346 [M+H]⁺.

### tert-Butyl-(2R)-4-(3-hydroxy-4-nitrophenyl)-2-methyl-3,6-dihydro-2H-pyridine-1-carboxylate (VIIg)

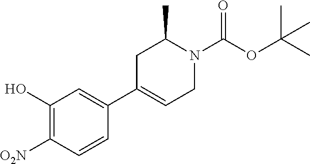 [SMILES: C[C@@H]1CC(c2ccc([N+](=O)[O-])c(O)c2)=CCN1C(=O)OC(C)(C)C]

Following general procedure A, VId (1.10 g, 2.63 mmol) and 5-bromo-2-nitrophenol (0.520 g, 2.37 mmol) afforded VIIg as a yellow oil (1:1 regioisomeric mixture, 0.544 g, 62%). ¹H NMR (400 MHz, CDCl₃) δ 10.64 (bs, 1H), 8.05 (d, J=8.9 Hz, 1H), 7.10 (d, J=1.9 Hz, 1H), 7.01 (dd, J=8.9, 2.0 Hz, 1H), 6.22-6.17 (m, 1H), 4.78-4.54 (m, 1H), 4.38-4.15 (m, 1H), 3.03-2.87 (m, 1H), 2.63-2.50 (m, 1H), 2.39-2.29 (m, 1H), 1.49 (s, 9H), 1.28 (d, J=6.8 Hz, 3H). UPLC/MS (method B): Rt 1.33 min, 1.55 min. MS (ES) C₁₇H₂₂N₂O₅ requires 334, found 335 [M+H]⁺.

### tert-Butyl (2R)-4-(4-amino-3-hydroxyphenyl)-2-methylpiperidine-1-carboxylate (VIIg)

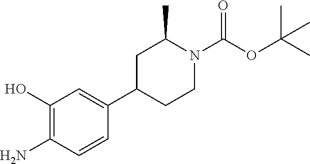 [SMILES: C[C@@H]1CC(c2ccc(N)c(O)c2)CCN1C(=O)OC(C)(C)C]

Following general procedure B, (Method A, step 1), VIIg (0.544 g, 1.63 mmol) afforded VIIIg which was used in the next step without further purification. UPLC/MS (method A): Rt 2.00 min. MS (ES) requires 306, found 307 [M+H]⁺.

### tert-Butyl (2R)-2-methyl-4-(2-oxo-3H-1,3-benzoxazol-6-yl)piperidine-1-carboxylate (IXg)

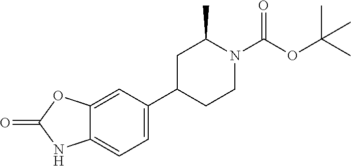 [SMILES: C[C@@H]1CC(c2ccc3[nH]c(=O)oc3c2)CCN1C(=O)OC(C)(C)C]

Following procedure B (step 2), VIIIh (0.490 g, 1.6 mmol) afforded IXg as a yellow oil (0.400 g, 75%). ¹H NMR (400 MHz, CDCl₃) δ 8.16 (s, 1H), 7.07-7.05 (m, 1H), 7.04-7.01 (m, 1H), 6.90-6.86 (m, 1H), 4.00-3.92 (m, 1H), 3.81 (dd, J=13.9, 7.5, 2.9 Hz, 1H), 3.24 (ddd, J=14.0, 9.8, 6.5 Hz, 1H), 2.77 (qd, J=11.5, 10.5, 3.0 Hz, 1H), 2.16 (tt, J=12.9, 6.4 Hz, 1H), 1.90 (ddd, J=13.2, 6.0, 2.7 Hz, 1H), 1.79 (td, J=13.1, 5.4 Hz, 1H), 1.56 (dddd, J=13.4, 10.2, 7.4, 4.2 Hz, 1H), 1.48 (s, 9H), 1.20 (d, J=6.3 Hz, 3H). UPLC/MS (method A): Rt 2.17 min. MS (ES) C₁₈H₂₄N₂O₄ requires 332, found 333 [M+H]⁺.

### tert-Butyl (2R)-2-methyl-4-(3-methyl-2-oxo-1,3-benzoxazol-6-yl)piperidine-1-carboxylate (XIh)

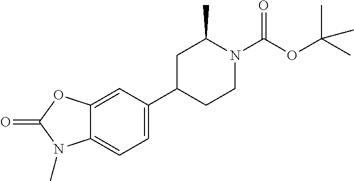 [SMILES: C[C@@H]1CC(c2ccc3c(c2)oc(=O)n3C)CCN1C(=O)OC(C)(C)C]

Following general procedure C (step 1), IXg (0.610 g, 1.81 mmol) and MeI (0.39 g, 2.72 mmol) afforded XIh which was used in the next step without further purification. ¹H NMR (400 MHz, CDCl₃) δ 7.07-7.04 (m, 1H), 7.04-6.99 (m, 1H), 6.91-6.82 (m, 1H), 4.56-4.45 (m, 1H), 4.02-3.88 (m, 1H), 3.80 (ddd, J=13.9, 6.4, 3.7 Hz, 1H), 3.38 (d, J=2.0 Hz, 3H), 3.28-3.16 (m, 1H), 2.77 (dd, J=12.3, 7.4 Hz, 1H), 2.24-2.07 (m, 1H), 1.89 (ddq, J=9.6, 4.5, 1.7 Hz, 1H), 1.60-1.51 (m, 1H), 1.48 (d, J=1.9 Hz, 9H), 1.20 (dd, J=6.4, 1.7 Hz, 3H). UPLC/MS (method B): Rt 1.23 min. MS (ES) C₁₉H₂₆N₂O₄ requires 346, found 347 [M+H]⁺.

### 3-Methyl-6-[(2R)-2-methyl-4-piperidyl]-1,3-benzoxazol-2-one hydrochloride (XIIh)

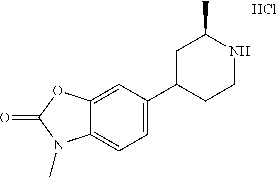 [SMILES: C[C@@H]1CC(c2ccc3c(c2)oc(=O)n3C)CCN1 ; Cl]

Following general procedure C (step 2), XIh (0.070 g, 0.2 mmol) afforded XIIh which was purified by trituration with Et₂O (0.055 g, 97%). UPLC/MS (method A): Rt 1.08, 1.17 min. MS (ES) C₁₄H₁₈N₂O₂ requires 246, found 247 [M+H]⁺.

### (2R)-2-Methyl-4-(3-methyl-2-oxo-1,3-benzoxazol-6-yl)-N-(4-phenylbutyl)piperidine-1-carboxamide

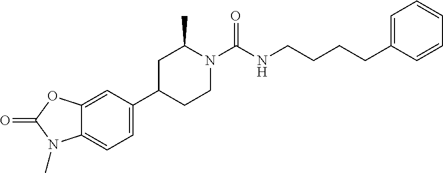 [SMILES: C[C@@H]1CC(c2ccc3c(c2)oc(=O)n3C)CCN1C(=O)NCCCCc1ccccc1]

Following general procedure D (Method A), XIIh (0.050 g, 0.17 mmol) and 4-phenylbutyl isocyanate (0.07 g, 0.40 mmol) afforded the title compound as a white solid (70:30, cis/trans diastereomeric mixture, 0.040 g, 52%). ¹H NMR (400 MHz, DMSO-d₆) δ 7.30-7.22 (m, 4.38H), 7.22-7.06 (m, 7.30H), 6.38 (t, J=5.4 Hz, 1H), 6.28 (t, J=5.5 Hz, 1H), 4.42-4.33 (m, 0.3H), 3.92-3.80 (m, 1.3H), 3.62 (ddd, J=3.3, 6.9, 13.6 Hz, 1H), 3.31 (s, 4.28H), 3.14 (ddd, J=14.2, 9.5, 6.2 Hz, 1H), 3.09-3.03 (m, 2.6H), 3.00-2.91 (m, 0.3H), 2.91-2.80 (m, 0.4H), 2.66 (dq, J=12.9, 7.4, 5.7 Hz, 1H), 2.58 (t, J=7.5 Hz, 3H), 1.98 (dq, J=16.1, 7.6 Hz, 1H), 1.84-1.36 (m, 9.8H), 1.13 (d, J=6.8 Hz, 1.18H), 1.08 (d, J=6.3 Hz, 3H). UPLC/MS (method A): Rt 2.23 min. MS (ES) C₂₅H₃₁N₃O₃ requires 421, found 422 [M+H]⁺.

### Example 18: (2R)-2-Methyl-4-(3-methyl-2-oxo-1,3-benzoxazol-6-yl)-N-(2-phenylethyl)piperidine-1-carboxamide

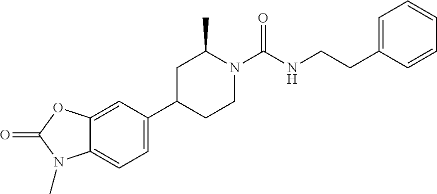 [SMILES: C[C@@H]1CC(c2ccc3c(c2)oc(=O)n3C)CCN1C(=O)NCCc1ccccc1]

Following general procedure D (Method A), XIIh (0.050 g, 0.18 mmol) and 2-phenylethyl isocyanate (0.09 g, 0.36 mmol) afforded the title compound as a white solid (70:30, cis/trans diastereomeric mixture, 0.060 g, 90%). ¹H NMR (400 MHz, DMSO-d₆) δ 7.32-7.24 (m, 4.41H), 7.23-7.07 (m, 7.14H), 6.50 (d, J=5.2 Hz, 0.45H), 6.36 (t, J=5.4 Hz, 1H), 4.38 (s, 0.46H), 3.94-3.81 (m, 1.48H), 3.72-3.52 (m, 1.13H), 3.31 (s, 3H), 3.29-3.08 (m, 4.29H), 3.00-2.81 (m, 1H), 2.78-2.68 (m, 2.8H), 2.70-2.59 (m, 1.33H), 2.03-1.89 (m, 1H), 1.84-1.57 (m, 3.5H), 1.55-1.39 (m, 1.6H), 1.13 (d, J=6.8 Hz, 1.23H), 1.08 (d, J=6.3 Hz, 3H). UPLC/MS (method A): Rt 2.03 min. MS (ES) C₂₃H₂₇N₃O₃ requires 393, found 394 [M+H]⁺.

### Example 19: N-iso-Butyl (2R)-methyl-4-(3-methyl-2-oxo-1,3-benzoxazol-6-yl)piperidine-1-carboxamide

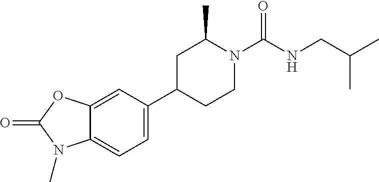 [SMILES: CC(C)CNC(=O)N1CCC(c2ccc3c(c2)oc(=O)n3C)C[C@H]1C]

Following general procedure D (Method B), XIIh (0.050 g, 0.18 mmol) and isobutylamine (0.04 g, 0.54 mmol) afforded the title compound as a white solid (70:30, cis trans diastereomeric mixture, 0.050 g, 76%). ¹H NMR (400 MHz, DMSO-d₆) δ 7.26 (s, 1H), 7.18-7.07 (m, 2H), 6.40 (t, J=5.6 Hz, 0.3H), 6.31 (t, J=5.6 Hz, 1H), 4.47-4.35 (m, 0.3H), 3.95-3.91 (m, 0.3H), 3.91-3.80 (m, 1H), 3.65 (ddd, J=13.6, 6.9, 3.5 Hz, 1H), 3.32 (s, 3H), 3.18 (ddd, J=14.1, 9.3, 6.1 Hz, 1H), 3.01-2.93 (m, 0.3H), 2.92-2.75 (m, 2.8H), 2.74-2.60 (m, 1H), 1.99 (dq, J=15.9, 7.6 Hz, 1H), 1.85-1.58 (m, 4H), 1.58-1.46 (m, 1H), 1.49-1.39 (m, 0.3H), 1.15 (d, J=6.8 Hz, 1.2H), 1.11 (d, J=6.3 Hz, 3H), 0.83 (d, J=6.7 Hz, 6H), 0.82 (d, J=6.7 Hz, 2.6H). UPLC/MS (method A): Rt 1.93 min. MS (ES) C₁₉H₂₇N₃O₃ requires 345, found 346 [M+H]⁺.

### Example 20: (2S)-methyl-4-(3-methyl-2-oxo-1,3-benzoxazol-6-yl)-N-(4-phenylbutyl)piperidine-1-carboxamide

### tert-Butyl (2S)-methyl-4-(((trifluoromethyl)sulfonyl)oxy)-3,6-dihydropyridine-1(2H)-carboxylate (VIe)

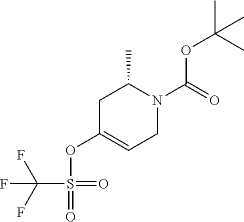 [SMILES: C[C@H]1CC(OS(=O)(=O)C(F)(F)F)=CCN1C(=O)OC(C)(C)C]

Following general procedure A (step 1), Vf (0.213 g, 1.0 mmol) afforded VIe as a colorless oil (1:1 regioisomeric mixture, 0.320 g, 93%). ¹H NMR (400 MHz, CDCl₃) δ 5.78-5.67 (m, 1H), 4.83-4.56 (m, 1H), 4.42 (d, J=18.9 Hz, 0.5H), 4.35-4.17 (m, 0.5H), 3.69-3.57 (m, 0.5H), 2.99 (t, J=12.7 Hz, 0.5H), 2.81 (ddq, 1=16.9, 6.7, 3.4 Hz, 0.5H), 2.58 (dddt, J=17.1, 11.5, 5.8, 2.7 Hz, 0.5H), 2.25-2.15 (m, 0.5H), 2.11-2.05 (m, 0.5H), 1.47 (s, 4.5H), 1.47 (s, 4.5H), 1.24 (d, J=6.8 Hz, 1.5H), 1.18 (d, J=6.9 Hz, 1.5H). UPLC/MS (method B): Rt 1.61 min. MS (ES) C₁₂H₁₈F₃NO₅S requires 345, found 346 [M+H]⁺.

### tert-Butyl (2S)-4-(3-hydroxy-4-nitrophenyl)-2-methyl-3,6-dihydro-2H-pyridine-1-carboxylate (VIIh)

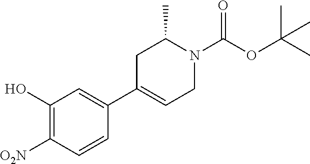 [SMILES: C[C@H]1CC(c2ccc([N+](=O)[O-])c(O)c2)=CCN1C(=O)OC(C)(C)C]

Following general procedure A (step 2), VIe (0.32 g, 0.93 mmol), and 5-bromo-2-nitrophenol (0.223 g, 1.02 mmol) afforded VIIh as a yellow solid (60:40 regioisomeric mixture, 0.115 g, 37%). ¹H NMR (400 MHz, CDCl₃) δ 10.66 (s, 0.4H), 10.65 (s, 0.6H), 8.06 (d, J=8 Hz, 0.4H), 8.05 (d, J=8 Hz, 0.6H), 7.12-7.08 (m, 1H), 7.05-6.98 (m, 1H), 6.29-6.23 (m, 0.4H), 6.23-6.16 (m, 0.6H), 4.79-4.57 (m, 1H), 4.51-4.37 (m, 0.4H), 4.34-4.20 (m, 0.6H), 3.80-3.71 (m, 0.4H), 3.03-2.89 (m, 0.6H), 2.85-2.75 (m, 0.4H), 2.63-2.50 (m, 0.6H), 2.37-2.29 (m, 0.6H), 2.24 (d, J=16.6 Hz, 0.4H), 1.49 (s, 5.4H), 1.49 (s, 3.6H), 1.28 (d, J=6.8 Hz, 1.8H), 1.15 (d, J=6.8 Hz, 1.2H). UPLC/MS (method B): Rt 1.59 min. MS (ES) C₁₇H₂₂N₂O₅ requires 334, found 333 [M−H]⁻.

### tert-Butyl 4-(4-amino-3-hydroxyphenyl)-(2S)-methylpiperidine-1-carboxylate (VIIIh)

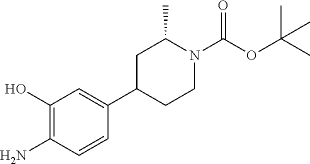 [SMILES: C[C@H]1CC(c2ccc(N)c(O)c2)CCN1C(=O)OC(C)(C)C]

Following general procedure B (Method A, step 1), VIIh (0.215 g, 0.64 mmol) afforded VIIh which was used in the next step without further purification. UPLC/MS (method A): Rt 2.04 min. MS (ES) C₁₇H₂₆N₂O₃ requires 306, found 307 [M+H]⁺.

### tert-Butyl (2S)-methyl-4-(2-oxo-3H-1,3-benzoxazol-6-yl)piperidine-1-carboxylate (IXh)

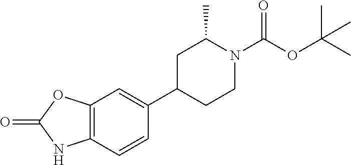 [SMILES: C[C@H]1CC(c2ccc3[nH]c(=O)oc3c2)CCN1C(=O)OC(C)(C)C]

Following general procedure B (step 2), VIIIh (0.196 g, 0.64 mmol) afforded IXh as a colorless oil (0.105 g, 49%). ¹H NMR (400 MHz, CDCl₃) δ 7.67 (br s, 1H), 7.06 (s, 1H), 7.02-6.91 (m, 2H), 4.02-3.91 (m, 1H), 3.81 (ddd, J=13.9, 7.5, 3.3 Hz, 1H), 3.25 (ddd, J=13.9, 9.7, 6.4 Hz, 1H), 2.84-2.72 (m, 1H), 2.25-2.10 (m, 1H), 1.95-1.87 (m, 1H), 1.87-1.74 (m, 1H), 1.64-1.54 (m, 1H), 1.49 (s, 9H), 1.21 (d, J=6.4 Hz, 3H). UPLC/MS (method B): Rt 0.97 min. MS (ES) C₁₈H₂₄N₂O₄ requires 332, found 333 [M+H]⁺.

### tert-Butyl (2S)-methyl-4-(3-methyl-2-oxo-1,3-benzoxazol-6-yl)piperidine-1-carboxylate (XIi)

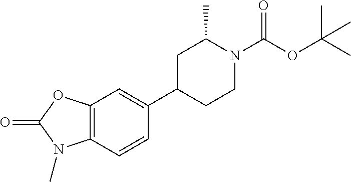 [SMILES: C[C@H]1CC(c2ccc3c(c2)oc(=O)n3C)CCN1C(=O)OC(C)(C)C]

Following general procedure C (step 1), IXh (0.105 g, 0.32 mmol) and MeI (0.77 g, 0.47 mmol) afforded XIi which was used in the next step without further purification. UPLC/MS (method B): Rt 1.28 min. MS (ES) C₁₉H₂₆N₂O₄ requires 346, found 347 [M+H]⁺.

### 6-[(2S)-Methyl-4-piperidyl]-3H-1,3-benzoxazol-2-one hydrochloride (XIi)

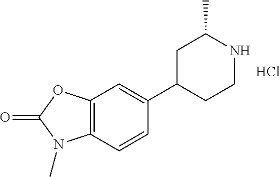 [SMILES: C[C@H]1CC(c2ccc3c(c2)oc(=O)n3C)CCN1 ; Cl]

Following general procedure C (step 2), IXh (0.100 g, 0.290 mmol) afforded XIIi which was used in the next step without further purification (70:30, cis/trans diastereomeric mixture, white solid). ¹H NMR (400 MHz, DMSO-d₆) δ 9.33-8.62 (m, 2H), 7.28-7.07 (m, 3H), 3.68-3.54 (m, 0.3H), 3.35 (m overlapped with H₂O signal, 0.7H), 3.32 (s, 3H), 3.29-3.18 (m, 0.7H), 3.18-3.03 (m, 0.9H), 3.03-2.87 (m, 1.4H), 2.13-2.01 (m, 0.3H), 2.00-1.87 (m, 2H), 1.87-1.73 (m, 1H), 1.65 (q, J1=12.6 Hz, 0.7H), 1.37 (d, J=6.9 Hz, 0.9H), 1.27 (d, J=6.4 Hz, 2.1H). UPLC/MS (method A): Rt 1.12 min. MS (ES) C₁₄H₁₈N₂O₂ requires 246, found 247 [M+H]⁺.

### (2S)-Methyl-4-(3-methyl-2-oxo-1,3-benzoxazol-6-yl)-N-(4-phenylbutyl)piperidine-t-carboxamide

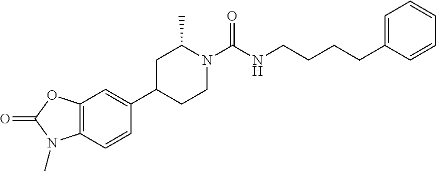 [SMILES: C[C@H]1CC(c2ccc3c(c2)oc(=O)n3C)CCN1C(=O)NCCCCc1ccccc1]

Following general procedure D (Method A), XIIi (0.030 g, 0.122 mmol) and 4-phenylbutyl isocyanate (0.023 g, 0.134 mmol) afforded the title compound as a white solid (70:30, cis trans diastereomeric mixture, 0.038 g, 74%). ¹H NMR (400 MHz, DMSO-d₆) δ 7.31-7.06 (m, 8H), 6.38 (t, J=5.6 Hz, 0.3H), 6.28 (t, J=5.6 Hz, 0.7H), 4.44-4.31 (m, 0.3H), 3.93-3.79 (m, 1H), 3.62 (ddd, J=13.8, 7.1, 3.4 Hz, 0.7H), 3.33 (s, 3H), 3.20-3.10 (m, 0.7H), 3.10-2.96 (m, 1.3H), 2.96-2.81 (m, 0.3H), 2.70-2.62 (m, 0.7H), 2.58 (t, J=7.7 Hz, 2H), 1.98 (dq, J=15.7, 7.8 Hz, 0.7H), 1.83-1.33 (m, 7.3H), 1.13 (d, J=6.8 Hz, 0.9H), 1.08 (d, J=6.2 Hz, 2.1H). UPLC/MS (method B): Rt 1.06 min. MS (ES) C₂₅H₃₁N₃O₃ requires 421, found 422 [M+H]⁺.

### Example 21: (2S)-Methyl-4-(3-methyl-2-oxo-1,3-benzoxazol-6-yl)-N-(2-phenylethyl)piperidine-carboxamide

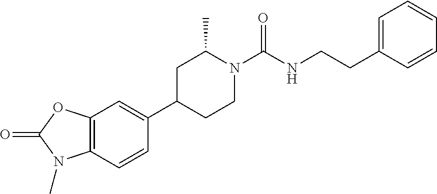 [SMILES: C[C@H]1CC(c2ccc3c(c2)oc(=O)n3C)CCN1C(=O)NCCc1ccccc1]

Following general procedure D (Method A), XIi (0.015 g, 0.05 mmol) and 2-phenylethyl isocyanate (0.009 g, 0.0009 mL, 0.064 mmol) afforded the title compound as a white solid (0.011 g, 64%). 1H NMR (400 MHz, DMSO-d₆) δ 7.34-7.24 (m, 3H), 7.23-7.09 (m, 5H), 6.37 (t, J=5.5 Hz, 1H), 3.87 (dt, J=10.1, 6.2 Hz, 1H), 3.64 (ddd, J=14.0, 7.2, 3.3 Hz, 1H), 3.31 (s, 3H), 3.29-3.20 (m, 2H), 3.20-3.10 (m, 1H), 2.75 (t, 0.1=7.4 Hz, 2H), 2.70-2.60 (m, 1H), 2.04-1.91 (m, 1H), 1.84-1.75 (m, 1H), 1.64 (td, J=12.8, 9.9 Hz, 1H), 1.50 (dddd, J=13.3, 9.8, 6.1, 3.3 Hz, 1H), 1.09 (d, J=6.2 Hz, 3H). UPLC/MS (method A): Rt 2.04 min. MS (ES) C₂₃H₂₇N₃O₃ requires 393, found 394 [M+H]⁺.

### Example 22: N-iso-Butyl (2S)-methyl-4-(3-methyl-2-oxo-1,3-benzoxazol-6-yl)piperidine-1-carboxamide

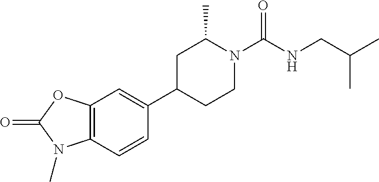 [SMILES: CC(C)CNC(=O)N1CCC(c2ccc3c(c2)oc(=O)n3C)C[C@@H]1C]

Following general procedure D (Method B), XIIi (0.020 g, 0.07 mmol) and isobutyl amine (0.015 g, 0.21 mmol) afforded the title compound as a white solid (0.012 g, 50%). ¹H NMR (400 MHz, DMSO-d₆) δ 7.28-7.24 (m, 1H), 7.17-7.08 (m, 2H), 6.31 (t, J=5.7 Hz, 1H), 3.86 (dt, J=10.0, 6.2 Hz, 1H), 3.64 (ddd, J=13.8, 7.0, 3.6 Hz, 1H), 3.31 (s, 3H), 3.17 (ddd, J=13.8, 9.3, 6.0 Hz, 1H), 2.90 (ddd, J=12.8, 6.9, 5.8 Hz, 1H), 2.80 (ddd, J=12.8, 7.0, 5.5 Hz, 1H), 2.72-2.60 (m, 1H), 2.05-1.92 (m, 1H), 1.83-1.75 (m, 1H), 1.75-1.58 (m, 2H), 1.52 (dddd, J=13.3, 9.8, 6.0, 3.6 Hz, 1H), 1.10 (d, J=6.2 Hz, 3H), 0.83 (d, J=6.7 Hz, 6H). UPLC/MS (method A): Rt 1.90 min. MS (ES) C₁₉H₂₇N₃O₃ requires 345, found 346 [M+H]⁺.

### Example 23: 2,2-Dimethyl-4-(2-oxo-3H-1,3-benzoxazol-6-yl)-N-(4-phenylbutyl)piperidine-1-carboxamide

### tert-Butyl 2,2-dimethyl-4-(((trifluoromethyl)sulfonyl)oxy)-3,6-dihydropyridine-1(2H)-carboxylate (VIf)

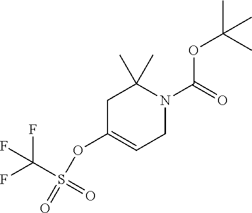 [SMILES: CC(C)(C)OC(=O)N1CC=C(OS(=O)(=O)C(F)(F)F)CC1(C)C]

Following general procedure A (step 1), Vg (0.227 g, 1.0 mmol) afforded VIf as a colorless oil (0.328 g, 91%). ¹H NMR (400 MHz, CDCl₃) δ 5.77 (tt, J=3.8, 1.2 Hz, 1H), 4.07 (dt, J=3.8, 2.6 Hz, 2H), 2.39 (dt, J=2.6, 1.4 Hz, 2H), 1.49 (s, 6H), 1.46 (s, 9H). UPLC/MS (method B): Rt 1.92 min. MS (ES) C₁₃H₂₀F₃NO₅S requires 359, found 360 [M+H]⁺.

### tert-Butyl 4-(3-hydroxy-4-nitrophenyl)-6,6-dimethylcyclohex-3-ene-1-carboxylate (VIIi)

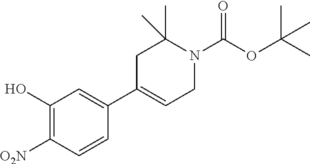 [SMILES: CC(C)(C)OC(=O)N1CC=C(c2ccc([N+](=O)[O-])c(O)c2)CC1(C)C]

Following general procedure A (step 2), VIf (0.328 g, 0.91 mmol) and 5-bromo-2-nitrophenol (0.179 g, 0.82 mmol) afforded VIIi as a yellow solid (0.300 g, 46%). ¹H NMR (400 MHz, CDCl₃) δ 10.66 (s, 1H), 8.06 (d, J=8.9 Hz, 1H), 7.10 (d, J=1.9 Hz, 1H), 7.03 (dd, J=8.9, 2.0 Hz, 1H), 6.41 (tt, J=4.4, 1.0 Hz, 1H), 4.12 (m, 2H), 2.50 (m, 2H), 1.49 (s, 8H), 1.47 (s, 6H). UPLC/MS (method B): Rt 1.82 min. MS (ES) C₁₈H₂₄N₂O₅ requires 348, found 349 [M+H]⁺.

### tert-Butyl 4-(4-amino-3-hydroxyphenyl)-2,2-dimethylpiperidine-1-carboxylate (VIIHi)

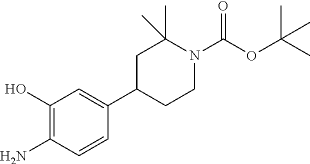 [SMILES: CC(C)(C)OC(=O)N1CCC(c2ccc(N)c(O)c2)CC1(C)C]

Following general procedure B (Method A, step 1), VIIi (0.168 g, 0.48 mmol) afforded VIIi which was used in the next step without further purification. UPLC/MS (method A): Rt 0.96 min. MS (ES) C₁₈H₂₈N₂O₃ requires 320, found 321 [M+H]⁺.

### tert-Butyl 2,2-dimethyl-4-(2-oxo-3H-1,3-benzoxazol-6-yl)piperidine-1-carboxylate (IXi)

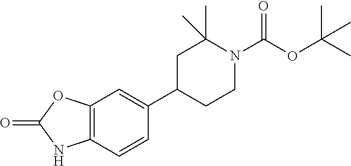 [SMILES: CC(C)(C)OC(=O)N1CCC(c2ccc3[nH]c(=O)oc3c2)CC1(C)C]

Following general procedure B (step 2), VIIi (0.169 g, 0.53 mmol) afforded IXi as a white solid (0.176 g, 59%). ¹H NMR (400 MHz, CDCl₃) δ 7.94 (s, 1H), 7.07 (s, 1H), 7.02-6.94 (m, 2H), 3.98 (dt, J=13.7, 4.7 Hz, 1H), 3.19 (ddd, J=14.0, 10.7, 3.7 Hz, 1H), 2.92-2.79 (m, 1H), 2.00-1.92 (m, 1H), 1.74 (t, J=13.2 Hz, 1H), 1.69-1.58 (m, 2H), 1.54 (s, 6H), 1.48 (s, 9H). UPLC/MS (method A): Rt 2.36 min. MS (ES) C₁₉H₂₆N₂O₄ requires 346, found 347 [M+H]⁺.

### 6-(2,2-dimethyl-4-piperidyl)-3H-1,3-benzoxazol-2-one (Xc)

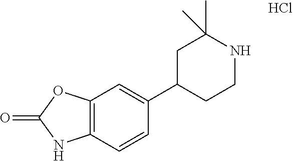 [SMILES: CC1(C)CC(c2ccc3[nH]c(=O)oc3c2)CCN1 ; Cl]

Following general procedure C (step 2), Xc (0.100 g, 0.290 mmol). The residue was used in the next step without further purification (white solid). UPLC/MS (method A): Rt 1.07 min. MS (ES) C₁₄H₁₈N₂O₂ requires 246, found 247 [M+H]⁺.

### 2,2-Dimethyl-4-(2-oxo-3H-1,3-benzoxazol-6-yl)-N-(4-phenylbutyl)piperidine-1-carboxamide

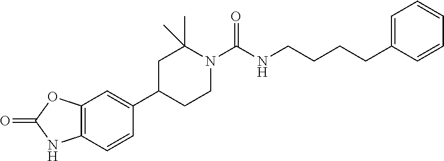 [SMILES: CC1(C)CC(c2ccc3[nH]c(=O)oc3c2)CCN1C(=O)NCCCCc1ccccc1]

Following general procedure D (Method A), Xc (0.035 g, 0.36 mmol.) and 4-phenylbutyl isocyanate (0.024 g, 0.14 mmol) afforded the title compound as a white solid (0.006 g, 10%). ¹H NMR (400 MHz, CDCl₃) δ 8.85 (s, 1H), 7.30-7.27 (m, 1H), 7.21-7.14 (m, 4H), 7.08 (s, 1H), 7.00 (s, 2H), 4.54 (t, J1=6.3 Hz, 1H), 3.52 (dt, J=10.8, 6.7 Hz, 1H), 3.31-3.10 (m, 3H), 2.93-2.78 (m, 1H), 2.64 (t, J=7.5 Hz, 2H), 2.01-1.88 (m, 1H), 1.77-1.46 (m, 7H), 1.57 (s, 3H), 1.40 (s, 3H). UPLC/MS (method B): Rt 1.09 min. MS (ES) C₂₅H₃₁N₃O₃ requires 421, found 422 [M+H]⁺.

### Example 24: 2,2-Dimethyl-4-(3-methyl-2-oxo-1,3-benzoxazol-6-yl)-N-(4-phenylbutyl)piperidine-1-carboxamide

### tert-Butyl 2,2-dimethyl-4-(3-methyl-2-oxo-1,3-benzoxazol-6-yl)piperidine-1-carboxylate (XIj)

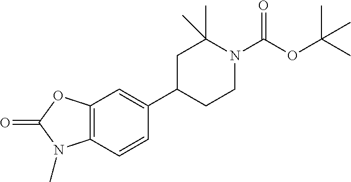 [SMILES: Cn1c(=O)oc2cc(C3CCN(C(=O)OC(C)(C)C)C(C)(C)C3)ccc21]

Following general procedure C (step 1), XIj (0.117 g, 0.34 mmol) and MeI (0.082 g, 0.51 mmol) afforded XIj which was used in the next step without purification. UPLC/MS (method A): Rt 2.58 min. MS (ES) C₂₀H₂₈N₂O₄ requires 360, found 361 [M+H]⁺.

### 6-(2,2-Dimethyl-4-piperidyl)-3-methyl-1,3-benzoxazol-2-one hydrochloride (XIIj)

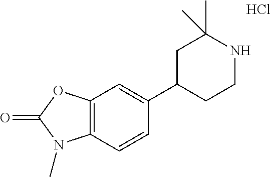 [SMILES: Cn1c(=O)oc2cc(C3CCNC(C)(C)C3)ccc21 ; Cl]

Following general procedure C (step 2), XIj (0.122 g, 0.34 mmol) afforded XIIj as a white solid (0.90 g, 90%). UPLC/MS (method A): Rt 1.15 min. MS (ES) C₁₅H₂₀N₂O₂ requires 260, found 261 [M+H]⁺.

### 2,2-Dimethyl-4-(3-methyl-2-oxo-1,3-benzoxazol-6-yl)-N-(4-phenylbutyl)piperidine-1-carboxamide

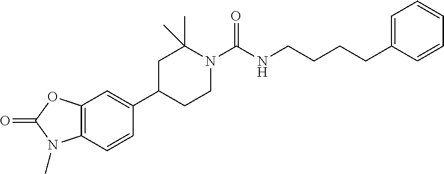 [SMILES: Cn1c(=O)oc2cc(C3CCN(C(=O)NCCCCc4ccccc4)C(C)(C)C3)ccc21]

Following general procedure D (Method A), XIIj (0.080 g, 0.27 mmol) and 4-phenylbutyl isocyanate (0.052 g, 0.29 mmol) afforded the compound as a white solid (0.066 g, 57%). ¹H NMR (400 MHz, DMSO-d₆) δ 7.31-7.24 (m, 3H), 7.23-7.10 (m, 5H), 6.47 (t, J=5.5 Hz, 1H), 3.61 (dt, J=12.9, 4.0 Hz, 1H), 3.33 (s, 3H), 3.06-2.97 (m, 3H), 2.85 (tt, J=12.1, 3.8 Hz, 1H), 2.58 (t, J=7.6 Hz, 2H), 1.85-1.79 (m, 1H), 1.65-1.49 (m, 5H), 1.46 (s, 3H), 1.41 (m, 2H), 1.31 (s, 3H). UPLC/MS (method A): Rt 2.45 min. MS (ES) C₂₆H₃₃N₃O₃ requires 435, found 436 [M+H]⁺.

### Example 25: 2,2-Dimethyl-4-(3-methyl-2-oxo-1,3-benzoxazol-6-yl)-N-(2-phenylethyl)piperidine-1-carboxamide

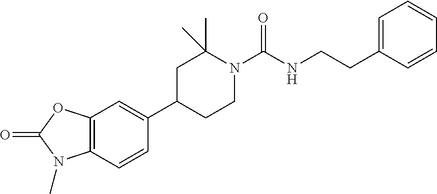 [SMILES: Cn1c(=O)oc2cc(C3CCN(C(=O)NCCc4ccccc4)C(C)(C)C3)ccc21]

Following general procedure D (Method A), XIIj (0.012 g, 0.04 mmol) and 2-phenylethyl isocyanate (0.007 g, 0.048 mmol) afforded the title compound as a white solid (0.007 g, 45%). ¹H NMR (400 MHz, DMSO-d₆) δ 7.33-7.24 (m, 3H), 7.23-7.08 (m, 5H), 6.56 (t, J=5.5 Hz, 1H), 3.59 (dt, J=12.9, 4.1 Hz, 1H), 3.33 (s, 3H), 3.27-3.13 (m, 2H), 2.97 (td, J=12.3, 3.0 Hz, 1H), 2.85 (tt, J=12.1, 3.8 Hz, 1H), 2.78-2.63 (m, 2H), 1.85-1.76 (m, 1H), 1.66-1.49 (m, 3H), 1.47 (s, 3H), 1.32 (s, 3H). UPLC/MS (method A): Rt 2.25 min. MS (ES) C₂₄H₂₉N₃O₃ requires 407, found 408 [M+H]⁺.

### Example 26: 2,2-Dimethyl-4-(3-methyl-2-oxo-1,3-benzoxazol-6-yl)-N-(3-phenylpropyl)piperidine-1-carboxamide

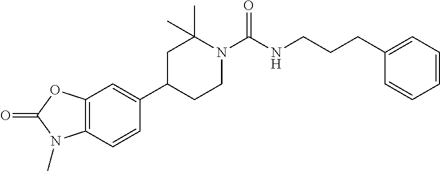 [SMILES: Cn1c(=O)oc2cc(C3CCN(C(=O)NCCCc4ccccc4)C(C)(C)C3)ccc21]

Following general procedure D (Method A), XIIj (0.030 g, 0.106 mmol) and 3-phenylpropyl isocyanate (0.031 g, 0.13 mmol) afforded the title compound as a white solid (0.025 g, 56%). ¹H NMR (400 MHz, DMSO-d₆) δ 7.32-7.23 (m, 3H), 7.23-7.08 (m, 5H), 6.52 (br s, 1H), 3.62 (dt, J=12.9, 4.1 Hz, 1H), 3.32 (s, 3H), 3.09-2.93 (m, 3H), 2.85 (tt, J=12.5, 4.0 Hz, 1H), 2.56 (t, J=7.7 Hz, 2H), 1.87-1.78 (m, 1H), 1.75-1.49 (m, 5H), 1.46 (s, 3H), 1.31 (s, 3H). UPLC/MS (method A): Rt 2.32 min. MS (ES) C₂₅H₃N₃O₃ requires 421, found 422 [M+H]⁺.

### Example 27: N-(2-Benzyloxyethyl)-2,2-dimethyl-4-(3-methyl-2-oxo-1,3-benzoxazol-6-yl)piperidine-1-carboxamide

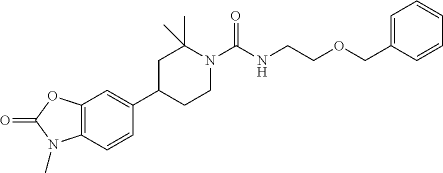 [SMILES: Cn1c(=O)oc2cc(C3CCN(C(=O)NCCOCc4ccccc4)C(C)(C)C3)ccc21]

Following general procedure D, XIIj (0.032 g, 0.103 mmol) and 2-benzyloxy ethanamine (0.042 g, 0.83 mmol) afforded the title compound as a white solid (0.022 g, 46%). ¹H NMR (400 MHz, DMSO-d₆) δ 7.39-7.23 (m, 6H), 7.18-7.08 (m, 2H), 6.51 (t, J=5.6 Hz, 1H), 4.47 (s, 2H), 3.62 (dt, J=12.9, 4.1 Hz, 1H), 3.43 (t, J=6.1 Hz, 2H), 3.19 (qd, J=6.0, 3.6 Hz, 2H), 3.06-2.95 (m, 1H), 2.84 (ddt, J=12.2, 7.7, 3.9 Hz, 1H), 1.87-1.76 (m, 1H), 1.66-1.48 (m, 3H), 1.46 (s, 3H), 1.31 (s, 3H). UPLC/MS (method A): Rt 2.20 min. MS (ES) C₂₅H₃₁N₃O₄ requires 437, found 438 [M+H]⁺.

### Example 28: 2,2-Dimethyl-4-(3-methyl-2-oxo-1,3-benzoxazol-6-yl)-N-pentylpiperidine-1-carboxamide

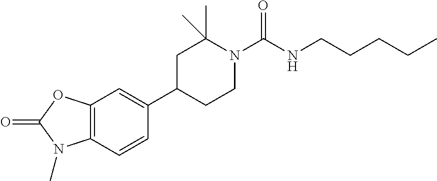 [SMILES: CCCCCNC(=O)N1CCC(c2ccc3c(c2)oc(=O)n3C)CC1(C)C]

Following general procedure D (Method A), XIIj (0.025 g, 0.08 mmol) and penthyl isocyanate (0.011 g, 0.095 mmol) afforded the title compound as a white solid (0.019 g, 58%). ¹H NMR (400 MHz, DMSO-d₆) δ 7.19-7.07 (m, 2H), 6.43 (t, J=5.5 Hz, 1H), 3.60 (dt, J=12.8, 4.0 Hz, 1H), 3.33 (s, 3H), 3.04-2.90 (m, 3H), 2.84 (ddd, J=12.3, 8.3, 3.8 Hz, 1H), 1.86-1.77 (m, 1H), 1.66-1.48 (m, 3H), 1.45 (s, 3H), 1.43-1.35 (m, 2H), 1.34-1.18 (m, 7H), 0.86 (t, J=7.0 Hz, 3H). UPLC/MS (method A): Rt 2.30 min. MS (ES) C₂₁H₃₁N₃O₃ requires 373, found 374 [M+H]⁺.

### Example 29: N-(2-Cyclopropylethyl)-2,2-dimethyl-4-(3-methyl-2-oxo-1,3-benzoxazol-6-yl)piperidine-1-carboxamide

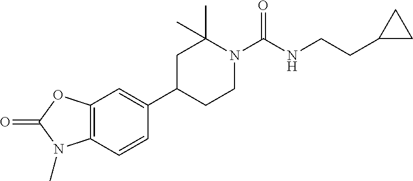 [SMILES: Cn1c(=O)oc2cc(C3CCN(C(=O)NCCC4CC4)C(C)(C)C3)ccc21]

Following general procedure D (Method C), XIIj (0.055 g, 0.18 mmol) and 2-cyclopropylethanamine hydrochloride (0.029 g, 0.24 mmol) afforded the title compound as a white solid (0.007 g, 10%). ¹H NMR (400 MHz, DMSO-d₆) δ 7.25 (s, 1H), 7.20-7.10 (m, 2H), 6.45 (t, J=5.5 Hz, 1H), 3.62 (dt, J=13.0, 4.1 Hz, 1H), 3.13-2.94 (m, 3H), 2.85 (tt, J=12.1, 3.8 Hz, 1H), 1.89-1.76 (m, 1H), 1.67-1.49 (m, 3H), 1.46 (s, 3H), 1.38-1.25 (m, 5H), 0.74-0.57 (m, 1H), 0.47-0.30 (m, 2H), 0.11-0.08 (m, 2H). UPLC/MS (method A): Rt 2.14 min. MS (ES) C₂₁H₂₉N₃O₃ requires 371, found 372 [M+H]⁺.

### Example 30: N-(3-methoxypropyl)-2,2-dimethyl-4-(3-methyl-2-oxo-1,3-benzoxazol-6-yl)piperidine-1-carboxamide

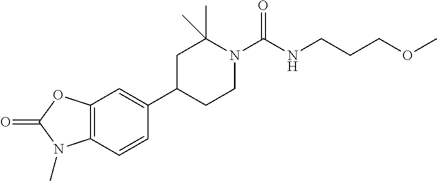 [SMILES: COCCCNC(=O)N1CCC(c2ccc3c(c2)oc(=O)n3C)CC1(C)C]

Following general procedure D (Method B), XIIj (0.030 g, 0.101 mmol) and 3-methoxypropyl amine (0.054 g, 0.61 mmol) afforded the title compound as a white solid (0.008 g, 21%). ¹H NMR (400 MHz, DMSO-d₆) δ 7.25 (d, J=1.4 Hz, 1H), 7.18-7.09 (m, 2H), 6.44 (t, J=5.4 Hz, 1H), 3.60 (dt, J=12.9, 4.1 Hz, 1H), 3.35-3.31 (m, 5H), 3.22 (s, 3H), 3.06-2.96 (m, 3H), 2.84 (ddd, J=12.3, 8.3, 3.8 Hz, 1H), 1.87-1.77 (m, 1H), 1.67-1.48 (m, 6H), 1.46 (s, 3H), 1.31 (s, 3H). UPLC/MS (method A): Rt 1.81 min. MS (ES) C₂₀H₂₉N₃O₄ requires 375, found 376 [M+H]⁺.

### Example 31: N-(4-Cyclopropylbutyl)-2,2-dimethyl-4-(3-methyl-2-oxo-1,3-benzoxazol-6-yl)piperidine-1-carboxamide

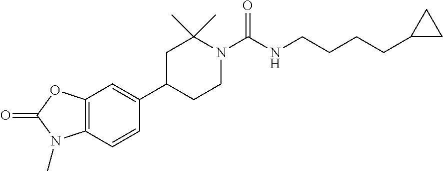 [SMILES: Cn1c(=O)oc2cc(C3CCN(C(=O)NCCCCC4CC4)C(C)(C)C3)ccc21]

Following general procedure D (Method C), XXIIj (0.052 g, 0.17 mmol) and 4-cyclopropylbutan-1-amine (0.038 g, 0.34 mmol) afforded the title compound as a white solid (0.019 g, 27%). ¹H NMR (400 MHz, DMSO-d₆) δ 7.25 (s, 1H), 7.20-7.06 (m, 2H), 6.44 (t, J=5.6 Hz, 1H), 3.61 (d, J=12.8 Hz, 1H), 3.07-2.91 (m, 3H), 2.85 (t, J=12.3 Hz, 1H), 1.90-1.76 (m, 1H), 1.67-1.50 (m, 3H), 1.46 (s, 3H), 1.37 (d, J=41.4 Hz, 7H), 1.23-1.13 (m, 2H), 0.79-0.54 (m, 1H), 0.45-0.31 (m, 2H), 0.08-0.05 (m, 2H). UPLC/MS (method A): Rt 2.42 min. MS (ES) C₂₃H₃₃N₃O₃ requires 399, found 400 [M+H]⁺.

### Example 32: 4-[3-[2-(Dimethylamino)ethyl]-2-oxo-1,3-benzoxazol-6-yl]-2,2-dimethyl-N-(4-phenylbutyl)piperidine-1-carboxamide

### tert-Butyl 4-[3-[2-(dimethylamino)ethyl]-2-oxo-1,3-benzoxazol-6-yl]-2,2-dimethyl-piperidine-1-carboxylate (XIk)

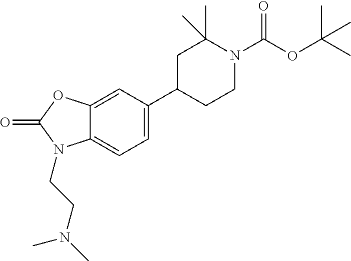 [SMILES: CN(C)CCn1c(=O)oc2cc(C3CCN(C(=O)OC(C)(C)C)C(C)(C)C3)ccc21]

To a solution of IXi (0.080 g, 0.23 mmol) in DMF (0.2 M) was added K₂CO₃ (0.095 g, 0.69 mmol) and 1,2-dibromoethane (0.349 g, 1.84 mmol) at RT and the reaction was stirred at 60° C. for 3 h. The mixture was poured into ice and the precipitate was filtered off, solubilized in DCM and dried over Na₂SO₄. After evaporation of the solvent, tert-butyl 4-[3-(2-bromoethyl)-2-oxo-1,3-benzoxazol-6-yl]-2,2-dimethyl-piperidine was used in the next step without further purification. ¹H NMR (400 MHz, CDCl₃) δ 7.11-7.08 (m, 1H), 7.07-7.03 (m, 1H), 7.01-6.96 (m, 1H), 4.21 (t, J=6.5 Hz, 2H), 3.98 (dt, J=13.7, 4.7 Hz, 1H), 3.70-3.63 (m, 5H), 3.19 (ddd, J=14.0, 10.7, 3.7 Hz, 1H), 2.87-2.81 (m, 1H), 2.01-1.91 (m, 1H), 1.56 (s, 3H), 1.48 (s, 9H), 1.37 (s, 3H). UPLC/MS (method B): Rt 1.84 min. MS (ES) C₂₁H₂₉BrN₂O₄ requires 453, found 454 [M+H]⁺.

To a solution of tert-butyl 4-[3-(2-bromoethyl)-2-oxo-1,3-benzoxazol-6-yl]-2,2-dimethyl-piperidine (0.104 g, 0.23 mmol) in DMF (0.2 M) was added K₂CO₃ (0.095 g, 0.69 mmol) and dimethylamine (0.103 g, 2.3 mmol) and the reaction was stirred at 60° C. for 2 h and then cooled to RT, poured into ice and the precipitate was filtered off. The residue was used in the next step without further purification. ¹H NMR (400 MHz, CDCl₃) δ 7.46 (d, J=8.1 Hz, 1H), 7.14-7.07 (m, 2H), 4.50 (t, J1=7.2 Hz, 2H), 3.98 (dt, J=13.7, 4.7 Hz, 1H), 3.44-3.33 (m, 2H), 3.18 (ddd, J=14.0, 10.7, 3.7 Hz, 1H), 2.87 (s, 6H), 1.99-1.91 (m, 1H), 1.73 (t, J=13.1 Hz, 1H), 1.64-1.59 (m, 1H), 1.56 (s, 3H), 1.48 (s, 10H), 1.37 (s, 3H). UPLC/MS (method A): Rt 2.24 min. MS (ES) C₂₃H₃₅N₃O₄ requires 417, found 418 [M+H]⁺.

### 3-[2-(Dimethylamino)ethyl]-6-(2,2-dimethyl-4-piperidyl)-1,3-benzoxazol-2-one dihydrochloride (XIIk)

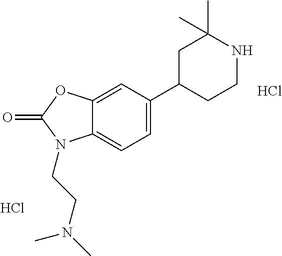 [SMILES: Cl ; CN(C)CCn1c(=O)oc2cc(C3CCNC(C)(C)C3)ccc21 ; Cl]

Following general procedure C (step 2), XIk (0.096 g, 0.23 mmol) afforded XIIk as a yellow solid (0.085 g, 95%). ¹H NMR (400 MHz, DMSO-dh) S 10.57-10.16 (m, 1H), 9.30-8.95 (m, 2H), 7.42-7.36 (m, 1H), 7.25 (s, 1H), 7.16-7.09 (m, 1H), 4.23 (t, J=6.3 Hz, 2H), 3.48-3.42 (m, 2H), 3.21-3.02 (m, 3H), 2.88-2.82 (m, 6H), 1.96-1.72 (m, 4H), 1.39 (s, 6H). UPLC/MS (method A): Rt 1.00 min. MS (ES) C₁₈H₂₇N₃O₂ requires 317, found 318 [M+H]⁺.

### 4-[3-[2-(Dimethylamino)ethyl]-2-oxo-1,3-benzoxazol-6-yl]-2,2-dimethyl-N-(4-phenylbutyl)piperidine-1-carboxamide

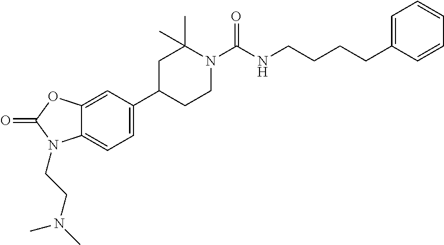 [SMILES: CN(C)CCn1c(=O)oc2cc(C3CCN(C(=O)NCCCCc4ccccc4)C(C)(C)C3)ccc21]

Following general procedure D (Method A), XIIk (0.040 g, 0.103 mmol) and 4-phenylbutyl isocyanate (0.019 g, 0.113 mmol) afforded the title compound as a gummy solid (0.009 g, 18%). ¹H NMR (400 MHz, DMSO-d₆) δ 7.31-7.13 (m, 7H), 7.09 (dd, J=8.2, 1.6 Hz, 1H), 6.46 (t, J=5.5 Hz, 1H), 3.88 (t, J=6.3 Hz, 2H), 3.60 (dt, J=12.8, 4.1 Hz, 1H), 3.07-2.92 (m, 3H), 2.89-2.78 (m, 1H), 2.62-2.53 (m, 4H), 2.16 (s, 6H), 1.86-1.77 (m, 1H), 1.67-1.48 (m, 5H), 1.45 (s, 3H), 1.43-1.35 (m, 2H), 1.30 (s, 3H). UPLC/MS (method A): Rt 2.17 min. MS (ES) C₂₉H₄₀N₄O₃ requires 492, found 493 [M+H]⁺.

### Example 33: 3-(3-Methyl-2-oxo-1,3-benzoxazol-6-yl)-N-(4-phenylbutyl)-8-azabicyclo[3.2.1]octane-8-carboxamide

### tert-Butyl 3-(trifluoromethylsulfonyloxy)-8-azabicyclo[3.2.1]oct-3-ene-8-carboxylate (VIg)

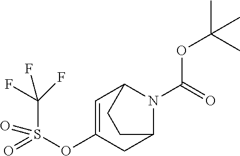 [SMILES: CC(C)(C)OC(=O)N1C2C=C(OS(=O)(=O)C(F)(F)F)CC1CC2]

Following general procedure A (step 1), Vi (0.95 g, 4.2 mmol) afforded VIg as a colorless oil (1.24 g, 83%). ¹H NMR (400 MHz, CDCl₃) δ 6.09 (d, J=4.0 Hz, 1H), 4.46 (s, 2H), 3.21-2.90 (m, 1H), 2.23 (s, 1H), 2.09 (d, J=16.4 Hz, 1H), 2.05-1.94 (m, 2H), 1.85-1.65 (m, 1H), 1.46 (s, 9H). UPLC/MS (method B): Rt 1.61 min. MS (ES) C₁₃H₁₈F₃NO₅S requires 357, found 358 [M+H]⁺.

### tert-Butyl 3-(3-hydroxy-4-nitrophenyl)-8-azabicyclo[3.2.1]oct-3-ene-8-carboxylate (VIIj)

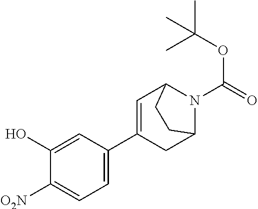 [SMILES: CC(C)(C)OC(=O)N1C2C=C(c3ccc([N+](=O)[O-])c(O)c3)CC1CC2]

Following general procedure A (step 2), VIg (0.60 g, 1.7 mmol) and 5-bromo-2-nitrophenol (0.33 g, 1.53 mmol) afforded VIIj as yellow solid (0.33 g, 56%). ¹H NMR (400 MHz, CDCl₃) δ 10.62 (s, 1H), 8.02 (d, J=8.9 Hz, 1H), 7.06 (d, J=1.7 Hz, 1H), 7.00 (dd, J=9.0, 1.9 Hz, 1H), 6.67 (d, J=5.1 Hz, 1H), 4.59-4.43 (m, 2H), 3.08 (d, J=15.5 Hz, 1H), 2.31-2.13 (m, 2H), 2.10-1.86 (m, 2H), 2.03-1.86 (m, 2H), 1.73-1.64 (m, 1H), 1.45 (s, 9H). UPLC/MS (method B): Rt 1.46 min. MS (ES) C₁₈H₂₂N₂O₅ requires 346, found 347 [M+H]⁺.

### tert-Butyl-3-(4-amino-3-hydroxyphenyl)-8-azabicyclo[3.2.1]octane-8-carboxylate (VIIIj)

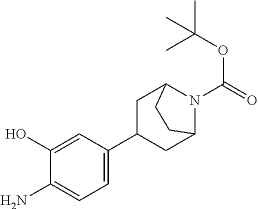 [SMILES: CC(C)(C)OC(=O)N1C2CCC1CC(c1ccc(N)c(O)c1)C2]

Following general procedure B (Method A, step 1), VIIj (0.33 g, 0.96 mmol) afforded VIIIj which was used in the next step without further purification. UPLC/MS (method A): Rt 2.01 min. MS (ES) C₁₈H₂₆N₂O₃ requires 318, found 319 [M+H]⁺.

### tert-Butyl 3-(2-oxo-3H-1,3-benzoxazol-6-yl)-8-azabicyclo[3.2.1]octane-8-carboxylate (IXj)

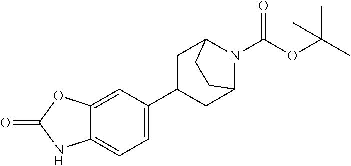 [SMILES: CC(C)(C)OC(=O)N1C2CCC1CC(c1ccc3[nH]c(=O)oc3c1)C2]

Following general procedure B (step 2), VIIIj (0.300 g, 0.94 mmol) afforded IXj as a yellow oil (0.050 g, 15%). ¹H NMR (400 MHz, CDCl₃) δ 8.52 (s, 1H), 7.10-6.91 (m, 3H), 4.43-4.20 (m, 2H), 2.74-2.40 (m, 3H), 2.09-1.96 (m, 2H), 1.64-1.57 (m, 2H), 1.55-1.44 (m, 2H), 1.43 (s, 3H), 1.24 (s, 6H). UPLC/MS (method A): Rt 2.18 min. MS (ES) C₁₉H₂₄N₂O₄ requires 344, found 345 [M+H]⁺.

### tert-Butyl 3-(3-methyl-2-oxo-1,3-benzoxazol-6-yl)-8-azabicyclo[3.2.1]octane-8-carboxylate (XII)

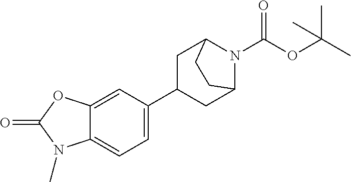 [SMILES: Cn1c(=O)oc2cc(C3CC4CCC(C3)N4C(=O)OC(C)(C)C)ccc21]

Following general procedure C (step 1), IXj (0.050 g, 0.13 mmol) and MeI (0.03 g, 0.2 mmol) afforded XII which was used in the next step without further purification. UPLC/MS (method A): Rt 2.39 min. MS (ES) C₂₀H₂₆N₂O₄ requires 358, found 359 [M+H]⁺.

### 6-(8-Azabicyclo[3.2.1]octan-3-yl)-3-methyl-1,3-benzoxazol-2-one hydrochloride (XIII)

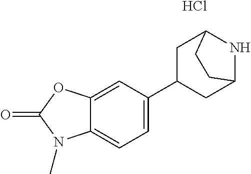 [SMILES: Cl ; Cn1c(=O)oc2cc(C3CC4CCC(C3)N4)ccc21]

Following general procedure C (step 2), XII (0.06 g, 0.167 mmol) afforded XIII as a white solid (0.04 g, 92%). (UPLC/MS (method A): Rt 1.10 min. MS (ES) C₁₅H₁₈N₂O₂ requires 258, found 259 [M+H]⁺.

### 3-(3-Methyl-2-oxo-1,3-benzoxazol-6-yl)-N-(4-phenylbutyl)-8-azabicyclo[3.2.1]octane-8-carboxamide

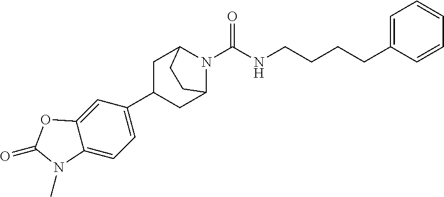 [SMILES: Cn1c(=O)oc2cc(C3CC4CCC(C3)N4C(=O)NCCCCc3ccccc3)ccc21]

Following general procedure D (Method A), XIII (0.045 g, 0.14 mmol) and 4-phenylbutyl isocyanate (0.05 g, 0.28 mmol) afforded the title compound as a white solid (85:15, endo:exo stereoisomers, 0.035 g, 62%). ¹H NMR (400 MHz, DMSO-d₆) δ 7.29-7.21 (m, 3.4H), 7.21-7.07 (m, 5.0H), 7.05-7.00 (m, 1.2H), 6.45 (t, J=5.7 Hz, 1H), 6.44-6.42 (m, 0.15H), 4.26-4.17 (m, 2.3H), 3.31-3.29 (m, 3.5H), 3.20-3.12 (m, 0.2H), 3.09 (q, J=6.7 Hz, 2H), 2.63-2.56 (m, 2.3H), 2.55-2.45 (m, 1H), 2.30 (dt, J=14.5, 7.5 Hz, 2H), 1.92-1.65 (m, 3.3H), 1.65-1.52 (m, 4.8H), 1.50-1.40 (m, 4.4H). UPLC/MS (method A): Rt 2.20 min. MS (ES) C₂₆H₃₁N₃O₃ requires 433, found 434 [M+H]⁺.

### Example 34: 8-(3-Methyl-2-oxo-1,3-benzoxazol-6-yl)-N-(4-phenylbutyl)-2-oxa-5-azaspiro[3.5]nonane-5-carboxamide

### Benzyl 8-(trifluoromethylsulfonyloxy)-2-oxa-5-azaspiro[3.5]non-7-ene-5-carboxylate (VIh)

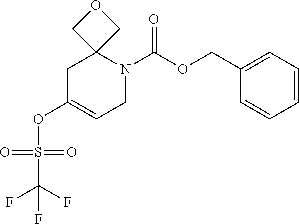 [SMILES: O=C(OCc1ccccc1)N1CC=C(OS(=O)(=O)C(F)(F)F)CC12COC2]

Following general procedure A, Vi (0.246 g, 0.89 mmol) afforded VIh as a colorless oil (0.220 g, 61%). ¹H NMR (400 MHz, CDCl₃) δ 7.42-7.28 (m, 5H), 5.78 (tt, J=3.2, 1.4 Hz, 1H), 5.13 (s, 2H), 4.82 (d, J=6.6 Hz, 2H), 4.31 (s, 2H), 4.08 (q, J=2.8 Hz, 2H), 2.95 (s, 2H). UPLC/MS (method B): Rt 1.24 min. MS (ES) C₁₆H₁₆F₃NO₆S requires 407, found 408 [M+H]⁺.

### Benzyl 8-(3-hydroxy-4-nitro-phenyl)-2-oxa-5-azaspiro[3.5]non-7-ene-5-carboxylate (VIIk)

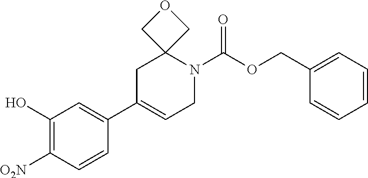 [SMILES: O=C(OCc1ccccc1)N1CC=C(c2ccc([N+](=O)[O-])c(O)c2)CC12COC2]

Following general procedure A, VIh (0.220 g, 0.54 mmol) and 5-bromo-2-nitrophenol (0.141 g, 0.65 mmol) afforded VIIk as a yellow oil (0.192 g, 90%). ¹H NMR (400 MHz, CDCl₃) δ 10.63 (s, 1H), 8.10-8.03 (m, 1H), 7.41-7.30 (m, 5H), 7.11 (d, J=2.0 Hz, 1H), 7.00 (dd, J=8.9, 2.0 Hz, 1H), 6.27-6.18 (m, 1H), 5.12 (s, 2H), 4.89 (d, J=6.5 Hz, 2H), 4.31 (s, 2H), 4.18 (q, J=2.8 Hz, 2H), 3.01 (s, 2H). UPLC/MS (method B): Rt 1.12 min. MS (ES) C₂₁H₂₀N₂O₆ requires 396, found 395 [M−H]⁻.

### Benzyl 8-(4-amino-3-hydroxy-phenyl)-2-oxa-5-azaspiro[3.5]non-7-ene-5-carboxylate (VII′a)

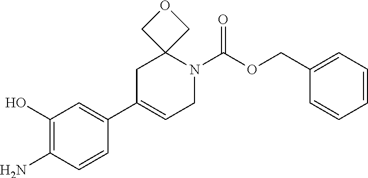 [SMILES: Nc1ccc(C2=CCN(C(=O)OCc3ccccc3)C3(COC3)C2)cc1O]

Following general procedure B, step 1 (Method C), VIIk (0.182 g, 0.46 mmol) afforded VII′a which was used in the next step without further purification. UPLC/MS (method A): Rt 1.86 min. MS (ES) C₂₁H₂₂N₂O₄ requires 366, found 367 [M+H]⁺.

### Benzyl 8-(2-oxo-3H-1,3-benzoxazol-6-yl)-2-oxa-5-azaspiro[3.5]non-7-ene-5-carboxylate (XIVb)

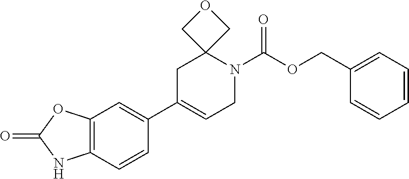 [SMILES: O=C(OCc1ccccc1)N1CC=C(c2ccc3[nH]c(=O)oc3c2)CC12COC2]

Following general procedure B, step 2, VII′a (0.287 g, 0.46 mmol) afforded XIVb as a colorless oil (0.106 g, 56%). ¹H NMR (400 MHz, CDCl₃) δ 8.44 (br s, 1H), 7.41-7.30 (m, 5H), 7.24-7.22 (m, 1H), 7.18 (dd, J=8.2, 1.7 Hz, 1H), 7.05-7.00 (m, 1H), 6.01-5.94 (m, 1H), 5.13 (s, 2H), 4.90 (d, J=6.4 Hz, 2H), 4.33 (s, 2H), 4.18-4.13 (m, 2H), 3.01 (s, 2H). UPLC/MS (method A): Rt 1.96 min. MS (ES) C₂₂H₂₀N₂O₅ requires 392, found 393 [M+H]⁺.

### Benzyl 8-(3-methyl-2-oxo-1,3-benzoxazol-6-yl)-2-oxa-5-azaspiro[3.5]non-7-ene-5-carboxylate (XVb)

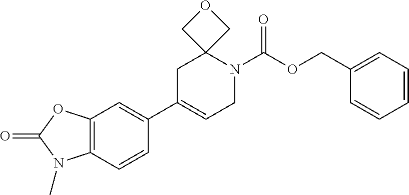 [SMILES: Cn1c(=O)oc2cc(C3=CCN(C(=O)OCc4ccccc4)C4(COC4)C3)ccc21]

Following general procedure C, step 1, XIVb (0.106 g, 0.27 mmol) and MeI (0.350 g, 2.16 mmol) afforded XVb which was used in the next step without purification. ¹H NMR (400 MHz, CDCl₃) δ 7.42-7.32 (m, 5H), 7.25-7.20 (m, 2H), 6.95 (d, J=8.0 Hz, 1H), 6.01-5.94 (m, 1H), 5.15 (s, 2H), 4.92 (d, J=6.4 Hz, 2H), 4.35 (s, 2H), 4.16 (d, J=3.1 Hz, 2H), 3.44 (s, 3H), 3.04 (s, 2H). UPLC/MS (method A): Rt 2.13 min. MS (ES) C₂₃H₂₂N₂O₅ requires 406, found 407 [M+H]⁺.

### 3-Methyl-6-(2-oxa-5-azaspiro[3.5]nonan-8-yl)-1,3-benzoxazol-2-one (XIIm)

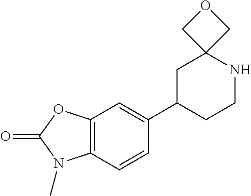 [SMILES: Cn1c(=O)oc2cc(C3CCNC4(COC4)C3)ccc21]

Following general procedure B, (Method E), XIVb (0.110 g, 0.27 mmol) afforded XIIm which was used in the next step without purification. ¹H NMR (400 MHz, CDCl₃) δ 7.12-7.02 (m, 2H), 6.95-6.86 (m, 1H), 4.68 (d, J=6.2 Hz, 1H), 4.58 (dd, J=6.2, 1.5 Hz, 1H), 3.39 (s, 3H), 3.10 (ddd, J=11.9, 4.1, 2.5 Hz, 1H), 2.79 (td, J=12.0, 2.7 Hz, 1H), 2.62 (tt, J=12.4, 3.6 Hz, 1H), 2.39-2.27 (m, 1H), 1.86-1.55 (m, 5H). UPLC/MS (method A): Rt 1.05 min. MS (ES) C₁₅H₁₈N₂O₃ requires 274, found 275 [M+H]⁺.

### 8-(3-Methyl-2-oxo-1,3-benzoxazol-6-yl)-N-(4-phenylbutyl)-2-oxa-5-azaspiro[3.5]nonane-5-carboxamide

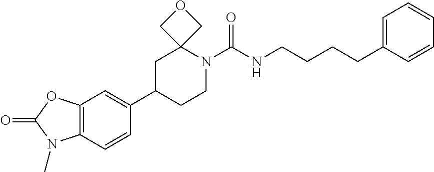 [SMILES: Cn1c(=O)oc2cc(C3CCN(C(=O)NCCCCc4ccccc4)C4(COC4)C3)ccc21]

Following general procedure D (Method A), XIIm (0.030 g, 0.11 mmol) and 4-phenylbutyl isocyanate (0.021 g, 0.12 mmol) afforded the title compound as a white solid (0.016 g, 36%). ¹H NMR (400 MHz, DMSO-d₆) δ 7.29-7.22 (m, 2H), 7.20-7.11 (m, 5H), 7.06 (dd, J=8.1, 1.6 Hz, 1H), 6.77 (t, J=5.6 Hz, 1H), 4.70 (d, J=7.1 Hz, 1H), 4.49-4.37 (m, 2H), 4.19 (d, J=7.0 Hz, 1H), 3.73 (d, J=15.1 Hz, 1H), 3.32 (s, 3H), 3.13 (dt, J=12.9, 6.5 Hz, 1H), 3.05-2.90 (m, 2H), 2.70-2.54 (m, 3H), 2.24-2.12 (m, 1H), 1.97-1.86 (m, 1H), 1.62-1.50 (m, 3H), 1.49-1.38 (m, 2H), 1.31 (td, J=12.7, 3.5 Hz, 1H). UPLC/MS (method A): Rt 2.08 min. MS (ES) C₂₆H₃₁N₃O₄ requires 449, found 450 [M+H]⁺.

### Example 35: 4-(3-Methyl-2-oxo-1,3-benzoxazol-6-yl)-2-oxo-N-(4-phenylbutyl)piperidine-1-carboxamide

### tert-Butyl 4-(3-benzyloxy-4-nitro-phenyl)-6-oxo-2,3-dihydropyridine-1-carboxylate (VI)

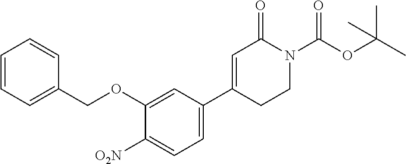 [SMILES: CC(C)(C)OC(=O)N1CCC(c2ccc([N+](=O)[O-])c(OCc3ccccc3)c2)=CC1=O]

Following general procedure A, VI′i (0.96 g, 0.297 mmol) and 2-benzyloxy-4-bromo-1-nitrobenzene (0.100 g, 0.327 mmol) afforded VIB as a yellow solid (0.102 g, 74%). ¹H NMR (400 MHz, CDCl₃) δ 7.92 (d, J=8.5 Hz, 1H), 7.52-7.47 (m, 2H), 7.43 (ddd, J=7.5, 6.6, 1.4 Hz, 2H), 7.40-7.35 (m, 1H), 7.22 (d, J=1.8 Hz, 1H), 7.16 (dd, J=8.4, 1.8 Hz, 1H), 6.33-6.30 (m, 1H), 5.30 (s, 2H), 4.02 (t, J=6.4 Hz, 2H), 2.78 (td, J=6.5, 1.4 Hz, 2H), 1.59 (s, 9H). UPLC/MS (method B): Rt 2.55 min. MS (ES) C₂₃H₂₄N₂O₆ requires 424, found 425 [M+H]⁺.

### tert-Butyl 4-(4-amino-3-hydroxy-phenyl)-2-oxo-piperidine-1-carboxylate (VIIIk)

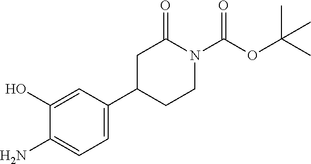 [SMILES: CC(C)(C)OC(=O)N1CCC(c2ccc(N)c(O)c2)CC1=O]

Following general procedure B (Method A), VI (0.50 g, 1.18 mmol) afforded VIIIk which was used in the next step without further purification. UPLC/MS (method A): Rt 1.65 min. MS (ES) C₁₆H₂₂N₂O₄ requires 306, found 307 [M+H]⁺.

### tert-Butyl 2-oxo-4-(2-oxo-3H-1,3-benzoxazol-6-yl)piperidine-1-carboxylate (IXk)

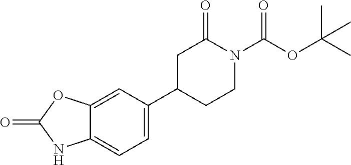 [SMILES: CC(C)(C)OC(=O)N1CCC(c2ccc3[nH]c(=O)oc3c2)CC1=O]

Following general procedure B, VIIk (0.360 g, 1.18 mmol) afforded IXk as a yellow oil (0.300 g, 70%). UPLC/MS (method A): Rt 1.77 min. MS (ES) C₁₇H₂₀N₂O₅ requires 332, found 333 [M+H]⁺.

### tert-Butyl 4-(3-methyl-2-oxo-1,3-benzoxazol-6-yl)-2-oxo-piperidine-1-carboxylate (XIm)

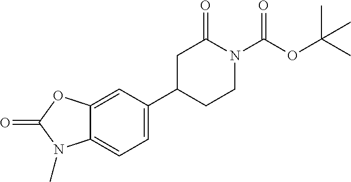 [SMILES: Cn1c(=O)oc2cc(C3CCN(C(=O)OC(C)(C)C)C(=O)C3)ccc21]

Following general procedure C, IXk (0.310 g, 0.93 mmol) and CH₃I (0.2 g, 0.09 mL, 1.4 mmol) afforded XIm as a white solid. ¹H NMR (400 MHz, CDCl₃) δ 7.10-7.05 (m, 1H), 7.03-7.00 (m, 1H), 6.92 (d, J=8.0 Hz, 1H), 3.88 (ddd, J=12.9, 5.0, 4.1 Hz, 1H), 3.61 (ddd, 1=12.9, 10.9, 4.3 Hz, 1H), 3.39 (s, 3H), 3.22-3.09 (m, 1H), 2.84 (ddd, J=17.1, 5.4, 2.0 Hz, 1H), 2.59 (dd, J=17.1, 11.2 Hz, 1H), 2.27-2.15 (m, 1H), 1.95 (dtd, J=13.6, 11.0, 5.0 Hz, 1H), 1.54 (s, 9H). UPLC/MS (method A): Rt 1.94 min. MS (ES) C₁₈H₂₂N₂O₅ requires 346, found 345 [M+H]⁺.

### 3-Methyl-6-(2-oxo-4-piperidyl)-1,3-benzoxazol-2-one (XIIn)

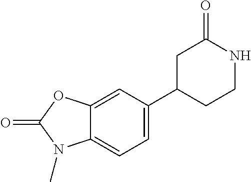 [SMILES: Cn1c(=O)oc2cc(C3CCNC(=O)C3)ccc21]

Following general procedure C, XIm (0.045 g, 0.130 mmol) afforded XIIn as a white solid (0.028 g, 87%). ¹H NMR (400 MHz, DMSO-d₆) δ 7.65 (s, 1H), 7.31 (d, J=1.4 Hz, 1H), 7.21-7.12 (m, 2H), 3.32 (s, 3H), 3.21 (qd, J=6.6, 4.1 Hz, 2H), 3.12-3.01 (m, 1H), 2.43-2.23 (m, 2H), 1.93-1.78 (m, 2H). (UPLC/MS (method A): Rt 1.21 min. MS (ES) C₁₃H₁₄N₂O₃ requires 246, found 247 [M+H]⁺.

### 4-(3-Methyl-2-oxo-1,3-benzoxazol-6-yl)-2-oxo-N-(4-phenylbutyl)piperidine-1-carboxamide

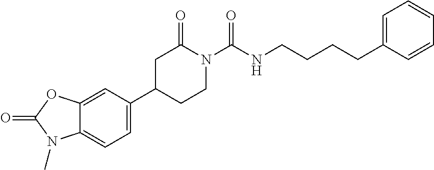 [SMILES: Cn1c(=O)oc2cc(C3CCN(C(=O)NCCCCc4ccccc4)C(=O)C3)ccc21]

Following general procedure D (Method A), XIIn (0.025 g, 0.102 mmol) and 4-phenylbutyl isocyanate (0.02 g, 0.112 mmol) the title compound as a white solid (0.030 g, 70%). ¹H NMR (400 MHz, CDCl₃) δ 9.40-9.30 (m, 1H), 7.30-7.27 (m, 1H), 7.21-7.14 (m, 3H), 7.08-7.05 (m, 1H), 7.03-7.00 (m, 1H), 6.92 (d, J=8.0 Hz, 1H), 4.16 (ddd, J=13.7, 5.0, 4.2 Hz, 1H), 3.63 (ddd, J=13.6, 10.9, 4.3 Hz, 1H), 3.40 (s, 3H), 3.34 (q, J=6.6 Hz, 2H), 3.24-3.05 (m, 1H), 2.88 (ddd, J=17.7, 5.8, 2.0 Hz, 1H), 2.69-2.50 (m, 3H), 2.24 (dtd, J=14.2, 4.2, 2.0 Hz, 1H), 1.91 (dtd, J=13.8, 11.0, 5.0 Hz, 1H), 1.74-1.59 (m, 4H). UPLC/MS (method A): Rt 2.36 min. MS (ES) C₂₄H₂₇N₃O₄ requires 421, found 422 [M+H]⁺.

### Example 36: 2-(2-Oxo-3H-1,3-benzoxazol-6-yl)-N-(4-phenylbutyl)piperidine-1-carboxamide

### tert-Butyl N-[5-oxo-5-(2-oxo-3H-1,3-benzoxazol-6-yl)pentyl]carbamate (XXIIa)

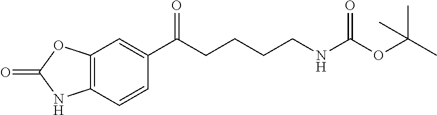 [SMILES: CC(C)(C)OC(=O)NCCCCC(=O)c1ccc2[nH]c(=O)oc2c1]

Following general procedure G, XXa (2.8 g, 14.0 mmol) and 6-bromo-3H-1,3-benzoxazol-2-one (0.6 g, 2.80 mmol) afforded XXIIa as a white powder (0.594 g, 63%). ¹H NMR (400 MHz, DMSO-d₆) δ 11.99 (bs, 1H), 7.85-7.80 (m, 2H), 7.19 (d, J=8.6 Hz, 1H), 6.79 (t, J=5.8 Hz, 1H), 3.05-2.85 (m, 4H), 1.65-1.50 (m, 2H), 1.50-1.40 (m, 2H), 1.37 (s, 9H). UPLC/MS (method A): Rt 1.84 min. MS (ES) C₁₇H₂₂N₂O₅ requires 334, found 335 [M+H]⁺.

### 6-(2-Piperidyl)-3H-1,3-benzoxazol-2-one (XXIIIa)

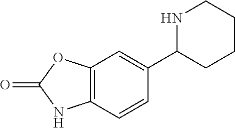 [SMILES: O=c1[nH]c2ccc(C3CCCCN3)cc2o1]

To a suspension of XXIIa (0.297 g, 0.89 mmol) in DCM (0.1 M) was added TFA (2.0 g, 17.8 mmol) and the reaction mixture was stirred at RT for 1 h. After evaporation of the solvent, the residue was used in the next step without further purification. UPLC/MS (method A): Rt 0.89 min. MS (ES) C₁₂H₁₄N₂O₃ requires 234, found 235 [M+H]⁺.

To a solution of compound from Step 1 in ACN (0.1 M) was added NaBH(OAc)₃ (0.566 g, 2.67 mmol) and the reaction mixture was stirred at RT for 30 min and then quenched with the addition of MeOH and diluted with EA. The organic phase was washed with a saturated aqueous NaHCO₃ solution, brine and dried over Na₂SO₄. After evaporation of the solvent, the residue was purified by SCX to afford XXIIIa. UPLC/MS (method A): Rt 0.94 min. MS (ES) C₁₂H₁₄N₂O₂ requires 218, found 219 [M+H]⁺.

### 2-(2-Oxo-3H-1,3-benzoxazol-6-yl)-N-(4-phenylbutyl)piperidine-1-carboxamide

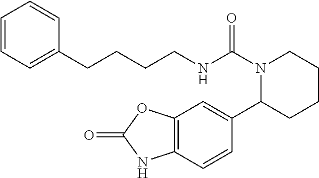 [SMILES: O=C(NCCCCc1ccccc1)N1CCCCC1c1ccc2[nH]c(=O)oc2c1]

Following general procedure D (Method A), XXIIIa (0.025 g, 0.1 mmol) and 4-phenylbutyl isocyanate (0.018 g, 0.1 mmol) afforded the title compound as a white solid (0.086 g, 91%). ¹H NMR (400 MHz, DMSO-d₆) δ 11.55 (s, 1H), 7.26 (dd, J=7.9, 6.9 Hz, 2H), 7.16 (dt, J=7.9, 1.4 Hz, 3H), 7.05 (d, J=8.1 Hz, 2H), 6.95 (dd, J=8.0, 1.4 Hz, 1H), 6.49 (t, J=5.5 Hz, 1H), 5.35-5.30 (m, 1H), 3.86 (d, J=13.4 Hz, 1H), 3.15-3.00 (m, 2H), 2.75-2.60 (m, 1H), 2.60-2.50 (m, 2H), 2.27 (d, J=13.9 Hz, 1H), 1.80-1.65 (m, 1H), 1.60-1.20 (m, 8H). UPLC/MS (method A): Rt 2.07 min. MS (ES) C₂₃H₂₇N₃O₃ requires 393, found 394 [M+H]⁺.

### Example 37: 2-(3-Methyl-2-oxo-1,3-benzoxazol-6-yl)-N-(4-phenylbutyl)piperidine-1-carboxamide

### tert-Butyl N-[5-(3-methyl-2-oxo-1,3-benzoxazol-6-yl)-5-oxo-pentyl]carbamate (XXIVa)

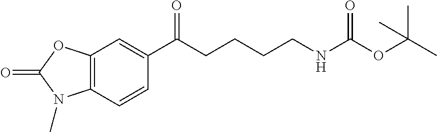 [SMILES: Cn1c(=O)oc2cc(C(=O)CCCCNC(=O)OC(C)(C)C)ccc21]

Following general procedure C (step 1), XXIIa (0.030 g, 0.090 mmol) and CH₃I (0.020 g, 0.14 mmol) afforded XXIVa which was used in the next step without further purification. ¹H NMR (400 MHz, DMSO-d₆) δ 7.93 (dd, J=8.2, 1.6 Hz, 1H), 7.88 (d, J=1.5 Hz, 1H), 7.37 (d, J=8.2 Hz, 1H), 6.79 (d, J=6.5 Hz, 1H), 3.39 (s, 3H), 3.05-2.90 (m, 4H), 1.65-1.50 (m, 2H), 1.50-1.40 (m, 2H), 1.37 (s, 9H). UPLC/MS (method A): Rt 1.99 min. MS (ES) C₁₈H₂₄N₂O₅ requires 348, found 349 [M+H]⁺.

### 3-Methyl-6-(2-piperidyl)-1,3-benzoxazol-2-one (XXVa)

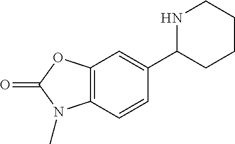 [SMILES: Cn1c(=O)oc2cc(C3CCCCN3)ccc21]

To a suspension of XXIVa (0.10 g, 0.28 mmol) in DCM (0.1 M) was added TFA (0.43 mL, 5.6 mmol) and the reaction mixture was stirred at RT for 1 h. After evaporation of the solvent, the residue was used in the next step without further purification. UPLC/MS (method A): Rt 1.07 min. MS (ES) C₁₈H₂₄N₂O₅ requires 248, found 249 [M+H]⁺.

To a solution of compound from Step 1 (0.070 g, 0.28 mmol) in ACN (0.1 M) was added NaBH(OAc)₃ (0.178 g, 0.84 mmol) and the reaction mixture was stirred at RT for 30 min and then quenched with MeOH, diluted with EA, washed with saturated aq. NaHCO₃ solution, brine and dried over Na₂SO₄. After evaporation of the solvent, the residue was purified by SCX to afford XXVa (0.062 g, 92%). ¹H NMR (400 MHz, DMSO-d₆) δ 9.35 (s, 1H), 7.65 (d, J=1.5 Hz, 1H), 7.44 (dd, J=8.1, 1.6 Hz, 1H), 7.31 (d, J=8.1 Hz, 1H) 4.30-4.25 (m, 1H), 3.35 (s, 3H), 3.10-2.95 (m, 1H), 2.00-1.75 (m, 6H), 1.70-1.50 (m, 1H). UPLC/MS (method A): Rt 1.07 min. MS (ES) C₁₃H₁₆N₂O₂ requires 232, found 233 [M+H]⁺.

### 2-(3-Methyl-2-oxo-1,3-benzoxazol-6-yl)-N-(4-phenylbutyl)piperidine-1-carboxamide

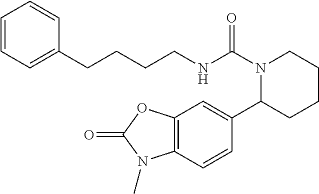 [SMILES: Cn1c(=O)oc2cc(C3CCCCN3C(=O)NCCCCc3ccccc3)ccc21]

Following general procedure D (Method A), XXVa (0.070 g, 0.026 mmol) and 4-phenylbutyl isocyanate (0.050 g, 0.29 mmol) afforded the title compound as a white solid (0.098 g, 93%). ¹H NMR (400 MHz, CDCl₃) δ 9.78 (s, 1H), 7.35-7.25 (m, 2H), 7.25-7.15 (m, 3H), 7.00 (s, 1H), 6.93 (d, J=8.2 Hz, 1H), 6.83 (d, J=8.1 Hz, 1H), 5.35-5.25 (m, 1H), 4.75-4.65 (m, 1H), 3.82 (d, J=12.9 Hz, 1H), 3.32 (d, J=6.5 Hz, 2H), 3.04 (td, J=13.3, 12.2, 3.7 Hz, 1H), 2.63 (t, J=7.4 Hz, 2H), 2.25-2.15 (m, 1H), 2.05-1.90 (m, 2H), 1.90-1.85 (m, 1H), 1.75-1.50 (m, 7H), 1.50-1.35 (m, 1H). UPLC/MS (method A): Rt 2.23 min. MS (ES) C₂₄H₂₉N₃O₃ requires 407, found 408 [M+H]⁺.

### Example 38: 3-(3-Methyl-2-oxo-1,3-benzoxazol-6-yl)-N-(4-phenylbutyl)morpholine-4-carboxamide

### tert-Butyl N-[2-[2-oxo-2-(2-oxo-3H-1,3-benzoxazol-6-yl)ethoxy]ethyl]carbamate (XXIId)

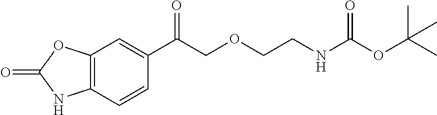 [SMILES: CC(C)(C)OC(=O)NCCOCC(=O)c1ccc2[nH]c(=O)oc2c1]

Following general procedure G, XXe (1.18 g, 5.84 mmol) and 6-bromo-3H-1,3-benzoxazol-2-one (0.250 g, 1.17 mmol) afforded XXIId as a white solid (0.233 g, 59%). ¹H NMR (400 MHz, CDCl₃) δ 9.42 (s, 1H), 7.89-7.74 (m, 2H), 7.18 (d, J=8.1 Hz, 1H), 5.25 (bs, 1H), 4.78 (s, 2H), 3.70 (t, J=5.1 Hz, 2H), 3.42 (t, J=5.1 Hz, 2H), 1.47 (s, 9H). UPLC/MS (method A): Rt 1.64 min. MS (ES) C₁₆H₂₀N₂O₆ requires 336, found 337 [M+H]⁺.

### tert-Butyl N-[2-[2-(3-methyl-2-oxo-1,3-benzoxazol-6-yl)-2-oxo-ethoxy]ethyl]carbamate (XXIVd)

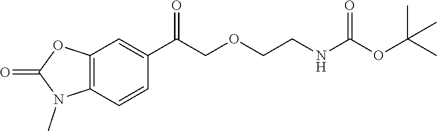 [SMILES: Cn1c(=O)oc2cc(C(=O)COCCNC(=O)OC(C)(C)C)ccc21]

To a solution (0.2 M) of XXIId (0.130 g, 0.39 mmol) in anydrous DMF was added MeI (0.110 g, 0.77 mmol) and K₂CO₃ (0.040 g, 0.29 mmol) and the reaction mixture was stirred at RT for 3 h. The reaction mixture was diluted with DCM, washed with brine, dried over Na₂SO₄ and concentrated to afford XXIVd which was used in the next step without further purification. ¹H NMR (400 MHz, CDCl₃) δ 7.89 (d, J=7.6 Hz, 1H), 7.82 (s, 1H), 7.05 (d, J=8.1 Hz, 1H), 5.15 (bs, 1H), 4.77 (s, 2H), 3.68 (t, J=5.1 Hz, 2H), 3.48 (s, 3H), 3.40 (d, J=5.3 Hz, 2H), 1.47 (s, 9H). UPLC/MS (method A): Rt 1.78 min. MS (ES) C₁₇H₂₂N₂O₆ requires 350, found 351 [M+H]⁺.

### 3-Methyl-6-morpholin-3-yl-1,3-benzoxazol-2-one (XXVd)

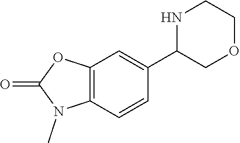 [SMILES: Cn1c(=O)oc2cc(C3COCCN3)ccc21]

To a suspension of XXIVd (0.110 g, 0.314 mmol) in DCM (0.1 M) was added TFA (0.537 g, 0.36 mL, 4.71 mmol) and the reaction mixture was stirred at RT for 1 h. After evaporation of the solvent, the residue was used in the next step without further purification. UPLC/MS (method A): Rt 0.95 min. MS (ES) C₁₂H₁₄N₂O₃ requires 250, found 251 [M+H]⁺.

To a solution of compound from Step/in ACN (0.1 M) was added NaBH(OAc)₃ (0.2 g, 0.942 mmol) and the reaction mixture was stirred at RT for 30 min and then quenched with the addition of MeOH and diluted with EA. The organic phase was washed with saturated aq. NaHCO₃ solution, brine and dried over Na₂SO₄ and concentrated to afford XXVd which was used in the next step without further purification. ¹H NMR (400 MHz, DMSO-d₆) δ 9.87 (s, 2H), 7.69 (d, J1=1.6 Hz, 1H), 7.49 (dd, J=8.1, 1.6 Hz, 1H), 7.34 (d, J=8.1 Hz, 1H), 4.50 (dd, J=10.7, 3.7 Hz, 1H), 4.11-3.73 (m, 4H), 3.36 (s, 3H), 3.30-3.18 (m, 2H). UPLC/MS (method A): Rt 0.91 min. MS (ES) C₁₂H₁₄N₂O₃.ClH requires 234, found 235 [M+H]⁺.

### 3-(3-Methyl-2-oxo-1,3-benzoxazol-6-yl)-N-(4-phenylbutyl)morpholine-4-carboxamide

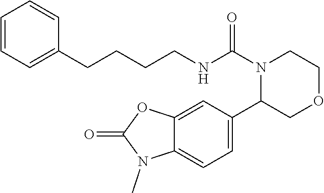 [SMILES: Cn1c(=O)oc2cc(C3COCCN3C(=O)NCCCCc3ccccc3)ccc21]

Following general procedure D (Method A), XXVd (0.052 g, 0.19 mmol) and 4-phenylbutyl isocyanate (0.037 g, 0.21 mmol) afforded the title compound as a white solid (0.067 g, 84%). ¹H NMR (400 MHz, DMSO-d₆) δ 7.34-7.23 (m, 3H), 7.22-7.09 (m, 5H), 6.56 (t, 0.1=5.5 Hz, 1H), 5.10 (d, J=3.3 Hz, 1H), 4.28 (d, J=11.9 Hz, 1H), 3.91-3.58 (m, 3H), 3.44 (td, J=11.5, 3.1 Hz, 1H), 3.32 (s, 3H), 3.19-2.88 (m, 3H), 2.59-2.52 (m, 2H), 1.59-1.35 (m, 4H). UPLC/MS (method A): Rt 2.01 min. MS (ES) C₂₃H₂₇N₃O₄ requires 409, found 410 [M+H]⁺.

### Example 39: 2-(3-Methyl-2-oxo-1,3-benzoxazol-6-yl)-N-(4-phenylbutyl)piperazine-1-carboxamide

### Benzyl N-[2-(tert-butoxycarbonylamino)ethyl]-N-[2-oxo-2-(2-oxo-3H-1,3-benzoxazol-6-yl)ethyl]carbamate (XXIIb)

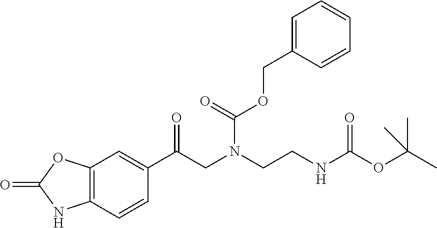 [SMILES: CC(C)(C)OC(=O)NCCN(CC(=O)c1ccc2[nH]c(=O)oc2c1)C(=O)OCc1ccccc1]

Following general procedure G, XXb (0.640 g, 1.91 mmol) and 6-bromo-3H-1,3-benzoxazol-2-one (0.1 g, 0.47 mmol) afforded XXIIb as a white solid (0.158 g, 71%). 1H NMR (400 MHz, DMSO-d₆) δ 7.90-7.80 (m, 2H), 7.45-7.30 (m, 3H), 7.30-7.15 (m, 4H), 6.85-6.65 (m, 1H), 5.20-4.95 (m, 2H), 4.90-4.75 (m, 2H), 3.45-3.30 (m, 2H), 3.20-3.05 (m, 2H), 1.37 (s, 9H). UPLC/MS (method A): Rt 2.07 min. MS (ES) C₂₄H₂₇N₃O₇ requires 469, found 470 [M+H]⁺.

### tert-Butyl N-[5-(3-methyl-2-oxo-1,3-benzoxazol-6-yl)-5-oxo-pentyl]carbamate (XXIVb)

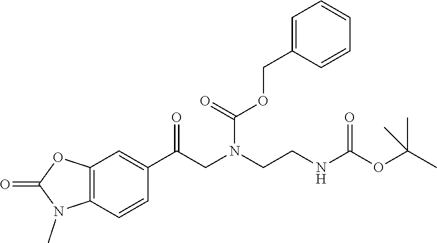 [SMILES: Cn1c(=O)oc2cc(C(=O)CN(CCNC(=O)OC(C)(C)C)C(=O)OCc3ccccc3)ccc21]

Following general procedure C (step 1), XXIIb (0.150 g, 0.32 mmol) and MeI (0.091 g, 0.64 mmol) afforded XXIVb which was used in the next step without further purification. ¹H NMR (400 MHz, CDCl₃) δ 7.95-7.70 (m, 2H), 7.45-7.30 (m, 3H), 7.30-7.20 (m, 2H), 7.10-7.00 (m, 1H), 5.25-5.00 (m, 3H), 4.80-4.60 (m, 2H), 3.60-3.50 (m, 2H), 3.48 (s, 3H), 3.40-3.20 (m, 2H), 1.42 (s, 9H). UPLC/MS (method A): Rt 2.19 min. MS (ES) C₂₅H₂₉N₃O₇ requires 483, found 484 [M+H]⁺.

### 3-Methyl-6-(2-piperidyl)-1,3-benzoxazol-2-one (XXVb)

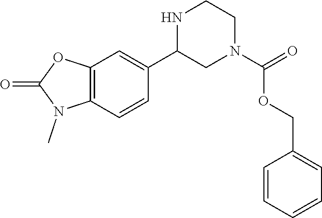 [SMILES: Cn1c(=O)oc2cc(C3CN(C(=O)OCc4ccccc4)CCN3)ccc21]

To a suspension of XXIVb (0.145 g, 0.30 mmol) in DCM (0.1 M) was added TFA (0.684 g, 6.0 mmol) and the reaction mixture was stirred at RT for 1 h. After evaporation of the solvent, the residue was used in the next step without further purification. UPLC/MS (method A): Rt 1.53 min. MS (ES) C₂₀H₂₁N₃O₅ requires 383, found 384 [M+H]⁺.

To a solution of compound from Step 1 in ACN (0.1 M) was added NaBH(OAc)₃ (0.191 g, 0.90 mmol) and the reaction mixture was stirred at RT for 30 min and then quenched with the addition of MeOH and diluted with EA. The organic phase was washed with saturated aq. NaHCO₃ solution, brine, dried over Na₂SO₄ and concentrated to afford XXVb which was used in the next step without further purification. ¹H NMR (400 MHz, CDCl₃) δ 7.40-7.30 (m, 6H), 7.23 (dd, J=8.0, 1.5 Hz, 1H), 6.90 (d, J=8.0 Hz, 1H), 5.16 (s, 2H), 4.25-4.10 (m, 2H), 3.85-3.70 (m, 1H), 3.39 (s, 3H), 3.20-3.00 (m, 2H), 3.00-2.70 (m, 2H), 2.30-2.10 (bs, 1H). UPLC/MS (method A): Rt 1.70 min. MS (ES) C₂₀H₂₁N₃O₄ requires 367, found 368 [M+H]⁺.

### Benzyl 3-(3-methyl-2-oxo-1,3-benzoxazol-6-yl)-4-(4-phenylbutylcarbamoyl)piperazine-1-carboxylate (XXVIIa)

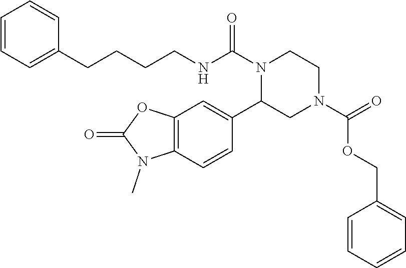 [SMILES: Cn1c(=O)oc2cc(C3CN(C(=O)OCc4ccccc4)CCN3C(=O)NCCCCc3ccccc3)ccc21]

Following general procedure D (Method A), XXVb (0.110 g, 0.030 mmol) and 4-phenylbutyl isocyanate (0.060 g, 0.33 mmol) afforded XXVIIIa as a white solid (0.137 g, 84%). ¹H NMR (400 MHz, CDCl₃) δ 7.40-7.25 (m, 7H), 7.20-7.05 (m, 5H), 6.90-6.70 (m, 1H), 5.20-5.10 (m, 2H), 5.10-4.95 (m, 1H), 4.35-4.15 (m, 1H), 4.10-3.90 (m, 1H), 3.90-3.55 (m, 2H), 3.50-3.40 (m, 3H), 3.34 (s, 3H), 3.30-3.10 (m, 2H), 2.65-2.45 (m, 2H), 1.60-1.35 (m, 4H). UPLC/MS (method A): Rt 2.29 min. MS (ES) C₃₁H₃₄N₄O₅ requires 542, found 543 [M+H]⁺.

### 2-(3-Methyl-2-oxo-1,3-benzoxazol-6-yl)-N-(4-phenylbutyl)piperazine-1-carboxamide

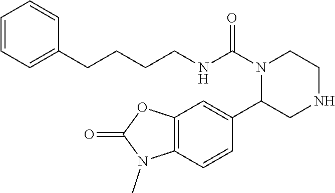 [SMILES: Cn1c(=O)oc2cc(C3CNCCN3C(=O)NCCCCc3ccccc3)ccc21]

Following general procedure B (method B), XXVIIIa (0.135 g, 0.25 mmol) afforded the title compound as a white solid (0.069, 68%). ¹H NMR (400 MHz, DMSO-d₆) δ 7.30 (s, 1H), 7.25 (dd, J=8.5, 6.5 Hz, 2H), 7.19-7.10 (m, 5H), 6.45 (t, J=5.5 Hz, 1H), 5.11 (s, 1H), 3.66 (dd, J=12.6, 3.7 Hz, 1H), 3.40-3.30 (m, 1H), 3.38 (s, 3H), 3.15-2.95 (m, 2H), 2.95-2.85 (m, 2H), 2.85-2.75 (m, 2H), 2.65-2.55 (m, 3H), 1.60-1.30 (m, 4H). UPLC/MS (method A): Rt 1.66 min. MS (ES) C₂₃H₂₈N₄O₃ requires 408, found 409 [M+H]⁺.

### Example 40: 4-Methyl-2-(3-methyl-2-oxo-1,3-benzoxazol-6-yl)-N-(4-phenylbutyl)piperazine-1-carboxamide

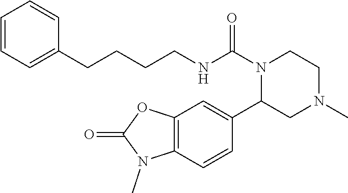 [SMILES: CN1CCN(C(=O)NCCCCc2ccccc2)C(c2ccc3c(c2)oc(=O)n3C)C1]

To a solution of 2-(3-methyl-2-oxo-1,3-benzoxazol-6-yl)-N-(4-phenylbutyl) piperazine-1-carboxamide (0.030 g, 0.073 mmol) in ACN (0.2 M) was added formaldehyde (37% aq. solution; 0.008 g, 0.28 mmol) and NaBH(AcO)₃ (0.30 g, 0.14 mmol) and the mixture was stirred at RT for 2 h and then diluted with EA, washed with saturated aq. NaHCO₃ solution, brine and dried over Na₂SO₄. The solvent was reduced in vacuo to afford the title compound as a white solid (0.029 g, 94%). ¹H NMR (400 MHz, CDCl₃) δ 7.40-7.35 (m, 1H), 7.30-7.20 (m, 4H), 7.20-7.15 (m, 1H), 7.15-7.10 (m, 2H), 6.85 (d, J=8.1 Hz, 1H), 5.25 (s, 1H), 4.50-4.35 (m, 1H), 3.64 (d, J=13.1 Hz, 1H), 3.34 (s, 3H), 3.30-3.05 (m, 3H), 2.85-2.70 (m, 1H), 2.65-2.55 (m, 2H), 2.55-2.45 (m, 1H), 2.31 (s, 3H), 2.25-2.10 (m, 1H), 1.70-1.45 (m, 4H). UPLC/MS (method A): Rt 1.87 min. MS (ES) C₂₄H₃₀N₄O₃ requires 422, found 423 [M+H]⁺.

### Example 41: 4-Methyl-3-(3-methyl-2-oxo-1,3-benzoxazol-6-yl)-N-(4-phenylbutyl)piperazine-1-carboxamide

### Benzyl 4-methyl-3-(3-methyl-2-oxo-1,3-benzoxazol-6-yl)piperazine-1-carboxylate (XXVIa)

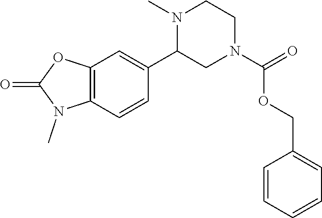 [SMILES: CN1CCN(C(=O)OCc2ccccc2)CC1c1ccc2c(c1)oc(=O)n2C]

To a solution of XXVb (0.050 g, 0.13 mmol) in ACN (0.2 M) 37% aqueous solution of formaldehyde (0.016 g, 0.039 mL, 0.52 mmol) and NaBH(AcO)₃ (0.055 g, 0.26 mmol) were added. The mixture was stirred at RT for 2 h and then diluted with EA, washed with saturated aqueous NaHCO₃ solution, brine, dried over Na₂SO₄ and concentrated to afford XXVIa which was used in the next step without further purification. ¹H NMR (400 MHz, CDCl₃) δ 7.45-7.30 (m, 5H), 7.25-7.15 (m, 1H), 6.91 (d, J=7.9 Hz, 1H), 5.15 (s, 2H), 4.15-4.00 (m, 2H), 3.40 (s, 3H), 3.35-3.05 (m, 1H), 3.05-2.75 (m, 3H), 2.40-2.15 (m, 1H), 2.05 (s, 3H), 1.90-1.50 (m, 1H). UPLC/MS (method A): Rt 2.03 min. MS (ES) C₂₁H₂₃N₃O₄ requires 381, found 382 [M+H]⁺.

### 3-Methyl-6-(1-methylpiperazin-2-yl)-1,3-benzoxazol-2-one (XXVIIa)

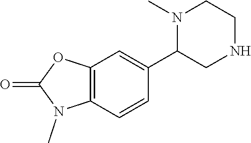 [SMILES: CN1CCNCC1c1ccc2c(c1)oc(=O)n2C]

Following general procedure B (method A), XXVIa (0.044 g, 0.12 mmol) afforded XXVIIa which was used in the next step without further purification. UPLC/MS (method A): Rt 0.94 min. MS (ES) C₁₃H₁₇N₃O₂ requires 247, found 248 [M+H]⁺.

### 4-Methyl-3-(3-methyl-2-oxo-1,3-benzoxazol-6-yl)-N-(4-phenylbutyl)piperazine-1-carboxamide

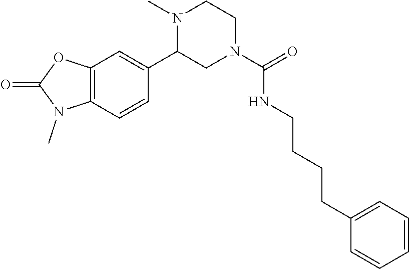 [SMILES: CN1CCN(C(=O)NCCCCc2ccccc2)CC1c1ccc2c(c1)oc(=O)n2C]

Following general procedure D (Method A), XXVIIa (0.025 g, 0.10 mmol) and 4-phenylbutyl isocyanate (0.019 g, 0.11 mmol) afforded the title compound as a white solid (0.021 g, 50%). ¹H NMR (400 MHz, CDCl₃) δ 7.30-7.25 (m, 3H), 7.25-7.20 (m, 1H), 7.20-7.15 (m, 3H), 6.92 (d, J=7.9 Hz, 11H), 4.45-4.35 (m, 11H), 3.90-3.70 (m, 2H), 3.39 (s, 3H), 3.30-3.20 (m, 2H), 3.20-3.10 (m, 1H), 3.05-2.90 (m, 2H), 2.90-2.75 (m, 1H), 2.70-2.60 (m, 2H), 2.40-2.02 (m, 1H), 2.05 (s, 3H), 1.75-1.50 (m 4H). UPLC/MS (method A): Rt 1.99 min. MS (ES) C₂₄H₃₀N₄O₃ requires 422, found 423 [M+H]⁺.

### Example 42: 3,3-Dimethyl-5-(3-methyl-2-oxo-1,3-benzoxazol-6-yl)-N-(4-phenylbutyl) morpholine-4-carboxamide

### tert-Butyl 3,3-dimethyl-5-oxo-morpholine-4-carboxylate (XXf)

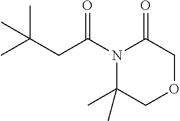 [SMILES: CC(C)(C)CC(=O)N1C(=O)COCC1(C)C]

To a solution of 5,5-dimethylmorpholinone (1.0 g, 7.8 mmol) in anhydrous THF (0.3M) nBuLi was added dropwise (2.5 M in hexanes, 3.41 mL) at −78° C. under N₂ atmosphere. After 30 min, a solution of Boc₂O (6.75 g, 30.96 mmol) in anhydrous THF was added at −78° C. The reaction mixture was allowed to warm to RT overnight, diluted with EA, washed with saturated aqueous NaHCO₃ solution, brine, dried over Na₂SO₄ and concentrated to afford XXf as white solid (1.6 g, 90° %). ¹H NMR (400 MHz, CDCl₃) δ 4.20 (s, 2H), 3.58 (s, 2H), 1.54 (s, 9H), 1.44 (s, 6H). UPLC/MS (method A): Rt 1.78 min. MS (ES) C₁₁H₁₉NO₄ requires 229, found 230 [M+H]⁺.

### [2-(tert-Butoxycarbonylamino)-2-methyl-propyl]-N-methoxy-N-methyl-carbamate (XXIa)

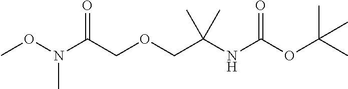 [SMILES: CON(C)C(=O)COCC(C)(C)NC(=O)OC(C)(C)C]

Step 1: To a solution of XXf (1.6 g, 6.98 mmol) in THF:H₂O (3:1, 0.3 M) was added LiOH (0.334 g, 13.94 mmol) and the reaction mixture was stirred 3 h at RT and then diluted with DCM. The pH of the aqueous layer was adjusted to about 4 using 5% aq. citric acid. The aqueous layer was extracted with DCM and dried over Na₂SO₄ to afford 2-[2-(tert-butoxycarbonylamino)-2-methyl-propoxy]acetic acid which was used in the next step without further purification. ¹H NMR (400 MHz, DMSO-d₆) δ 8.24 (bs, 1H), 3.52 (s, 2H), 3.19 (s, 2H), 1.35 (s, 9H), 1.17 (s, 6H). UPLC/MS (method A): Rt 1.23 min. MS (ES) C₁₁H₂₁NO₅ requires 247, found 248 [M+H]⁺, 246 [M−H]⁻.

Step 2: To a solution of 2-[2-(tert-butoxycarbonylamino)-2-methyl-propoxy]acetic acid (0.500 g, 2.02 mmol) in DMF (0.3M) N,O-dimethylhydroxylamine hydrochloride (0.250 g, 2.42 mmol), HATU (0.920 g, 2.42 mmol) and DIPEA (1.86 mL, 2.63 mmol) were added. The reaction mixture was stirred on at RT, diluted with EA, washed with saturated aq. NH₄Cl solution, brine, dried over Na₂SO₄ and concentrated to afford XXIa as a white solid (0.56 g, 96%). ¹H NMR (400 MHz, CDCl₃) δ 5.58 (bs, 1H), 4.29 (s, 2H), 3.66 (s, 3H), 3.43 (s, 2H), 3.17 (s, 3H), 1.42 (s, 9H), 1.31 (s, 6H). UPLC/MS (method A): Rt 1.88 min. MS (ES) C₁₃H₁₆N₂O₅ requires 290, found 291 [M−H]⁻.

### tert-Butyl N-[1,1-dimethyl-2-[2-oxo-2-(2-oxo-3H-1,3-benzoxazol-6-yl)ethoxy]ethyl]carbamate (XXIIe)

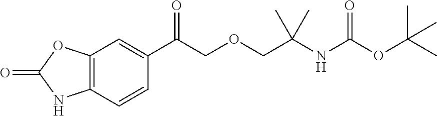 [SMILES: CC(C)(COCC(=O)c1ccc2[nH]c(=O)oc2c1)NC(=O)OC(C)(C)C]

Following general procedure G, XXIa (0.500 g, 1.72 mmol) and 6-bromo-3H-1,3-benzoxazol-2-one (0.250 g, 1.17 mmol) afforded XXIIe as a transparent oil (0.120 g, 30%). ¹H NMR (400 MHz, CDCl₃) δ 9.98 (bs, 1H), 7.79 (dd, J=1.9, 10.2 Hz, 2H), 7.15 (d, J=8.1 Hz, 1H), 4.75 (s, 2H), 3.54 (s, 2H), 1.44 (s, 9H), 1.32 (s, 6H). UPLC/MS (method A): Rt 1.96 min. MS (ES) C₁₈H₂₄N₂O₆ requires 364, found 363 [M−H]⁻.

### tert-Butyl N-[1,1-dimethyl-2-[2-(3-methyl-2-oxo-1,3-benzoxazol-6-yl)-2-oxo-ethoxy]ethyl]carbamate (XXIVe)

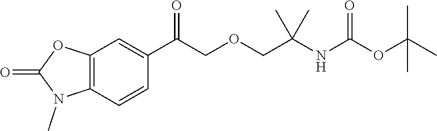 [SMILES: Cn1c(=O)oc2cc(C(=O)COCC(C)(C)NC(=O)OC(C)(C)C)ccc21]

To a solution of XXIIe (0.120 g, 0.33 mmol) in anydrous DMF (0.1 M) MeI (0.12 g, 0.83 mmol, 0.05 mL) and K₂CO₃ (0.030 g, 0.25 mmol) were added and the reaction mixture was stirred at RT overnight. The reaction mixture was diluted DCM, brine, dried over Na₂SO₄ and concentrated to afford XXIVe which was used in the next step without further purification. ¹H NMR (400 MHz, CDCl₃) δ 7.87 (dd, J=8.2, 1.5 Hz, 1H), 7.80 (d, J=1.3 Hz, 1H), 7.02 (d, J=8.2 Hz, 1H), 5.09 (bs, 1H), 4.73 (s, 2H), 3.53 (s, 2H), 3.45 (s, 3H), 1.43 (s, 9H), 1.32 (s, 6H). UPLC/MS (method A): Rt 2.18 min. MS (ES) C₁₉H₂₆N₂O₆ requires 378, found 379 [M+H]⁺.

### 3-Methyl-6-(5,5-dimethylmorpholin-3-yl)-1,3-benzoxazol-2-one trifluoroacetic acid (XXVe)

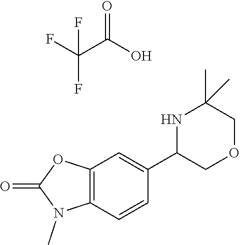 [SMILES: Cn1c(=O)oc2cc(C3COCC(C)(C)N3)ccc21 ; O=C(O)C(F)(F)F]

Step 1: To a suspension of XXIVe (0.174 g, 0.24 mmol) in DCM (0.1 M) TFA (0.155 g, 0.4 mL, 4.80 mmol) was added and the reaction mixture was stirred at RT for 1 h. After evaporation of the solvent, the residue was used in the next step without further purification. UPLC/MS (method A): Rt 1.63 min. MS (ES) C₁₄H₁₆N₂O₃ requires 260, found 261 [M+H]⁺.

Step 2: To a solution of compound from Step 1 in DCE (0.1 M) NaBH(OAc)₃ (0.152 g, 0.72 mmol) was added and the reaction mixture was stirred at RT for 30 min and then quenched with the addition of MeOH. The solvent was removed under reduced pressure and the crude was used in the next step without further purification. UPLC/MS (method A): Rt 1.05 min. MS (ES) C₁₄H₁₈N₂O₃ requires 262, found 263 [M+H]⁺.

### 3,3-Dimethyl-5-(3-methyl-2-oxo-1,3-benzoxazol-6-yl)-N-(4-phenylbutyl) morpholine-4-carboxamide

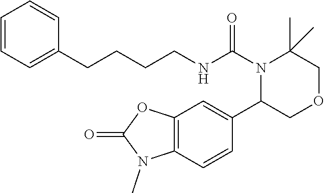 [SMILES: Cn1c(=O)oc2cc(C3COCC(C)(C)N3C(=O)NCCCCc3ccccc3)ccc21]

Following general procedure D (Method A), XXVe (0.045 g, 0.12 mmol) and 4-phenylbutyl isocyanate (0.011 g, 0.06 mmol) afforded the title compound as a white solid (0.013 g, 25%). ¹H NMR (400 MHz, DMSO-d₆) δ 7.34-7.30 (m, 1H), 7.30-7.00 (m, 8H), 4.46 (dd, J=9.6, 3.9 Hz, 1H), 3.75 (dd, J=11.2, 3.9 Hz, 1H), 3.47-3.33 (m, 3H), 2.87 (ddq, J=25.9, 13.2, 6.6 Hz, 2H), 2.44-2.36 (m, 2H), 1.40-1.16 (m, 10H). UPLC/MS (method A): Rt 2.19 min. MS (ES) C₂₁H₃₁N₃O₄ requires 437, found 438 [M+H]⁺.

### Example 43: 3,3-Dimethyl-5-(3-methyl-2-oxo-1,3-benzoxazol-6-yl)-N-(4-phenylbutyl)morpholine-4-carboxamide

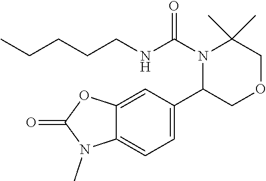 [SMILES: CCCCCNC(=O)N1C(c2ccc3c(c2)oc(=O)n3C)COCC1(C)C]

Following general procedure D (Method A), XXVe (0.015 g, 0.04 mmol) and n-pentyl isocyanate (0.011 g, 0.04 mmol) afforded the title compound as a white solid (0.010 g, 60%). ¹H NMR (400 MHz, DMSO-d₆) δ 7.34-7.31 (m, 1H), 7.24-7.17 (m, 1H), 7.14 (d, J=8.1 Hz, 1H), 7.03 (t, 1=5.8 Hz, 1H), 4.44 (dd, J=9.8, 4.0 Hz, 1H), 3.75 (dd, J=11.1, 4.0 Hz, 1H), 3.47-3.28 (m, 6H), 2.98-2.71 (m, 2H), 1.22 (d, J=8.4 Hz, 5H), 1.20-0.82 (m, 7H), 0.71 (t, J=7.3 Hz, 3H). UPLC/MS (method A): Rt 2.03 min. MS (ES) C₂₀H₂₉N₃O₄ requires 375, found 376 [M+H]⁺.

### Example 44: 2-(3-Methyl-2-oxo-1,3-benzoxazol-6-yl)-N-(4-phenylbutyl)pyrrolidine-1-carboxamide

### tert-Butyl N-[4-oxo-4-(2-oxo-3H-1,3-benzoxazol-6-yl)butyl]carbamate (XXIIf)

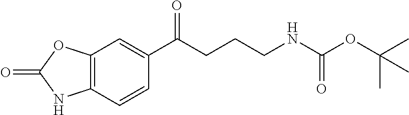 [SMILES: CC(C)(C)OC(=O)NCCCC(=O)c1ccc2[nH]c(=O)oc2c1]

Following general procedure G, XXg (1.30 g, 7.0 mmol) and 6-bromo-3H-1,3-benzoxazol-2-one (0.300 g, 1.40 mmol) afforded XXIIf as a transparent oil (0.225 g, 50%). ¹H NMR (400 MHz, DMSO-d₆) δ 7.87-7.66 (m, 2H), 7.23-7.14 (m, 1H), 6.90-6.78 (m, 1H), 3.67-3.57 (m, 2H), 3.06-2.90 (m, 2H), 1.86-1.59 (m, 2H), 1.36 (s, 9H). UPLC/MS (method A): Rt 1.74 min. MS (ES) C₁₆H₂₀N₂O₅ requires 320, found 321 [M−H]⁻.

### tert-Butyl N-[4-(3-methyl-2-oxo-1,3-benzoxazol-6-yl)-4-oxo-butyl]carbamate (XXIVf)

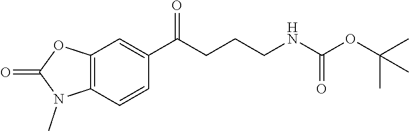 [SMILES: Cn1c(=O)oc2cc(C(=O)CCCNC(=O)OC(C)(C)C)ccc21]

To a solution of XXIIf (0.100 g, 0.31 mmol) in anydrous DMF (0.1 M) CH₃I (0.07 g, 0.47 mmol) and K₂CO₃ (0.032 g, 0.23 mmol) were added and the reaction mixture was stirred at RT overnight. The reaction mixture was diluted DCM, brine, dried over Na₂SO₄ and concentrated to afford XXIVf which was used in the next step without further purification. ¹H NMR (400 MHz, DMSO-d₆) δ 7.94-7.89 (m, 1H), 7.85-7.83 (m, 1H), 7.42-7.33 (m, 1H), 6.90-6.85 (m, 1H), 3.38 (s, 3H), 3.05-2.95 (m, 4H), 1.78-1.66 (m, 2H), 1.37 (s, 9H). UPLC/MS (method A): Rt 1.90 min. MS (ES) C₁₇H₂₂N₂O₅ requires 334, found 335 [M+H]⁺.

### 3-Methyl-6-pyrrolidin-2-yl-1,3-benzoxazol-2-one trifluoroacetic acid (XXVf)

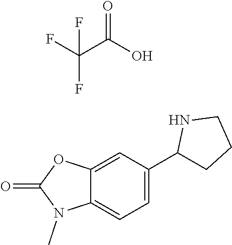 [SMILES: O=C(O)C(F)(F)F ; Cn1c(=O)oc2cc(C3CCCN3)ccc21]

To a suspension of XXIVf (0.095 g, 0.28 mmol) in DCM (0.1 M) TFA (0.648 g, 0.4 mL, 5.70 mmol) was added and the reaction mixture was stirred at RT for 1 h. After concentration the residue was used in the next step without further purification. UPLC/MS (method A): Rt 1.01 min. MS (ES) C₁₂H₁₄N₂O₃ requires 234, found 235 [M+H]⁺. To a solution of compound from Step 1 in DCE (0.1 M) NaBH(OAc)₃ (0.178 g, 0.56 mmol) was added. The reaction mixture was stirred at RT for 30 min and then quenched with the addition of MeOH. The solvent was removed under reduced pressure and triturated with Et₂₀ to afford XXVf as a white solid (0.091 g, 97%). ¹H NMR (400 MHz, DMSO-d₆) δ 9.59 (bs, 1H), 8.75 (bs, 1H), 7.54-7.51 (m, 1H), 7.40-7.15 (m, 2H), 4.67-4.54 (m, 1H), 3.37 (s, 3H), 3.36-3.25 (m, 2H), 2.42-2.30 (m, 1H), 2.20-1.90 (m, 2H). UPLC/MS (method A): Rt 0.97 min. MS (ES) C₁₂H₁₄N₂O₂ requires 218, found 219 [M+H]⁺.

### 2-(3-Methyl-2-oxo-1,3-benzoxazol-6-yl)-N-(4-phenylbutyl)pyrrolidine-1-carboxamide

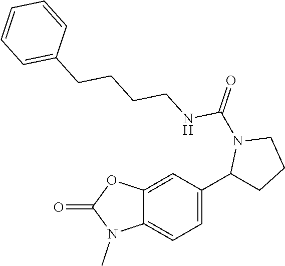 [SMILES: Cn1c(=O)oc2cc(C3CCCN3C(=O)NCCCCc3ccccc3)ccc21]

Following general procedure D (Method A), XXVf (0.090 g, 0.27 mmol) and 4-phenylbutyl isocyanate (0.053 g, 0.30 mmol) afforded the title compound as a white solid (0.094 g, 89%). ¹H NMR (400 MHz, CDCl₃) δ 7.47-7.40 (m, 2H), 7.37-7.32 (m, 1H), 7.30-7.25 (m, 4H), 7.07-7.02 (m, 1H), 5.01-4.95 (m, 1H), 3.87-3.76 (m, 2H), 3.51 (s, 3H), 3.42-3.25 (m, 2H), 2.75-2.65 (m, 2H), 2.61-2.48 (m, 1H), 2.18-1.94 (m, 4H), 1.71-1.54 (m, 4H). UPLC/MS (method A): Rt 2.07 min. MS (ES) C₂₃H₂₇N₃O₃ requires 393, found 394 [M+H]⁺.

### Example 45: 4-(2-Oxo-3H-1,3-benzoxazol-6-yl)-N-(4-phenylbutyl)piperazine-1-carboxamide

### tert-Butyl 4-(3-hydroxy-4-nitrophenyl)piperazine-1-carboxylate (XXXIa)

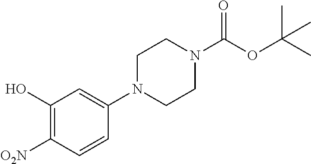 [SMILES: CC(C)(C)OC(=O)N1CCN(c2ccc([N+](=O)[O-])c(O)c2)CC1]

Following general procedure F, XXXa (3.56 g, 19.14 mmol) and 5-fluoro-2-nitrophenol (2.0 g, 12.73 mmol) afforded XXXIa as a yellow solid. The residue was used in the next step without further purification. ¹H NMR (400 MHz, DMSO-d₆) δ 10.95 (s, 1H), 7.89 (d, J=9.7 Hz, 1H), 6.64 (dd, J=9.7, 2.8 Hz, 1H), 6.42 (d, J=2.7 Hz, 1H), 3.54-3.41 (m, 8H), 1.43 (s, 9H). UPLC/MS (method A): Rt 2.36 min. MS (ES) C₁₅H₂₁N₃O₅ requires 323, found 324 [M+H]⁺.

### tert-Butyl 4-(4-amino-3-hydroxyphenyl)piperazine-1-carboxylate (XXXIIa)

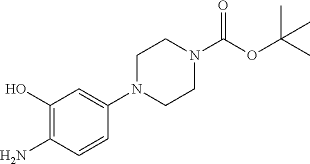 [SMILES: CC(C)(C)OC(=O)N1CCN(c2ccc(N)c(O)c2)CC1]

Following general procedure B (Method A, step 1), XXXIa (4.1 g, 12.73 mmol) afforded XXXIIa which was used in the next step without further purification. UPLC/MS (method A): Rt 1.77 min. MS (ES) C₁₅H₂₃N₃O₃ requires 293, found 294 [M+H]⁺.

### tert-Butyl 4-(2-oxo-3H-1,3-benzoxazol-6-yl)piperazine-1-carboxylate (XXXIa)

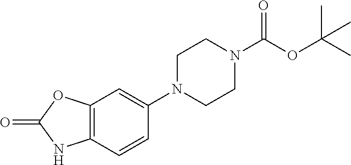 [SMILES: CC(C)(C)OC(=O)N1CCN(c2ccc3[nH]c(=O)oc3c2)CC1]

Following general procedure B (step 2), XXXIIa (3.7 g, 12.73 mmol) afforded XXXIIIa as a pink solid (3.05 g, 75%). ¹H NMR (400 MHz, DMSO-d₆) δ 11.34 (s, 1H), 7.00 (d, J=2.2 Hz, 1H), 6.94 (d, J=8.5 Hz, 1H), 6.74 (dd, J=8.6, 2.3 Hz, 1H), 3.57-3.41 (m, 4H), 3.08-2.93 (m, 4H), 1.42 (s, 9H). UPLC/MS (method A): Rt 2.01 min. MS (ES) C₁₆H₂₁N₃O₄ requires 319, found 320 [M+H]⁺.

### 6-Piperazin-1-yl-3H-1,3-benzoxazol-2-one hydrochloride (XXXIVa)

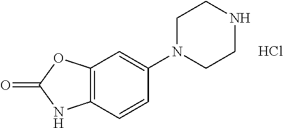 [SMILES: O=c1[nH]c2ccc(N3CCNCC3)cc2o1 ; Cl]

Following general procedure C (step 2), XXXIIIa (0.070 g, 0.274 mmol) afforded XXXIVa as a gray solid (0.056 g, 93%). ¹H NMR (600 MHz, DMSO-d₆) δ 11.47 (s, 1H), 9.24 (s, 2H), 7.07 (d, J=2.2 Hz, 1H), 6.99 (d, J=8.5 Hz, 1H), 6.79 (dd, J=8.5, 2.3 Hz, 1H), 3.39-3.27 (m, 4H), 3.25-3.18 (m, 4H). UPLC/MS (method B): Rt 1.34 min. MS (ES) C₁₁H₁₃N₃O₂ requires 219, found 220 [M+H]⁺.

### 4-(2-Oxo-3H-1,3-benzoxazol-6-yl)-N-(4-phenylbutyl)piperazine-1-carboxamide

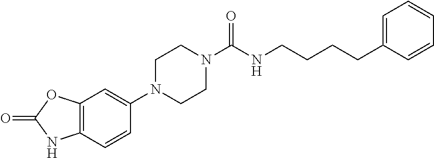 [SMILES: O=C(NCCCCc1ccccc1)N1CCN(c2ccc3[nH]c(=O)oc3c2)CC1]

Following general procedure D (Method A), XXXVIa (0.050 g, 0.18 mmol) and 4-phenylbutyl isocyanate (0.06 g, 0.35 mmol) afforded the title compound as a white solid (0.030 g, 48%). ¹H NMR (400 MHz, DMSO-d₆) δ 11.17 (s, 1H), 7.31-7.22 (m, 2H), 7.22-7.12 (m, 3H), 7.00 (d, J=2.3 Hz, 1H), 6.94 (d, J=8.5 Hz, 1H), 6.75 (dd, J=8.6, 2.3 Hz, 1H), 6.56 (t, J=5.5 Hz, 1H), 3.41 (t, J=5.1 Hz, 4H), 3.12-2.92 (m, 6H), 2.58 (t, J=7.5 Hz, 2H), 1.65-1.49 (m, 2H), 1.49-1.35 (m, 2H). UPLC/MS (method A): Rt 1.94 min. MS (ES) C₂₂H₂₆N₄O₃ requires 394, found 395 [M+H]⁺, 393 [M−H]⁻.

### Example 46: 4-(3-Methyl-2-oxo-1,3-benzoxazol-6-yl)-N-(4-phenylbutyl)piperazine-1-carboxamide

### tert-Butyl 4-(3-methyl-2-oxo-1,3-benzoxazol-6-yl)piperazine-1-carboxylate (XXXVa)

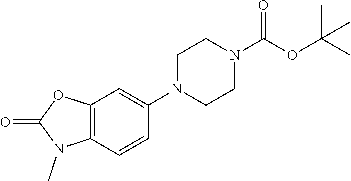 [SMILES: Cn1c(=O)oc2cc(N3CCN(C(=O)OC(C)(C)C)CC3)ccc21]

Following general procedure C (step 1), XXXIIIa (0.065 g, 0.204 mmol) and MeI (0.116 g, 0.814 mmol) afforded XXXVa as a pink solid (0.055 g, 82%). The residue was used in the next step without further purification. ¹H NMR (400 MHz, CDCl₃) δ 7.03-6.73 (m, 3H), 3.76-3.55 (m, 4H), 3.39 (s, 3H), 3.10 (q, J=5.1 Hz, 4H), 1.51 (s, 9H). UPLC/MS (method A): Rt 2.09 min. MS (ES) C₁₇H₂₃N₃O₄ requires 333, found 334 [M+H]⁺.

### 3-Methyl-6-piperazin-1-yl-1,3-benzoxazol-2-one hydrochloride (XXXVIa)

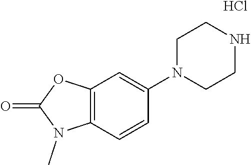 [SMILES: Cl ; Cn1c(=O)oc2cc(N3CCNCC3)ccc21]

Following general procedure C (step 2), XXXVa (0.052 g, 0.156 mmol) afforded XXXVIa as a gray solid (0.045 g, 95%). ¹H NMR (400 MHz, DMSO-d₆) δ 9.31 (br s, 2H), 7.20-7.08 (m, 2H), 6.88 (dd, J=8.6, 2.3 Hz, 1H), 3.41-3.28 (m, 7H), 3.21 (q, J=4.8 Hz, 4H). UPLC/MS (method A): Rt 0.91 min. MS (ES) C₁₂H₁₅N₃O₂ requires 233, found 234 [M+H]⁺.

### 4-(3-Methyl-2-oxo-1,3-benzoxazol-6-yl)-N-(4-phenylbutyl)piperazine-1-carboxamide

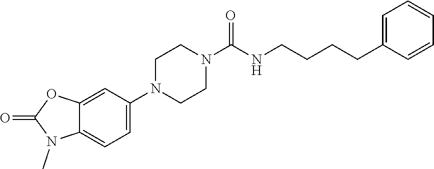 [SMILES: Cn1c(=O)oc2cc(N3CCN(C(=O)NCCCCc4ccccc4)CC3)ccc21]

Following general procedure D (Method A), XXXVIa (0.040 g, 0.131 mmol) and 4-phenylbutyl isocyanate (0.025 g, 0.144 mmol) afforded the title compound as a white solid (0.040 g, 75%). ¹H NMR (400 MHz, CDCl₃) δ 7.34-7.25 (m, 2H), 7.24-7.15 (m, 3H), 6.94-6.84 (m, 2H), 6.84-6.74 (m, 1H), 4.53 (t, J=5.7 Hz, 1H), 3.64-3.48 (m, 4H), 3.39 (s, 3H), 3.35-3.24 (m, 2H), 3.18-3.03 (m, 4H), 2.67 (t, J=7.5 Hz, 2H), 1.81-1.64 (m, 2H), 1.64-1.52 (m, 2H). UPLC/MS (method A): Rt 2.05 min. MS (ES) C₂₃H₂N₄O₃ requires 408, found 409 [M+H]⁺.

### Example 47: 4-[3-[2-(Dimethylamino)ethyl]-2-oxo-1,3-benzoxazol-6-yl]-N-(4-phenylbutyl)piperazine-1-carboxamide

### tert-Butyl 4-[3-[2-(dimethylamino)ethyl]-2-oxo-1,3-benzoxazol-6-yl]piperazine-1-carboxylate (XXXVb)

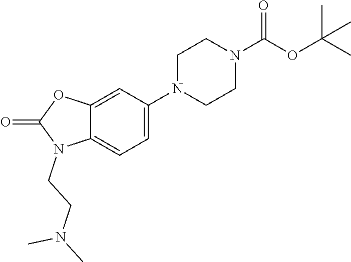 [SMILES: CN(C)CCn1c(=O)oc2cc(N3CCN(C(=O)OC(C)(C)C)CC3)ccc21]

Step 1: To a solution of XXXIIIa (0.250 g, 0.78 mmol) in DMF (0.2 M) was added K₂CO₃ (0.325 g, 2.35 mmol) and 1,2-dibromoethane (0.054 mL, 6.26 mmol) at RT and the reaction was stirred at 60° C. for 3 h. The mixture was poured into ice and the precipitate was filtered, solubilized in DCM and dried over Na₂SO₄. After evaporation of the solvent, tert-butyl 4-[3-(2-bromoethyl)-2-oxo-1,3-benzoxazol-6-yl]piperazine-1-carboxylate was used in the next step without further purification (0.294 g, 88%). ¹H NMR (400 MHz, CDCl₃) δ 7.02-6.89 (m, 2H), 6.88-6.76 (m, 1H), 4.22 (t, J=6.6 Hz, 2H), 3.74-3.53 (m, 6H), 3.18-2.99 (m, 4H), 1.51 (s, 9H). UPLC/MS (method A): Rt 2.30 min. MS (ES) C₁₈H₂₄BrN₃O₄ requires 425, found 426 [M+H]⁺.

Step 2: To a solution of the compound from Step 1 (0.080 g, 0.19 mmol) in DMF (0.2 M) was added K₂CO₃ (0.078 g, 0.56 mmol) and NHMe₂ (0.94 mL, 1.877 mmol) at RT and the reaction was stirred at 60° C. for 2 h and then cooled to RT, poured into ice and the precipitate was filtered off. The residue was used in the next step without further purification. ¹H NMR (400 MHz, CDCl₃) δ 7.02 (d, J=8.5 Hz, 1H), 6.88 (d, J=2.2 Hz, 1H), 6.78 (dd, J=8.5, 2.3 Hz, 1H), 4.02 (t, J=6.9 Hz, 2H), 3.66-3.56 (m, 4H), 3.14-3.03 (m, 4H), 2.82 (t, J=6.0 Hz, 2H), 2.44 (s, 6H), 1.51 (s, 9H). UPLC/MS (method A): Rt 1.69 min. MS (ES) C₂₀H₃₀N₄O₄ requires 390, found 391 [M+H]⁺.

### 3-[2-(Dimethylamino)ethyl]-6-piperazin-1-yl-1,3-benzoxazol-2-one dihydrochloride (XXXVIb)

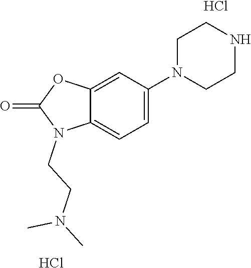 [SMILES: Cl ; CN(C)CCn1c(=O)oc2cc(N3CCNCC3)ccc21 ; Cl]

Following general procedure C (step 2), XXXVb (0.055 g, 0.141 mmol) afforded XXXVIb as a gray solid (0.050 g, 98%). ¹H NMR (400 MHz, DMSO-d₆) δ 10.70 (br s, 1H), 9.45 (br s, 2H), 7.34 (d, J=8.6 Hz, 1H), 7.16 (d, J=2.2 Hz, 1H), 6.89 (dd, J=8.6, 2.3 Hz, 1H), 4.22 (t, J=6.3 Hz, 2H), 3.49-3.37 (m, 2H), 3.39-3.28 (m, 4H), 3.25-3.15 (m, 4H), 2.85 (s, 3H), 2.83 (s, 3H). UPLC/MS (method A): Rt 0.46 min. MS (ES) C₁₅H₂₂N₄O₂ requires 290, found 291 [M+H]⁺.

### 4-[3-[2-(Dimethylamino)ethyl]-2-oxo-1,3-benzoxazol-6-yl]-N-(4-phenylbutyl)piperazine-1-carboxamide

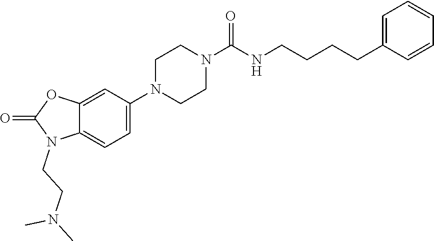 [SMILES: CN(C)CCn1c(=O)oc2cc(N3CCN(C(=O)NCCCCc4ccccc4)CC3)ccc21]

Following general procedure D (Method A), XXXVIb (0.048 g, 0.132 mmol) and 4-phenylbutyl isocyanate (0.025 g, 0.145 mmol) afforded the title compound as a white solid (0.040 g, 65%). ¹H NMR (400 MHz, DMSO-d₆) δ 7.33-7.23 (m, 2H), 7.25-7.11 (m, 4H), 7.06 (d, J=2.2 Hz, 1H), 6.82 (dd, J=8.7, 2.3 Hz, 1H), 6.56 (t, J=5.5 Hz, 1H), 3.86 (t, J=6.2 Hz, 2H), 3.55-3.36 (m, 4H), 3.25-2.88 (m, 6H), 2.63-2.52 (m, 4H), 2.16 (s, 6H), 1.67-1.50 (m, 2H), 1.50-1.35 (m, 2H). UPLC/MS (method A): Rt 1.80 min. MS (ES) C₂₆H₃₅N₅O₃ requires 465, found 466 [M+H]⁺.

### Example 48: (2R)-2-Methyl-4-(3-methyl-2-oxo-1,3-benzoxazol-6-yl)-N-(4-phenylbutyl)piperazine-1-carboxamide

### tert-Butyl (2R)-4-(3-hydroxy-4-nitrophenyl)-2-methylpiperazine-1-carboxylate (XXXIb)

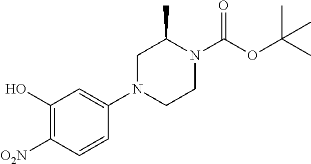 [SMILES: C[C@@H]1CN(c2ccc([N+](=O)[O-])c(O)c2)CCN1C(=O)OC(C)(C)C]

Following general procedure F, XXXb (0.89 g, 4.77 mmol) and 5-fluoro-2-nitrophenol (0.5 g, 3.18 mmol) afforded XXXIb as an orange solid (0.865 g, 81%). ¹H NMR (400 MHz, CDCl₃) δ 11.25 (s, 1H), 7.96 (d, J=9.6 Hz, 1H), 6.37 (dd, J=9.7, 2.7 Hz, 1H), 6.25 (d, J=2.7 Hz, 1H), 4.38-4.26 (m, 1H), 3.91 (dt, J=13.6, 4.0 Hz, 1H), 3.80-3.69 (m, 1H), 3.67-3.57 (m, 1H), 3.42-3.30 (m, 2H), 3.19 (ddd, J=12.3, 10.3, 3.8 Hz, 1H), 1.48 (s, 9H), 1.20 (d, J=6.7 Hz, 3H). UPLC/MS (method A): Rt 2.39 min. MS (ES) C₁₆H₂₃N₃O₅ requires 337, found 338 [M+H]⁺.

### tert-Butyl (2R)-4-(4-amino-3-hydroxyphenyl)-2-methylpiperazine-1-carboxylate (XXXIIb)

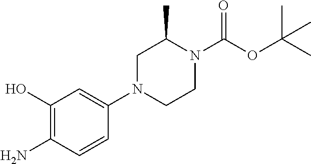 [SMILES: C[C@@H]1CN(c2ccc(N)c(O)c2)CCN1C(=O)OC(C)(C)C]

Following general procedure B (Method A), XXXIb (0.400 g, 1.186 mmol) afforded XXXIIb which was used in the next step without further purification. UPLC/MS (method A): Rt 1.82 min. MS (ES) C₁₆H₂₅N₃O₃ requires 307, found 308 [M+H]⁺.

### tert-Butyl (2R)-2-methyl-4-(2-oxo-3H-1,3-benzoxazol-6-yl)piperazine-1-carboxylate (XXXIIIb)

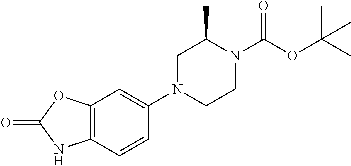 [SMILES: C[C@@H]1CN(c2ccc3[nH]c(=O)oc3c2)CCN1C(=O)OC(C)(C)C]

Following general procedure B (step 2), XXXIIb (0.365 g, 1.186 mmol) afforded XXXIIIb as a pink solid (0.192 g, 49%). ¹H NMR (400 MHz, CDCl₃) δ 8.61 (bs, 1H), 7.00-6.91 (m, 1H), 6.90-6.82 (m, 1H), 6.80-6.70 (m, 1H), 4.36 (s, 1H), 3.97 (d, J=13.2 Hz, 1H), 3.39 (d, J=11.8 Hz, 1H), 3.34-3.20 (m, 2H), 2.99-2.88 (m, 1H), 2.83-2.68 (m, 1H), 1.49 (s, 9H), 1.34 (d, J=6.8 Hz, 3H). UPLC/MS (method A): Rt 2.07 min. MS (ES) C₁₇H₂₃N₃O₄ requires 333, found 334 [M+H]⁺.

### tert-Butyl (2R)-2-methyl-4-(3-methyl-2-oxo-1,3-benzoxazol-6-yl)piperazine-1-carboxylate (XXXVc)

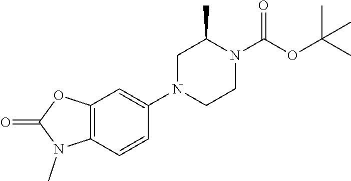 [SMILES: C[C@@H]1CN(c2ccc3c(c2)oc(=O)n3C)CCN1C(=O)OC(C)(C)C]

Following general procedure C (step 1), XXXIIIb (0.192 g, 0.58 mmol) and MeI (0.14 g, 0.86 mmol) afforded XXXVc as a white solid (0.160 g, 79%). ¹H NMR (400 MHz, CDCl₃) δ 7.05-6.66 (m, 3H), 4.37 (s, 1H), 3.98 (d, J=13.1 Hz, 1H), 3.45-3.20 (m, 5H), 3.02-2.90 (m, 1H), 2.86-2.70 (m, 1H), 1.49 (s, 9H), 1.36 (d, J=6.6 Hz, 3H). UPLC/MS (method A): Rt 2.26 min. MS (ES) C₁₈H₂₅N₃O₄ requires 347, found 348 [M+H]⁺.

### 3-Methyl-6-[(3R)-3-methylpiperazin-1-yl]-1,3-benzoxazol-2-one hydrochloride (XXXVIc)

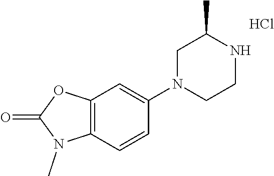 [SMILES: C[C@@H]1CN(c2ccc3c(c2)oc(=O)n3C)CCN1 ; Cl]

Following general procedure C (step 2), XXXVb (0.160 g, 0.46 mmol) afforded XXXVIc as a white solid (0.09 g, 68%). ¹H NMR (400 MHz, CDCl₃) δ 9.98-9.17 (m, 2H), 7.25-7.10 (m, 2H), 6.98-6.83 (m, 1H), 3.75-3.59 (m, 2H), 3.41-3.25 (m, 5H), 3.16-2.94 (m, 2H), 2.87-2.74 (m, 1H), 1.31 (d, J=6.5 Hz, 3H). UPLC/MS (method A): Rt 0.99 min. MS (ES) C₁₃H₁₇N₃O₂ requires 247, found 248 [M+H]⁺.

### (2R)-2-Methyl-4-(3-methyl-2-oxo-1,3-benzoxazol-6-yl)-N-(4-phenylbutyl)piperazine-1-carboxamide

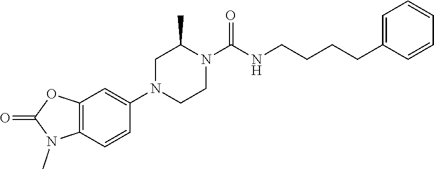 [SMILES: C[C@@H]1CN(c2ccc3c(c2)oc(=O)n3C)CCN1C(=O)NCCCCc1ccccc1]

Following general procedure D (Method A), XXXVIc (0.03 g, 0.160 mmol) and 4-phenylbutyl isocyanate (0.020 g, 0.12 mmol) afforded the title compound as a white solid (0.013 g, 29%). ¹H NMR (400 MHz, DMSO-d₆) δ 7.31-7.23 (m, 2H), 7.22-7.13 (m, 3H), 7.09 (d, J=8.6 Hz, 1H), 7.03 (d, J=2.2 Hz, 1H), 6.81 (dd, J=8.6, 2.3 Hz, 1H), 6.47 (t, J=5.5 Hz, 1H), 4.26-4.17 (m, 1H), 3.78 (d, J=13.1 Hz, 1H), 3.48 (d, 0.1=11.5 Hz, 1H), 3.39 (d, J=11.7 Hz, 1H), 3.29 (s, 3H), 3.12-2.98 (m, 3H), 2.72 (dd, J=11.8, 3.7 Hz, 1H), 2.61-2.54 (m, 2H), 2.54-2.52 (m, 1H), 1.60-1.50 (p, J=7.0 Hz, 2H), 1.42 (p, J1=7.0 Hz, 2H), 1.16 (d, J=6.6 Hz, 3H). UPLC/MS (method A): Rt 2.16 min. MS (ES) C₂₄H₃₀N₄O₃ requires 422, found 423 [M+H]⁺.

### Example 49: (2S)-2-Methyl-4-(3-methyl-2-oxo-1,3-benzoxazol-6-yl)-N-(4-phenylbutyl)piperazine-1-carboxamide

### tert-Butyl (2S)-4-(3-hydroxy-4-nitrophenyl)-2-methylpiperazine-1-carboxylate (XXXIc)

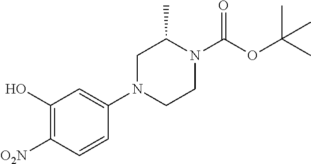 [SMILES: C[C@H]1CN(c2ccc([N+](=O)[O-])c(O)c2)CCN1C(=O)OC(C)(C)C]

Following general procedure F, XXXc (1.27 g, 6.36 mmol) and 5-fluoro-2-nitrophenol (0.5 g, 3.18 mmol) afforded XXXIc as a yellow solid. The residue was used in the next step without further purification. ¹H NMR (400 MHz, CDCl₃) δ 11.25 (s, 1H), 7.96 (d, J=9.6 Hz, 1H), 6.36 (dd, J=9.7, 2.7 Hz, 1H), 6.25 (d, J=2.7 Hz, 1H), 4.37-4.27 (m, 1H), 3.91 (dt, J=13.4, 3.7 Hz, 1H), 3.75 (dt, J=12.4, 3.4 Hz, 1H), 3.62 (dd, J=13.3, 2.0 Hz, 1H), 3.42-3.29 (m, 2H), 3.19 (td, J=11.5, 10.5, 3.8 Hz, 1H), 1.48 (s, 9H), 1.20 (d, J=6.7 Hz, 3H). UPLC/MS (method B): Rt 1.26 min. MS (ES) C₁₆H₂₃N₃O₅ requires 337, found 338 [M+H]⁺.

### tert-Butyl (2S)-4-(4-amino-3-hydroxyphenyl)-2-methylpiperazine-1-carboxylate (XXXIIc)

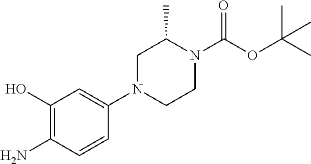 [SMILES: C[C@H]1CN(c2ccc(N)c(O)c2)CCN1C(=O)OC(C)(C)C]

Following general procedure B (Method A), XXXIc (1.43 g, 4.24 mmol) afforded which XXXIIc was used in the next step without further purification. UPLC/MS (method A): Rt 1.84 min. MS (ES) C₁₆H₂₅N₃O₃ requires 307, found 308 [M+H]⁺. tert-Butyl (2S)-2-methyl-4-(2-oxo-3H-1,3-benzoxazol-6-yl)piperazine-1-carboxylate (XXXIIIc)

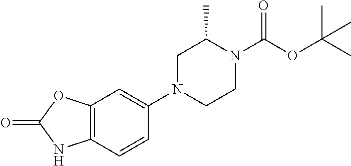 [SMILES: C[C@H]1CN(c2ccc3[nH]c(=O)oc3c2)CCN1C(=O)OC(C)(C)C]

Following general procedure B (step 2), XXXIIc (1.29 g, 4.24 mmol) afforded XXXIIIc as a pink solid (0.260 g, 200%). ¹H NMR (400 MHz, CDCl₃) δ 9.71 (s, 1H), 6.96 (d, J=8.5 Hz, 1H), 6.81 (d, J=1.9 Hz, 1H), 6.71 (dd, J=8.5, 2.0 Hz, 1H), 4.35 (s, 1H), 3.95 (d, J=13.3 Hz, 1H), 3.43-3.31 (m, 1H), 3.31-3.18 (m, 2H), 2.90 (dd, J=11.8, 3.8 Hz, 1H), 2.71 (td, J=11.6, 3.5 Hz, 1H), 1.48 (s, 9H), 1.31 (d, J=6.7 Hz, 3H). UPLC/MS (method A): Rt 2.08 min. MS (ES) C₁₇H₂₃N₃O₄ requires 333, found 334 [M+H]⁺.

### tert-Butyl (2S)-2-methyl-4-(3-methyl-2-oxo-1,3-benzoxazol-6-yl)piperazine-1-carboxylate (XXXVd)

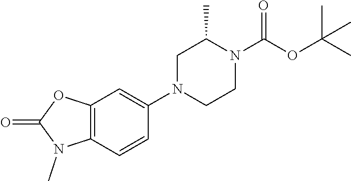 [SMILES: C[C@H]1CN(c2ccc3c(c2)oc(=O)n3C)CCN1C(=O)OC(C)(C)C]

Following general procedure C (step 1), XXXIIIe (0.260 g, 0.78 mmol) and MeI (0.55 g, 3.90 mmol) afforded XXXVd as a pink solid. The residue was used in the next step without further purification. UPLC/MS (method A): Rt 2.27 min. MS (ES) C₁₈H₂₅N₃O₄ requires 347, found 348 [M+H]⁺.

### 3-Methyl-6-[(3S)-3-methylpiperazin-1-yl]-1,3-benzoxazol-2-one hydrochloride (XXXVId)

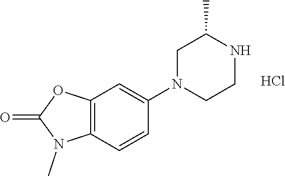 [SMILES: C[C@H]1CN(c2ccc3c(c2)oc(=O)n3C)CCN1 ; Cl]

Following general procedure C (step 2), XXXVc (0.310 g, 0.89 mmol) afforded XXXVId as a gray solid (0.245 g, 97%). ¹H NMR (400 MHz, DMSO-d₆) δ 9.76-9.58 (m, 1H), 9.49-9.27 (m, 1H), 7.16-7.10 (m, 2H), 6.88 (dd, J=8.5, 2.2 Hz, 1H), 3.74-3.59 (m, 2H), 3.42-3.26 (m, 2H), 3.30 (s, 3H), 3.16-2.94 (m, 2H), 2.85-2.74 (m, 1H), 1.31 (d, J=6.5 Hz, 3H). (UPLC/MS (method A): Rt 0.96 min. MS (ES) C₁₃H₁₇N₃O₂ requires 247, found 248 [M+H]⁺.

### (2S)-2-Methyl-4-(3-methyl-2-oxo-1,3-benzoxazol-6-yl)-N-(4-phenylbutyl)piperazine-1-carboxamide

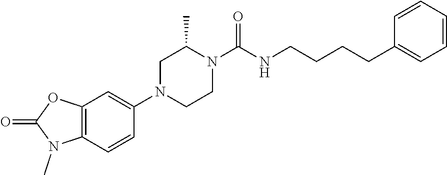 [SMILES: C[C@H]1CN(c2ccc3c(c2)oc(=O)n3C)CCN1C(=O)NCCCCc1ccccc1]

Following general procedure D (Method A), XXXVId (0.050 g, 0.18 mmol) and 4-phenylbutyl isocyanate (0.06 g, 0.35 mmol) afforded the title compound as a white solid (30 mg, 45%). ¹H NMR (400 MHz, DMSO-d₆) δ 7.27 (t, J=7.4 Hz, 2H), 7.22-7.13 (m, 3H), 7.09 (d, 0.1=8.6 Hz, 1H), 7.03 (d, J=2.1 Hz, 1H), 6.81 (dd, J=8.6, 2.2 Hz, 1H), 6.47 (t, J=5.4 Hz, 1H), 4.26-4.17 (m, 1H), 3.82-3.74 (m, 1H), 3.51-3.44 (m, 1H), 3.43-3.35 (m, 1H), 3.29 (s, 3H), 3.14-2.96 (m, 3H), 2.72 (dd, J=11.8, 3.6 Hz, 1H), 2.62-2.54 (m, 2H), 2.55-2.52 (m, 1H), 1.55 (p, J1=7.3, 8.0 Hz, 2H), 1.42 (p, J=7.0 Hz, 2H), 1.16 (d, J=6.6 Hz, 3H). UPLC/MS (method A): Rt 2.17 min. MS (ES) C₂₄H₃₀N₄O₃ requires 422, found 423 [M+H]⁺.

### Example 50: 2,2-Dimethyl-4-(3-methyl-2-oxo-1,3-benzoxazol-6-yl)-N-(4-phenylbutyl)piperazine-1-carboxamide

### tert-Butyl 4-(3-hydroxy-4-nitrophenyl)-2,2-dimethylpiperazine-1-carboxylate (XXXId)

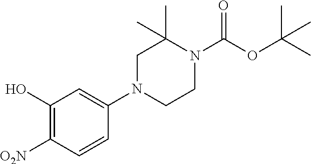 [SMILES: CC(C)(C)OC(=O)N1CCN(c2ccc([N+](=O)[O-])c(O)c2)CC1(C)C]

Following general procedure F, XXXd (0.6 g, 2.8 mmol) and 5-fluoro-2-nitrophenol (0.66 g, 4.2 mmol) afforded XXXId as a yellow solid (0.436 g, 83%). ¹H NMR (400 MHz, CDCl₃) 11.33 (s, 1H), 7.97 (d, J=9.6 Hz, 1H), 6.30 (dd, J=9.7, 2.7 Hz, 1H), 6.15 (d, J=2.7 Hz, 1H), 3.88 (t, J=5.7 Hz, 2H), 3.59-3.46 (m, 4H), 1.50 (s, 9H), 1.42 (s, 6H). 5 UPLC/MS (method A): Rt 2.51 min. MS (ES) C₁₇H₂₅N₃O₅ requires 351, found 352 [M+H]⁺.

### tert-Butyl 4-(4-amino-3-hydroxyphenyl)-2,2-dimethyl-piperazine-1-carboxylate (XXXIId)

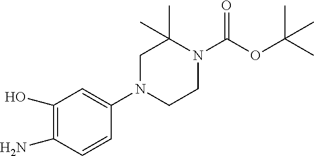 [SMILES: CC(C)(C)OC(=O)N1CCN(c2ccc(N)c(O)c2)CC1(C)C]

Following general procedure B (Method C), XXXId (0.436 g, 1.24 mmol) afforded XXXIId which was used in the next step without further purification. UPLC/MS (method A): Rt 1.64 min. MS (ES) C₁₇H₂₇N₃O₃ requires 321, found 322 [M+H]⁺.

### tert-Butyl 2,2-dimethyl-4-(2-oxo-3H-1,3-benzoxazol-6-yl)piperazine-1-carboxylate (XXXIIId)

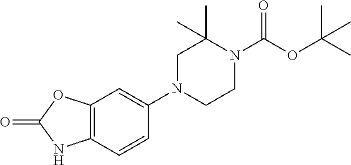 [SMILES: CC(C)(C)OC(=O)N1CCN(c2ccc3[nH]c(=O)oc3c2)CC1(C)C]

Following general procedure B (step 2), XXXIId (0.4 g, 1.24 mmol) and CDI (1.01 g, 6.2 mmol) afforded XXIIId as a lilac solid (0.316 g, 73%). ¹H NMR (400 MHz, CDCl₃) δ 8.30 (s, 1H), 6.92 (d, J=8.6 Hz, 1H), 6.77-6.71 (m, 1H), 6.65-6.56 (m, 1H), 3.81 (t, J=8.0 Hz, 2H), 3.35 (t, J=5.5 Hz, 2H), 3.21 (s, 2H), 1.50 (s, 9H), 1.46 (s, 6H). UPLC/MS (method A): Rt 2.28 min. MS (ES) C₁₈H₂₅N₃O₄ requires 347, found 348 [M+H]⁺.

### tert-Butyl 2,2-dimethyl-4-(3-methyl-2-oxo-1,3-benzoxazol-6-yl)piperazine-1-carboxylate (XXXVe)

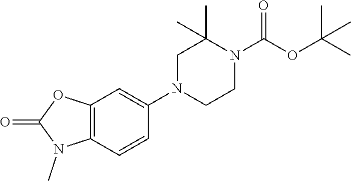 [SMILES: Cn1c(=O)oc2cc(N3CCN(C(=O)OC(C)(C)C)C(C)(C)C3)ccc21]

Following general procedure C (step 1), XXXIIId (0.100 g, 0.288 mmol) and MeI (0.031 mL, 0.434 mmol) afforded XXXVe which was used in the next step without further purification. ¹H NMR (400 MHz, CDCl₃) δ 6.82 (d, J=8.5 Hz, 1H), 6.68 (d, J=2.3 Hz, 1H), 6.57 (dd, J=8.6, 2.3 Hz, 1H), 3.78 (t, J=5.6 Hz, 2H), 3.35 (s, 3H), 3.32 (t, J=5.6 Hz, 2H), 3.20 (s, 2H), 1.49 (s, 9H), 1.44 (s, 6H. UPLC/MS (method A): Rt 2.58 min. MS (ES) C₁₉H₂₇N₃O₄ requires 361, found 362 [M+H]⁺.

### 6-(3,3-Dimethylpiperazin-1-yl)-3-methyl-1,3-benzoxazol-2-one hydrochloride (XXXVIe)

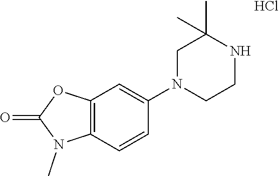 [SMILES: Cn1c(=O)oc2cc(N3CCNC(C)(C)C3)ccc21 ; Cl]

Following general procedure C (step 2), XXXVe (0.104 g, 0.288 mmol) afforded XXXVIe which was used in the next step without further purification ¹H NMR (400 MHz, DMSO-d₆) ¹H NMR (400 MHz, DMSO-d₆) δ 9.44-9.28 (m, 2H), 7.13 (d, J=8.5 Hz, 1H), 7.09 (d, J=2.2 Hz, 1H), 6.85 (dd, J=8.6, 2.3 Hz, 1H), 3.30 (s, 3H), 3.29-3.20 (m, 4H), 3.13 (s, 2H), 1.40 (s, 6H). 5 UPLC/MS (method A): Rt 1.17 min. MS (ES) C₁₄H₁₉N₃O₂ requires 261, found 262 [M+H]⁺.

### 2,2-Dimethyl-4-(3-methyl-2-oxo-1,3-benzoxazol-6-yl)-N-(4-phenylbutyl)piperazine-1-carboxamide

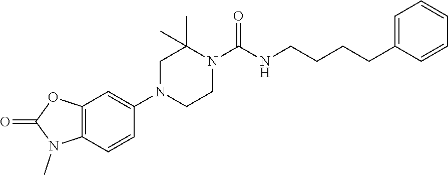 [SMILES: Cn1c(=O)oc2cc(N3CCN(C(=O)NCCCCc4ccccc4)C(C)(C)C3)ccc21]

Following general procedure D (Method A), XXXVIe (0.05 g, 0.172 mmol) and 4-phenylbutyl isocyanate (0.036 g, 0.260 mmol) afforded the title compound as a white solid (0.045 g, 60%). ¹H NMR (400 MHz, DMSO-d₆) δ 7.31-7.25 (m, 2H), 7.22-7.15 (m, 3H), 7.07 (d, J=8.6 Hz, 1H), 6.93 (d, J=2.2 Hz, 1H), 6.69 (dd, J=8.6, 2.3 Hz, 1H), 6.36 (t, J=5.5 Hz, 1H), 3.46 (t, 0.1=5.5 Hz, 2H), 3.29 (s, 3H), 3.23-3.14 (m, 4H), 3.09 (s, 2H), 3.02 (q, J1=6.6 Hz, 2H), 2.58 (t, J=7.6 Hz, 2H), 1.56 (p, J=7.5 Hz, 2H), 1.43 (p, J=7.6 Hz, 2H), 1.37 (s, 6H). UPLC/MS (method A): Rt 2.32 min. MS (ES) C₂₅H₃₂N₄O₃ requires 436, found 437 [M+H]⁺.

### Example 51: 2,2-Dimethyl-4-(3-Methyl-2-oxo-1,3-benzoxazol-6-yl)-N-(2-phenylethyl)piperazine-1-carboxamide

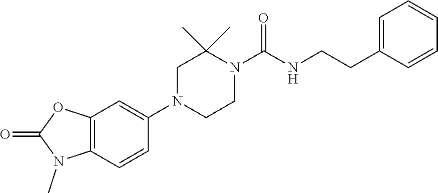 [SMILES: Cn1c(=O)oc2cc(N3CCN(C(=O)NCCc4ccccc4)C(C)(C)C3)ccc21]

Following general procedure D (Method A), XXXVIe (0.028 g, 0.090 mmol) and 4-phenylethyl isocyanate (0.010 g, 0.070 mmol) afforded the title compound as a white solid (0.022 g, 57%). ¹H NMR (400 MHz, DMSO-d₆) δ 7.37-7.27 (m, 2H), 7.25-7.14 (m, 3H), 7.07 (d, J=8.6 Hz, 1H), 6.93 (d, J=2.3 Hz, 1H), 6.70 (dd, J=8.7, 2.3 Hz, 1H), 6.46 (t, J=5.4 Hz, 1H), 3.45 (t, J=5.5 Hz, 2H), 3.26-3.16 (m, 4H), 3.10 (s, 2H), 2.78-2.68 (m, 2H), 1.37 (s, 6H). UPLC/MS (method A): Rt 2.13 min. MS (ES) C₂₃H₂₈N₄O₃ requires 408, found 409 [M+H]⁺.

### Example 52: N-(4-Cyclopropylbutyl)-2,2-dimethyl-4-(3-methyl-2-oxo-1,3-benzoxazol-6-yl)piperazine-1-carboxamide

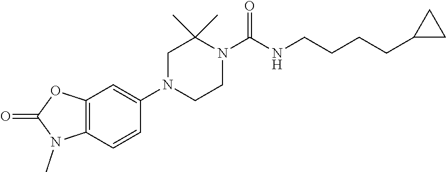 [SMILES: Cn1c(=O)oc2cc(N3CCN(C(=O)NCCCCC4CC4)C(C)(C)C3)ccc21]

Following general procedure D (Method C), XXXVIe (0.040 g, 0.130 mmol) and 4-cyclopropylbutan-1-amine (0.029 g, 0.256 mmol) afforded the title compound as a white solid (0.026 g, 49%). ¹H NMR (400 MHz, DMSO-d₆) δ 7.07 (d, J=8.6 Hz, 1H), 6.93 (d, J=2.3 Hz, 1H), 6.70 (dd, J=8.6, 2.3 Hz, 1H), 6.34 (t, J=5.4 Hz, 1H), 3.47 (t, J=5.5 Hz, 2H), 3.29 (s, 3H), 3.19 (t, J=5.5 Hz, 2H), 3.09 (s, 2H), 2.98 (q, J=6.6 Hz, 2H), 1.49-1.38 (m, 2H), 1.38 (s, 6H), 1.38-1.28 (m, 2H), 1.18 (q, J=7.0 Hz, 2H), 0.73-0.59 (m, 1H), 0.47-0.28 (m, 2H), 0.08-0.09 (m, 2H). UPLC/MS (method A): Rt 2.30 min. MS (ES) C₂₂H₃₂N₄O₃ requires 400, found 401 [M+H]⁺.

### Example 53: N-(2-Cyclopropylethyl)-2,2-dimethyl-4-(3-methyl-2-oxo-1,3-benzoxazol-6-yl)piperazine-1-carboxamide

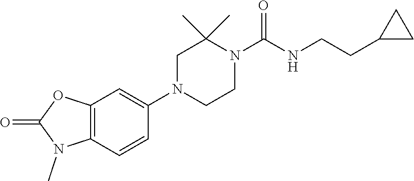 [SMILES: Cn1c(=O)oc2cc(N3CCN(C(=O)NCCC4CC4)C(C)(C)C3)ccc21]

Following general procedure D (Method C), XXXVIe (0.040 g, 0.130 mmol) and 2-cyclopropylethanamine (0.022 g, 0.258 mmol) afforded the title compound as a white solid (0.012 g, 24%). ¹H NMR (400 MHz, DMSO-d₆) δ 7.07 (d, J=8.5 Hz, 1H), 6.93 (d, J=2.2 Hz, 1H), 6.69 (dd, J=8.6, 2.3 Hz, 1H), 6.34 (t, J=5.4 Hz, 1H), 3.47 (t, J=5.4 Hz, 2H), 3.29 (s, 3H), 3.19 (t, J=5.5 Hz, 2H), 3.14-2.97 (m, 4H), 1.37 (s, 6H), 1.35-1.26 (m, 2H), 0.80-0.56 (m, 1H), 0.51-0.24 (m, 2H), 0.12-0.15 (m, 2H). UPLC/MS (method A): Rt 2.01 min. MS (ES) C₂₀H₂₈N₄O₃ requires 372, found 373 [M+H]⁺.

### Example 54: 2,2-Dimethyl-4-(3-methyl-2-oxo-1,3-benzoxazol-6-yl)-N-pentyl-piperazine-1-carboxamide

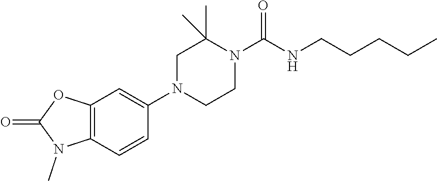 [SMILES: CCCCCNC(=O)N1CCN(c2ccc3c(c2)oc(=O)n3C)CC1(C)C]

Following general procedure D (Method A), XXXVIe (0.028 g, 0.090 mmol) and butyl isocyanate (0.010 g, 0.070 mmol) afforded the title compound as a white solid (0.022 g, 57%). ¹H NMR (400 MHz, DMSO-d₆) δ 7.25 (d, J=1.4 Hz, 1H), 7.18-7.09 (m, 2H), 6.44 (t, J=5.4 Hz, 1H), 3.60 (dt, J=12.9, 4.1 Hz, 1H), 3.35-3.31 (m, 5H), 3.22 (s, 3H), 3.06-2.96 (m, 3H), 2.84 (ddd, J=12.3, 8.3, 3.8 Hz, 1H), 1.87-1.77 (m, 1H), 1.67-1.48 (m, 6H), 1.46 (s, 3H), 1.31 (s, 3H). UPLC/NMS (method A): Rt 1.81 min. MS (ES) C₂₀H₂₉N₃O₄ requires 375, found 376 [M+H]⁺.

### Example 55: N-(2-Ethoxyethyl)-2,2-dimethyl-4-(3-methyl-2-oxo-1,3-benzoxazol-6-yl)piperazine-1 carboxamide

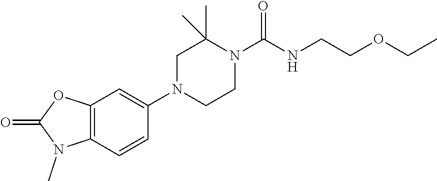 [SMILES: CCOCCNC(=O)N1CCN(c2ccc3c(c2)oc(=O)n3C)CC1(C)C]

Following general procedure D (Method B), XXXVIe (0.029 g, 0.097 mmol) and 2-ethoxyethylamine (0.052 g, 0.585 mmol) afforded the title compound as a white solid (0.002 g, 6%). ¹H NMR (400 MHz, CDCl₃) δ 6.83 (d, J=8.5 Hz, 1H), 6.71 (d, J=2.3 Hz, 1H), 6.59 (dd, J=8.6, 2.3 Hz, 1H), 4.87 (s, 1H), 3.65-3.60 (m, 2H), 3.56-3.49 (m, 4H), 3.42 (q, J=5.1 Hz, 2H), 3.36 (s, 3H), 3.34-3.28 (m, 2H), 3.13 (s, 2H), 1.49 (s, 6H), 1.21 (t, J=7.0 Hz, 3H). UPLC/MS (method A): Rt 1.77 min. MS (ES) C₁₉H₂₈N₄O₄ requires 376, found 377 [M+H]⁺.

### Example 56: N-(3-Methoxypropyl)-2,2-dimethyl-4-(3-methyl-2-oxo-1,3-benzoxazol-6-yl)piperazine-1-carboxamide

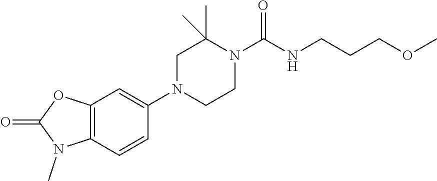 [SMILES: COCCCNC(=O)N1CCN(c2ccc3c(c2)oc(=O)n3C)CC1(C)C]

Following general procedure D (Method B), XXXVIe (0.025 g, 0.084 mmol) and 3-methoxypropylamine (0.045 g, 0.504 mmol) afforded the title compound as a white solid (0.012 g, 38%). ¹H NMR (400 MHz, CDCl₃) δ 6.82 (d, J=8.5 Hz, 1H), 6.71 (d, J=2.3 Hz, 1H), 6.59 (dd, J=8.5, 2.3 Hz, 1H), 5.22 (s, 1H), 3.62-3.57 (m, 2H), 3.52 (t, J=5.6 Hz, 2H), 3.38-3.32 (m, 2H), 3.36 (s, 3H), 3.35 (s, 3H), 3.32-3.27 (m, 2H), 3.13 (s, 2H), 1.81 (p, J=5.8 Hz, 2H), 1.49 (s, 6H). UPLC/MS (method A): Rt 1.73 min. MS (ES) C₁₉H₂₈N₄O₄ requires 376, found 377 [M+H]⁺.

### Example 57: N-(2-Benzyloxyethyl)-2,2-dimethyl-4-(3-methyl-2-oxo-1,3-benzoxazol-6-yl)piperazine-1-carboxamide

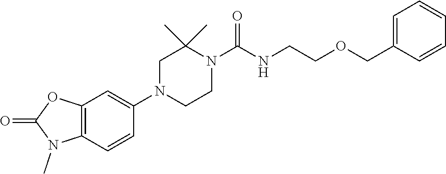 [SMILES: Cn1c(=O)oc2cc(N3CCN(C(=O)NCCOCc4ccccc4)C(C)(C)C3)ccc21]

Following general procedure D (Method C), XXXVIe (0.044 g, 0.148 mmol) and 2-benzyloxyethanamine (0.068 g, 0.450 mmol) afforded the title compound as a white solid (0.028 g, 43%). ¹H NMR (400 MHz, DMSO-d₆) δ 7.43-7.23 (m, 5H), 7.07 (d, J=8.6 Hz, 1H), 6.93 (d, J=2.3 Hz, 1H), 6.69 (dd, J=8.6, 2.3 Hz, 1H), 6.43 (t, J=5.5 Hz, 1H), 4.49 (s, 2H), 3.46 (dt, J=17.1, 5.7 Hz, 4H), 3.29 (s, 3H), 3.26-3.16 (m, 4H), 3.11 (s, 2H), 1.37 (s, 6H). UPLC/MS (method A): Rt 2.07 min. MS (ES) C₂₄H₃₀N₄O₄ requires 438, found 437 [M−H]⁻.

### Example 58: 4-[3-[2-(Dimethylamino)ethyl]-2-oxo-1,3-benzoxazol-6-yl]-2,2-dimethyl-N-(4-phenylbutyl)piperazine-1-carboxamide

### tert-Butyl 4-[3-[2-(dimethylamino)ethyl]-2-oxo-1,3-benzoxazol-6-yl]-2,2-dimethyl-piperazine-1-carboxylate (XXXVf)

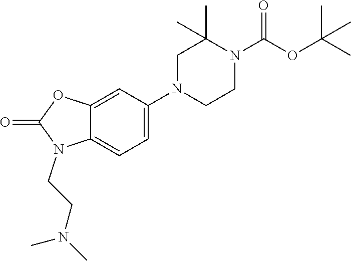 [SMILES: CN(C)CCn1c(=O)oc2cc(N3CCN(C(=O)OC(C)(C)C)C(C)(C)C3)ccc21]

Step 1: To a solution of XXXd (0.080 g, 0.23 mmol) in DMF (0.2 M) were added K₂CO₃ (0.095 g, 0.69 mmol) and 1,2-dibromoethane (0.346 g, 1.84 mmol) at RT and the reaction was stirred at 60° C. for 3 h. The mixture was poured into ice and the precipitate was filtered, solubilized in DCM and dried over Na₂SO₄. After evaporation of the solvent, tert-butyl 4-[3-(2-bromoethyl)-2-oxo-1,3-benzoxazol-6-yl]-2,2-dimethylpiperazine-1-carboxylate was used in the next step without further purification. ¹H NMR (400 MHz, CDCl₃) δ 6.92 (d, J=8.6 Hz, 1H), 6.71 (d, J=2.3 Hz, 1H), 6.59 (dd, J=8.6, 2.4 Hz, 1H), 4.18 (t, J=6.6 Hz, 2H), 3.80 (dd, J=6.3, 5.0 Hz, 2H), 3.65 (t, J=6.6 Hz, 2H), 3.34 (t, J=5.6 Hz, 2H), 3.22 (s, 2H), 1.49 (s, 9H), 1.45 (s, 6H). UPLC/MS (method A): Rt 2.60 min. MS (ES) C₂₀H₂₈BrN₃O₄ requires 454, found 455 [M+H]⁺.

Step 2: To a solution of tert-butyl 4-[3-(2-bromoethyl)-2-oxo-1,3-benzoxazol-6-yl]-2,2-dimethylpiperazine-1-carboxylate (0.105 g, 0.23 mmol) in DMF (0.2 M) were added K₂CO₃ (0.095 g, 0.69 mmol) and NHMe₂ (2M in THF, 1.0 mL, 2.3 mmol) at RT and the reaction was stirred at 60° C. for 2 h and then cooled to RT, poured into ice and the precipitate was filtered. The residue was used in the next step without further purification. ¹H NMR (400 MHz, CDCl₃) δ 7.29 (s, 1H), 6.63 (d, J=2.3 Hz, 1H), 6.57 (dd, J=8.6, 2.4 Hz, 1H), 4.37 (s, 2H), 3.78 (t, J=5.6 Hz, 2H), 3.32 (t, J=5.6 Hz, 2H), 3.23 (s, 2H), 2.86-2.57 (m, 8H), 1.49 (s, 9H), 1.42 (s, 6H). UPLC/MS (method A): Rt 2.05 min. MS (ES) C₂₂H₃₄N₄O₄ requires 418, found 419 [M+H]⁺.

### 3-[2-(Dimethylamino)ethyl]-6-(3,3-dimethylpiperazin-1-yl)-1,3-benzoxazol-2-one dihydrochloride (XXXVIf)

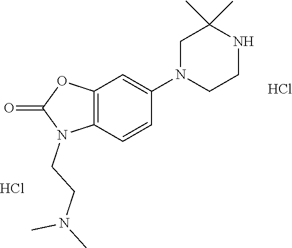 [SMILES: Cl ; CN(C)CCn1c(=O)oc2cc(N3CCNC(C)(C)C3)ccc21 ; Cl]

Following general procedure C (step 2), XXXVf (0.096 g, 0.23 mmol) afforded XXXVIf as a gray solid (0.068 g, 76%). H NMR (400 MHz, DMSO-d₆) δ 10.19 (bs, 2H), 9.32 (bs, 2H), 7.29 (d, J=8.2 Hz, 1H), 7.14 (d, J=2.2 Hz, 1H), 6.87 (dd, J=8.6, 2.2 Hz, 1H), 4.20 (t, J=6.1 Hz, 2H), 3.53-3.39 (s, 2H), 3.43 (m, 2H), 3.31-3.20 (m, 4H), 3.14 (s, 2H), 2.90-2.80 (m, 6H), 1.40 (s, 6H). UPLC/MS (method A): Rt 0.92 min. MS (ES) C₁₇H₂₆N₄O₂ requires 318, found 319 [M+H]⁺.

### 4-[3-[2-(Dimethylamino)ethyl]-2-oxo-1,3-benzoxazol-6-yl]-2,2-dimethyl-N-(4-phenylbutyl)piperazine-1-carboxamide

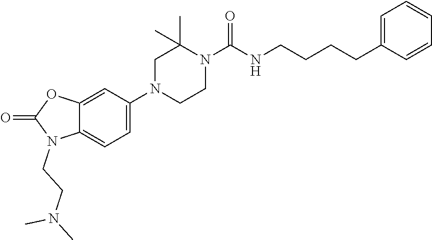 [SMILES: CN(C)CCn1c(=O)oc2cc(N3CCN(C(=O)NCCCCc4ccccc4)C(C)(C)C3)ccc21]

Following general procedure D (method A), XXXVIf (0.035 g, 0.09 mmol) and 4-phenylbutyl isocyanate (0.017 g, 0.096 mmol) afforded the title compound as a white solid (0.012 g, 27%). ¹H NMR (400 MHz, DMSO-d₆) δ 7.27 (t, J=7.5 Hz, 2H), 7.21-7.09 (m, 3H), 6.91 (d, J=2.3 Hz, 1H), 6.67 (dd, J=8.7, 2.3 Hz, 1H), 6.35 (t, J=5.5 Hz, 1H), 3.84 (t, J=6.2 Hz, 2H), 3.45 (t, J=5.5 Hz, 2H), 3.18 (t, J=5.4 Hz, 2H), 3.07 (s, 2H), 3.01 (td, J=7.0, 5.4 Hz, 2H), 2.62-2.51 (m, 4H), 2.16 (s, 6H), 1.61-1.50 (m, 2H), 1.42 (q, J=7.4 Hz, 2H), 1.36 (s, 6H). UPLC/MS (method A): Rt 2.05 min. MS (ES) C₂₈H₃₉N₅O₃ requires 493, found 494 [M+H]⁺.

### Example 59: 2,6-Dimethyl-4-(3-methyl-2-oxo-1,3-benzoxazol-6-yl)-N-(4-phenylbutyl)piperazine-1-carboxamide

### tert-Butyl 4-(3-hydroxy-4-nitrophenyl)-2,6-dimethyl-piperazine-1-carboxylate (XXXIe)

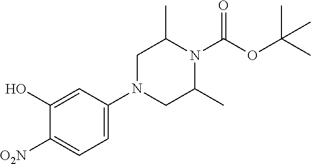 [SMILES: CC1CN(c2ccc([N+](=O)[O-])c(O)c2)CC(C)N1C(=O)OC(C)(C)C]

Step 1: Following general procedure F, XXXe (0.78 g, 6.81 mmol) and 5-fluoro-2-nitrophenol (1.0 g, 6.4 mmol) afforded 5-(3,5-dimethylpiperazin-1-yl)-2-nitrophenol as a white powder. The residue was used in the next step without further purification. ¹H NMR (400 MHz, CDCl₃) δ 7.93 (d, J=9.7 Hz, 1H), 6.43 (dd, J=9.7, 2.7 Hz, 1H), 6.29 (d, J=2.7 Hz, 1H), 3.76 (dd, J=12.6, 2.0 Hz, 2H), 2.94 (dtt, J=12.6, 6.3, 3.2 Hz, 2H), 2.56 (dd, J=12.6, 10.7 Hz, 2H), 1.15 (d, J=6.3 Hz, 6H). UPLC/MS (method A): Rt 1.19 min. MS (ES) C₁₂H₁₇N₃O₃ requires 251, found 252 [M+H]⁺.

Step 2: To a solution of 5-(3,5-dimethylpiperazin-1-yl)-2-nitrophenol (1.0 g, 3.98 mmol) in THF (0.1 M) was added Boc₂O (0.87 g, 3.98 mmol) in the presence of Et₃N (0.050 g, 0.7 mL, 4.78 mmol) and the reaction mixture was stirred at RT for 1 h. Then, the reaction mixture was diluted with EA, washed with saturated aqueous NaHCO₃ solution, brine and dried over Na₂SO₄. After evaporation of the solvent, the residue was purified by column chromatography (SiO₂), eluting with Cy/EA (7:3) to afford XXXIe as an orange oil (0.9 g, 64%). UPLC/MS (method A): Rt 2.50 min. MS (ES) C₁₇H₂₅N₃O₅ requires 351, found 352 [M+H]⁺.

### tert-Butyl 4-(4-amino-3-hydroxyphenyl)-2,6-dimethylpiperazine-1-carboxylate (XXXIIe)

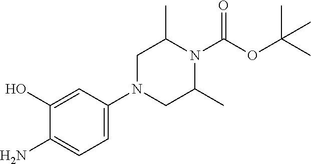 [SMILES: CC1CN(c2ccc(N)c(O)c2)CC(C)N1C(=O)OC(C)(C)C]

Following general procedure B (Method C), XXXIe (0.70 g, 1.99 mmol) afforded XXXIIe was used in the next step without further purification. UPLC/MS (method A): Rt 1.62 min. MS (ES) C₁₇H₂₇N₃O₃ requires 321, found 322 [M+H]⁺. tert-Butyl 2,6-dimethyl-4-(2-oxo-3H-1,3-benzoxazol-6-yl)piperazine-1-carboxylate (XXXIIIe)

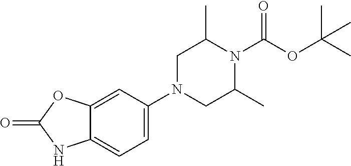 [SMILES: CC1CN(c2ccc3[nH]c(=O)oc3c2)CC(C)N1C(=O)OC(C)(C)C]

Following general procedure B (step 2), XXXIIe (0.6 g, 1.86 mmol) afforded XXXIIIe as a pink oil (0.5 g, 72%). ¹H NMR (400 MHz, CDCl₃) δ 8.69 (bs, 1H), 6.96 (d, J1=8.48 Hz, 1H), 6.84 (d, J=2.1 Hz, 1H), 6.74 (dd, J=8.5, 2.2 Hz, 1H), 6.82 (q, J=6.8 Hz, 2H), 3.24 (d, J=11.7 Hz, 2H), 2.87 (dd, J=11.7, 4.3 Hz, 2H), 1.50 (s, 9H), 1.37 (d, J=6.8 Hz, 6H). UPLC/MS (method A): Rt 2.22 min. MS (ES) C₁₈H₂₅N₃O₄ requires 347, found 348 [M+H]⁺.

### tert-Butyl 2,6-dimethyl-4-(3-methyl-2-oxo-1,3-benzoxazol-6-yl)piperazine-1-carboxylate (XXXVg)

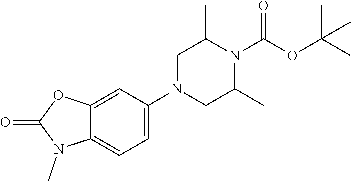 [SMILES: CC1CN(c2ccc3c(c2)oc(=O)n3C)CC(C)N1C(=O)OC(C)(C)C]

Following general procedure C (step 1), XXXIIIe (0.2 g, 0.58 mmol) and MeI (0.41 g, 2.90 mmol) afforded XXXVg as a white solid (0.19 g, 91%). UPLC/MS (method B): Rt 1.30 min. MS (ES) C₁₉H₂₇N₃O₄ requires 361, found 362 [M+H]⁺.

### 6-(3,5-Dimethylpiperazin-1-yl)-3-methyl-1,3-benzoxazol-2-one hydrochloride (XXXVIg)

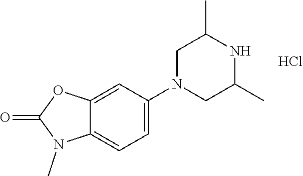 [SMILES: CC1CN(c2ccc3c(c2)oc(=O)n3C)CC(C)N1 ; Cl]

Following general procedure C (step 2), XXXVe (0.190 g, 0.53 mmol) afforded XXXVIg which was used in the next step without further purification. ¹H NMR (400 MHz, DMSO-d₆) δ 9.96-9.80 (m, 1H), 9.35-9.18 (m, 1H), 7.15 (s, 1H), 7.13 (d, =6.7 Hz, 1H), 6.90 (dd, J=8.6, 2.3 Hz, 1H), 3.72 (d, J=11.3 Hz, 2H), 3.39-3.28 (m, 2H), 3.29 (s, 3H), 2.83-2.74 (m, 2H), 1.32 (d, J=6.5 Hz, 6H). UPLC/MS (method A): Rt 1.03 min. MS (ES) C₁₄H₁₉N₃O₂ requires 261, found 262 [M+H]⁺.

### 2,6-Dimethyl-4-(3-methyl-2-oxo-1,3-benzoxazol-6-yl)-N-(4-phenylbutyl)piperazine-1-carboxamide

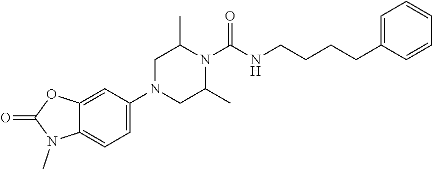 [SMILES: CC1CN(c2ccc3c(c2)oc(=O)n3C)CC(C)N1C(=O)NCCCCc1ccccc1]

Following general procedure D (Method A), XXXVIg (0.040 g, 0.12 mmol) and 4-phenylbutyl isocyanate (0.040 g, 0.04 mL, 0.24 mmol) afforded the title compound as white solid (0.030 g mg, 50%). ¹H NMR (400 MHz, CDCl₃) δ 7.32-7.25 (m, 2H), 7.21-7.15 (m, 2H), 6.87-6.82 (m, 2H), 6.76 (dd, J=8.4, 1.7 Hz, 1H), 4.45-4.36 (m, 1H), 4.18-4.08 (m, 2H), 3.37 (s, 3H), 3.31 (q, J=6.5 Hz, 2H), 3.25 (d, J=11.6 Hz, 2H), 2.89 (dd, J=11.3, 3.8 Hz, 2H), 2.66 (t, J=7.5 Hz, 2H), 1.62 (ddt, J=36.2, 14.4, 7.3 Hz, 4H), 1.39 (d, J=6.8 Hz, 6H). UPLC/MS (method A): Rt 2.27 min. MS (ES) C₂₅H₃₂N₄O₃ requires 436, found 437 [M+H]⁺.

### Example 60: 7-(3-Methyl-2-oxo-1,3-benzoxazol-6-yl)-N-(4-phenylbutyl)-4,7-diazaspiro[2.5]octane-4-carboxamide

### tert-Butyl 7-(3-hydroxy-4-nitrophenyl)-4,7-diazaspiro[2.5]octane-4-carboxylate (XXXI)

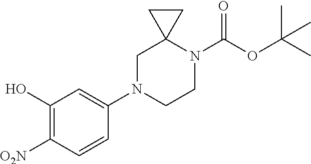 [SMILES: CC(C)(C)OC(=O)N1CCN(c2ccc([N+](=O)[O-])c(O)c2)CC12CC2]

Following general procedure F, XXXf (0.30 g, 1.4 mmol) and 5-fluoro-2-nitrophenol (0.33 g, 2.1 mmol) afforded XXXIf as an orange solid (0.471 g, 96%). ¹H NMR (400 MHz, CDCl₃) δ 11.19 (s, 1H), 7.94 (d, J=9.7 Hz, 1H), 6.37 (dd, J=9.7, 2.8 Hz, 1H), 6.25 (d, J=2.7 Hz, 1H), 3.72-3.67 (m, 2H), 3.48-3.41 (m, 2H), 3.23 (s, 2H), 1.48 (s, 9H), 1.12-1.05 (m, 2H), 0.87-0.82 (m, 2H). UPLC/MS (method A): Rt 1.48 min. MS (ES) C₁₇H₂₃N₃O₅ requires 349, found 350 [M+H]⁺.

### tert-Butyl 7-(4-amino-3-hydroxy-phenyl)-4,7-diazaspiro[2.5]octane-4-carboxylate (XXXIIf)

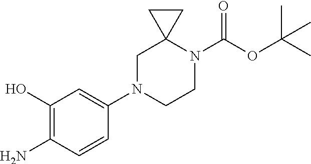 [SMILES: CC(C)(C)OC(=O)N1CCN(c2ccc(N)c(O)c2)CC12CC2]

Following general procedure B (Method C), XXXIf (0.47 g, 1.35 mmol) afforded XXXIIf which was used in the next step without further purification. UPLC/MS (method A): Rt 1.65 min. MS (ES) C₁₇H₂₅N₃O₃ requires 319, found 320 [M+H]⁺.

### tert-Butyl 7-(2-oxo-3H-1,3-benzoxazol-6-yl)-4,7-diazaspiro[2.5]octane-4-carboxylate (XXXIIIf)

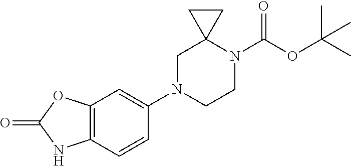 [SMILES: CC(C)(C)OC(=O)N1CCN(c2ccc3[nH]c(=O)oc3c2)CC12CC2]

Following general procedure B, XXXIIf (0.43 g, 1.35 mmol) and CDI (1.09 g, 6.75 mmol) afforded XXIIIf as a pink solid (0.192 g, 50%). ¹H NMR (400 MHz, DMSO-d₆) δ 11.28 (bs, 1H), 6.95 (d, J=2.3 Hz, 1H), 6.90 (d, J=8.5 Hz, 1H), 6.68 (dd, J=8.6, 2.3 Hz, 1H), 3.61-3.51 (m, 2H), 3.08-2.98 (m, 2H), 2.88 (s, 2H), 1.40 (s, 9H), 0.97-0.89 (m, 2H), 0.86-0.78 (m, 2H). UPLC/MS (method A): Rt 2.08 min. MS (ES) C₁₈H₂₃N₃O₄ requires 345, found 346 [M+H]˜.

### tert-Butyl 7-(3-methyl-2-oxo-1,3-benzoxazol-6-yl)-4,7-diazaspiro[2.5]octane-4-carboxylate (XXXVh)

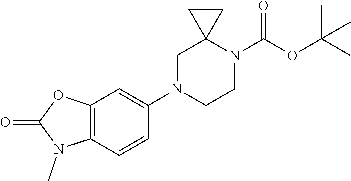 [SMILES: Cn1c(=O)oc2cc(N3CCN(C(=O)OC(C)(C)C)C4(CC4)C3)ccc21]

Following general procedure C, XXXIIIf (0.050 g, 0.15 mmol) and CH₃I (0.036 g, 0.016 mL, 0.22 mmol) afforded XXXVh which was used in the next step without further purification. ¹H NMR (400 MHz, DMSO-d₆) δ 7.07 (d, J=8.6 Hz, 1H), 7.01 (d, J=2.2 Hz, 1H), 6.78 (dd, J=8.6, 2.3 Hz, 1H), 3.61-3.54 (m, 2H), 3.28 (s, 3H), 3.08-3.01 (m, 2H), 2.91 (s, 2H), 1.40 (s, 9H), 0.96-0.90 (m, 2H), 0.87-0.81 (m, 2H). UPLC/MS (method A): Rt 2.27 min. MS (ES) C₁₉H₂₅N₃O₄ requires 359, found 360 [M+H]⁺.

### 6-(4,7-Diazaspiro[2.5]octan-7-yl)-3-methyl-1,3-benzoxazol-2-one hydrochloride (XXXVIh)

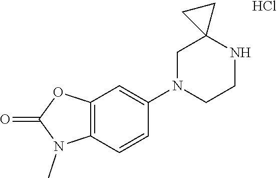 [SMILES: Cn1c(=O)oc2cc(N3CCNC4(CC4)C3)ccc21 ; Cl]

Following general procedure C, XXXVf (0.053 g, 0.15 mmol) afforded XXXVIh which was used in the next step without further purification ¹H NMR (400 MHz, DMSO-d₆) δ 9.73 (bs, 2H), 7.12 (d, J=8.6 Hz, 1H), 7.10 (d, J=2.2 Hz, 1H), 6.85 (dd, J=8.6, 2.3 Hz, 1H), 3.41-3.33 (m, 2H), 3.30 (s, 3H), 3.23 (s, 2H), 1.14-1.09 (m, 2H), 0.94-0.86 (m, 2H). UPLC/MS (method A): Rt 1.04 min. MS (ES) C₁₄H₁₇N₃O₂ requires 259, found 260 [M+H]⁺.

### 7-(3-Methyl-2-oxo-1,3-benzoxazol-6-yl)-N-(4-phenylbutyl)-4,7-diazaspiro[2.5]octane-4-carboxamide (Example 60)

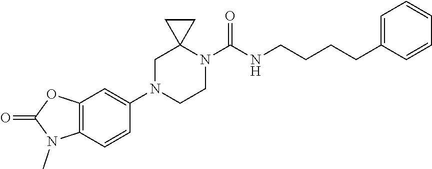 [SMILES: Cn1c(=O)oc2cc(N3CCN(C(=O)NCCCCc4ccccc4)C4(CC4)C3)ccc21]

Following general procedure D (Method A), XXXVIf (0.028 g, 0.095 mmol) and 4-phenylbutyl isocyanate (0.020 g, 0.114 mmol) afforded the title compound as a white solid (0.014 g, 34%). ¹H NMR (400 MHz, DMSO-d₆) δ 7.25-7.19 (m, 2H), 7.17-7.12 (m, 3H), 7.07 (d, J=8.6 Hz, 1H), 6.98 (d, J=2.3 Hz, 1H), 6.75 (dd, J=8.6, 2.3 Hz, 1H), 6.34 (t, J=5.8 Hz, 1H), 3.60 (s, 2H), 3.28 (s, 3H), 3.10 (q, J=6.5 Hz, 2H), 2.99-2.93 (m, 2H), 2.91 (s, 2H), 2.55 (t, J=7.5 Hz, 2H), 1.58-1.37 (m, 4H), 0.97-0.79 (m, 4H). UPLC/MS (method A): Rt 2.19 min. MS (ES) C₂₅H₃₀N₄O₃ requires 434, found 435 [M+H]⁺.

### Example 61: 3-(3-Methyl-2-oxo-1,3-benzoxazol-6-yl)-N-(4-phenylbutyl)-3,8-diazabicyclo[3.2.1]octane-8-carboxamide

### tert-Butyl 3-(3-hydroxy-4-nitrophenyl)-3,8-diazabicyclo[3.2.1]octane-8-carboxylate (XXXIg)

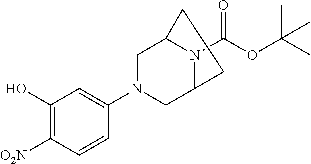 [SMILES: CC(C)(C)OC(=O)N1C2CCC1CN(c1ccc([N+](=O)[O-])c(O)c1)C2]

Following general procedure F, XXXg (0.99 g, 4.66 mmol) and 5-fluoro-2-nitrophenol (0.95 g, 6.06 mmol) afforded XXXIg as a yellow solid (1.63 g, 94%). ¹H NMR (400 MHz, CDCl₃) δ 11.20 (s, 1H), 7.98 (d, J=9.6 Hz, 1H), 6.41 (dd, 1=9.7, 2.8 Hz, 1H), 6.30 (d, J1=2.7 Hz, 1H), 4.52-4.28 (m, 2H), 3.59 (d, J=2.2 Hz, 1H), 3.56 (d, J=2.2 Hz, 1H), 3.25 (s, 1H), 3.22 (s, 1H), 2.09-1.93 (m, 2H), 1.81-1.71 (m, 2H), 1.51 (s, 9H). UPLC/MS (method A): Rt 2.44 min. MS (ES) C₁₇H₂₃N₃O₅ requires 349, found 350 [M+H]⁺.

### tert-Butyl 3-(4-amino-3-hydroxyphenyl)-3,8-diazabicyclo[3.2.1]octane-8-carboxylate (XXXIIg)

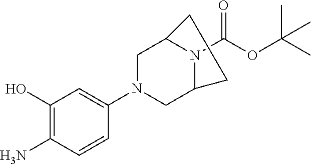 [SMILES: CC(C)(C)OC(=O)N1C2CCC1CN(c1ccc(N)c(O)c1)C2]

Following general procedure B (Method C), XXXIg (0.250 g, 0.72 mmol) afforded XXXIIg which was used in the next step without further purification. UPLC/MS (method A): Rt 1.85 min. MS (ES) C₁₇H₂₅N₃O₃ requires 319, found 320 [M+H]⁺. tert-Butyl 3-(2-oxo-3H-1,3-benzoxazol-6-yl)-3,8-diazabicyclo[3.2.1]octane-8-carboxylate (XXXIIg)

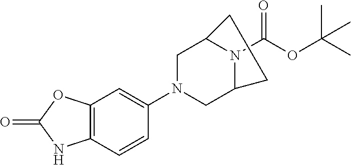 [SMILES: CC(C)(C)OC(=O)N1C2CCC1CN(c1ccc3[nH]c(=O)oc3c1)C2]

Following general procedure B (step 2), XXXIIg (0.230 g, 0.720 mmol) afforded XXIXh as a light pink solid (0.134 g, 54%). ¹H NMR (400 MHz, CDCl₃) δ 8.34 (s, 1H), 6.94 (d, J=8.7 Hz, 1H), 6.88 (s, 1H), 6.77 (d, J=8.7 Hz, 1H), 4.61-4.35 (m, 2H), 3.48-3.28 (m, 2H), 3.14-3.01 (m, 2H), 2.06-1.97 (m, 4H), 1.50 (s, 9H). UPLC/MS (method A): Rt 2.09 min. MS (ES) C₁₈H₂₃N₃O₄ requires 345, found 346 [M+H]⁺.

### tert-Butyl 3-(3-methyl-2-oxo-1,3-benzoxazol-6-yl)-3,8-diazabicyclo[3.2.1]octane-8-carboxylate (XXXVi)

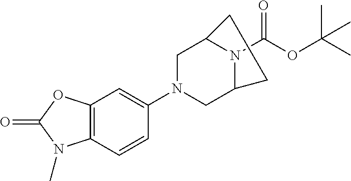 [SMILES: Cn1c(=O)oc2cc(N3CC4CCC(C3)N4C(=O)OC(C)(C)C)ccc21]

Following general procedure C (step 1), XXXIIIg (0.210 g, 0.608 mmol) and MeI (0.129 g, 0.912 mmol) afforded XXXVi as a white solid (0.180 g, 83%). ¹H NMR (400 MHz, CDCl₃) δ 6.91-6.81 (m, 2H), 6.81-6.72 (m, 1H), 4.50-4.26 (m, 2H), 3.38 (s, 3H), 3.37-3.30 (m, 2H), 3.14-2.88 (m, 2H), 2.07-1.91 (m, 4H). UPLC/MS (method A): Rt 2.28 min. MS (ES) C₁₉H₂₅N₃O₄ requires 359, found 360 [M+H]⁺.

### 6-(3,8-Diazabicyclo[3.2.1]octan-3-yl)-3-methyl-1,3-benzoxazol-2-one hydrochloride (XXXVIi)

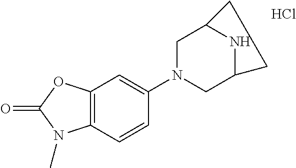 [SMILES: Cn1c(=O)oc2cc(N3CC4CCC(C3)N4)ccc21 ; Cl]

Following general procedure C (step 2), XXXVi (0.175 g, 0.487 mmol) afforded XXXVIi as a white solid (0.045 g, 95%). ¹H NMR (400 MHz, DMSO-d₆) δ 9.66-9.37 (m, 2H), 7.11 (d, J=8.6 Hz, 1H), 7.04 (d, J=2.3 Hz, 1H), 6.77 (dd, J=8.6, 2.3 Hz, 1H), 4.13-4.08 (m, 2H), 3.60-3.50 (m, 2H), 3.30 (s, 3H), 3.16-3.08 (m, 2H), 2.06-1.86 (m, 4H). UPLC/MS (method A): Rt 1.07 min. MS (ES) C₁₄H₁₇N₃O₂ requires 259, found 260 [M+H]⁺.

### 3-(3-Methyl-2-oxo-1,3-benzoxazol-6-yl)-N-(4-phenylbutyl)-3,8-diazabicyclo[3.2.1]octane-8-carboxamide

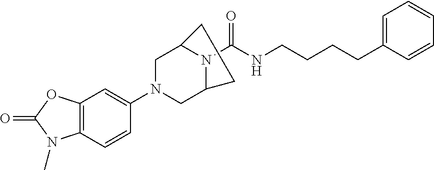 [SMILES: Cn1c(=O)oc2cc(N3CC4CCC(C3)N4C(=O)NCCCCc3ccccc3)ccc21]

Following general procedure D (Method A), XXXVIi (0.040 g, 0.120 mmol) and 4-phenylbutyl isocyanate (0.023 g, 0.132 mmol) afforded the title compound as a white solid (0.032 g, 61%). ¹H NMR (400 MHz, DMSO-d₆) δ 7.27-7.20 (m, 2H), 7.20-7.11 (m, 3H), 7.07 (d, J=8.6 Hz, 1H), 6.95 (d, J=2.3 Hz, 1H), 6.71 (dd, J=8.7, 2.3 Hz, 1H), 6.62 (t, 0.1=5.7 Hz, 1H), 4.43-4.23 (m, 2H), 3.37 (dd, J=11.2, 2.4 Hz, 2H), 3.29 (s, 3H), 3.14-3.02 (m, 2H), 2.76 (dd, J=11.1, 2.0 Hz, 2H), 2.60-2.52 (m, 2H), 1.85-1.66 (m, 4H), 1.61-1.48 (m, 2H), 1.48-1.35 (m, 2H). UPLC/MS (method A): Rt 2.15 min. MS (ES) C₂₅H₃₀N₄O₃ requires 434, found 435 [M+H]⁺.

### Example 62: 8-(3-Methyl-2-oxo-1,3-benzoxazol-6-yl)-7-oxo-N-(4-phenylbutyl)-2-oxa-5,8-diazaspiro[3.5]nonane-5-carboxamide

### Benzyl 7-oxo-2-oxa-5,8-diazaspiro[3.5]nonane-5-carboxylate (XXXh)

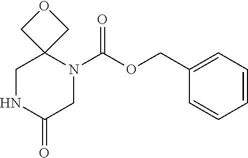 [SMILES: O=C1CN(C(=O)OCc2ccccc2)C2(CN1)COC2]

To a solution of 2-oxa-5,8-diazaspiro[3.5]nonan-7-one (0.22 g, 1.55 mmol) and DIPEA (0.40 g, 3.10 mmol) in anydrous DCM (0.1 M) was added benzyl chloroformate (0.53 g, 3.10 mmol) at 0° C. and the reaction mixture was stirred at RT for 2 h. The reaction mixture was diluted with DCM, washed with a saturated aq. NaHCO₃ solution, brine and dried over Na₂SO₄ to afford XXXh as a yellow solid (0.320 g, 75%). ¹H NMR (400 MHz, DMSO-d₆) δ 8.04 (s, 1H), 7.51-7.29 (m, 5H), 5.12 (s, 2H), 4.86 (d, J=6.5 Hz, 2H), 4.27 (d, J=6.5 Hz, 2H), 3.93 (s, 2H), 3.61 (d, J=2.6 Hz, 2H). UPLC/MS (method A): Rt 1.41 min. MS (ES) C₁₄H₁₆N₂O₄ requires 276, found 277 [M+H]⁺.

### Benzyl 8-(3-benzyloxy-4-nitrophenyl)-7-oxo-2-oxa-5,8-diazaspiro[3.5]nonane-5-carboxylate (XXXIh)

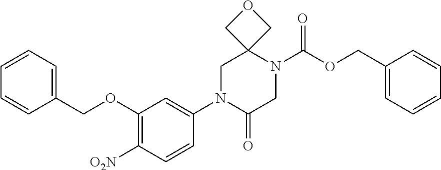 [SMILES: O=C1CN(C(=O)OCc2ccccc2)C2(COC2)CN1c1ccc([N+](=O)[O-])c(OCc2ccccc2)c1]

Following general procedure E, XXXh (0.215 g, 0.78 mmol) afforded XXXIh as a yellow solid (0.222 g, 56%). ¹H NMR (400 MHz, CDCl₃) δ 7.98 (d, J=8.8 Hz, 1H), 7.52-7.46 (m, 2H), 7.46-7.33 (m, 8H), 7.26 (d, J=2.1 Hz, 1H), 6.96 (dd, J=8.8, 2.2 Hz, 1H), 5.25 (s, 2H), 5.23 (s, 2H), 5.13 (d, J=6.6 Hz, 2H), 4.39 (d, J=6.7 Hz, 2H), 4.31 (s, 2H), 4.17 (s, 2H). UPLC/MS (method A): Rt 2.40 min. MS (ES) C₂₇H₂₅N₃O₇ requires 503, found 504 [M+H]⁺.

### Benzyl 8-(4-amino-3-benzyloxyphenyl)-7-oxo-2-oxa-5,8-diazaspiro[3.5]nonane-5-carboxylate (XXXIIh)

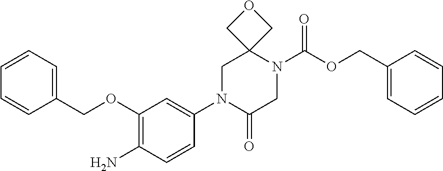 [SMILES: Nc1ccc(N2CC3(COC3)N(C(=O)OCc3ccccc3)CC2=O)cc1OCc1ccccc1]

Following general procedure B (Method C), step 1 using XXXIh (0.265 g, 0.53 mmol). The residue was used in the next step without further purification. ¹H NMR (400 MHz, CDCl₃) δ 7.55-7.31 (m, 10H), 6.82-6.72 (m, 2H), 6.68 (dd, J=8.2, 2.2 Hz, 1H), 5.21 (s, 2H), 5.09 (d, J=13.4 Hz, 4H), 4.42 (d, J=6.5 Hz, 2H), 4.27 (s, 2H), 4.10 (s, 2H), 3.93 (s, 2H), 3.81-3.73 (m, 1H), 1.92-1.83 (m, 1H). UPLC/MS (method A): Rt 2.15 min. MS (ES) C₂₇H₂₇N₃O₅ requires 473, found 474 [M+H]⁺.

### Benzyl 8-[4-[benzyl(methyl)amino]-3-benzyloxyphenyl]-7-oxo-2-oxa-5,8-diazaspiro[3.5]nonane-5-carboxylate (XXXVIIa)

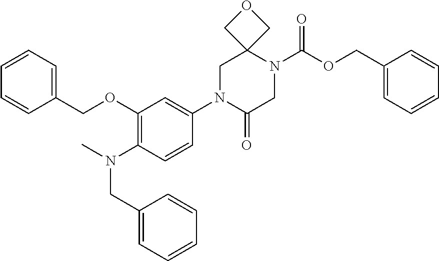 [SMILES: CN(Cc1ccccc1)c1ccc(N2CC3(COC3)N(C(=O)OCc3ccccc3)CC2=O)cc1OCc1ccccc1]

To a solution of XXXIIh (0.240 g, 0.51 mmol) in DCE (0.1 M), benzaldehyde (0.10 g, 0.91 mmol) and TFA (0.014 g, 0.01 mL, 0.127 mmol) were added at RT followed by NaBH(AcO)₃ (0.32 g, 1.52 mmol). After 2 h additional NaBH(AcO)₃ (0.32 g, 1.52 mmol) was added followed by formaldehyde 37% in water (0.76 g, 25.34 mmol) and the reaction was stirred at RT for 2 h. The reaction was quenched with MeOH (15 mL), then pH was neutralized with saturated aqueous NaHCO₃ solution, washed with brine and dried over Na₂SO₄ to afford XXXVII as a white waxy solid (0.285 g, 94%). ¹H NMR (400 MHz, CDCl₃) δ 7.45-7.33 (m, 10H), 7.27-7.20 (m, 5H), 6.95 (d, J=8.5 Hz, 1H), 6.90 (d, J=2.4 Hz, 1H), 6.80 (dd, J=8.4, 2.4 Hz, 1H), 5.22 (s, 2H), 5.18-5.05 (m, 4H), 4.43 (d, J=6.6 Hz, 2H), 4.29 (s, 2H), 4.26 (s, 2H), 4.13 (s, 2H), 2.70 (s, 3H). UPLC/MS (method B): Rt 1.88 min. MS (ES) C₃₅H₃₅N₃O₅ requires 577, found 578 [M+H]⁺.

### 8-[3-Hydroxy-4-(methylamino)phenyl]-2-oxa-5,8-diazaspiro[3.5]nonan-7-one (XXXVIIIa)

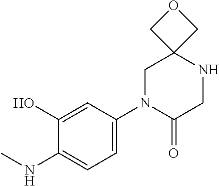 [SMILES: CNc1ccc(N2CC3(COC3)NCC2=O)cc1O]

Following general procedure B (method E), XXXVIIa (0.285 g, 0.493 mmol) afforded XXXVIIIa which was used in the next step without further purification. ¹H NMR (400 MHz, DMSO-d₆) δ 9.35 (s, 1H), 6.66-6.51 (m, 2H), 6.47-6.32 (m, 1H), 4.79 (bs, 1H), 4.49 (d, J=6.2 Hz, 2H), 4.44 (d, J=6.2 Hz, 2H), 3.76 (s, 2H), 3.38 (s, 2H), 2.72 (s, 3H). UPLC/MS (method A): Rt 1.01 min. MS (ES) C₁₃H₁₇N₃O₃ requires 263, found 264 [M+H]⁺.

### 8-(3-Methyl-2-oxo-1,3-benzoxazol-6-yl)-2-oxa-5,8-diazaspiro[3.5]nonan-7-one (XXXVIj)

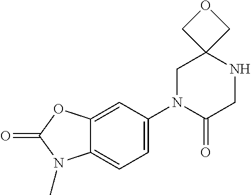 [SMILES: Cn1c(=O)oc2cc(N3CC4(COC4)NCC3=O)ccc21]

Following general procedure B, XXXVIIIa (0.129 g, 0.493 mmol) afforded XXXVIj as a white solid (0.030 g, 60%). ¹H NMR (400 MHz, CDCl₃) δ 7.20 (d, J=1.9 Hz, 1H), 7.14 (dd, J=8.3, 2.0 Hz, 1H), 7.01 (d, J=8.3 Hz, 1H), 4.68 (d, J=6.8 Hz, 2H), 4.66-4.59 (m, 2H), 3.99 (s, 2H), 3.78 (s, 2H), 3.44 (s, 3H). UPLC/MS (method A): Rt 0.95 min. MS (ES) C₁₄H₁₅N₃O₄ requires 289, found 290 [M+H]⁺.

### 8-(3-Methyl-2-oxo-1,3-benzoxazol-6-yl)-7-oxo-N-(4-phenylbutyl)-2-oxa-5,8-diazaspiro[3.5]nonane-5-carboxamide

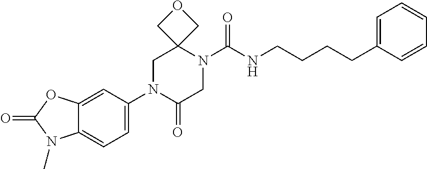 [SMILES: Cn1c(=O)oc2cc(N3CC4(COC4)N(C(=O)NCCCCc4ccccc4)CC3=O)ccc21]

Following general procedure D (Method A), XXXVIj (0.030 g, 0.101 mmol) and 4-phenylbutyl isocyanate (0.019 g, 0.111 mmol) afforded the title compound as a white solid (0.038 g, 80%). ¹H NMR (400 MHz, DMSO-d₆) δ 7.38 (d, J=1.9 Hz, 1H), 7.31-7.22 (m, 3H), 7.23-7.09 (m, 4H), 6.99 (t, J=5.5 Hz, 1H), 4.77 (d, J=6.8 Hz, 2H), 4.45 (d, J=6.9 Hz, 2H), 4.06 (d, J=10.2 Hz, 4H), 3.36 (s, 3H), 3.08 (q, J=6.5 Hz, 2H), 2.59 (t, J=7.6 Hz, 2H), 1.65-1.50 (m, 2H), 1.51-1.36 (m, 2H). UPLC/MS (method A): Rt 1.86 min. MS (ES) C₂₅H₂₈N₄O₅ requires 464, found 465 [M+H]⁺.

### Example 63: 4-(3-Methyl-2-oxo-1,3-benzoxazol-6-yl)-3-oxo-N-(4-phenylbutyl)piperazine-1-carboxamide

### tert-Butyl 4-(3-benzyloxy-4-nitrophenyl)-3-oxopiperazine-1-carboxylate (XXXIi)

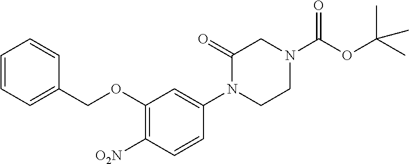 [SMILES: CC(C)(C)OC(=O)N1CCN(c2ccc([N+](=O)[O-])c(OCc3ccccc3)c2)C(=O)C1]

Following general procedure E, XXXi (0.750 g, 3.83 mmol) and 2-benzyloxy-4-bromo-1-nitrobenzene (1.42 g, 4.6 mmol) afforded XXXIi as a yellow solid (0.750 g, 40%). ¹H NMR (400 MHz, CDCl₃) δ 7.94 (d, J=8.8 Hz, 1H), 7.49-7.44 (m, 2H), 7.43-7.37 (m, 2H), 7.37-7.27 (m, 2H), 6.95 (dd, J=8.8, 2.0 Hz, 1H), 5.22 (s, 2H), 4.27 (s, 2H), 3.82-3.71 (m, 4H), 1.50 (s, 9H). UPLC/MS (method A): Rt 2.34 min. MS (ES) C₂H₂₅N₃O₆ requires 427, found 428 [M+H]⁺.

### tert-Butyl 4-(4-amino-3-hydroxyphenyl)-3-oxo-piperazine-1-carboxylate (XXXIIi)

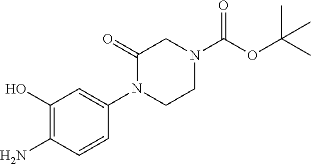 [SMILES: CC(C)(C)OC(=O)N1CCN(c2ccc(N)c(O)c2)C(=O)C1]

Following general procedure B (Method B), XXXIi (0.750 g, 1.76 mmol) afforded XXXIIi which was used in the next step without further purification. UPLC/MS (method A): Rt 1.47 min. MS (ES) C₁₅H₂₁N₃O₄ requires 307, found 308 [M+H]⁺.

### tert-Butyl 3-oxo-4-(2-oxo-3H-1,3-benzoxazol-6-yl)piperazine-1-carboxylate (XXXIIIh)

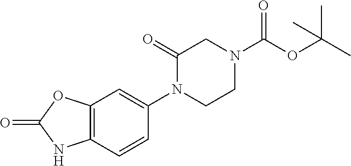 [SMILES: CC(C)(C)OC(=O)N1CCN(c2ccc3[nH]c(=O)oc3c2)C(=O)C1]

Following general procedure B (step 2), XXXIIi (0.54 g, 1.75 mmol) afforded XXXIIIh as a violet oil (0.40 g, 68%). ¹H NMR (400 MHz, CDCl₃) δ 9.29 (bs, 1H), 7.11 (d, J=1.8 Hz, 1H), 6.97 (dd, J=8.3, 2.0 Hz, 1H), 6.84 (d, J=8.3 Hz, 1H), 4.28 (s, 2H), 3.76 (dt, J=41.9, 4.9 Hz, 4H), 1.51 (s, 9H). UPLC/MS (method A): Rt 1.58 min. MS (ES) C₁₆H₁₉N₃O₅ requires 333, found 334 [M+H]⁺.

### tert-Butyl 4-(3-methyl-2-oxo-1,3-benzoxazol-6-yl)-3-oxo-piperazine-1-carboxylate (XXXVj)

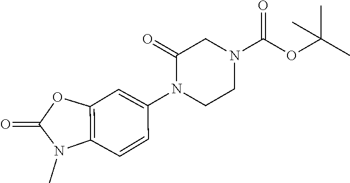 [SMILES: Cn1c(=O)oc2cc(N3CCN(C(=O)OC(C)(C)C)CC3=O)ccc21]

Following general procedure C (step 1), XXXIIIi (0.200 g, 0.6 mmol) and MeI (0.34 g, 2.40 mmol) afforded XXXVj as a whitish solid (0.200 g, 99%). UPLC/MS (method A): Rt 1.72 min. MS (ES) C₁₉H₂₇N₃O₄ requires 347, found 348 [M+H]⁺.

### 3-Methyl-6-(2-oxopiperazin-1-yl)-1,3-benzoxazol-2-one hydrochloride (XXXVIk)

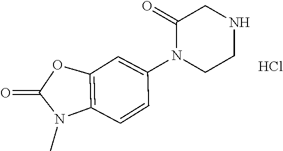 [SMILES: Cn1c(=O)oc2cc(N3CCNCC3=O)ccc21 ; Cl]

Following general procedure C (step 2), XXXVk (0.20 g, 0.6 mmol) afforded XXXVIk as white solid (0.150 g, 88%). UPLC/MS (method A): Rt 0.75 min. MS (ES) C₁₂H₁₃N₃O₃ requires 247, found 248 [M+H]⁺.

### 4-(3-Methyl-2-oxo-1,3-benzoxazol-6-yl)-3-oxo-N-(4-phenylbutyl)piperazine-1-carboxamide

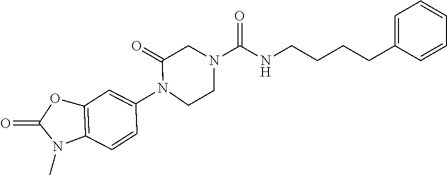 [SMILES: Cn1c(=O)oc2cc(N3CCN(C(=O)NCCCCc4ccccc4)CC3=O)ccc21]

Following general procedure D (Method A), XXXVIk (0.050 g, 0.6 mmol) and 4-phenylbutyl isocyanate (0.34 g, 2.40 mmol) afforded the title compound as white solid (40 mg, 53%). ¹H NMR (400 MHz, CDCl₃) δ 7.32-7.26 (m, 2H), 7.22-7.14 (m, 3H), 7.10 (d, J=8.2 Hz, 1H), 6.97 (d, J=8.2 Hz, 1H), 4.56 (bs, 1H), 4.13 (s, 2H), 3.78 (dd, J=30.8, 4.8 Hz, 4H), 3.40 (s, 3H), 3.28 (t, J=6.8 Hz, 2H), 2.64 (t, J=7.4 Hz, 2H), 1.74-1.48 (m, 4H). UPLC/MS (method A): Rt 1.83 min. MS (ES) C₂₃H₂₆N₄O₄ requires 422, found 423 [M+H]⁺.

### Example 64: 2,2-Dimethyl-4-(3-methyl-2-oxo-1,3-benzoxazol-6-yl)-3-oxo-N-(4-phenylbutyl) piperazine-1-carboxamide

### tert-Butyl 4-(4-amino-3-hydroxy-phenyl)-2,2-dimethyl-3-oxo-piperazine-1-carboxylate (XXXIj)

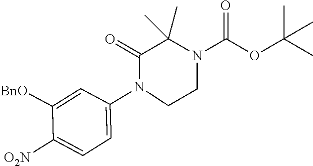 [SMILES: CC(C)(C)OC(=O)N1CCN(c2ccc([N+](=O)[O-])c(OCc3ccccc3)c2)C(=O)C1(C)C]

Following general procedure F, XXXj (1.0 g, 4.4 mmol) and 2-benzyloxy-4-bromo-1-nitrobenzene (1.6 g, 5.30 mmol) afforded XXXIj as a yellow solid (1.2 g, 60%). ¹H NMR (400 MHz, CDCl₃) δ 7.94 (d, J=8.8 Hz, 1H), 7.48 (d, J=7.1 Hz, 2H), 7.43-7.30 (m, 4H), 6.96 (dd, J=8.8, 2.1 Hz, 1H), 5.24 (s, 2H), 3.90-3.49 (m, 4H), 1.76 (s, 6H), 1.53 (s, 9H). UPLC/MS (method A): Rt 2.63 min. MS (ES) C₂₄H₂₉N₃O₆ requires 455, found 456 [M+H]⁺.

### tert-Butyl 4-(4-amino-3-hydroxy-phenyl)-2,2-dimethyl-3-oxo-piperazine-1-carboxylate (XXXIIj)

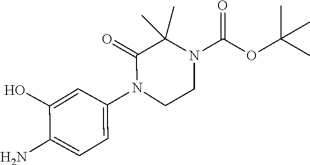 [SMILES: CC(C)(C)OC(=O)N1CCN(c2ccc(N)c(O)c2)C(=O)C1(C)C]

Following general procedure B (Method A), XXXIj (0.3 g, 0.66 mmol) afforded XXXIIj which was used in the next step without further purification. UPLC/MS (method A): Rt 1.73 min. MS (ES) C₁₇H₂₅N₃O₄ requires 335, found 336 [M+H]⁺. tert-Butyl 2,2-dimethyl-3-oxo-4-(2-oxo-3H-1,3-benzoxazol-6-yl)piperazine-1-carboxylate (XXXIIIi)

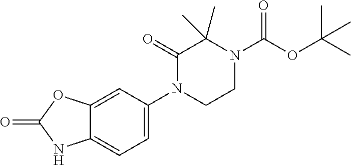 [SMILES: CC(C)(C)OC(=O)N1CCN(c2ccc3[nH]c(=O)oc3c2)C(=O)C1(C)C]

Following general procedure B, XXXIIj (0.22 g, 0.65 mmol) afforded XXXIIIi as a white solid (0.14 g, 59%). ¹H NMR (400 MHz, CDCl₃) δ 8.90 (bs, 1H), 7.09 (d, J=1.6 Hz, 1H), 6.92 (dd, J=8.3, 1.9 Hz, 1H), 6.79 (d, J=8.3 Hz, 1H), 3.88-3.79 (m, 2H), 3.69 (dd, J=5.9, 3.9 Hz, 2H), 1.79 (s, 6H), 1.53 (s, 9H). UPLC/MS (method A): Rt 1.89 min. MS (ES) C₁₈H₂₃N₃O₅ requires 365, found 366 [M+H]⁺.

### tert-Butyl 2,2-dimethyl-4-(3-methyl-2-oxo-1,3-benzoxazol-6-yl)-3-oxo-piperazine-1-carboxylate (XXXVk)

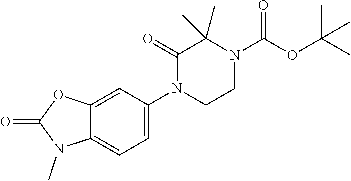 [SMILES: Cn1c(=O)oc2cc(N3CCN(C(=O)OC(C)(C)C)C(C)(C)C3=O)ccc21]

Following general procedure C, XXXIIIj (0.140 g, 0.38 mmol) and MeI (0.270 g, 1.90 mmol) afforded XXXVk which was used in the next step without further purification. ¹H NMR (400 MHz, CDCl₃) δ 7.17 (d, J=1.9 Hz, 1H), 7.09 (dd, J=8.3, 1.9 Hz, 1H), 6.95 (d, J=8.3 Hz, 1H), 3.81 (dd, J=5.9, 3.8 Hz, 2H), 3.71 (dd, J=5.9, 3.9 Hz, 2H), 3.40 (s, 3H), 1.75 (s, 6H), 1.52 (s, 9H). UPLC/MS (method A): Rt 2.01 min. MS (ES) C₁₉H₂₅N₃O₅ requires 375, found 376 [M+H]⁺.

### 6-(3,3-Dimethyl-2-oxo-piperazin-1-yl)-3-methyl-1,3-benzoxazol-2-one hydrochloride (XXXVI)

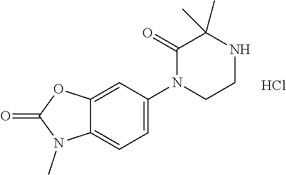 [SMILES: Cn1c(=O)oc2cc(N3CCNC(C)(C)C3=O)ccc21 ; Cl]

Following general procedure C, XXXVII (0.174 g, 0.46 mmol) afforded XXXVII as a white solid (0.110 g, 83%). UPLC/MS (method A): Rt 0.92 min. MS (ES) C₁₄H₁₇N₃O₃ requires 275, found 276 [M+H]⁺.

### 2,2-Dimethyl-4-(3-methyl-2-oxo-1,3-benzoxazol-6-yl)-3-oxo-N-(4-phenylbutyl)piperazine-1-carboxamide

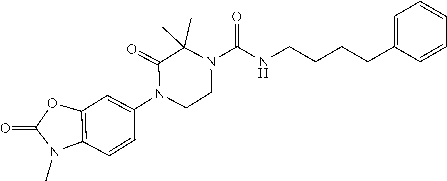 [SMILES: Cn1c(=O)oc2cc(N3CCN(C(=O)NCCCCc4ccccc4)C(C)(C)C3=O)ccc21]

Following general procedure D (Method A), XXXVII (0.030 g, 0.096 mmol) and 4-phenylbutyl isocyanate (0.03 g, 0.19 mmol) afforded the title compound as a white solid (0.021 g, 50%). ¹H NMR (400 MHz, CDCl₃) δ 7.29 (dd, J=8.4, 6.9 Hz, 2H), 7.23-7.14 (m, 4H), 7.10 (dd, J=8.3, 2.0 Hz, 1H), 6.96 (d, J=8.3 Hz, 1H), 4.63 (bs, 1H), 3.75 (dd, J=6.2, 3.5 Hz, 2H), 3.68 (dd, J=6.2, 3.5 Hz, 2H), 3.41 (s, 3H), 3.28 (t, J=7.0 Hz, 2H), 2.66 (t, J=7.4 Hz, 2H), 1.79 (s, 6H), 1.73-1.64 (m, 2H), 1.64-1.50 (m, 2H). UPLC/MS (method A): Rt 2.08 min. MS (ES) C₂₅H₃₀N₄O₄ requires 450, found 451 [M+H]⁺.

### Example 65: 2,2-Dimethyl-4-(3-methyl-2-oxo-1,3-benzoxazol-6-yl)-5-oxo-N-(4-phenylbutyl) piperazine-1-carboxamide

### tert-Butyl 4-(3-benzyloxy-4-nitrophenyl)-2,2-dimethyl-5-oxopiperazine-1-carboxylate (XXXIk)

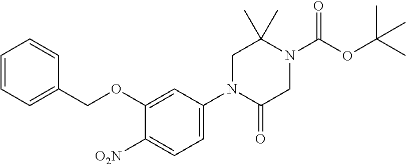 [SMILES: CC(C)(C)OC(=O)N1CC(=O)N(c2ccc([N+](=O)[O-])c(OCc3ccccc3)c2)CC1(C)C]

Following general procedure E, XXXk (0.1 g, 0.438 mmol) and 2-benzyloxy-4-bromo-1-nitrobenzene (0.162 g, 0.526 mmol) afforded XXXIk as a yellow solid (0.093 g, 47%). ¹H NMR (400 MHz, CDCl₃) δ 7.95 (d, J=8.8 Hz, 1H), 7.50-7.43 (m, 2H), 7.40 (m, 0.1=6.4, 1.0 Hz, 2H), 7.34 (m, 2H), 6.90 (dd, J=8.8, 2.2 Hz, 1H), 5.25 (s, 2H), 4.25 (s, 2H), 3.63 (s, 2H), 1.50 (s, 9H), 1.48 (s, 6H). UPLC/MS (method A): Rt 2.57 min. MS (ES) C₂₄H₂₉N₃O₆ requires 455, found 456 [M+H]⁺.

### tert-Butyl 4-(4-amino-3-hydroxyphenyl)-2,2-dimethyl-5-oxopiperazine-1-carboxylate (XXXIIk)

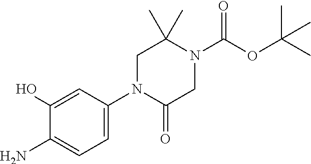 [SMILES: CC(C)(C)OC(=O)N1CC(=O)N(c2ccc(N)c(O)c2)CC1(C)C]

Following general procedure B (Method E), XXXIk (0.090 g, 0.198 mmol) afforded XXXIIk which used in the next step without further purification. UPLC/MS (method A): Rt 1.79 min. MS (ES) C₁₇H₂₁N₃O₄ requires 335, found 336 [M+H]⁺.

### tert-Butyl 2,2-dimethyl-5-oxo-4-(2-oxo-3H-1,3-benzoxazol-6-yl)piperazine-1-carboxylate (XXXIIIj)

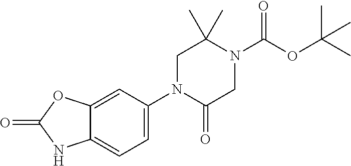 [SMILES: CC(C)(C)OC(=O)N1CC(=O)N(c2ccc3[nH]c(=O)oc3c2)CC1(C)C]

Following general procedure B, XXXIIk (0.211 g, 0.630 mmol) afforded XXXIIIj as a white solid (0.148 g, 0.410 mmol, 65%). ¹H NMR (400 MHz, CDCl₃) δ 8.51 (s, 1H), 7.18 (d, J=2.0 Hz, 1H), 7.03 (dd, J=8.4, 2.0 Hz, 1H), 6.94 (d, J=8.3 Hz, 1H), 4.25 (s, 2H), 3.63 (s, 2H), 1.53 (s, 6H), 1.51 (s, 9H). UPLC/MS (method A): Rt 1.92 min. MS (ES) C₁₈H₂₃N₃O₅ requires 361, found 362 [M+H]⁺.

### tert-Butyl 2,2-dimethyl-4-(3-methyl-2-oxo-1,3-benzoxazol-6-yl)-5-oxo-piperazine-1-carboxylate (XXXVI)

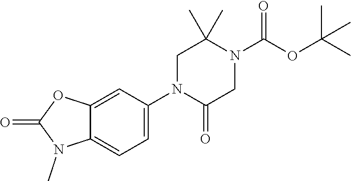 [SMILES: Cn1c(=O)oc2cc(N3CC(C)(C)N(C(=O)OC(C)(C)C)CC3=O)ccc21]

Following general procedure C, XXXIIIk (0.097 g, 0.268 mmol) afforded XXXVI which was used in the next step without further purification. ¹H NMR (400 MHz, CDCl₃) δ 7.21 (d, J=2.0 Hz, 1H), 7.14 (dd, J=8.3, 2.0 Hz, 1H), 6.96 (d, J=8.3 Hz, 1H), 4.23 (s, 2H), 3.64 (s, 2H), 3.41 (s, 3H), 1.53 (s, 6H), 1.50 (s, 9H). UPLC/MS (method A): Rt 2.03 min. MS (ES) C₁₉H₂₅N₃O₅ requires 375, found 376 [M+H]⁺.

### 6-(5,5-Dimethyl-2-oxo-piperazin-1-yl)-3-methyl-1,3-benzoxazol-2-one hydrochloride (XXXVIm)

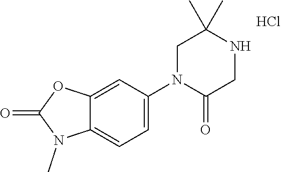 [SMILES: Cn1c(=O)oc2cc(N3CC(C)(C)NCC3=O)ccc21 ; Cl]

Following general procedure C, XXXVm (0.097 g, 0.268 mmol) afforded XXXVIm as a white solid (0.067 g, 80%). UPLC/MS (method A): Rt 1.09 min. MS (ES) C₁₄H₁₇N₃O₃ requires 275, found 276 [M+H]⁺.

### 2,2-Dimethyl-4-(3-methyl-2-oxo-1,3-benzoxazol-6-yl)-5-oxo-N-(4-phenylbutyl)piperazine-1-carboxamide

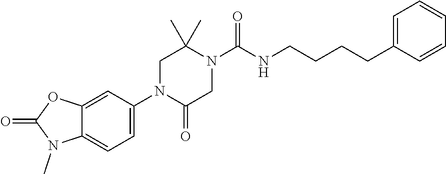 [SMILES: Cn1c(=O)oc2cc(N3CC(C)(C)N(C(=O)NCCCCc4ccccc4)CC3=O)ccc21]

Following procedure D (Method A), XXXVIm (0.040 g, 0.128 mmol) afforded the title compound as a white solid (0.040 g, 0.089 mmol, 70%). ¹H NMR (400 MHz, DMSO-d₆) δ 7.38 (d, J=1.9 Hz, 1H), 7.31-7.23 (m, 3H), 7.22-7.13 (m, 4H), 6.29 (t, J=5.4 Hz, 1H), 4.00 (s, 2H), 3.68 (s, 2H), 3.34 (s, 3H), 3.03 (q, J=6.5 Hz, 2H), 2.58 (t, J=7.6 Hz, 2H), 1.56 (p, J=7.2 Hz, 2H), 1.47-1.39 (m, 2H), 1.43 (s, 6H). UPLC/MS (method A): Rt 2.09 min. MS (ES) C₂₅H₃₀N₄O₄ requires 450, found 451 [M+H]⁺.

### Example 66: (3aS,6aR)- and (3aR,6aS)-2-(3-Methyl-2-oxo-1,3-benzoxazol-6-yl)-N-(4-phenylbutyl)-1,3,3a,4,6,6a-hexahydropyrrolo[3,4-c]pyrrole-5-carboxamide tert-Butyl (3aS,6aR) and (3aR,6aS)-2-(3-hydroxy-4-nitrophenyl)-1,3,3a,4,6,6a-hexahydropyrrolo[3,4-c]pyrrole-5-carboxylate (XXXI′a)

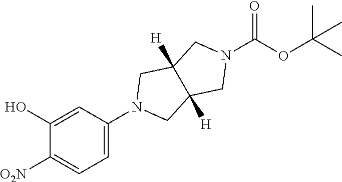 [SMILES: CC(C)(C)OC(=O)N1C[C@@H]2CN(c3ccc([N+](=O)[O-])c(O)c3)C[C@@H]2C1]

Following general procedure F, XXX′a (0.100 g, 0.471 mmol) and 5-fluoro-2-nitrophenol (0.074 g, 0.471 mmol) afforded XXXI′a as a yellow solid (0.132 g, 80%). ¹H NMR (400 MHz, CDCl₃) δ 11.37 (s, 1H), 7.95 (d, J=9.5 Hz, 1H), 6.15 (dd, J=2.6, 9.5 Hz, 1H), 6.00 (d, J=2.5 Hz, 1H), 3.67 (dd, J=7.0, 11.1 Hz, 4H), 3.33 (dd, J=4.1, 10.8 Hz, 4H), 3.12-2.93 (m, 2H), 1.46 (s, 9H). UPLC/MS (method A): Rt 2.31 min. MS (ES) C₁₇H₃₃N₃O₅ requires 349, found 350 [M+H]⁺.

### tert-Butyl (3aS,6aR) and (3aR,6aS)-2-(4-amino-3-hydroxyphenyl)-1,3,3a,4,6,6a-hexahydropyrrolo[3,4-c]pyrrole-5-carboxylate (XXXII′a)

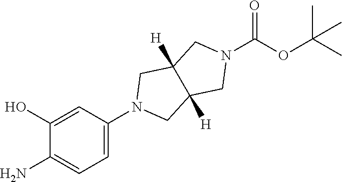 [SMILES: CC(C)(C)OC(=O)N1C[C@@H]2CN(c3ccc(N)c(O)c3)C[C@@H]2C1]

Following general procedure B (Method A), XXXI′a (0.132 g, 0.378 mmol) afforded XXXII′a which was used in the next step without further purification. UPLC/MS (method A): Rt 1.45 min. MS (ES) C₁₇H₂₅N₃O₃ requires 319, found 377 [M+AcO]⁺.

### tert-Butyl (3aS,6aR)- and (3aR,6aS)-2-(2-oxo-3H-1,3-benzoxazol-6-yl)-1,3,3a,4,6,6a-hexahydropyrrolo[3,4-c]pyrrole-5-carboxylate (XXXIII′a)

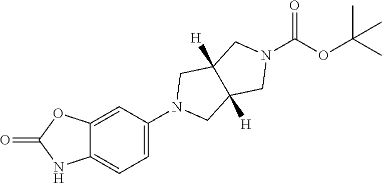 [SMILES: CC(C)(C)OC(=O)N1C[C@@H]2CN(c3ccc4[nH]c(=O)oc4c3)C[C@@H]2C1]

Following general procedure B (step 2), XXXII′a (0.120 g, 0.378 mmol) afforded XXXIII′a as a purple solid (0.083 g, 63%). ¹H NMR (400 MHz, CDCl₃) δ 8.56 (bs, 1H), 6.91 (d, J=8.5 Hz, 1H), 6.50 (s, 1H), 6.35 (d, J=7.6 Hz, 1H), 3.72-3.59 (m, 2H), 3.58-3.46 (m, 2H), 3.42-3.23 (m, 2H), 3.22-3.13 (m, 2H), 3.10-2.96 (m, 2H), 1.46 (s, 9H). UPLC/MS (method A): Rt 1.99 min. MS (ES) C₁₈H₂₃N₃O₄ requires 345, found 346 [M+H]⁺.

### tert-Butyl (3aS,6aR)- and (3aR,6aS)-2-(3-methyl-2-oxo-1,3-benzoxazol-6-yl)-1,3,3a,4,6,6a-hexahydropyrrolo[3,4-c]pyrrole-5-carboxylate (XXXV′a)

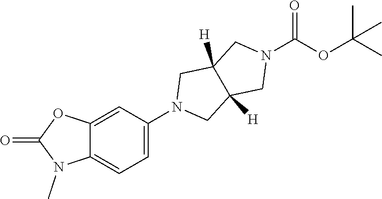 [SMILES: Cn1c(=O)oc2cc(N3C[C@H]4CN(C(=O)OC(C)(C)C)C[C@H]4C3)ccc21]

Following general procedure C (step 1), XXXIII′a (0.035 g, 0.101 mmol) and MeI (0.072 g, 0.505 mmol) afforded XXXV′a as a white solid (0.02 g, 0.056 mmol, 55%). ¹H NMR (400 MHz, CDCl₃) δ 6.80 (d, J=8.5 Hz, 1H), 6.50 (d, J=2.1 Hz, 1H), 6.37 (dd, J=1.9, 8.5 Hz, 1H), 3.70-3.60 (m, 2H), 3.55-3.47 (m, 2H), 3.35 (s, 3H), 3.22-3.15 (m, 2H), 3.03-2.98 (m, 2H), 1.45 (s, 9H). UPLC/MS (method A): Rt 2.19 min. MS (ES) C₁₉H₂₅N₃O₄ requires 359, found 360 [M+H]⁺.

### 6-[(3aS,6aR)- and (3aR,6aS)-2,3,3a,4,6,6a-Hexahydro-1H-pyrrolo[3,4-c]pyrrol-5-yl]-3-methyl-1,3-benzoxazol-2-one hydrochloride (XXXVI′a)

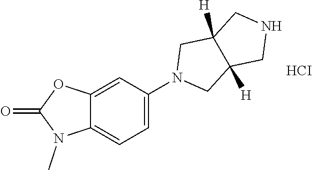

Following general procedure C (step 2), XXXV′a (0.20 g, 0.056 mmol) afforded XXXVI′a as a violet solid (0.010 g, 60%). UPLC/MS (method A): Rt 1.08 min. MS (ES) C₁₄H₁₇N₃O₂ requires 259, found 260 [M+H]⁺.

### (3aS,6aR)- and (3aR,6aS)-2-(3-Methyl-2-oxo-1,3-benzoxazol-6-yl)-N-(4-phenylbutyl)-1,3,3a,4,6,6a-hexahydropyrrolo[3,4-c]pyrrole-5-carboxamide

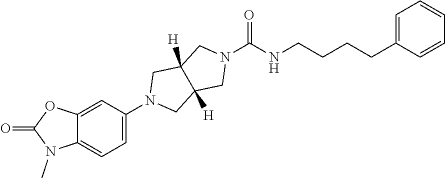 [SMILES: Cn1c(=O)oc2cc(N3C[C@H]4CN(C(=O)NCCCCc5ccccc5)C[C@H]4C3)ccc21]

Following general procedure D (Method A), XXXVI′a (0.010 g, 0.034 mmol) and 4-phenylbutyl isocyanate (0.010 g, 0.062 mmol) afforded the title compound as white solid (10 mg, 71%). ¹H NMR (400 MHz, CDCl₃) δ 7.28-7.23 (m, 2H), 7.17 (t, J=7.3 Hz, 3H), 6.80 (d, J=8.5 Hz, 1H), 6.47 (d, J=2.2 Hz, 1H), 6.33 (dd, J=2.2, 8.5 Hz, 1H), 4.17 (t, J=5.51 Hz, 1H), 3.64 (dd, J=7.4, 10.0 Hz, 2H), 3.51 (dd, J=7.2, 9.3 Hz, 2H), 3.35 (s, 3H), 3.34 (dd, J=4.0, 10.2 Hz, 2H), 3.27 (q, J=7.0 Hz, 2H), 3.22 (dd, J=3.8, 9.5 Hz, 2H), 3.14-3.02 (m, 2H), 2.65 (t, J=7.5 Hz, 2H), 1.74-1.62 (m, 2H), 1.62-1.51 (m, 2H). UPLC/MS (method A): Rt 2.10 min. MS (ES) C₂₅H₃₀N₄O₃ requires 434, found 435 [M+H]⁺.

### Example 67: 7-(2-Oxo-3H-1,3-benzoxazol-6-yl)-N-(4-phenylbutyl)-2,7-diazaspiro[3.5]nonane-2-carboxamide

### 6-(2,7-Diazaspiro[3.5]nonan-7-yl)-3H-1,3-benzoxazol-2-one hydrochloride (XXXIV″a)

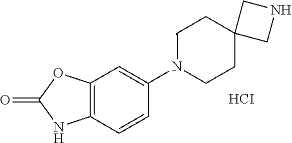

Following general procedure C, XXXII″a (0.050 g, 0.139 mmol) afforded XXXIV″a which was used in the next step without further purification. MS (ES) C₁₄H₁₇N₃O₂ requires 259, found 260 [M+H]⁺.

### 7-(2-Oxo-3H-1,3-benzoxazol-6-yl)-N-(4-phenylbutyl)-2,7-diazaspiro[3.5]nonane-2-carboxamide

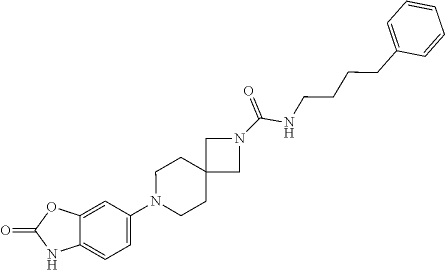 [SMILES: O=C(NCCCCc1ccccc1)N1CC2(CCN(c3ccc4[nH]c(=O)oc4c3)CC2)C1]

Following general procedure D (Method A), XXXIV″a (0.036 g, 0.139 mmol) and 4-phenylbutyl isocyanate (0.027 g, 0.152 mmol) afforded the title compound as a pinkish solid (0.022 g, 34%). ¹H NMR (400 MHz, DMSO-d₆) δ 11.25-10.26 (bs, 1H), 7.31-7.23 (m, 2H), 7.20-7.13 (m, 3H), 6.95 (d, J=2.3 Hz, 1H), 6.90 (d, J=8.5 Hz, 1H), 6.71 (dd, J=8.6, 2.3 Hz, 1H), 6.20 (t, J=5.8 Hz, 1H), 3.50 (s, 4H), 3.03-2.95 (m, 6H), 2.56 (t, J=7.6 Hz, 2H), 1.80-1.70 (m, 4H), 1.60-1.47 (m, 2H), 1.44-1.33 (m, 2H). UPLC/MS (method A): Rt 1.98 min. MS (ES) C₂₅H₃₀N₄O₃ requires 434, found 435 [M+H]⁺.

### Example 68: 7-(3-methyl-2-oxo-1,3-benzoxazol-6-yl)-N-(4-phenylbutyl)-2,7-diazaspiro[3.5]nonane-2-carboxamide

### tert-Butyl 7-(3-hydroxy-4-nitrophenyl)-2,7-diazaspiro[3.5]nonane-2-carboxylate (XXXI″a)

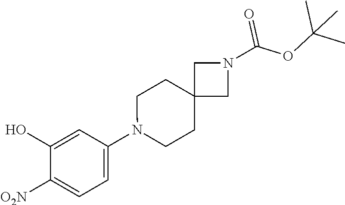 [SMILES: CC(C)(C)OC(=O)N1CC2(CCN(c3ccc([N+](=O)[O-])c(O)c3)CC2)C1]

Following general procedure F, XXX″a (0.30 g, 1.33 mmol) and 5-fluoro-2-nitrophenol (0.312 g, 1.99 mmol) XXXI″a as a yellow solid (0.387 g, 80%). ¹H NMR (400 MHz, CDCl₃) 11.22 (s, 1H), 7.93 (d, J=9.7 Hz, 1H), 6.43 (dd, J=9.7, 2.7 Hz, 1H), 6.31 (d, J=2.7 Hz, 1H), 3.70 (s, 4H), 3.46-3.36 (m, 4H), 1.89-1.79 (m, 4H), 1.45 (s, 9H). S UPLC/MS (method A): Rt 2.43 min. MS (ES) C₁₈H₂₅N₃O₅ requires 363, found 364 [M+H]⁺.

### tert-Butyl 7-(4-amino-3-hydroxyphenyl)-2,7-diazaspiro[3.5]nonane-2-carboxylate (XXXII″a)

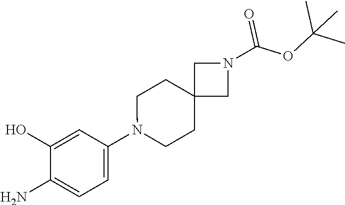 [SMILES: CC(C)(C)OC(=O)N1CC2(CCN(c3ccc(N)c(O)c3)CC2)C1]

Following general procedure B (Method C), XXXI″a (0.387 g, 1.07 mmol) afforded XXXII″a which was used in the next step without further purification. UPLC/MS (method A): Rt 1.72 min. MS (ES) C₁₈H₂₇N₃O₃ requires 333, found 334 [M+H]⁺. tert-Butyl 7-(2-Oxo-3H-1,3-benzoxazol-6-y)-2,7-diazaspiro[3.5]nonane-2-carboxylate (XXXIII″a)

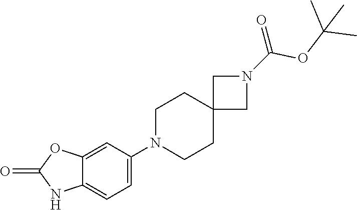 [SMILES: CC(C)(C)OC(=O)N1CC2(CCN(c3ccc4[nH]c(=O)oc4c3)CC2)C1]

Following general procedure B, XXXII″a (0.36 g, 1.07 mmol) and CDI (0.87 g, 5.35 mmol) afforded XXIII″a as a pink solid (0.192 g, 50%). ¹H NMR (400 MHz, DMSO-d₆) δ 11.26 (bs, 1H), 6.95 (d, J=2.3 Hz, 1H), 6.90 (d, J=8.5 Hz, 1H), 6.71 (dd, J=8.6, 2.3 Hz, 1H), 3.58 (s, 4H), 3.00 (s, 4H), 1.77 (t, J=5.5 Hz, 4H), 1.38 (s, 9H). UPLC/MS (method A): Rt 2.00 min. MS (ES) C₁₉H₂₅N₃O₄ requires 359, found 360 [M+H]⁺.

### tert-Butyl 7-(3-methyl-2-oxo-1,3-benzoxazol-6-yl)-2,7-diazaspiro[3.5]nonane-2-carboxylate (XXXV′a)

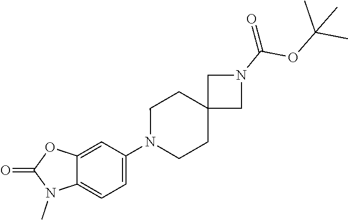 [SMILES: Cn1c(=O)oc2cc(N3CCC4(CC3)CN(C(=O)OC(C)(C)C)C4)ccc21]

Following general procedure C, XXXIII″a (0.050 g, 0.14 mmol) and CH₃I (0.023 g, 0.010 mL, 0.14 mmol) afforded XXXV″a as a pink solid (0.026 g, 50%). ¹H NMR (400 MHz, DMSO-d₆) δ 7.06 (d, 0.1=8.6 Hz, 1H), 7.01 (d, J=2.2 Hz, 1H), 6.80 (dd, J=8.6, 2.3 Hz, 1H), 3.58 (s, 4H), 3.03 (s, 4H), 1.77 (t, J=5.5 Hz, 4H), 1.38 (s, 9H). UPLC/MS (method A): Rt 2.20 min. MS (ES) C₂₀H₂₇N₃O₄ requires 373, found 374 [M+H]⁺.

### 6-(2,7-Diazaspiro[3.5]nonan-7-yl)-3-methyl-1,3-benzoxazol-2-one hydrochloride (XXXVI″a)

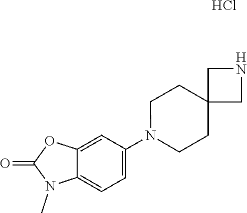 [SMILES: Cl ; Cn1c(=O)oc2cc(N3CCC4(CC3)CNC4)ccc21]

Following general procedure C, XXXV″a (0.026 g, 0.07 mmol) afforded XXXVI″a which was used in the next step without further purification. UPLC/MS (method A): Rt 1.05 min. MS (ES) C₁₅H₁₉N₃O₂ requires 273, found 274 [M+H]⁺.

### 7-(3-Methyl-2-oxo-1,3-benzoxazol-6-yl)-N-(4-phenylbutyl)-2,7-diazaspiro[3.5]nonane-2-carboxamide

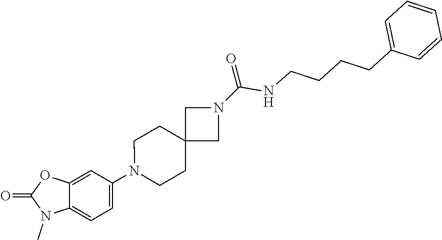 [SMILES: Cn1c(=O)oc2cc(N3CCC4(CC3)CN(C(=O)NCCCCc3ccccc3)C4)ccc21]

Following general procedure D (Method A), XXXVI″a (0.024 g, 0.07 mmol) and 4-phenylbutyl isocyanate (0.015 g, 0.084 mmol) afforded the title compound as a white solid (0.022 g, 70%). ¹H NMR (400 MHz, DMSO-d₆) δ 7.31-7.24 (m, 2H), 7.21-7.14 (m, 3H), 7.06 (d, J=8.6 Hz, 1H), 7.02 (d, J=2.2 Hz, 1H), 6.81 (dd, J=8.6, 2.3 Hz, 1H), 6.20 (t, J=5.8 Hz, 1H), 3.51 (s, 4H), 3.28 (s, 3H), 3.06-2.94 (m, 6H), 2.56 (t, 0.1=7.6 Hz, 2H), 1.76 (t, J=5.5 Hz, 4H), 1.60-1.48 (m, 2H), 1.44-1.34 (m, 2H). UPLC/MS (method A): Rt 2.10 min. MS (ES) C₂₆H₃₂N₄O₃ requires 434, found 435 [M+H]⁺.

### Example 69: 2-(2-Oxo-3H-1,3-benzoxazol-6-yl)-N-(4-phenylbutyl)-2,7-diazaspiro[3.5]nonane-7-carboxamide

### tert-Butyl 2-(3-hydroxy-4-nitrophenyl)-2,7-diazaspiro[3.5]nonane-7-carboxylate (XXXVII″b)

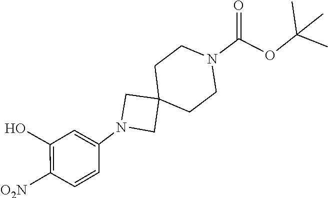 [SMILES: CC(C)(C)OC(=O)N1CCC2(CC1)CN(c1ccc([N+](=O)[O-])c(O)c1)C2]

Following general procedure F, XXX″b (0.3 g, 1.33 mmol) and 5-fluoro-2-nitrophenol (0.27 g, 1.72 mmol) afforded XXXVII″b as a yellow solid (0.415 g, 86%). ¹H NMR (400 MHz, CDCl₃) δ 11.46 (s, 1H), 7.96 (d, J=9.3 Hz, 1H), 5.97 (dd, J=9.4, 2.4 Hz, 1H), 5.84 (d, J=2.4 Hz, 1H), 3.80 (s, 4H), 3.50-3.40 (m, 4H), 1.91-1.77 (m, 4H). UPLC/MS (method A): Rt 2.51 min. MS (ES) C₁₈H₂₅N₃O₅ requires 336, found 364 [M+H]⁺.

### tert-Butyl 2-(4-amino-3-hydroxyphenyl)-2,7-diazaspiro[3.5]nonane-7-carboxylate (XXXII″b)

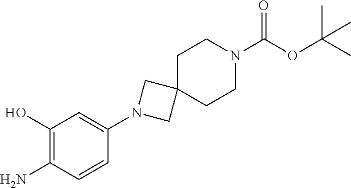 [SMILES: CC(C)(C)OC(=O)N1CCC2(CC1)CN(c1ccc(N)c(O)c1)C2]

Following general procedure B (Method C), XXXI″b (0.40 g, 1.10 mmol) afforded XXXII″b which was used in the next step without further purification. UPLC/MS (method A): Rt 1.58 min. MS (ES) C₁₈H₂₇N₃O₃ requires 333, found 332 [M+H]⁺.

### tert-Butyl 2-(2-oxo-3H-1,3-benzoxazol-6-yl)-2,7-diazaspiro[3.5]nonane-7-carboxylate (XXXIII″b)

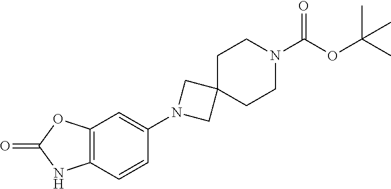 [SMILES: CC(C)(C)OC(=O)N1CCC2(CC1)CN(c1ccc3[nH]c(=O)oc3c1)C2]

Following general procedure B, XXXII″b (0.37 g, 1.10 mmol) and CDI (0.89 g, 5.50 mmol) afforded XXXIII″b as a pink solid (0.22 g, 56%). ¹H NMR (400 MHz, CDCl₃) S 8.36 (s, 1H), 6.91 (d, J=8.4 Hz, 1H), 6.42 (s, 1H), 6.28 (d, J=8.4 Hz, 1H), 3.66 (s, 4H), 3.55-3.38 (m, 4H), 1.90-1.77 (m, 4H), 1.49 (s, 9H). UPLC/MS (method A): Rt 2.11 min. MS (ES) C₁₉H₂₅N₃O₄ requires 359, found 360 [M+H]⁺.

### 6-(2,7-Diazaspiro[3.5]nonan-2-yl)-3H-1,3-benzoxazol-2-one hydrochloride (XXXIV″b)

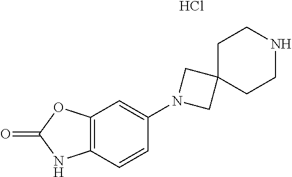 [SMILES: Cl ; O=c1[nH]c2ccc(N3CC4(CCNCC4)C3)cc2o1]

Following general procedure C, XXXIII″b (0.050 g, 0.139 mmol) afforded XXXIV″b which was used in the next step without further purification. ¹H NMR (400 MHz, DMSO-dc) S 11.37 (s, 1H), 8.94 (bs, 2H), 6.95 (d, J=8.2 Hz, 1H), 6.60 (s, 1H), 6.35 (d, J=8.3 Hz, 1H), 3.68 (s, 4H), 3.04 (s, 4H), 1.98 (t, J=5.6 Hz, 4H). UPLC/MS (method A): Rt 0.89 min. MS (ES) C₁₄H₁₇N₃O₂ requires 259, found 260 [M+H]⁺.

### 2-(2-Oxo-3H-1,3-benzoxazol-6-yl)-N-(4-phenylbutyl)-2,7-diazaspiro[3.5]nonane-7-carboxamide

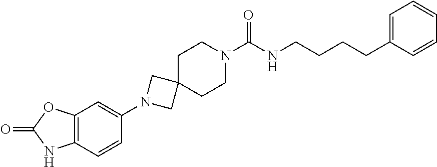 [SMILES: O=C(NCCCCc1ccccc1)N1CCC2(CC1)CN(c1ccc3[nH]c(=O)oc3c1)C2]

Following general procedure D (Method A), XXXIV″b (0.036 g, 0.139 mmol) and 4-phenylbutyl isocyanate (0.027 g, 0.152 mmol) afforded the title compound as a pinkish solid (0.025 g, 41%). ¹H NMR (400 MHz, DMSO-d₆) δ 11.18 (bs, 1H), 7.32-7.24 (m, 2H), 7.23-7.13 (m, 3H), 6.88 (d, J=8.3 Hz, 1H), 6.45 (t, J=5.5 Hz, 1H), 6.41 (d, J=2.1 Hz, 1H), 6.17 (dd, 1=8.4, 2.1 Hz, 1H), 3.52 (s, 4H), 3.29-3.20 (m, 4H), 3.04 (q, J=6.8 Hz, 2H), 2.58 (t, J=7.6 Hz, 2H), 1.72-1.61 (m, 4H), 1.61-1.47 (m, 2H), 1.47-1.34 (m, 2H). UPLC/MS (method A): Rt 2.06 min. MS (ES) C₂₅H₃₀N₄O₃ requires 434, found 435 [M+H]⁺.

### Example 70: 2-(3-Methyl-2-oxo-1,3-benzoxazol-6-yl)-N-(4-phenylbutyl)-2,7-diazaspiro[3.5]nonane-7-carboxamide

### tert-Butyl 2-(3-methyl-2-oxo-1,3-benzoxazol-6-yl)-2,7-diazaspiro[3.5]nonane-7-carboxylate (XXXV″b)

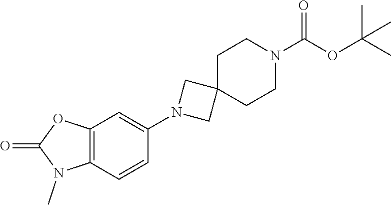 [SMILES: Cn1c(=O)oc2cc(N3CC4(CCN(C(=O)OC(C)(C)C)CC4)C3)ccc21]

Following general procedure C, XXXIII″b (0.047 g, 0.131 mmol) and CH₃I (0.020 g, 0.144 mmol) afforded XXXV″b as a pink powder (0.037 g, 75%). ¹H NMR (400 MHz, CDCl₃) δ 6.81 (d, J=8.4 Hz, 1H), 6.41 (d, J=2.1 Hz, 1H), 6.29 (d, 0.1=8.3 Hz, 1H), 3.64 (s, 4H), 3.50-3.40 (m, 4H), 3.37 (s, 3H), 1.88-1.75 (m, 4H), 1.49 (s, 9H). UPLC/MS (method A): Rt 2.31 min. MS (ES) C₂₀H₂₇N₃O₄ requires 373, found 374 [M+H]⁺.

### 6-(2,7-Diazaspiro[3.5]nonan-2-yl)-3-methyl-1,3-benzoxazol-2-one hydrochloride (XXXVI′b)

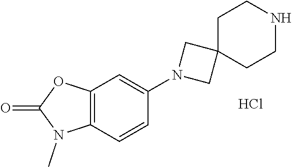 [SMILES: Cn1c(=O)oc2cc(N3CC4(CCNCC4)C3)ccc21 ; Cl]

Following general procedure C, XXXV″b (0.035 g, 0.094 mmol) afforded XXXVI′b as a white solid (0.035 g, quantitative). ¹H NMR (400 MHz, DMSO-d₆) δ 8.94 (bs, 2H), 7.10 (d, J=8.4 Hz, 1H), 6.59 (d, J=2.1 Hz, 1H), 6.37 (d, J=8.4 Hz, 1H), 3.66 (s, 4H), 3.29 (s, 3H), 3.04 (s, 4H), 1.97 (t, J=5.7 Hz, 4H). UPLC/MS (method A): Rt 1.08 min. MS (ES) C₁₅H₁₉N₃O₂ requires 273, found 274 [M+H]⁺.

### 2-(3-Methyl-2-oxo-1,3-benzoxazol-6-yl)-N-(4-phenylbutyl)-2,7-diazaspiro[3.5]nonane-7-carboxamide

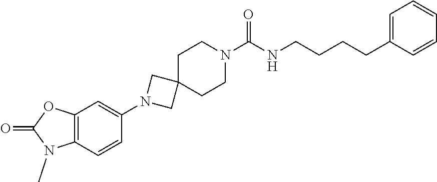 [SMILES: Cn1c(=O)oc2cc(N3CC4(CCN(C(=O)NCCCCc5ccccc5)CC4)C3)ccc21]

Following general procedure D (Method A), XXXVI″b (0.030 g, 0.097 mmol) and 4-phenylbutyl isocyanate (0.019 g, 0.107 mmol) afforded the title compound as a yellowish solid (32 mg, 70%). ¹H NMR (400 MHz, DMSO-d₆) δ 7.28 (t, J=7.5 Hz, 2H), 7.25-7.11 (m, 3H), 7.05 (d, J=8.4 Hz, 1H), 6.53-6.41 (m, 2H), 6.25 (dd, J=8.4, 2.1 Hz, 1H), 3.54 (s, 4H), 3.31-3.22 (m, 9H), 3.04 (q, J=6.6 Hz, 2H), 2.58 (t, J=7.6 Hz, 2H), 1.64 (t, J=5.5 Hz, 4H), 1.61-1.49 (m, 2H), 1.48-1.34 (m, 2H). UPLC/MS (method A): Rt 2.19 min. MS (ES) C₂₆H₃₂N₄O₃ requires 448, found 449 [M+H]⁺.

### Example 71: 9-(3-Methyl-2-oxo-1,3-benzoxazol-6-yl)-N-(4-phenylbutyl)-3,9-diazaspiro[5.5]undecane-3-carboxamide

### tert-Butyl 9-(3-hydroxy-4-nitrophenyl)-3,9-diazaspiro[5.5]undecane-3-carboxylate (XXXVII″c)

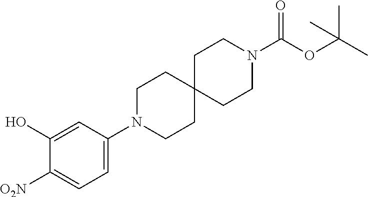 [SMILES: CC(C)(C)OC(=O)N1CCC2(CC1)CCN(c1ccc([N+](=O)[O-])c(O)c1)CC2]

Following general procedure F, XXX″c (0.40 g, 1.57 mmol) and 5-fluoro-2-nitrophenol (0.37 g, 2.36 mmol) afforded XXXVII″c as an orange solid (0.595 g, 97%). ¹H NMR (400 MHz, CDCl₃) δ 11.28 (bs, 1H), 7.93 (d, J=9.7 Hz, 1H), 6.41 (dd, J=9.7, 2.7 Hz, 1H), 6.28 (d, J=2.7 Hz, 1H), 3.51-3.33 (m, 8H), 1.66-1.61 (m, 4H), 1.50 (t, J=5.9 Hz, 4H), 1.46 (s, 9H). UPLC/MS (method A): Rt 2.62 min. MS (ES) C₂₀H₂₉N₃O₅ requires 391, found 392 [M+H]⁺.

### tert-Butyl 9-(4-amino-3-hydroxyphenyl)-3,9-diazaspiro[5.5]undecane-3-carboxylate (XXXII″c)

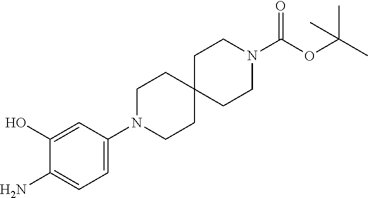 [SMILES: CC(C)(C)OC(=O)N1CCC2(CC1)CCN(c1ccc(N)c(O)c1)CC2]

Following general procedure B (Method C), XXXI″c (0.250 g, 0.64 mmol) afforded XXXII″c which was used in the next step without further purification. UPLC/MS (method A): Rt 1.70 min. MS (ES) C₂₀H₃₁N₃O₃ requires 361, found 362 [M+H]⁺.

### tert-Butyl 9-(2-oxo-3H-1,3-benzoxazol-6-yl)-3,9-diazaspiro[5.5]undecane-3-carboxylate (XXXIII′c)

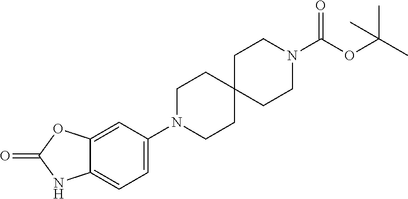 [SMILES: CC(C)(C)OC(=O)N1CCC2(CC1)CCN(c1ccc3[nH]c(=O)oc3c1)CC2]

Following general procedure B, XXXII″c (0.23 g, 0.64 mmol) and CDI (0.52 g, 3.2 mmol) afforded XXXIII″c as a pink solid (0.106 g, 43%). ¹H NMR (400 MHz, CDCl₃) δ 8.08 (bs, 1H), 6.91 (d, J=8.5 Hz, 1H), 6.85 (d, J=2.2 Hz, 1H), 6.74 (dd, J=8.6, 2.3 Hz, 1H), 3.48-3.36 (m, 4H), 3.14-3.07 (m, 4H), 1.70-1.63 (m, 4H), 1.58-1.54 (m, 2H), 1.52-1.47 (m, 2H), 1.46 (s, 9H). UPLC/MS (method A): Rt 2.19 min. MS (ES) C₂₁H₂₉N₃O₄ requires 387, found 388 [M+H]⁺.

### tert-Butyl 9-(3-methyl-2-oxo-1,3-benzoxazol-6-yl)-3,9-diazaspiro[5.5]undecane-3-carboxylate (XXXV′c)

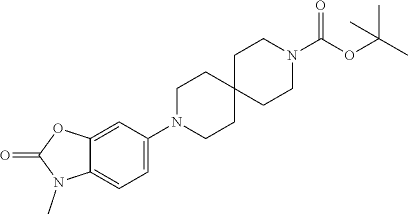 [SMILES: Cn1c(=O)oc2cc(N3CCC4(CCN(C(=O)OC(C)(C)C)CC4)CC3)ccc21]

Following general procedure C, XXXIII″c (0.050 g, 0.13 mmol) and CH₃I (0.042 g, 0.018 mL, 0.26 mmol) afforded XXXV′c which was used in the next step without further purification. UPLC/MS (method A): Rt 2.39 min. MS (ES) C₂₂H₃₁N₃O₄ requires 401, found 402 [M+H]⁺.

### 6-(3,9-Diazaspiro[5.5]undecan-3-yl)-3-methyl-1,3-benzoxazol-2-one hydrochloride (XXXVI″c)

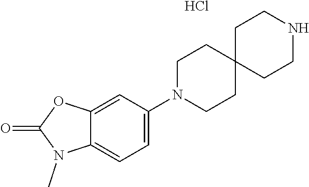 [SMILES: Cl ; Cn1c(=O)oc2cc(N3CCC4(CCNCC4)CC3)ccc21]

Following general procedure C, XXXVI″c (0.042 g, 0.10 mmol) afforded XXXVI″c which was used in the next step without further purification. UPLC/MS (method A): Rt 1.18 min MS (ES) C₁₇H₂₃N₃O₂ requires 301, found 302 [M+H]⁺.

### 9-(3-Methyl-2-oxo-1,3-benzoxazol-6-yl)-N-(4-phenylbutyl)-3,9-diazaspiro[5.5]undecane-3-carboxamide

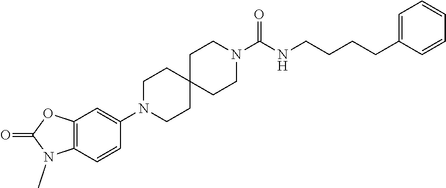 [SMILES: Cn1c(=O)oc2cc(N3CCC4(CCN(C(=O)NCCCCc5ccccc5)CC4)CC3)ccc21]

Following general procedure D (Method A), XXXVI″c (0.034 g, 0.10 mmol) and 4-phenylbutyl isocyanate (0.052 g, 0.30 mmol) afforded the title compound as a white solid (0.016 g, 34%). ¹H NMR (400 MHz, CDCl₃) δ 7.31-7.27 (m, 2H), 7.21-7.14 (m, 3H), 6.89-6.75 (m, 3H), 4.37 (t, J=5.4 Hz, 1H), 3.36 (s, 3H), 3.35-3.31 (m, 4H), 3.26 (q, J=6.6 Hz, 2H), 3.14-3.08 (m, 4H), 2.64 (t, J=7.5 Hz, 2H), 1.71-1.64 (m, 6H), 1.58-1.50 (m, 15H). UPLC/MS (method A): Rt 2.31 min. MS (ES) C₂₈H₃₆N₄O₃ requires 476, found 477 [M+H]⁺.

### Biological Activity Evaluation

The ability of exemplary compounds to inhibit acid ceramidase was measured. Experimental procedures and results are provided below.

### Part I: Assay Procedure

Cell lysates overexpressing acid ceramidase were used as the enzyme source for compound potency determination in a biochemical fluorescent assay. Briefly, compounds were preincubated with 10 μg protein of cell lysates in a dose-response manner for 1 hr at RT in the assay buffer containing 25 mM NaAC and 100 mM NaCl, pH 4.5. The reaction was initiated by the addition of substrate Rbm14-12 at a final concentration of 6.3 μM. The reaction was run at RT for 1 hr before it was stopped by the addition of the stopping buffer containing 20% methanol (v/v), 1 mg/ml NaIO₄, 0.1 M glycine, pH 10.6. The samples were incubated with the stopping buffer at RT for 1 hr to allow the fluorescent product to be formed. Finally the plate was read with SpectraMax i3 plate reader (Molecular Devices) at ex360 nm and em446 nm. Data were collected and used to determine the IC₅₀ values of compounds by curve fitting to the four-parameter inhibition equation.

### Part II: Results

Human acid ceramidse (hACR) inhibition values for tested compounds are provided in Table 1 below, along with c Log P, and compound solubility in water. The symbol “A” indicates inhibition of less than 0.2 μM; the symbol “B” indicates inhibition in the range of 0.2 μM up to 1 μM; and the symbol “C” indicates inhibition of greater than 1 μM.

TABLE 1

| | | | | | Solubility | |
|---|---|---|---|---|---|---|
| | Compound | Molecular | | | in H₂O | hACR |
| Example | Structure | Formula | MW | cLogP | (μM) | IC₅₀ |
| 1 | 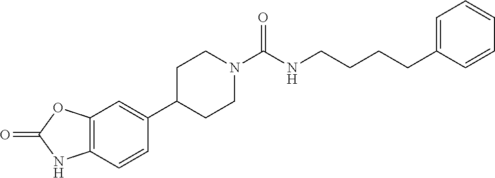 [SMILES: O=C(NCCCCc1ccccc1)N1CCC(c2ccc3[nH]c(=O)oc3c2)CC1] | C₂₃H₂₇N₃O₃ | 393.48 | 4.1 | 2.4 | B |
| 2 | 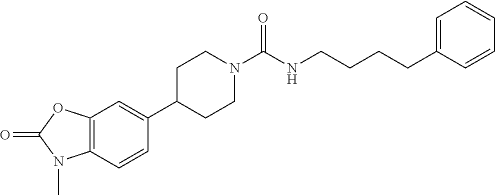 [SMILES: Cn1c(=O)oc2cc(C3CCN(C(=O)NCCCCc4ccccc4)CC3)ccc21] | C₂₄H₂₉N₃O₃ | 407.50 | 4.3 | N/A | C |
| 3 | 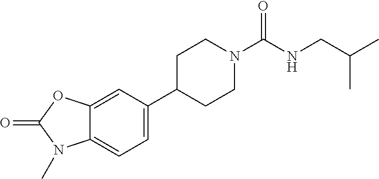 [SMILES: CC(C)CNC(=O)N1CCC(c2ccc3c(c2)oc(=O)n3C)CC1] | C₁₈H₂₅N₃O₃ | 331.41 | 2.7 | 126 | C |
| 4 | 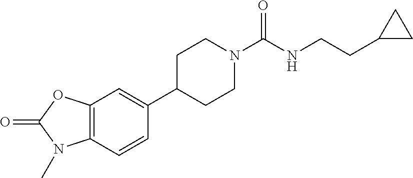 [SMILES: Cn1c(=O)oc2cc(C3CCN(C(=O)NCCC4CC4)CC3)ccc21] | C₁₉H₂₅N₃O₃ | 343.42 | 2.7 | N/A | C |
| 5 | 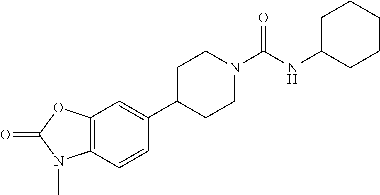 [SMILES: Cn1c(=O)oc2cc(C3CCN(C(=O)NC4CCCCC4)CC3)ccc21] | C₂₀H₂₇N₃O₃ | 357.45 | 3.4 | N/A | C |
| 6 | 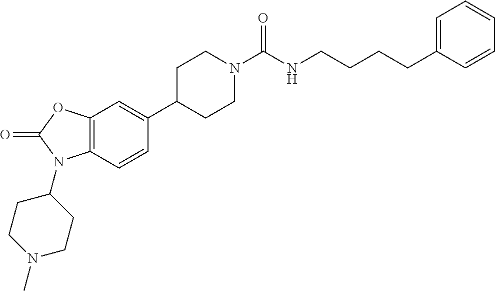 [SMILES: CN1CCC(n2c(=O)oc3cc(C4CCN(C(=O)NCCCCc5ccccc5)CC4)ccc32)CC1] | C₂₉H₃₈N₄O₃ | 490.64 | 4.4 | 10 | C |
| 7 | 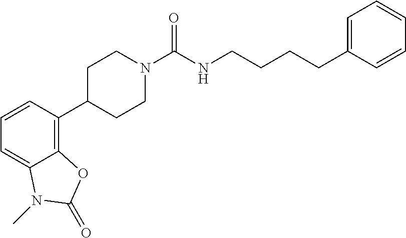 [SMILES: Cn1c(=O)oc2c(C3CCN(C(=O)NCCCCc4ccccc4)CC3)cccc21] | C₂₄H₂₉N₃O₃ | 407.50 | 4.3 | 13 | C |
| 8 | 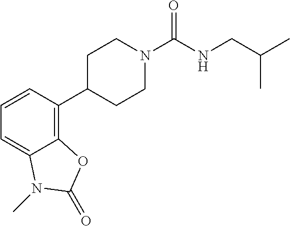 [SMILES: CC(C)CNC(=O)N1CCC(c2cccc3c2oc(=O)n3C)CC1] | C₁₈H₂₅N₃O₃ | 331.41 | 2.7 | 143 | C |
| 9 | 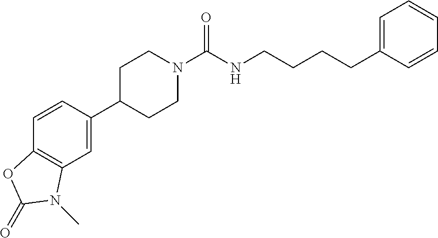 [SMILES: Cn1c(=O)oc2ccc(C3CCN(C(=O)NCCCCc4ccccc4)CC3)cc21] | C₂₄H₂₉N₃O₃ | 407.50 | 4.3 | 15 | C |
| 10 | 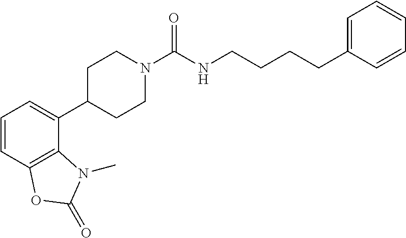 [SMILES: Cn1c(=O)oc2cccc(C3CCN(C(=O)NCCCCc4ccccc4)CC3)c21] | C₂₄H₂₉N₃O₃ | 407.50 | 4.3 | 9 | C |
| 11 | 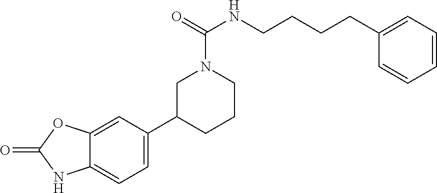 [SMILES: O=C(NCCCCc1ccccc1)N1CCCC(c2ccc3[nH]c(=O)oc3c2)C1] | C₂₃H₂₇N₃O₃ | 393.48 | 4.2 | N/A | C |
| 12 | 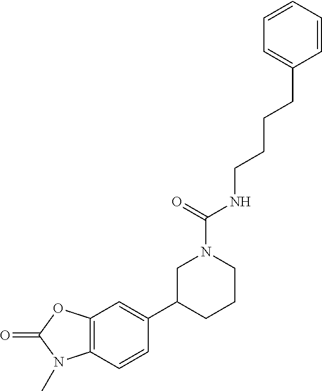 [SMILES: Cn1c(=O)oc2cc(C3CCCN(C(=O)NCCCCc4ccccc4)C3)ccc21] | C₂₄H₂₉N₃O₃ | 407.50 | 4.4 | 8 | C |
| 13 | 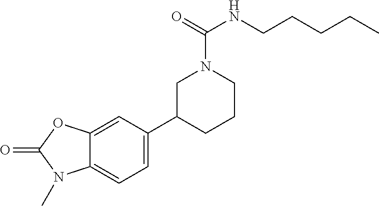 [SMILES: CCCCCNC(=O)N1CCCC(c2ccc3c(c2)oc(=O)n3C)C1] | C₁₉H₂₇N₃O₃ | 345.43 | 3.3 | N/A | C |
| 14 | 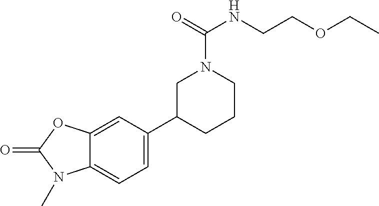 [SMILES: CCOCCNC(=O)N1CCCC(c2ccc3c(c2)oc(=O)n3C)C1] | C₁₈H₂₅N₃O₄ | 347.41 | 1.8 | N/A | C |
| 15 | 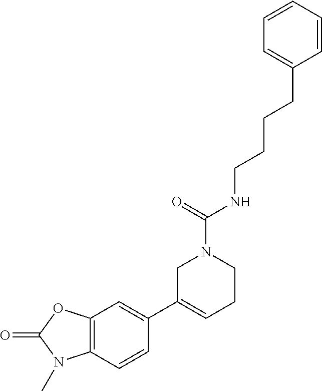 [SMILES: Cn1c(=O)oc2cc(C3=CCCN(C(=O)NCCCCc4ccccc4)C3)ccc21] | C₂₄H₂₇N₃O₃ | 405.49 | 4.2 | N/A | B |
| 16 | 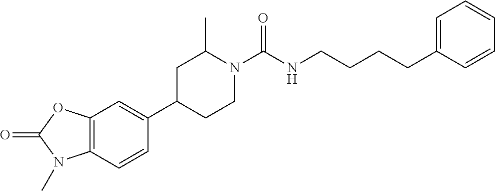 [SMILES: CC1CC(c2ccc3c(c2)oc(=O)n3C)CCN1C(=O)NCCCCc1ccccc1] | C₂₅H₃₁N₃O₃ | 421.53 | 4.7 | 2.2 | A |
| 17 | 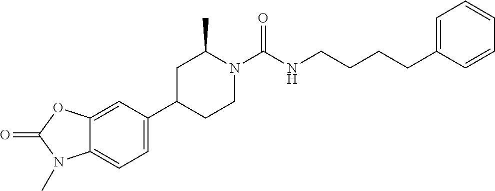 [SMILES: C[C@@H]1CC(c2ccc3c(c2)oc(=O)n3C)CCN1C(=O)NCCCCc1ccccc1] | C₂₅H₃₁N₃O₃ | 421.53 | 4.7 | N/A | A |
| 18 | 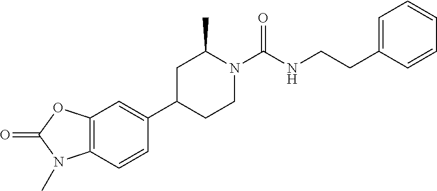 [SMILES: C[C@@H]1CC(c2ccc3c(c2)oc(=O)n3C)CCN1C(=O)NCCc1ccccc1] | C₂₃H₂₇N₃O₃ | 393.48 | 3.8 | N/A | B |
| 19 | 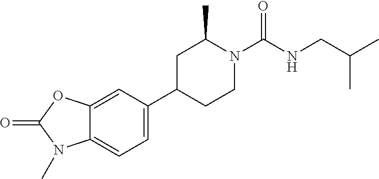 [SMILES: CC(C)CNC(=O)N1CCC(c2ccc3c(c2)oc(=O)n3C)C[C@H]1C] | C₁₉H₂₇N₃O₃ | 345.43 | 3.1 | N/A | C |
| 20 | 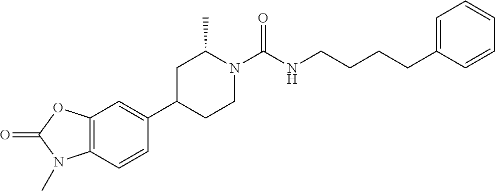 [SMILES: C[C@H]1CC(c2ccc3c(c2)oc(=O)n3C)CCN1C(=O)NCCCCc1ccccc1] | C₂₅H₃₁N₃O₃ | 421.53 | 4.7 | 10 | A |
| 21 | 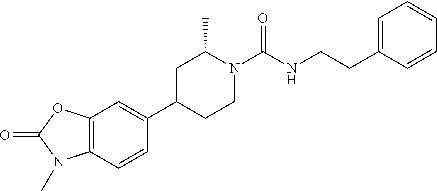 [SMILES: C[C@H]1CC(c2ccc3c(c2)oc(=O)n3C)CCN1C(=O)NCCc1ccccc1] | C₂₃H₂₇N₃O₃ | 393.48 | 3.8 | 56 | B |
| 22 | 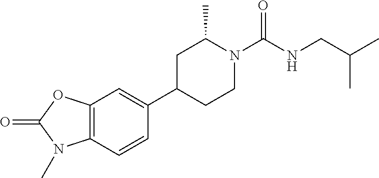 [SMILES: CC(C)CNC(=O)N1CCC(c2ccc3c(c2)oc(=O)n3C)C[C@@H]1C] | C₁₉H₂₇N₃O₃ | 345.43 | 3.1 | 235 | B |
| 23 | 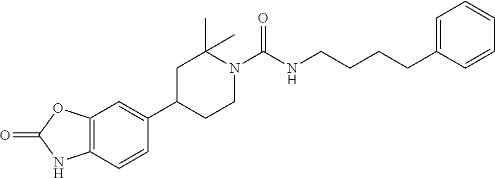 [SMILES: CC1(C)CC(c2ccc3[nH]c(=O)oc3c2)CCN1C(=O)NCCCCc1ccccc1] | C₂₅H₃₁N₃O₃ | 421.53 | 4.7 | N/A | A |
| 24 | 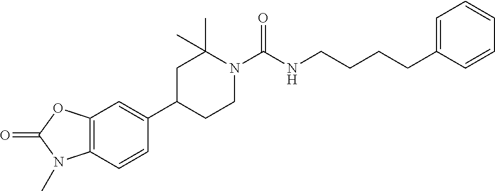 [SMILES: Cn1c(=O)oc2cc(C3CCN(C(=O)NCCCCc4ccccc4)C(C)(C)C3)ccc21] | C₂₆H₃₃N₃O₃ | 435.56 | 4.9 | 1.3 | A |
| 25 | 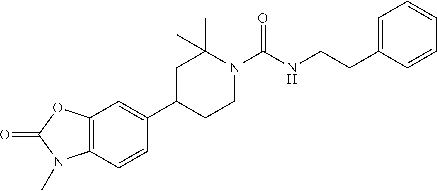 [SMILES: Cn1c(=O)oc2cc(C3CCN(C(=O)NCCc4ccccc4)C(C)(C)C3)ccc21] | C₂₄H₂₉N₃O₃ | 407.50 | 4.0 | 26 | A |
| 26 | 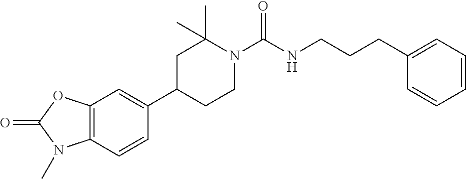 [SMILES: Cn1c(=O)oc2cc(C3CCN(C(=O)NCCCc4ccccc4)C(C)(C)C3)ccc21] | C₂₅H₃₁N₃O₃ | 421.53 | 4.4 | 5.8 | A |
| 27 | 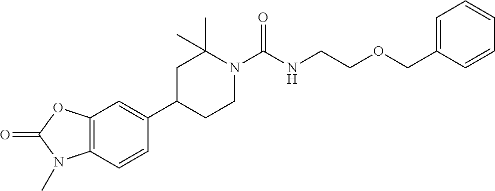 [SMILES: Cn1c(=O)oc2cc(C3CCN(C(=O)NCCOCc4ccccc4)C(C)(C)C3)ccc21] | C₂₅H₃₁N₃O₄ | 437.53 | 3.5 | 9.4 | A |
| 28 | 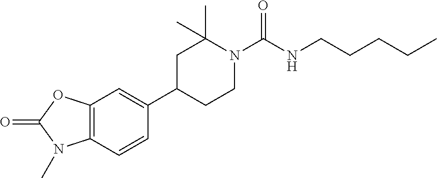 [SMILES: CCCCCNC(=O)N1CCC(c2ccc3c(c2)oc(=O)n3C)CC1(C)C] | C₂₁H₃₁N₃O₃ | 373.49 | 3.9 | 1.7 | A |
| 29 | 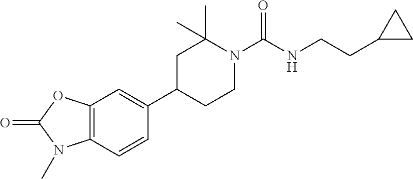 [SMILES: Cn1c(=O)oc2cc(C3CCN(C(=O)NCCC4CC4)C(C)(C)C3)ccc21] | C₂₁H₂₉N₃O₃ | 371.47 | 3.3 | 250 | B |
| 30 | 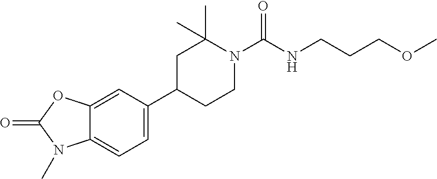 [SMILES: COCCCNC(=O)N1CCC(c2ccc3c(c2)oc(=O)n3C)CC1(C)C] | C₂₀H₂₉N₃O₄ | 375.46 | 2.0 | N/A | C |
| 31 | 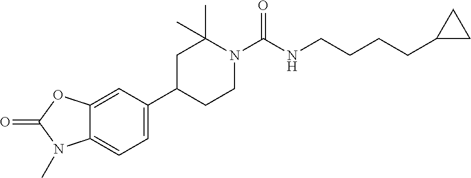 [SMILES: Cn1c(=O)oc2cc(C3CCN(C(=O)NCCCCC4CC4)C(C)(C)C3)ccc21] | C₂₃H₃₃N₃O₃ | 399.53 | 4.2 | 0.3 | A |
| 32 | 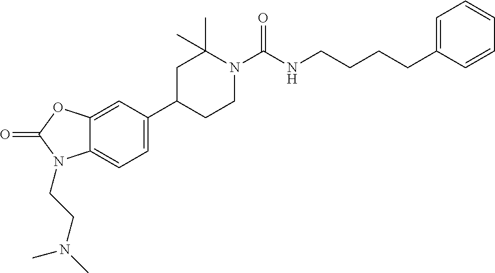 [SMILES: CN(C)CCn1c(=O)oc2cc(C3CCN(C(=O)NCCCCc4ccccc4)C(C)(C)C3)ccc21] | C₂₉H₄₀N₄O₃ | 492.65 | 5.0 | N/A | A |
| 33 | 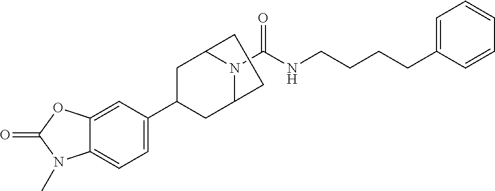 [SMILES: Cn1c(=O)oc2cc(C3CC4CCC(C3)N4C(=O)NCCCCc3ccccc3)ccc21] | C₂₆H₃₁N₃O₃ | 433.54 | 4.8 | 3.2 | A |
| 34 | 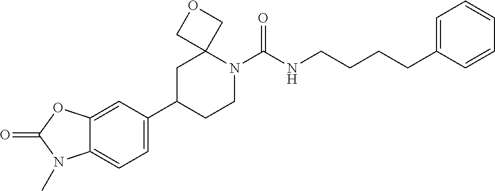 [SMILES: Cn1c(=O)oc2cc(C3CCN(C(=O)NCCCCc4ccccc4)C4(COC4)C3)ccc21] | C₂₆H₃₁N₃O₄ | 449.54 | 3.9 | N/A | C |
| 35 | 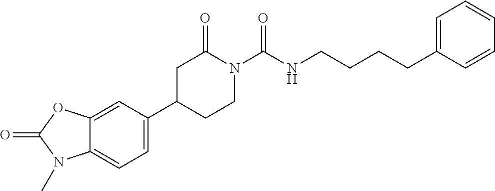 [SMILES: Cn1c(=O)oc2cc(C3CCN(C(=O)NCCCCc4ccccc4)C(=O)C3)ccc21] | C₂₄H₂₇N₃O₄ | 421.49 | 3.9 | <1 | C |
| 36 | 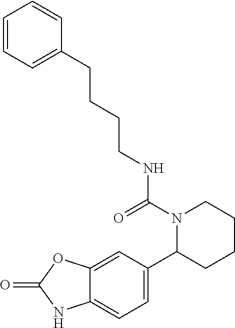 [SMILES: O=C(NCCCCc1ccccc1)N1CCCCC1c1ccc2[nH]c(=O)oc2c1] | C₂₃H₂₇N₃O₃ | 393.48 | 4.4 | N/A | C |
| 37 | 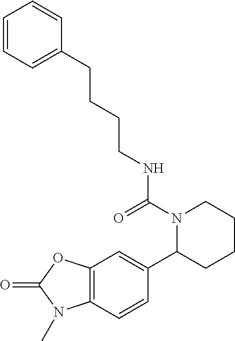 [SMILES: Cn1c(=O)oc2cc(C3CCCCN3C(=O)NCCCCc3ccccc3)ccc21] | C₂₄H₂₉N₃O₃ | 407.50 | 4.6 | 30 | C |
| 38 | 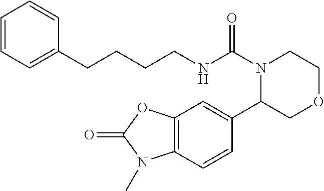 [SMILES: Cn1c(=O)oc2cc(C3COCCN3C(=O)NCCCCc3ccccc3)ccc21] | C₂₃H₂₇N₃O₄ | 409.48 | 3.4 | N/A | C |
| 39 | 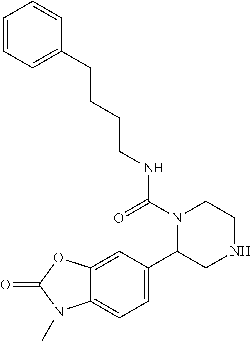 [SMILES: Cn1c(=O)oc2cc(C3CNCCN3C(=O)NCCCCc3ccccc3)ccc21] | C₂₃H₂₈N₄O₃ | 408.49 | 3.1 | 241 | C |
| 40 | 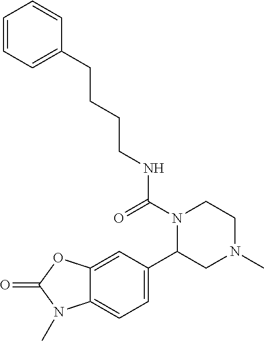 [SMILES: CN1CCN(C(=O)NCCCCc2ccccc2)C(c2ccc3c(c2)oc(=O)n3C)C1] | C₂₄H₃₀N₄O₃ | 422.52 | 3.7 | 224 | C |
| 41 | 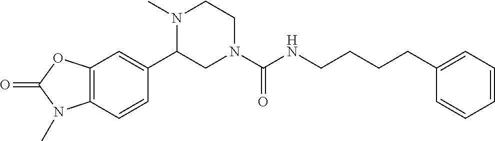 [SMILES: CN1CCN(C(=O)NCCCCc2ccccc2)CC1c1ccc2c(c1)oc(=O)n2C] | C₂₄H₃₀N₄O₃ | 422.52 | 3.7 | 135 | C |
| 42 | 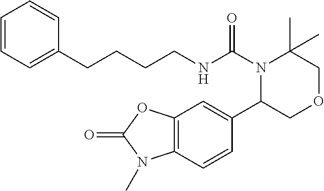 [SMILES: Cn1c(=O)oc2cc(C3COCC(C)(C)N3C(=O)NCCCCc3ccccc3)ccc21] | C₂₅H₃₁N₃O₄ | 437.53 | 4.0 | 14.8 | A |
| 43 | 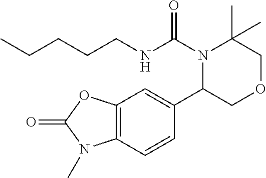 [SMILES: CCCCCNC(=O)N1C(c2ccc3c(c2)oc(=O)n3C)COCC1(C)C] | C₂₀H₂₉N₃O₄ | 375.46 | 3.0 | 69.1 | A |
| 44 | 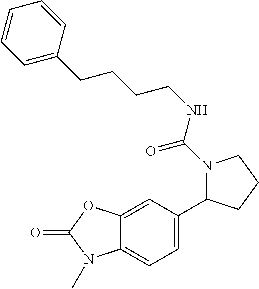 [SMILES: Cn1c(=O)oc2cc(C3CCCN3C(=O)NCCCCc3ccccc3)ccc21] | C₂₃H₂₇N₃O₃ | 393.48 | 4.2 | N/A | C |
| 45 | 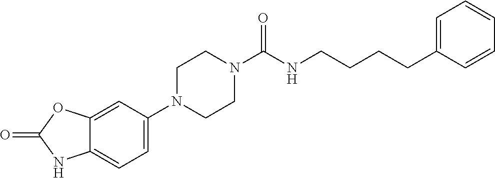 [SMILES: O=C(NCCCCc1ccccc1)N1CCN(c2ccc3[nH]c(=O)oc3c2)CC1] | C₂₂H₂₆N₄O₃ | 394.47 | 3.5 | N/A | C |
| 46 | 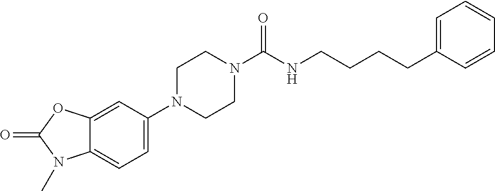 [SMILES: Cn1c(=O)oc2cc(N3CCN(C(=O)NCCCCc4ccccc4)CC3)ccc21] | C₂₃H₂₈N₄O₃ | 408.49 | 3.7 | 6 | B |
| 47 | 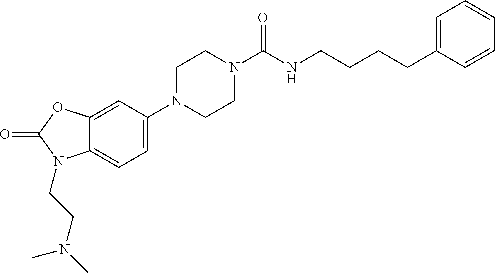 [SMILES: CN(C)CCn1c(=O)oc2cc(N3CCN(C(=O)NCCCCc4ccccc4)CC3)ccc21] | C₂₆H₃₅N₅O₃ | 465.59 | 3.9 | 250 | C |
| 48 | 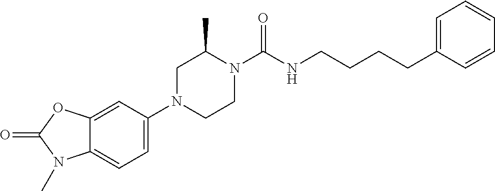 [SMILES: C[C@@H]1CN(c2ccc3c(c2)oc(=O)n3C)CCN1C(=O)NCCCCc1ccccc1] | C₂₄H₃₀N₄O₃ | 422.52 | 4.1 | 4.3 | B |
| 49 | 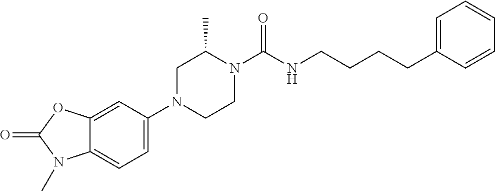 [SMILES: C[C@H]1CN(c2ccc3c(c2)oc(=O)n3C)CCN1C(=O)NCCCCc1ccccc1] | C₂₄H₃₀N₄O₃ | 422.52 | 4.1 | N/A | B |
| 50 | 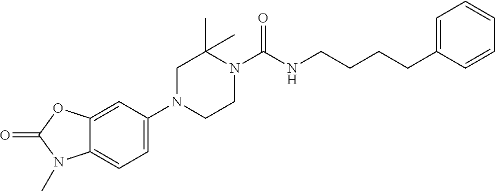 [SMILES: Cn1c(=O)oc2cc(N3CCN(C(=O)NCCCCc4ccccc4)C(C)(C)C3)ccc21] | C₂₅H₃₂N₄O₃ | 436.55 | 4.3 | 0.3 | A |
| 51 | 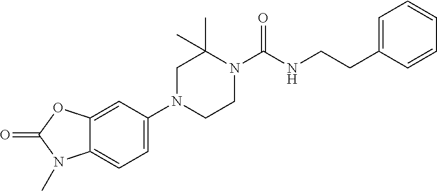 [SMILES: Cn1c(=O)oc2cc(N3CCN(C(=O)NCCc4ccccc4)C(C)(C)C3)ccc21] | C₂₃H₂₈N₄O₃ | 408.49 | 3.4 | 0.9 | B |
| 52 | 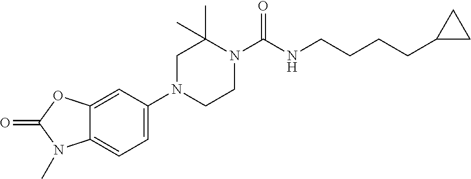 [SMILES: Cn1c(=O)oc2cc(N3CCN(C(=O)NCCCCC4CC4)C(C)(C)C3)ccc21] | C₂₂H₃₂N₄O₃ | 400.51 | 3.6 | 0.9 | A |
| 53 | 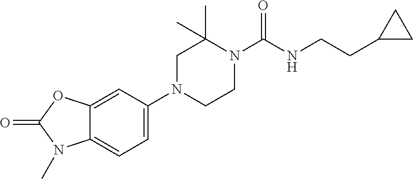 [SMILES: Cn1c(=O)oc2cc(N3CCN(C(=O)NCCC4CC4)C(C)(C)C3)ccc21] | C₂₀H₂₈N₄O₃ | 372.46 | 2.7 | 20.5 | C |
| 54 | 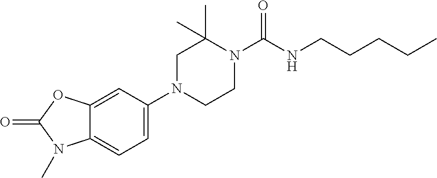 [SMILES: CCCCCNC(=O)N1CCN(c2ccc3c(c2)oc(=O)n3C)CC1(C)C] | C₂₀H₃₀N₄O₃ | 374.48 | 3.3 | 2.4 | B |
| 55 | 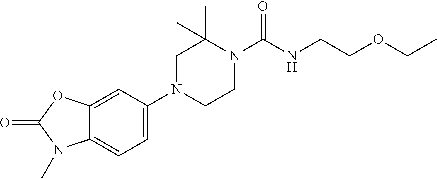 [SMILES: CCOCCNC(=O)N1CCN(c2ccc3c(c2)oc(=O)n3C)CC1(C)C] | C₁₉H₂₈N₄O₄ | 376.45 | 1.7 | N/A | C |
| 56 | 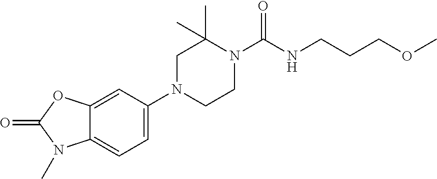 [SMILES: COCCCNC(=O)N1CCN(c2ccc3c(c2)oc(=O)n3C)CC1(C)C] | C₁₉H₂₈N₄O₄ | 376.45 | 1.4 | N/A | C |
| 57 | 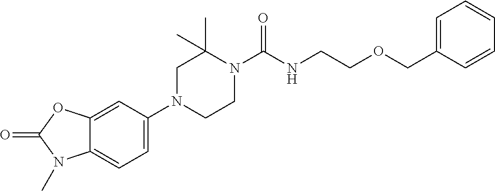 [SMILES: Cn1c(=O)oc2cc(N3CCN(C(=O)NCCOCc4ccccc4)C(C)(C)C3)ccc21] | C₂₄H₃₀N₄O₄ | 438.52 | 3.0 | 3.5 | B |
| 58 | 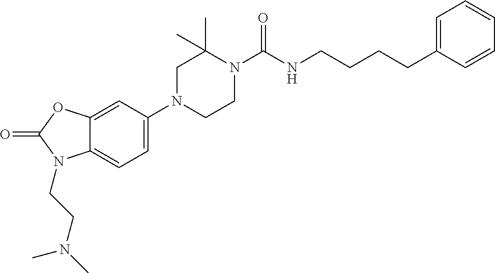 [SMILES: CN(C)CCn1c(=O)oc2cc(N3CCN(C(=O)NCCCCc4ccccc4)C(C)(C)C3)ccc21] | C₂₈H₃₉N₅O₃ | 493.64 | 4.5 | 41.8 | A |
| 59 | 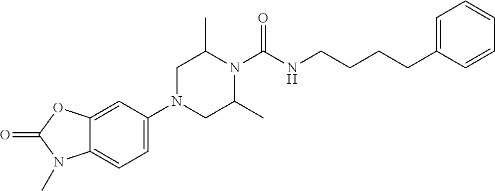 [SMILES: CC1CN(c2ccc3c(c2)oc(=O)n3C)CC(C)N1C(=O)NCCCCc1ccccc1] | C₂₅H₃₂N₄O₃ | 436.55 | 4.5 | 0.5 | A |
| 60 | 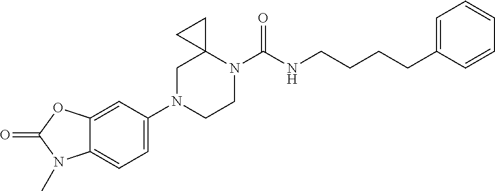 [SMILES: Cn1c(=O)oc2cc(N3CCN(C(=O)NCCCCc4ccccc4)C4(CC4)C3)ccc21] | C₂₅H₃₀N₄O₃ | 434.53 | 4.1 | N/A | C |
| 61 | 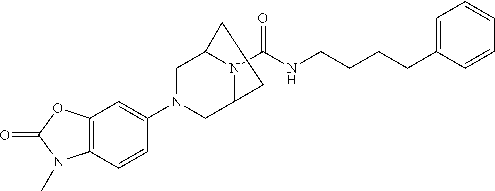 [SMILES: Cn1c(=O)oc2cc(N3CC4CCC(C3)N4C(=O)NCCCCc3ccccc3)ccc21] | C₂₅H₃₀N₄O₃ | 434.53 | 4.3 | N/A | C |
| 62 | 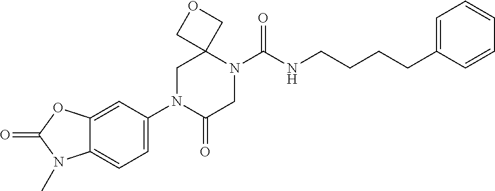 [SMILES: Cn1c(=O)oc2cc(N3CC4(COC4)N(C(=O)NCCCCc4ccccc4)CC3=O)ccc21] | C₂₅H₂₈N₄O₅ | 464.51 | 2.3 | N/A | B |
| 63 | 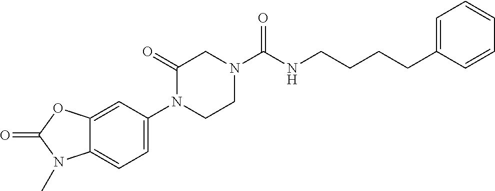 [SMILES: Cn1c(=O)oc2cc(N3CCN(C(=O)NCCCCc4ccccc4)CC3=O)ccc21] | C₂₃H₂₆N₄O₄ | 422.48 | 2.8 | 51.35 | C |
| 64 | 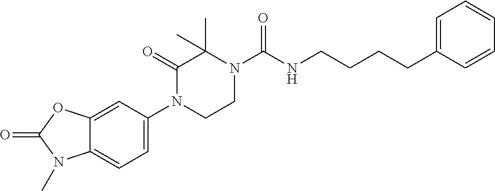 [SMILES: Cn1c(=O)oc2cc(N3CCN(C(=O)NCCCCc4ccccc4)C(C)(C)C3=O)ccc21] | C₂₅H₃₀N₄O₄ | 450.53 | 3.6 | 6.8 | A |
| 65 | 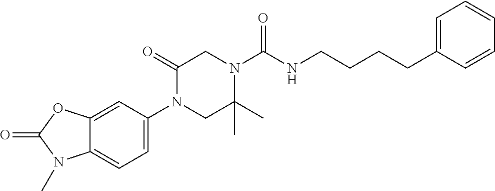 [SMILES: Cn1c(=O)oc2cc(N3CC(C)(C)N(C(=O)NCCCCc4ccccc4)CC3=O)ccc21] | C₂₅H₃₀N₄O₄ | 450.53 | 3.4 | 18.9 | A |
| 66 | 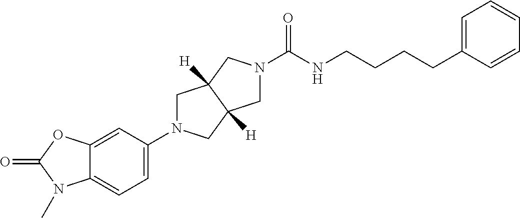 [SMILES: Cn1c(=O)oc2cc(N3C[C@H]4CN(C(=O)NCCCCc5ccccc5)C[C@H]4C3)ccc21] | C₂₅H₃₀N₄O₃ | 434.53 | 3.5 | N/A | A |
| 67 | 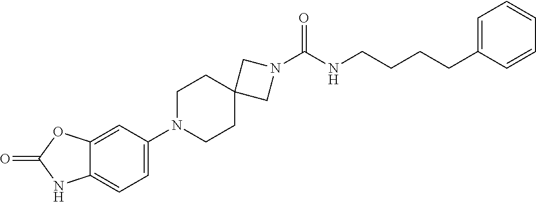 [SMILES: O=C(NCCCCc1ccccc1)N1CC2(CCN(c3ccc4[nH]c(=O)oc4c3)CC2)C1] | C₂₅H₃₀N₄O₃ | 434.53 | 3.8 | N/A | C |
| 68 | 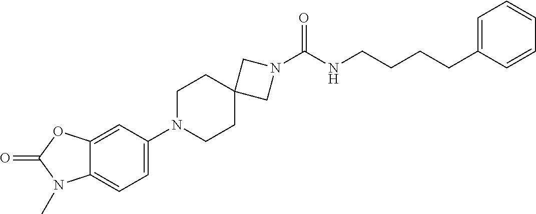 [SMILES: Cn1c(=O)oc2cc(N3CCC4(CC3)CN(C(=O)NCCCCc3ccccc3)C4)ccc21] | C₂₆H₃₂N₄O₃ | 448.56 | 4.0 | N/A | C |
| 69 | 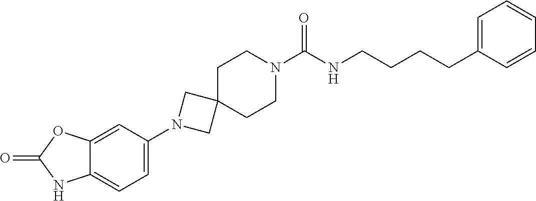 [SMILES: O=C(NCCCCc1ccccc1)N1CCC2(CC1)CN(c1ccc3[nH]c(=O)oc3c1)C2] | C₂₅H₃₀N₄O₃ | 434.53 | 3.8 | N/A | C |
| 70 | 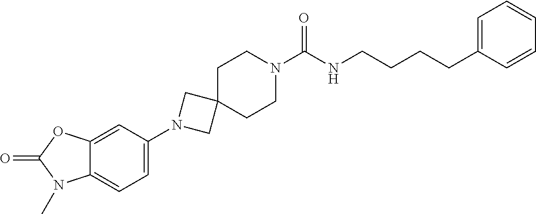 [SMILES: Cn1c(=O)oc2cc(N3CC4(CCN(C(=O)NCCCCc5ccccc5)CC4)C3)ccc21] | C₂₆H₃₂N₄O₃ | 448.56 | 4.0 | N/A | C |
| 71 | 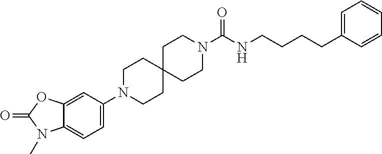 [SMILES: Cn1c(=O)oc2cc(N3CCC4(CCN(C(=O)NCCCCc5ccccc5)CC4)CC3)ccc21] | C₂₈H₃₆N₄O₃ | 476.61 | 4.7 | N/A | C |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound of formula (I): 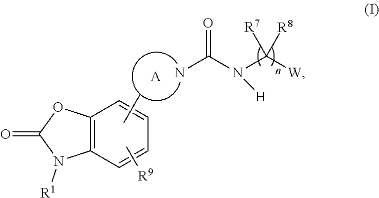
or a pharmaceutically acceptable salt thereof, wherein: 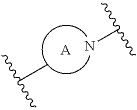
is a monocyclic or bicyclic (e.g., fused, spiro, bridged) heterocyclylene containing at least one N (including the depicted nitrogen) that is optionally substituted (e.g., with one or more substituents each independently selected from C₁₋₆alkyl and oxo);
R¹ is selected from the group consisting of hydrogen, C₁₋₆alkyl, C₁₋₆alkylene-NR^{a}₂, C₁₋₆alkylene-OR^{c}, 3-7 membered heterocyclyl, phenyl, C₃₋₇cycloalkyl, and 5-6 membered heteroaryl;
R⁷ and R⁸ are independently, for each occurrence, selected from the group consisting of hydrogen, C₁₋₆alkyl, C₁₋₆haloalkyl, and halogen; or R⁷ and R⁸ can be taken together to form C₃₋₇cycloalkylene;
R⁹ is selected from the group consisting of hydrogen, C₁₋₆alkyl, and halogen;
R^{a} is hydrogen or C₁₋₆alkyl;
R^{c} is selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂; and
n is an integer selected from 0 to 6, wherein
when n is an integer selected from 1 to 6, W is selected from the group consisting of hydrogen, halogen, phenyl, 5-6 membered heteroaryl, C₃₋₇cycloalkyl, 3-7 membered heterocyclyl, O—(C₁₋₆alkyl), O—(C₁₋₆haloalkyl), —O-phenyl, and O—(C₁₋₄alkylene)-phenyl; and
when n is 0, W is selected from the group consisting of hydrogen, C₃₋₇cycloalkyl, 3-7 membered saturated heterocyclyl, O—(C₁₋₆alkyl), O—(C₁₋₆haloalkyl), —O-phenyl, and O—(C₁₋₆alkylene)-phenyl;
wherein any aforementioned 3-7 membered heterocyclyl and phenyl are optionally substituted, and wherein the compound is not a compound selected from the group consisting of: 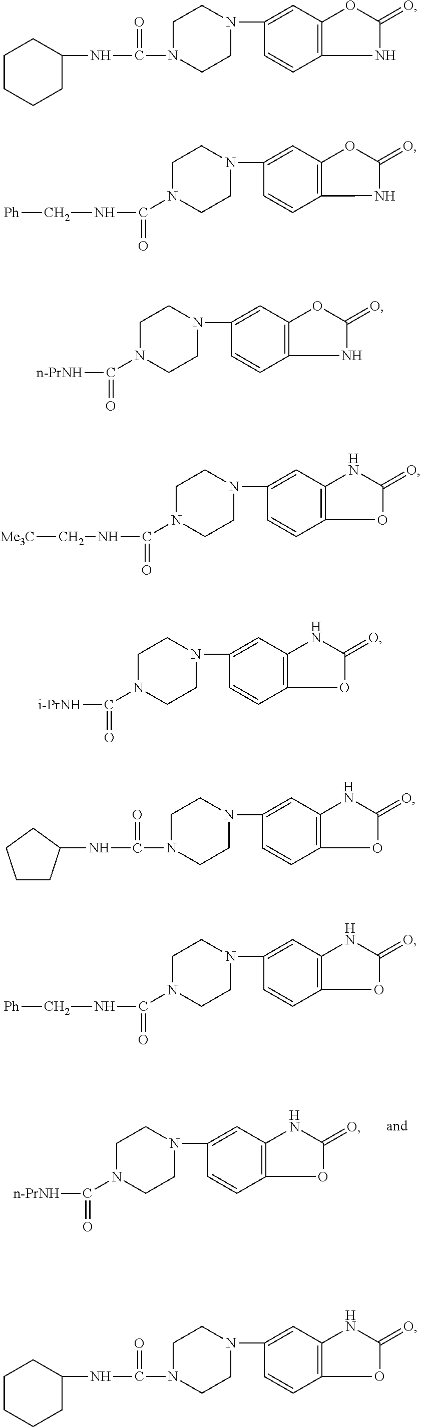
or a pharmaceutically acceptable salt thereof.

2. The compound of claim 1, wherein the compound is a compound of formula (I-a): 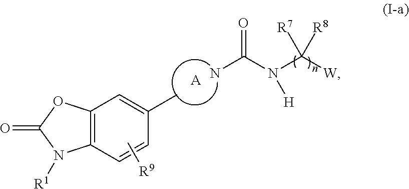
or a pharmaceutically acceptable salt thereof, wherein the variables are as defined in claim 1.

3. The compound of claim 1 or 2, wherein 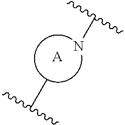
is a monocyclic heterocyclylene.

4. The compound of any one of claims 1-3, wherein 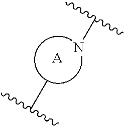
is selected from the group consisting of: 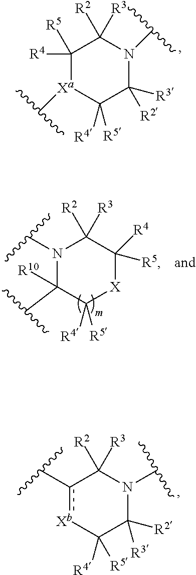
wherein
R², R³, R^{2′} and R^{3′} are independently selected from hydrogen or C₁₋₆alkyl, or
R² and R³ or R^{2′} and R^{3′} can be taken together to form C₃₋₇cycloalkylene, 3-7 membered heterocyclylene, or oxo; or
R⁴ and R⁵ are independently selected from hydrogen and C₁₋₆alkyl, or R⁴ and R⁵ can be taken together to form oxo;
R^{4′} and R^{5′} are independently selected from hydrogen and C₁₋₆alkyl, or R^{4′} and R^{5′} can be taken together to form oxo;
X is selected from the group consisting of CH₂, NR^{a}, and O;
X^{a} is selected from CH or N;
R¹⁰ is hydrogen or methyl;
R^{a} is hydrogen or C₁₋₆alkyl; [symbol]
indicates a single bond or double bond, and when a single bond, X^{b} is selected from the group consisting of CH₂, NR^{a}, and O, and when a double bond, X^{b} is CH; and
m is 0 or 1.

5. The compound of claim 4, wherein X^{a} is CH.

6. The compound of claim 4 or 5, wherein X^{b} is CH₂ or CH.

7. The compound of any one of claims 4-6, wherein X is CH₂.

8. The compound of any one of claims 4-6, wherein X is O.

9. The compound of any one of claims 4-8, wherein at least one of R² and R³ is methyl.

10. The compound of any one of claims 4-8, wherein at least one of R², R³, R^{2′} and R^{3′} is methyl.

11. The compound of any one of claims 4-8, wherein R² and R³ are methyl.

12. The compound of any one of claims 4-8, wherein R² and R³ are methyl, and R^{2′} and R^{3′} are hydrogen.

13. The compound of any one of claims 4-8, wherein R² and R³ are independently hydrogen or methyl.

14. The compound of any one of claims 4-8, wherein R², R³, and R^{2′} and R^{3′} are independently hydrogen or methyl.

15. The compound of any one of claims 4-8, wherein R² and R³ are taken together to form C₃₋₇cycloalkylene, 3-7 membered heterocyclylene, or oxo.

16. The compound of claim 15, wherein R² and R³ are taken together to form cyclopropylene, 4-membered heterocyclylene, or oxo.

17. The compound of any one of claims 4-8, wherein R² and R^{2′} are taken together to form a 5-7-membered heterocycle, and R³ and R^{3′} are hydrogen.

18. The compound of any one of claims 4-17, wherein R⁴ and R⁵ are hydrogen or taken together to form oxo.

19. The compound of any one of claims 4-17, wherein R⁴, R⁵, R^{4′} and R^{5′} are hydrogen.

20. The compound of any one of claims 4-17, wherein R² and R³ are methyl, and R^{2′}, R^{3′}, R⁴, R⁵, R^{4′} and R^{5′} are hydrogen.

21. The compound of any one of claims 4-20, wherein R² and R³ are methyl, and R⁴, R⁵, R^{4′} and R^{5′} are hydrogen.

22. The compound of any one of claims 4-20, wherein R² and R³ are methyl, R^{2′}, R^{3′}, R⁴, and R⁵ are hydrogen, and R^{4′} and R^{5′} are taken together to form oxo.

23. The compound of any one of claims 4-22, wherein m is 1.

24. The compound of any one of claims 4-23, wherein R¹⁰ is hydrogen.

25. The compound of claim 1, wherein the compound is a compound of formula (II): 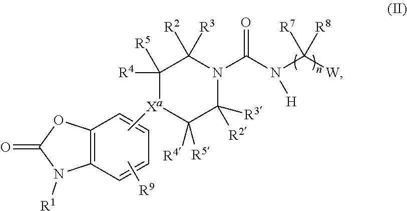
or a pharmaceutically acceptable salt thereof, wherein the variables are as defined in claim 1.

26. The compound of claim 1, wherein the compound is a compound of formula (II-a): 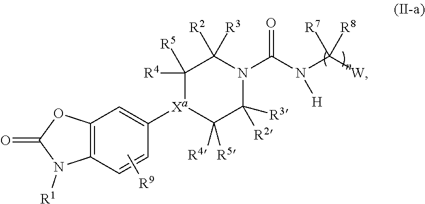
or a pharmaceutically acceptable salt thereof, wherein the variables are as defined in claim 1.

27. The compound of claim 1, wherein the compound is a compound of formula (III): 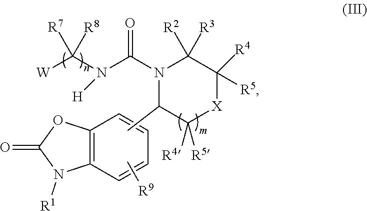
or a pharmaceutically acceptable salt thereof, wherein the variables are as defined in claim 1.

28. The compound of claim 1, wherein the compound is a compound of formula (III-a): 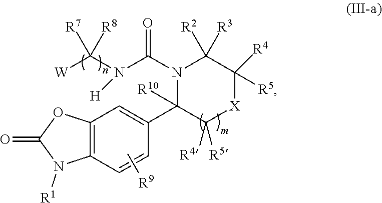
or a pharmaceutically acceptable salt thereof, wherein the variables are as defined in claim 1.

29. The compound of claim 1, wherein the compound is a compound of formula (IV): 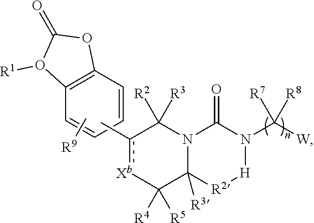
or a pharmaceutically acceptable salt thereof, wherein the variables are as defined in claim 1.

30. The compound of claim 1, wherein the compound is a compound of formula (IV-a): 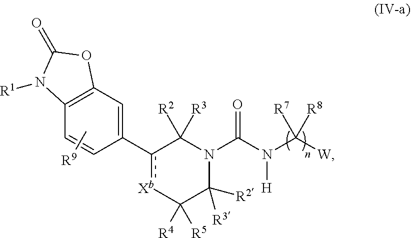
or formula (IV-b): 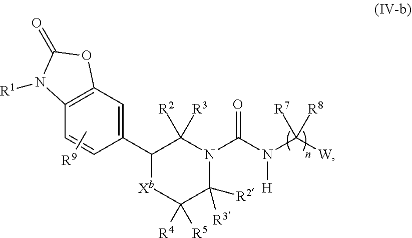
or a pharmaceutically acceptable salt thereof, wherein the variables are as defined in claim 1.

31. The compound of claim 1 or 2, wherein 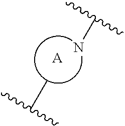
is a bicyclic heterocyclylene.

32. The compound of any one of claims 1, 2, and 31, wherein 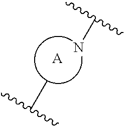
is a spiro-, fused-, or bridged-bicyclic heterocyclylene.

33. The compound of any one of claims 1, 2, 31, and 32, wherein 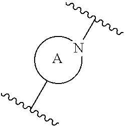
is selected from the group consisting of 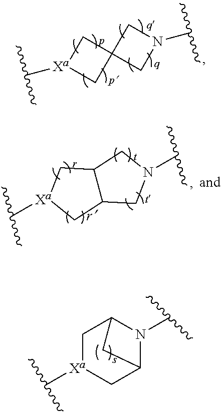
wherein X^{a} is selected from N or CH; p, p′, q, q′, r, r′, t, t′, and s are independently selected from 1 or 2.

34. The compound of claim 33, wherein 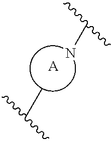
is selected from the group consisting of

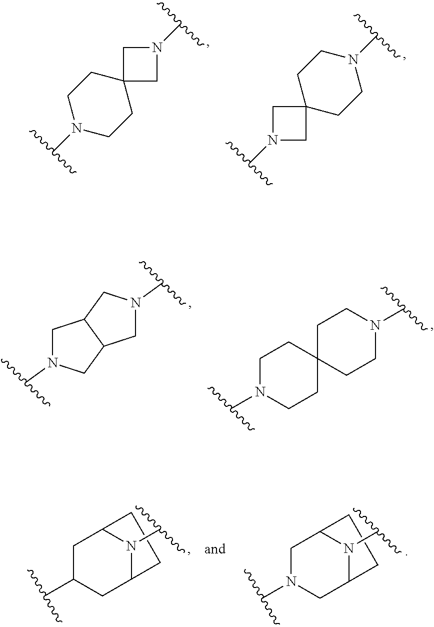 [SMILES: *N1CCC2(CC1)CN(*)C2 ; *N1CCC2(CC1)CN(*)C2 ; *N1CC2CN(*)CC2C1 ; *N1CCC2(CC1)CCN(*)CC2 ; *C1CC2CCC(C1)N2* ; *N1CC2CCC(C1)N2*]

35. The compound of claim 33 or 34, wherein 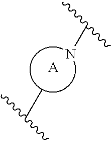
is

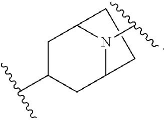 [SMILES: *C1CC2CCC(C1)N2*]

36. The compound of claim 1, wherein the compound is a compound of formula (I-b): 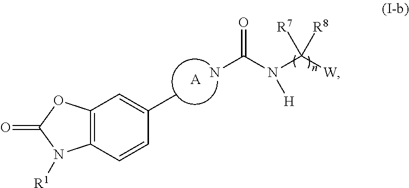
or a compound of formula (I-c): 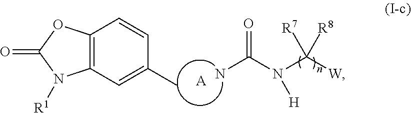
or a pharmaceutically acceptable salt thereof, wherein: 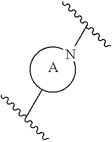
is a monocyclic or bicyclic (e.g., fused, spiro, bridged) saturated heterocyclylene containing at least one N (including the depicted nitrogen) that is optionally substituted (e.g., with one or more substituents each independently selected from C₁₋₆alkyl and oxo); wherein,
R₁ is selected from the group consisting of hydrogen, C₁₋₆alkyl, C₁₋₆alkylene-NR^{a}₂, C₁₋₆alkylene-OR^{c}, 3-7 membered heterocyclyl, phenyl, C₃₋₇cycloalkyl, and 5-6 membered heteroaryl;
R⁷ and R⁸ are independently, for each occurrence, selected from the group consisting of hydrogen, C₁₋₆alkyl, C₁₋₆haloalkyl, and halogen; or R⁷ and R⁸ can be taken together to form C₃₋₇cycloalkylene;
R^{a} is hydrogen or C₁₋₆alkyl; and
R^{c} is selected from the group consisting of C₁₋₆alkyl, C₁₋₆haloalkyl, C₃₋₇cycloalkyl, 3-7 membered heterocyclyl, 5-6 membered heteroaryl, phenyl, and C₁₋₆alkylene-N(R^{a})₂;
n is an integer selected from 0 to 6; wherein
when n is an integer selected from 1 to 6, W is selected from the group consisting of hydrogen, halogen, phenyl, 5-6 membered heteroaryl, C₃₋₇cycloalkyl, 3-7 membered heterocyclyl, O—(C₁₋₆alkyl), O—(C₁₋₆haloalkyl), —O-phenyl, and O—(C₁₋₆alkylene)-phenyl; and
when n is 0, W is selected from the group consisting of hydrogen, C₃₋₇cycloalkyl, 3-7 membered saturated heterocyclyl, O—(C₁₋₆alkyl), O—(C₁₋₆haloalkyl), —O-phenyl, and O—(C₁₋₆alkylene)-phenyl; wherein
any aforementioned 3-7 membered heterocyclyl and phenyl are optionally substituted, wherein, when 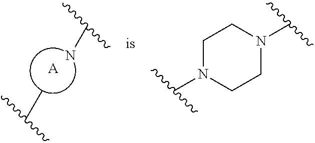
and n is 0 to 6, W is not hydrogen;
when 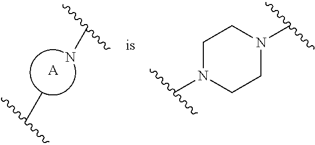
and n is 1, W is not phenyl; and
when 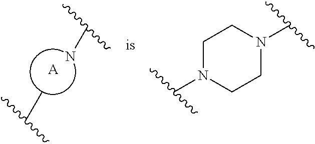
and n is 0, W is not C₃₋₇cycloalkyl.

37. The compound of claim 1, wherein the compound is a compound of (I-d): 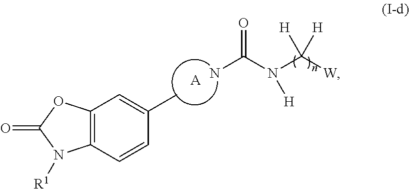
or a pharmaceutically acceptable salt thereof, wherein: 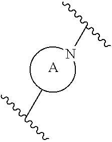
is a monocyclic or bicyclic (e.g., bridged) saturated heterocyclylene containing at least one N (including the depicted nitrogen) that is optionally substituted (e.g., with one or more substituents each independently selected from C₁₋₆alkyl);
R¹ is C₁₋₆alkyl;
W is a phenyl; and
n is 4.

38. The compound of any one of claims 1, 36, and 37, wherein 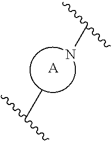
is selected from the group consisting of:

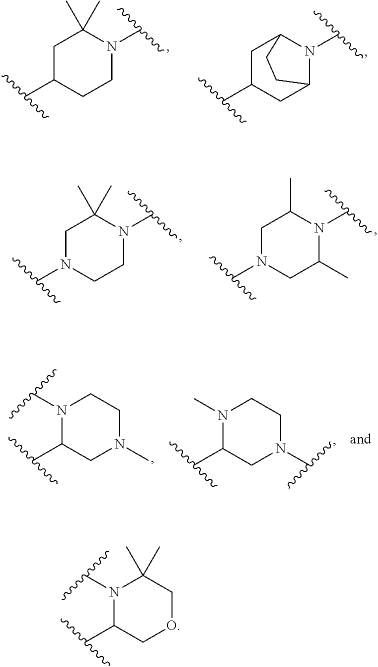

39. The compound of any one of claims 1-36 and 38, wherein Rt is selected from the group consisting of hydrogen, C₁₋₆alkyl, C₁₋₆alkylene-NR^{a}₂, and 3-7 membered heterocyclyl optionally substituted with methyl.

40. The compound of any one of claims 1-36 and 38, wherein R¹ is hydrogen or C₁₋₆alkyl.

41. The compound of any one of claims 1-36 and 38, wherein R¹ is selected from methyl and hydrogen.

42. The compound of any one of claims 1-36 and 38, wherein R¹ is C₁₋₆alkyl.

43. The compound of any one of claims 1-38, wherein R¹ is methyl.

44. The compound of any one of claims 1-36 and 38, wherein R¹ is selected from the group consisting of methyl, hydrogen, —CH₂CH₂N(CH₃)₂, and

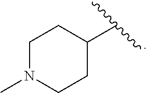 [SMILES: *C1CCN(C)CC1]

45. The compound of any one of claims 1-36 and 38-44, wherein R⁷ and R⁸ are independently hydrogen or methyl.

46. The compound of any one of claims 1-36 and 38-44, wherein R⁷ and R⁸ are both hydrogen.

47. The compound of any one of claims 1-35 and 38-46, wherein R⁹ is hydrogen.

48. The compound of any one of claims 1-36 and 38-47, wherein n is an integer selected from 1 to 6 and W is selected from the group consisting of methyl, phenyl, 5-6 membered heteroaryl, C₃₋₇cycloalkyl, 3-7 membered heterocyclyl, O—(C₁₋₆alkyl), and O—(C₁₋₆alkylene)-phenyl, wherein each aforementioned phenyl is optionally substituted with 1-3 substituents independently selected from the group consisting of C₁₋₆alkyl, halogen, and O—(C₁₋₆alkyl).

49. The compound of any one of claims 1-36 and 38-47, wherein n is an integer selected from 1 to 6 and W is selected from the group consisting of methyl, phenyl, pyridazinyl, cyclohexyl, ethoxy, methoxy, cyclopropyl, and —O—CH₂-phenyl, wherein each aforementioned phenyl is optionally substituted with 1-3 substituents independently selected from the group consisting of C₁₋₆alkyl, halogen, and O—(C₁₋₆alkyl).

50. The compound of any one of claims 1-36 and 38-49, wherein W is methyl or phenyl.

51. The compound of any one of claims 1-36 and 38-49, wherein W is phenyl.

52. The compound of any one of claims 1-36 and 38-49, wherein W is methyl.

53. The compound of any one of claims 1-36 and 38-52, wherein n is 2, 3, or 4.

54. The compound of any one of claims 1-36 and 38-52, wherein n is 2.

55. The compound of any one of claims 1-36 and 38-52, wherein n is 4.

56. The compound of any one of claims 1-36 and 38-55, wherein any aforementioned phenyl or 3-7 membered heterocyclyl is optionally substituted with 1-4 substituents independently, for each occurrence, selected from the group consisting of C₁₋₆alkyl, halogen, —O—C₁₋₆alkyl, and —CH₂N(R^{a})₂, wherein R^{a} is as defined in claim 1.

57. The compound of any one of claims 1-36 and 38-55, wherein any aforementioned phenyl or 3-7 membered heterocyclyl at W is optionally substituted with 1-3 substituents independently, for each occurrence, selected from the group consisting of C₁₋₆alkyl, halogen, —O—C₁₋₆alkyl, and —CH₂N(R^{a})₂, wherein R^{a} is as defined in claim 1.

58. The compound of any one of claims 1-36 and 38-55, wherein any aforementioned phenyl at W is optionally substituted with 1-2 of methyl.

59. A pharmaceutical composition comprising the compound of any one of claims 1-58 and a pharmaceutically acceptable carrier.

60. A method of treating a subject with cancer and in need thereof, the method comprising administering to the subject a therapeutically effective amount of the compound of any one of claims 1-58 or a pharmaceutical composition of claim 59.

61. A method of treating a subject with a lysosomal storage disorder and in need thereof, the method comprising administering to the subject a therapeutically effective amount of the compound of any one of claims 1-58 or a pharmaceutical composition of claim 59.

62. A method of treating a subject with a neurodegenerative disorder and in need thereof, the method comprising administering to the subject a therapeutically effective amount of the compound of any one of claims 1-58 or a pharmaceutical composition of claim 59.

63. The method of claim 60, wherein the cancer is melanoma.

64. The method of claim 61, wherein the lysosomal storage disorder is selected from the group consisting of: Krabbe disease, Fabry disease, Tay-Sachs disease, Pompe disease, Hunter's syndrome, Niemann Pick disease Types A and B, and Gaucher disease.

65. The method of claim 64, wherein the lysosomal storage disorder is Gaucher disease.

66. The method of claim 62, wherein the neurodegenerative disorder is selected from the group consisting of: Alzheimer's disease, Parkinson's disease, Huntington's disease, amyotrophic lateral sclerosis, Lewy body disease, dementia, and multiple system atrophy.

67. The method of claim 66, wherein the neurodegenerative disorder is Parkinson's disease.

68. The method of claim 66, wherein the neurodegenerative disorder is Lewy body disease.

69. The method of claim 66, wherein the neurodegenerative disorder is dementia.

70. The method of claim 66, wherein the neurodegenerative disorder is multiple system atrophy.

71. A method of treating a subject with an inflammatory disorder and in need thereof, the method comprising administering to the subject a therapeutically effective amount of the compound of any one of claims 1-58 or a pharmaceutical composition of claim 59.

72. The method of any one of claims 60-71, wherein the subject is human.

73. A compound of any one of claims 1-58 or a pharmaceutical composition of claim 59 for use in a method of treating a subject with cancer and in need thereof, the method comprising administering to the subject a therapeutically effective amount of the compound or the pharmaceutical composition.

74. A compound of any one of claims 1-58 or a pharmaceutical composition of claim 59 for use in a method of treating a subject with a lysosomal storage disorder and in need thereof, the method comprising administering to the subject a therapeutically effective amount of the compound or the pharmaceutical composition.

75. A compound of any one of claims 1-58 or a pharmaceutical composition of claim 59 for use in a method of treating a subject with a neurodegenerative disorder and in need thereof, the method comprising administering to the subject a therapeutically effective amount of the compound or the pharmaceutical composition.

76. A compound of any one of claims 1-58 or a pharmaceutical composition of claim 59 for use in a method of treating a subject with an inflammatory disorder and in need thereof, the method comprising administering to the subject a therapeutically effective amount of the compound or the pharmaceutical composition.

77. A compound of any one of claims 1-58 or a pharmaceutical composition of claim 59 for the manufacture of a medicament for treating a subject with cancer and in need thereof, the method comprising administering to the subject a therapeutically effective amount of the compound or the pharmaceutical composition.

78. A compound of any one of claims 1-58 or a pharmaceutical composition of claim 59 for the manufacture of a medicament for treating a subject with a lysosomal storage disorder and in need thereof, the method comprising administering to the subject a therapeutically effective amount of the compound or the pharmaceutical composition.

79. A compound of any one of claims 1-58 or a pharmaceutical composition of claim 59 for the manufacture of a medicament for treating a subject with a neurodegenerative disorder and in need thereof, the method comprising administering to the subject a therapeutically effective amount of the compound or the pharmaceutical composition.

80. A compound of any one of claims 1-58 or a pharmaceutical composition of claim 59 for the manufacture of a medicament for treating a subject with an inflammatory disorder and in need thereof, the method comprising administering to the subject a therapeutically effective amount of the compound or the pharmaceutical composition.
